(19) United States
(12) Patent Application Publication
(10) Pub. No.: US 2020/0277378 A1
(43) Pub. Date: Sep. 3, 2020

(54) COMBINATION THERAPIES

(71) Applicant: Novartis AG, Basel (CH)

(72) Inventors: Catherine Anne Sabatos-Peyton, Cambridge, MA (US); Jennifer Marie Mataraza, Cambridge, MA (US); Kenzie MacIsaac, Brookline, MA (US); Eunice Kwak, Sudbury, MA (US); Jason Faris, Reading, MA (US); Glenn Dranoff, Sudbury, MA (US); Marion Wiesmann, Basel (CH); Fiona Sharp, Cambridge, MA (US); Adam Crystal, Newton, MA (US); John Scott Cameron, Belmont, MA (US); Robert Martin Strieter, Falmouth, MA (US); Lilli Petruzzelli, Lexington, MA (US); Shamsah Kazani, Watertown, MA (US); David Andrew Sandham, Cambridge, MA (US); Neil John Press, Horsham (GB); Keith Hoffmaster, Hopkinton, MA (US)

(21) Appl. No.: 16/764,102

(22) PCT Filed: Nov. 16, 2018
(86) PCT No.: PCT/US2018/061534

§ 371 Date: May 14, 2020

Related U.S. Application Data
(60) Provisional application No. 62/587,370, filed on Nov. 16, 2017.
(60) Provisional application No. 62/645,588, filed on Mar. 20, 2018.
(60) Provisional application No. 62/703,736, filed on Jul. 26, 2018.

(51) Int. Cl. C07K 16/28; A61P 35/00; C07K 16/30

(52) U.S. Cl. CPC C07K 16/2818; C07K 16/2866; C07K 16/2878; A61K 2039/507; C07K 16/3015; C07K 16/3053; A61P 35/00

(57) ABSTRACT

Combination therapies comprising PD-1 inhibitors and other therapeutic agents used to treat or prevent cancerous conditions and disorders as follow. Combination therapies of three agents which are a PD-1 inhibitor, a CXCR2 inhibitor and a CSF-1/1R binding agent for treating a pancreatic cancer or a colorectal cancer. Combination therapies of three agents which are a PD-1 inhibitor, a CXCR2 inhibitor and an inhibitor of either TIM-3, C-MET or A2aR for treating a pancreatic cancer or a colorectal cancer. Combination therapies of three agents which are a PD-1 inhibitor, a LAG-3 inhibitor and (i) an inhibitor of either TGF-beta, TIM-3, C-MET, IL-1b or MEK or (ii) a GITR agonist or (iii) an A2aR antagonist or (iv) a CSF-1/1R binding agent for treating a breast cancer. Combination therapies of two agents which are a PD-1 inhibitor and a CXCR2 inhibitor for treating a pancreatic cancer, a colorectal cancer, a lung cancer or a breast cancer.

### CROSS REFERENCE TO RELATED APPLICATIONS

This application claims the benefit of U.S. Provisional Application No. 62/587,370, filed Nov. 16, 2017, U.S. Provisional Application No. 62/645,588, filed Mar. 20, 2018, and U.S. Provisional Application No. 62/703,736, filed Jul. 26, 2018. The contents of the aforementioned applications are hereby incorporated by reference in their entirety.

### SEQUENCE LISTING

The instant application contains Sequence Listings which have been submitted electronically in ASCII format and are hereby incorporated by reference in their entirety. Said ASCII copy, created on, Nov. 14, 2018, is named C2160-7021WO_SL.txt and is 287,976 bytes in size.

### BACKGROUND

The ability of T cells to mediate an immune response against an antigen requires two distinct signaling interactions (Viglietta et al. (2007) Neurotherapeutics 4:666-675; Korman et al. (2007) Adv. Immunol. 90:297-339). First, an antigen that has been arrayed on the surface of antigen-presenting cells (APC) is presented to an antigen-specific naive CD4⁺ T cell. Such presentation delivers a signal via the T cell receptor (TCR) that directs the T cell to initiate an immune response specific to the presented antigen. Second, various co-stimulatory and inhibitory signals mediated through interactions between the APC and distinct T cell surface molecules trigger the activation and proliferation of the T cells and ultimately their inhibition.

The immune system is tightly controlled by a network of costimulatory and co-inhibitory ligands and receptors. These molecules provide the second signal for T cell activation and provide a balanced network of positive and negative signals to maximize immune responses against infection, while limiting immunity to self (Wang et al. (2011) J. Exp. Med. 208(3):577-92; Lepenies et al. (2008) Endocrine, Metabolic & Immune Disorders—Drug Targets 8:279-288). Examples of costimulatory signals include the binding between the B7.1 (CD80) and B7.2 (CD86) ligands of the APC and the CD28 and CTLA-4 receptors of the CD4⁺ T-lymphocyte (Sharpe et al. (2002) Nature Rev. Immunol. 2:116-126; Lindley et al. (2009) Immunol. Rev. 229:307-321). Binding of B7.1 or B7.2 to CD28 stimulates T cell activation, whereas binding of B7.1 or B7.2 to CTLA-4 inhibits such activation (Dong et al. (2003) Immunolog. Res. 28(1):39-48; Greenwald et al. (2005) Ann. Rev. Immunol. 23:515-548). CD28 is constitutively expressed on the surface of T cells (Gross et al. (1992) J. Immunol. 149:380-388), whereas CTLA4 expression is rapidly up-regulated following T-cell activation (Linsley et al. (1996) Immunity 4:535-543).

Other ligands of the CD28 receptor include a group of related B7 molecules, also known as the “B7 Superfamily” (Coyle et al. (2001) Nature Immunol. 2(3):203-209; Sharpe et al. (2002) Nature Rev. Immunol. 2:116-126; Collins et al. (2005) Genome Biol. 6:223.1-223.7; Korman et al. (2007) Adv. Immunol. 90:297-339). Several members of the B7 Superfamily are known, including B7.1 (CD80), B7.2 (CD86), the inducible co-stimulator ligand (ICOS-L), the programmed death-1 ligand (PD-L1; B7-H1), the programmed death-2 ligand (PD-L2; B7-DC), B7-H3, B7-H4 and B7-H6 (Collins et al. (2005) Genome Biol. 6:223.1-223.7).

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8). Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43). PD-L1 is abundant in a variety of human cancers (Dong et al. (2002) Nat. Med. 8:787-9).

PD-1 is known as an immunoinhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100) Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

Glucocorticoid-induced TNFR-related protein (GITR) is a member of the Tumor Necrosis Factor Superfamily (TNFRSF). GITR expression is detected constitutively on murine and human CD4+CD25+ regulatory T cells which can be further increased upon activation. In contrast, effector CD4+CD25− T cells and CD8+CD25− T cells express low to undetectable levels of GITR, which is rapidly upregulated following T cell receptor activation. Expression of GITR has also been detected on activated NK cells, dendritic cells, and macrophages. Signal transduction pathway downstream of GITR has been shown to involve MAPK and the canonical NFκB pathways. Various TRAF family members have been implicated as signaling intermediates downstream of GITR (Nocentini et al. (2005) Eur. J. Immunol. 35:1016-1022).

Cellular activation through GITR is believed to serve several functions depending on the cell type and microenvironment including, but not limited to, costimulation to augment proliferation and effector function, inhibition of suppression by regulatory T cells, and protection from activation-induced cell death (Shevach and Stephens (2006) Nat. Rev. Immunol. 6:613-618). An agonistic monoclonal antibody against mouse GITR effectively induced tumor-specific immunity and eradicated established tumors in a mouse syngeneic tumor model (Ko et al. (2005) J. Exp. Med. 202:885-891).

Given the importance of immune checkpoint pathways in regulating an immune response, the need exists for developing novel combination therapies that activate the immune system.

### SUMMARY

Disclosed herein, inter alia, are methods and compositions comprising combination therapies, e.g., a combination comprising two or more (e.g., two, three, four, five, six, or more) therapeutic agents disclosed herein. The therapeutic agents can be chosen from one or more of: an inhibitor of an inhibitory molecule (e.g., an inhibitor of a checkpoint inhibitor), an activator of a costimulatory molecule, a chemotherapeutic agent, a targeted anti-cancer therapy, an oncolytic drug, a cytotoxic agent, or any of the therapeutic agents disclosed herein. In some embodiments, the therapeutic agent can be chosen from: a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a STING agonist, a Galectin inhibitor, a MEK inhibitor, an IL-15/IL-15RA complex, an IL-1β inhibitor, an MDM2 inhibitor, or any combination thereof.

The combinations described herein can provide a beneficial effect, e.g., in the treatment of a cancer, such as an enhanced anti-cancer effect, reduced toxicity, and/or reduced side effects. For example, a first therapeutic agent, e.g., any of the therapeutic agents disclosed herein, and a second therapeutic agent, e.g., the one or more additional therapeutic agents, or all, can be administered at a lower dosage than would be required to achieve the same therapeutic effect compared to a monotherapy dose. Thus, compositions and methods for treating proliferative disorders, including cancer, using the aforesaid combination therapies are disclosed.

Accordingly, in one aspect, the disclosure features a method of treating (e.g., inhibiting, reducing, ameliorating, or preventing) a disorder, e.g., a hyperproliferative condition or disorder (e.g., a cancer) in a subject. The method includes administering to the subject a combination comprising three or more (e.g., four, five, six, seven, eight, or more) therapeutic agents disclosed herein. In some embodiments, the therapeutic agent is chosen from: a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a STING agonist, a Galectin inhibitor, a MEK inhibitor, an IL-15/IL-15RA complex, an IL-1β inhibitor, an MDM2 inhibitor, or any combination thereof. In some embodiments, the cancer is chosen from a breast cancer (e.g., a triple negative breast cancer), a pancreatic cancer, a colorectal cancer (e.g., a microsatellite stable colorectal cancer (MSS CRC)), a skin cancer, a gastric cancer, a gastroesophageal cancer, or an ER+ cancer. In some embodiments, the skin cancer is a melanoma (e.g., a refractory melanoma). In some embodiments, the ER+ cancer is an ER+ breast cancer.

In some embodiments, the combination comprises:

(i) a PD-1 inhibitor, a SERD, and a CDK4/6 inhibitor, e.g., to treat an ER+ cancer or a breast cancer;

(ii) a PD-1 inhibitor, a CXCR2 inhibitor, a CSF-1/1R binding agent, and optionally, one or more (e.g., two or all) of a TIM-3 inhibitor, a c-MET inhibitor, or an A2aR antagonist, e.g., to treat a pancreatic cancer or a colorectal cancer;

(iii) a PD-1 inhibitor, a CXCR2 inhibitor, and one or more (e.g., two or all) of a TIM-3 inhibitor, a c-MET inhibitor, or an A2aR antagonist, e.g., to treat a pancreatic cancer or a colorectal cancer;

(iv) a PD-1 inhibitor, a GITR agonist, and one or more (e.g., two or all) of a TGF-β inhibitor, an A2aR antagonist, or a c-MET inhibitor, e.g., to treat a pancreatic cancer, a colorectal cancer, or a melanoma;

(v) a PD-1 inhibitor, a LAG-3 inhibitor, a GITR agonist, and one or more (e.g., two or all) of a TGF-β inhibitor, an A2aR antagonist, or a c-MET inhibitor, e.g., to treat a pancreatic cancer, a colorectal cancer, or a melanoma;

(vi) a PD-1 inhibitor, an A2aR antagonist, and one or both of a TGF-β inhibitor or a CSF-1/1R binding agent, e.g., to treat a pancreatic cancer, a colorectal cancer, or a melanoma;

(vii) a PD-1 inhibitor, a c-MET inhibitor, and one or more (e.g., two or all) of a TGF-β inhibitor, an A2aR antagonist, or a c-MET inhibitor, e.g., to treat a pancreatic cancer, a colorectal cancer, a gastric cancer, or a melanoma;

(viii) a PD-1 inhibitor, an IDO inhibitor, and one or more (e.g., two, three, four, or all) of a TGF-β inhibitor, an A2aR antagonist, a CSF-1/1R binding agent, a c-MET inhibitor, or a GITR agonist, e.g., to treat a pancreatic cancer, a colorectal cancer, a gastric cancer, or a melanoma;

(ix) a PD-1 inhibitor, a LAG-3 inhibitor, and one or more (e.g., two three, four, five, six or all) of a TGF-β inhibitor, a TIM-3 inhibitor, a c-MET inhibitor, an IL-1β inhibitor, a MEK inhibitor, a GITR agonist or a CSF-1/1R binding agent, e.g., to treat a breast cancer, e.g., a triple negative breast cancer (TNBC);

(x) a PD-1 inhibitor, a CSF-1/1R binding agent, and one or more of (e.g., two, three, or all) of a TGF-β inhibitor, a TIM-3 inhibitor, a c-MET inhibitor, or an IL-1β inhibitor, e.g., to treat a breast cancer (e.g., a TNBC);

(xi) a PD-1 inhibitor, an A2aR antagonist, and one or more (e.g., two three, four, five, or all) of a TGF-β inhibitor, a TIM-3 inhibitor, a c-MET inhibitor, an IL-1β inhibitor, an IL-15/IL-15RA complex, or a CSF-1/1R binding agent, e.g., to treat a breast cancer (e.g., a TNBC), a colorectal cancer (e.g., a microsatellite stable colorectal cancer (MSS CRC), a gastroesophageal cancer or a pancreatic cancer;

(xii) a PD-1 inhibitor, an IL-1β inhibitor, and one or more of (e.g., two, three, four or more) of a TGF-β inhibitor, an IL-15/IL-15RA complex, a c-MET inhibitor, a CSF-1/1R binding agent, or a TIM-3 inhibitor, e.g., to treat a colorectal cancer (e.g., a microsatellite stable colorectal cancer (MSS CRC), a gastroesophageal cancer or a pancreatic cancer;

(xiii) a PD-1 inhibitor, a MEK inhibitor, and one or more of (e.g., two, three, four or more) of a TGF-β inhibitor, an IL-15/IL-15RA complex, a c-MET inhibitor, a CSF-1/1R binding agent, or a TIM-3 inhibitor, e.g., to treat a a colorectal cancer (e.g., a microsatellite stable colorectal cancer (MSS CRC), a gastroesophageal cancer or a pancreatic cancer;

(xiv) an IL-1β inhibitor, an A2aR antagonist, and one or both of an IL-15/IL-15Ra complex or a TGF-β inhibitor, e.g., to treat a a colorectal cancer (e.g., a microsatellite stable colorectal cancer (MSS CRC), a gastroesophageal cancer or a pancreatic cancer;

(xv) an IL-15/IL-15Ra complex, and a TGF-β inhibitor, and one or more of (e.g., two, three, or more of) an IL-1β inhibitor, a CSF-1/1R binding agent, a c-MET inhibitor, or an A2aR antagonist, e.g., to treat a a colorectal cancer (e.g., a microsatellite stable colorectal cancer (MSS CRC), a gastroesophageal cancer or a pancreatic cancer;

(xvi) a PD-1 inhibitor, and a TIM-3 inhibitor, and one or more of (e.g., both), a STING agonist, or a CSF-1/1R binding agent, e.g., to treat a solid tumor, e.g., a pancreatic cancer, or a colon cancer;

(xvii) a PD-1 inhibitor, a TIM-3 inhibitor and an A2aR antagonist, and one or more of (e.g., both) a CSF-1/1R binding agent or a TGF-β inhibitor, e.g., to treat a solid tumor, e.g., a pancreatic cancer, or a colon cancer;

(xviii) a Galectin inhibitor, e.g., one or more of (e.g., both), a Galectin 1 inhibitor or a Galectin 3 inhibitor, and a PD-1 inhibitor, e.g., to treat a solid tumor or a hematological malignancy; or

(xix) a PD-1 inhibitor and CXCR2 inhibitor, e.g., to treat a solid tumor, e.g., a colorectal cancer (e.g., a microsatellite stable colorectal cancer (MSS CRC)), a lung cancer (e.g., a non-small cell lung cancer (NSCLC)) or a breast cancer (e.g., a TNBC).

In another aspect, the invention features a method of reducing an activity (e.g., growth, survival, or viability, or all), of a hyperproliferative (e.g., a cancer) cell. The method includes contacting the cell with a combination comprising three or more (e.g., four, five, six, seven, eight, or more) therapeutic agents disclosed herein. In some embodiments, the therapeutic agent is chosen from: a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a STING agonist, a Galectin inhibitor, a MEK inhibitor, an IL-15/IL-15RA complex, an IL-1β inhibitor, an MDM2 inhibitor, or any combination thereof.

In some embodiments, the combination comprises:

(i) a PD-1 inhibitor, a SERD, and a CDK4/6 inhibitor;

(ii) a PD-1 inhibitor, a CXCR2 inhibitor, a CSF-1/1R binding agent, and optionally, one or more (e.g., two or all) of a TIM-3 inhibitor, a c-MET inhibitor, or an A2aR antagonist;

(iii) a PD-1 inhibitor, a CXCR2 inhibitor, and one or more (e.g., two or all) of a TIM-3 inhibitor, a c-MET inhibitor, or an A2aR antagonist;

(iv) a PD-1 inhibitor, a GITR agonist, and one or more (e.g., two or all) of a TGF-β inhibitor, an A2aR antagonist, or a c-MET inhibitor;

(v) a PD-1 inhibitor, a LAG-3 inhibitor, a GITR agonist, and one or more (e.g., two or all) of a TGF-β inhibitor, an A2aR antagonist, or a c-MET inhibitor;

(vi) a PD-1 inhibitor, an A2aR antagonist, and one or both of a TGF-β inhibitor or a CSF-1/1R binding agent;

(vii) a PD-1 inhibitor, a c-MET inhibitor, and one or more (e.g., two or all) of a TGF-β inhibitor, an A2aR antagonist, or a c-MET inhibitor;

(viii) a PD-1 inhibitor, an IDO inhibitor, and one or more (e.g., two, three, four, or all) of a TGF-β inhibitor, an A2aR antagonist, a CSF-1/1R binding agent, a c-MET inhibitor, or a GITR agonist;

(ix) a PD-1 inhibitor, a LAG-3 inhibitor, and one or more (e.g., two three, four, five, six or all) of a TGF-β inhibitor, a TIM-3 inhibitor, a c-MET inhibitor, an IL-1β inhibitor, a MEK inhibitor, a GITR agonist or a CSF-1/1R binding agent, e.g., to treat a breast cancer, e.g., a triple negative breast cancer (TNBC);

(x) a PD-1 inhibitor, a CSF-1/1R binding agent, and one or more of (e.g., two, three, or all) of a TGF-β inhibitor, a TIM-3 inhibitor, a c-MET inhibitor, or an IL-1β inhibitor, e.g., to treat a breast cancer (e.g., a TNBC);

(xi) a PD-1 inhibitor, an A2aR antagonist, and one or more (e.g., two three, four, five, or all) of a TGF-β inhibitor, a TIM-3 inhibitor, a c-MET inhibitor, an IL-1β inhibitor, an IL-15/IL-15RA complex, or a CSF-1/1R binding agent, e.g., to treat a breast cancer (e.g., a TNBC), a colorectal cancer (e.g., a microsatellite stable colorectal cancer (MSS CRC), a gastroesophageal cancer or a pancreatic cancer;

(xii) a PD-1 inhibitor, an IL-1β inhibitor, and one or more of (e.g., two, three, four or more) of a TGF-β inhibitor, an IL-15/IL-15RA complex, a c-MET inhibitor, a CSF-1/1R binding agent, or a TIM-3 inhibitor, e.g., to treat a colorectal cancer (e.g., a microsatellite stable colorectal cancer (MSS CRC), a gastroesophageal cancer or a pancreatic cancer;

(xiii) a PD-1 inhibitor, a MEK inhibitor, and one or more of (e.g., two, three, four or more) of a a TGF-β inhibitor, an IL-15/IL-15RA complex, a c-MET inhibitor, a CSF-1/1R binding agent, or a TIM-3 inhibitor, e.g., to treat a colorectal cancer (e.g., a microsatellite stable colorectal cancer (MSS CRC), a gastroesophageal cancer or a pancreatic cancer;

(xiv) an IL-1β inhibitor, an A2aR antagonist, and one or both of an IL-15/IL-15Ra complex or a TGF-β inhibitor, e.g., to treat a a colorectal cancer (e.g., a microsatellite stable colorectal cancer (MSS CRC), a gastroesophageal cancer or a pancreatic cancer;

(xv) an IL-15/IL-15Ra complex, and a TGF-β inhibitor, and one or more of (e.g., two, three, or more) of an IL-1β inhibitor, a CSF-1/1R binding agent, a c-MET inhibitor, or an A2aR antagonist, e.g., to treat a a colorectal cancer (e.g., a microsatellite stable colorectal cancer (MSS CRC), a gastroesophageal cancer or a pancreatic cancer

(xvi) a PD-1 inhibitor, and a TIM-3 inhibitor, and one or more of (e.g., both), a STING agonist, or a CSF-1/1R binding agent, e.g., to treat a solid tumor, e.g., a pancreatic cancer, or a colon cancer;

(xvii) a PD-1 inhibitor, a TIM-3 inhibitor and an A2aR antagonist, and one or more of (e.g., both) a CSF-1/1R binding agent or a TGF-β inhibitor, e.g., to treat a solid tumor, e.g., a pancreatic cancer, or a colon cancer;

(xviii) a Galectin inhibitor, e.g., one or more of (e.g., both), a Galectin 1 inhibitor or a Galectin 3 inhibitor, and a PD-1 inhibitor, e.g., to treat a solid tumor or a hematological malignancy; or (xix) a PD-1 inhibitor and CXCR2 inhibitor, e.g., to treat a solid tumor, e.g., a colorectal cancer (e.g., a microsatellite stable colorectal cancer (MSS CRC)), a lung cancer (e.g., a non-small cell lung cancer (NSCLC)) or a breast cancer (e.g., a TNBC).

The method can be performed in a subject, e.g., as part of a therapeutic protocol. The cell can be a cancer cell, e.g., a cell from a cancer described herein, e.g., a breast cancer, a pancreatic cancer, a colorectal cancer (CRC), a skin cancer, a gastric cancer, a gastroesophageal cancer, or an ER+ cancer. In some embodiments, the skin cancer is a melanoma (e.g., a refractory melanoma). In some embodiments, the ER+ cancer is an ER+ breast cancer. In some embodiments, the breast cancer is a TNBC. In some embodiments, the CRC is a MSS CRC.

In some embodiments, a combination described herein is administered to a subject having a cancer, e.g., a cancer described herein. In some embodiments, the cancer has a high mutational burden, e.g., as disclosed in Alexandrov L. B. et al., (2013) Nature 500, 415-421 and Chalmers Z. R., et al., (2017) Genome Medicine 9:34. In some embodiments, the cancer is a breast cancer, a pancreatic cancer, a colorectal cancer (CRC), a skin cancer, a gastric cancer, a gastroesophageal cancer, or an ER+ cancer. In some embodiments, the skin cancer is a melanoma (e.g., a refractory melanoma). In some embodiments, the ER+ cancer is an ER+ breast cancer. In some embodiments, the breast cancer is a TNBC. In some embodiments, the CRC is a MSS CRC.

In certain embodiments of the methods disclosed herein, the method further includes determining one or more biomarkers (e.g., one or more biomarkers disclosed herein) in the subject. In one embodiment, the biomarker is determined in vivo, e.g., non-invasively. In other embodiments, the biomarker is determined in a sample (e.g., a tumor biopsy) acquired from the subject. In embodiments, responsive to a determination of the presence of one or more biomarkers, a combination of the therapeutic agents disclosed herein is administered to the subject.

In another aspect, the invention features a composition (e.g., one or more compositions or dosage forms), that includes a combination comprising three or more (e.g., four, five, six, seven, eight, or more) therapeutic agents disclosed herein. In some embodiments, the therapeutic agent is chosen from: a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a STING agonist, a Galectin inhibitor, a MEK inhibitor, an IL-15/IL-15RA complex, an IL-1β inhibitor, an MDM2 inhibitor, or any combination thereof.

In some embodiments, the combination comprises:

(i) a PD-1 inhibitor, a SERD, and a CDK4/6 inhibitor;

(ii) a PD-1 inhibitor, a CXCR2 inhibitor, a CSF-1/1R binding agent, and optionally, one or more (e.g., two or all) of a TIM-3 inhibitor, a c-MET inhibitor, or an A2aR antagonist;

(iii) a PD-1 inhibitor, a CXCR2 inhibitor, and one or more (e.g., two or all) of a TIM-3 inhibitor, a c-MET inhibitor, or an A2aR antagonist;

(iv) a PD-1 inhibitor, a GITR agonist, and one or more (e.g., two or all) of a TGF-β inhibitor, an A2aR antagonist, or a c-MET inhibitor;

(v) a PD-1 inhibitor, a LAG-3 inhibitor, a GITR agonist, and one or more (e.g., two or all) of a TGF-β inhibitor, an A2aR antagonist, or a c-MET inhibitor;

(vi) a PD-1 inhibitor, an A2aR antagonist, and one or both of a TGF-β inhibitor or a CSF-1/1R binding agent;

(vii) a PD-1 inhibitor, a c-MET inhibitor, and one or more (e.g., two or all) of a TGF-β inhibitor, an A2aR antagonist, or a c-MET inhibitor;

(viii) a PD-1 inhibitor, an IDO inhibitor, and one or more (e.g., two, three, four, or all) of a TGF-β inhibitor, an A2aR antagonist, a CSF-1/1R binding agent, a c-MET inhibitor, or a GITR agonist;

(ix) a PD-1 inhibitor, a LAG-3 inhibitor, and one or more (e.g., two three, four, five, six or all) of a TGF-β inhibitor, a TIM-3 inhibitor, a c-MET inhibitor, an IL-1β inhibitor, a MEK inhibitor, a GITR agonist or a CSF-1/1R binding agent, e.g., to treat a breast cancer, e.g., a triple negative breast cancer (TNBC);

(x) a PD-1 inhibitor, a CSF-1/1R binding agent, and one or more of (e.g., two, three, or all) of a TGF-β inhibitor, a TIM-3 inhibitor, a c-MET inhibitor, or an IL-1β inhibitor, e.g., to treat a breast cancer (e.g., a TNBC);

(xi) a PD-1 inhibitor, an A2aR antagonist, and one or more (e.g., two three, four, five, or all) of a TGF-β inhibitor, a TIM-3 inhibitor, a c-MET inhibitor, an IL-1β inhibitor, an IL-15/IL-15RA complex, or a CSF-1/1R binding agent, e.g., to treat a breast cancer (e.g., a TNBC), a colorectal cancer (e.g., a microsatellite stable colorectal cancer (MSS CRC), a gastroesophageal cancer or a pancreatic cancer;

(xii) a PD-1 inhibitor, an IL-1β inhibitor, and one or more of (e.g., two, three, four or more) of a TGF-β inhibitor, an IL-15/IL-15RA complex, a c-MET inhibitor, a CSF-1/1R binding agent, or a TIM-3 inhibitor, e.g., to treat a colorectal cancer (e.g., a microsatellite stable colorectal cancer (MSS CRC), a gastroesophageal cancer or a pancreatic cancer;

(xiii) a PD-1 inhibitor, a MEK inhibitor, and one or more of (e.g., two, three, four or more) of a TGF-β inhibitor, an IL-15/IL-15RA complex, a c-MET inhibitor, a CSF-1/1R binding agent, or a TIM-3 inhibitor, e.g., to treat a a colorectal cancer (e.g., a microsatellite stable colorectal cancer (MSS CRC), a gastroesophageal cancer or a pancreatic cancer;

(xiv) an IL-1β inhibitor, an A2aR antagonist, and one or both of an IL-15/IL-15Ra complex or a TGF-β inhibitor, e.g., to treat a a colorectal cancer (e.g., a microsatellite stable colorectal cancer (MSS CRC), a gastroesophageal cancer or a pancreatic cancer;

(xv) an IL-15/IL-15Ra complex, and a TGF-β inhibitor, and one or more of (e.g., two, three, or more) of an IL-1β inhibitor, a CSF-1/1R binding agent, a c-MET inhibitor, or an A2aR antagonist, e.g., to treat a a colorectal cancer (e.g., a microsatellite stable colorectal cancer (MSS CRC), a gastroesophageal cancer or a pancreatic cancer

(xvi) a PD-1 inhibitor, and a TIM-3 inhibitor, and one or more of (e.g., both), a STING agonist, or a CSF-1/1R binding agent, e.g., to treat a solid tumor, e.g., a pancreatic cancer, or a colon cancer;

(xvii) a PD-1 inhibitor, a TIM-3 inhibitor and an A2aR antagonist, and one or more of (e.g., both) a CSF-1/1R binding agent or a TGF-β inhibitor, e.g., to treat a solid tumor, e.g., a pancreatic cancer, or a colon cancer;

(xviii) a Galectin inhibitor, e.g., one or more of (e.g., both), a Galectin 1 inhibitor or a Galectin 3 inhibitor, and a PD-1 inhibitor, e.g., to treat a solid tumor or a hematological malignancy; or

(xix) a PD-1 inhibitor and CXCR2 inhibitor, e.g., to treat a solid tumor, e.g., a colorectal cancer (e.g., a microsatellite stable colorectal cancer (MSS CRC)), a lung cancer (e.g., a non-small cell lung cancer (NSCLC)) or a breast cancer (e.g., a TNBC).

In yet another aspect, the disclosure features a composition (e.g., one or more compositions or dosage forms as described herein), for use in treating a disorder, e.g., a cancer. In embodiments, the composition for use includes a composition (e.g., one or more compositions or dosage forms), that includes a combination comprising three or more (e.g., four, five, six, seven, eight, or more) therapeutic agents disclosed herein. In some embodiments, the therapeutic agent is chosen from: a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a STING agonist, a Galectin inhibitor, a MEK inhibitor, an IL-15/IL-15RA complex, an IL-1β inhibitor, an MDM2 inhibitor, or any combination thereof. In some embodiments, the cancer is chosen from a breast cancer, a pancreatic cancer, a colorectal cancer (CRC), a skin cancer, a gastric cancer, a gastroesophageal cancer, or an ER+ cancer. In some embodiments, the skin cancer is a melanoma (e.g., a refractory melanoma). In some embodiments, the ER+ cancer is an ER+ breast cancer. In some embodiments, the breast cancer is a TNBC. In some embodiments, the CRC is a MSS CRC.

In some embodiments, the combination comprises:

(i) a PD-1 inhibitor, a SERD, and a CDK4/6 inhibitor, e.g., to treat an ER+ cancer or a breast cancer;

(ii) a PD-1 inhibitor, a CXCR2 inhibitor, a CSF-1/1R binding agent, and optionally, one or more (e.g., two or all) of a TIM-3 inhibitor, a c-MET inhibitor, or an A2aR antagonist, e.g., to treat a pancreatic cancer or a colorectal cancer;

(iii) a PD-1 inhibitor, a CXCR2 inhibitor, and one or more (e.g., two or all) of a TIM-3 inhibitor, a c-MET inhibitor, or an A2aR antagonist, e.g., to treat a pancreatic cancer or a colorectal cancer;

(iv) a PD-1 inhibitor, a GITR agonist, and one or more (e.g., two or all) of a TGF-β inhibitor, an A2aR antagonist, or a c-MET inhibitor, e.g., to treat a pancreatic cancer, a colorectal cancer, or a melanoma;

(v) a PD-1 inhibitor, a LAG-3 inhibitor, a GITR agonist, and one or more (e.g., two or all) of a TGF-β inhibitor, an A2aR antagonist, or a c-MET inhibitor, e.g., to treat a pancreatic cancer, a colorectal cancer, or a melanoma;

(vi) a PD-1 inhibitor, an A2aR antagonist, and one or both of a TGF-β inhibitor or a CSF-1/1R binding agent, e.g., to treat a pancreatic cancer, a colorectal cancer, or a melanoma;

(vii) a PD-1 inhibitor, a c-MET inhibitor, and one or more (e.g., two or all) of a TGF-β inhibitor, an A2aR antagonist, or a c-MET inhibitor, e.g., to treat a pancreatic cancer, a colorectal cancer, a gastric cancer, or a melanoma;

(viii) a PD-1 inhibitor, an IDO inhibitor, and one or more (e.g., two, three, four, or all) of a TGF-β inhibitor, an A2aR antagonist, a CSF-1/1R binding agent, a c-MET inhibitor, or a GITR agonist, e.g., to treat a pancreatic cancer, a colorectal cancer, a gastric cancer, or a melanoma;

(ix) a PD-1 inhibitor, a LAG-3 inhibitor, and one or more (e.g., two three, four, five, six or all) of a TGF-β inhibitor, a TIM-3 inhibitor, a c-MET inhibitor, an IL-1β inhibitor, a MEK inhibitor or a GITR agonist or a CSF-1/1R binding agent, e.g., to treat a breast cancer, e.g., a triple negative breast cancer (TNBC);

(x) a PD-1 inhibitor, a CSF-1/1R binding agent, and one or more of (e.g., two, three, or all) of a TGF-β inhibitor, a TIM-3 inhibitor, a c-MET inhibitor, or an IL-1β inhibitor, e.g., to treat a breast cancer (e.g., a TNBC);

(xi) a PD-1 inhibitor, an A2aR antagonist, and one or more (e.g., two three, four, five, or all) of a TGF-β inhibitor, a TIM-3 inhibitor, a c-MET inhibitor, an IL-1β inhibitor, an IL-15/IL-15RA complex, or a CSF-1/1R binding agent, e.g., to treat a breast cancer (e.g., a TNBC), a colorectal cancer (e.g., a microsatellite stable colorectal cancer (MSS CRC), a gastroesophageal cancer or a pancreatic cancer;

(xii) a PD-1 inhibitor, an IL-1β inhibitor, and one or more of (e.g., two, three, four or more) of a TGF-β inhibitor, an IL-15/IL-15RA complex, a c-MET inhibitor, a CSF-1/1R binding agent, or a TIM-3 inhibitor, e.g., to treat a colorectal cancer (e.g., a microsatellite stable colorectal cancer (MSS CRC), a gastroesophageal cancer or a pancreatic cancer;

(xiii) a PD-1 inhibitor, a MEK inhibitor, and one or more of (e.g., two, three, four or more) of a a TGF-β inhibitor, an IL-15/IL-15RA complex, a c-MET inhibitor, a CSF-1/1R binding agent, or a TIM-3 inhibitor, e.g., to treat a a colorectal cancer (e.g., a microsatellite stable colorectal cancer (MSS CRC), a gastroesophageal cancer or a pancreatic cancer;

(xiv) an IL-1β inhibitor, an A2aR antagonist, and one or both of an IL-15/IL-15Ra complex or a TGF-β inhibitor, e.g., to treat a a colorectal cancer (e.g., a microsatellite stable colorectal cancer (MSS CRC), a gastroesophageal cancer or a pancreatic cancer;

(xv) an IL-15/IL-15Ra complex, and a TGF-β inhibitor, and one or more of (e.g., two, three, or more) of an IL-1β inhibitor, a CSF-1/1R binding agent, a c-MET inhibitor, or an A2aR antagonist, e.g., to treat a a colorectal cancer (e.g., a microsatellite stable colorectal cancer (MSS CRC), a gastroesophageal cancer or a pancreatic cancer

(xvi) a PD-1 inhibitor, and a TIM-3 inhibitor, and one or more of (e.g., both), a STING agonist, or a CSF-1/1R binding agent, e.g., to treat a solid tumor, e.g., a pancreatic cancer, or a colon cancer;

(xvii) a PD-1 inhibitor, a TIM-3 inhibitor and an A2aR antagonist, and one or more of (e.g., both) a CSF-1/1R binding agent or a TGF-β inhibitor, e.g., to treat a solid tumor, e.g., a pancreatic cancer, or a colon cancer;

(xviii) a Galectin inhibitor, e.g., one or more of (e.g., both), a Galectin 1 inhibitor or a Galectin 3 inhibitor, and a PD-1 inhibitor, e.g., to treat a solid tumor or a hematological malignancy; or

(xix) a PD-1 inhibitor and CXCR2 inhibitor, e.g., to treat a solid tumor, e.g., a colorectal cancer (e.g., a microsatellite stable colorectal cancer (MSS CRC)), a lung cancer (e.g., a non-small cell lung cancer (NSCLC)) or a breast cancer (e.g., a TNBC).

Formulations, e.g., dosage formulations, and kits, e.g., therapeutic kits, that includes a combination comprising three or more (e.g., four, five, six, seven, eight, or more) therapeutic agents disclosed herein, thereby reducing an activity in the cell, and (optionally) instructions for use, are also disclosed. In some embodiments, the therapeutic agent is chosen from: a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a MET inhibitor, a TGF-β inhibitor, an A2aR antagonist, an IDO inhibitor, a STING agonist, a Galectin inhibitor, a MEK inhibitor, an IL-15/IL-15RA complex, an IL-1β inhibitor, an MDM2 inhibitor, or any combination thereof.

In some embodiments, the combination comprises:

(i) a PD-1 inhibitor, a SERD, and a CDK4/6 inhibitor, e.g., to treat an ER+ cancer or a breast cancer;

(ii) a PD-1 inhibitor, a CXCR2 inhibitor, a CSF-1/1R binding agent, and optionally, one or more (e.g., two or all) of a TIM-3 inhibitor, a c-MET inhibitor, or an A2aR antagonist, e.g., to treat a pancreatic cancer or a colorectal cancer;

(iii) a PD-1 inhibitor, a CXCR2 inhibitor, and one or more (e.g., two or all) of a TIM-3 inhibitor, a c-MET inhibitor, or an A2aR antagonist, e.g., to treat a pancreatic cancer or a colorectal cancer;

(iv) a PD-1 inhibitor, a GITR agonist, and one or more (e.g., two or all) of a TGF-β inhibitor, an A2aR antagonist, or a c-MET inhibitor, e.g., to treat a pancreatic cancer, a colorectal cancer, or a melanoma;

(v) a PD-1 inhibitor, a LAG-3 inhibitor, a GITR agonist, and one or more (e.g., two or all) of a TGF-β inhibitor, an A2aR antagonist, or a c-MET inhibitor, e.g., to treat a pancreatic cancer, a colorectal cancer, or a melanoma;

(vi) a PD-1 inhibitor, an A2aR antagonist, and one or both of a TGF-β inhibitor or a CSF-1/1R binding agent, e.g., to treat a pancreatic cancer, a colorectal cancer, or a melanoma;

(vii) a PD-1 inhibitor, a c-MET inhibitor, and one or more (e.g., two or all) of a TGF-β inhibitor, an A2aR antagonist, or a c-MET inhibitor, e.g., to treat a pancreatic cancer, a colorectal cancer, a gastric cancer, or a melanoma;

(viii) a PD-1 inhibitor, an IDO inhibitor, and one or more (e.g., two, three, four, or all) of a TGF-β inhibitor, an A2aR antagonist, a CSF-1/1R binding agent, a c-MET inhibitor, or a GITR agonist, e.g., to treat a pancreatic cancer, a colorectal cancer, a gastric cancer, or a melanoma;

(ix) a PD-1 inhibitor, a LAG-3 inhibitor, and one or more (e.g., two three, four, five, six or all) of a TGF-β inhibitor, a TIM-3 inhibitor, a c-MET inhibitor, an IL-1β inhibitor, a MEK inhibitor, a GITR agonist or a CSF-1/1R binding agent, e.g., to treat a breast cancer, e.g., a triple negative breast cancer (TNBC);

(x) a PD-1 inhibitor, a CSF-1/1R binding agent, and one or more of (e.g., two, three, or all) of a TGF-β inhibitor, a TIM-3 inhibitor, a c-MET inhibitor, or an IL-1β inhibitor, e.g., to treat a breast cancer (e.g., a TNBC);

(xi) a PD-1 inhibitor, an A2aR antagonist, and one or more (e.g., two three, four, five, or all) of a TGF-β inhibitor, a TIM-3 inhibitor, a c-MET inhibitor, an IL-1β inhibitor, an IL-15/IL-15RA complex, or a CSF-1/1R binding agent, e.g., to treat a breast cancer (e.g., a TNBC), a colorectal cancer (e.g., a microsatellite stable colorectal cancer (MSS CRC), a gastroesophageal cancer or a pancreatic cancer;

(xii) a PD-1 inhibitor, an IL-1β inhibitor, and one or more of (e.g., two, three, four or more) of a TGF-β inhibitor, an IL-15/IL-15RA complex, a c-MET inhibitor, a CSF-1/1R binding agent, or a TIM-3 inhibitor, e.g., to treat a colorectal cancer (e.g., a microsatellite stable colorectal cancer (MSS CRC), a gastroesophageal cancer or a pancreatic cancer;

(xiii) a PD-1 inhibitor, a MEK inhibitor, and one or more of (e.g., two, three, four or more) of a a TGF-β inhibitor, an IL-15/IL-15RA complex, a c-MET inhibitor, a CSF-1/1R binding agent, or a TIM-3 inhibitor, e.g., to treat a colorectal cancer (e.g., a microsatellite stable colorectal cancer (MSS CRC), a gastroesophageal cancer or a pancreatic cancer;

(xiv) an IL-1β inhibitor, an A2aR antagonist, and one or both of an IL-15/IL-15Ra complex or a TGF-β inhibitor, e.g., to treat a a colorectal cancer (e.g., a microsatellite stable colorectal cancer (MSS CRC), a gastroesophageal cancer or a pancreatic cancer;

(xv) an IL-15/IL-15Ra complex, and a TGF-β inhibitor, and one or more of (e.g., two, three, or more) of an IL-1β inhibitor, a CSF-1/1R binding agent, a c-MET inhibitor, or an A2aR antagonist, e.g., to treat a a colorectal cancer (e.g., a microsatellite stable colorectal cancer (MSS CRC), a gastroesophageal cancer or a pancreatic cancer

(xvi) a PD-1 inhibitor, and a TIM-3 inhibitor, and one or more of (e.g., both), a STING agonist, or a CSF-1/1R binding agent, e.g., to treat a solid tumor, e.g., a pancreatic cancer, or a colon cancer;

(xvii) a PD-1 inhibitor, a TIM-3 inhibitor and an A2aR antagonist, and one or more of (e.g., both) a CSF-1/1R binding agent or a TGF-β inhibitor, e.g., to treat a solid tumor, e.g., a pancreatic cancer, or a colon cancer;

(xviii) a Galectin inhibitor, e.g., one or more of (e.g., both), a Galectin 1 inhibitor or a Galectin 3 inhibitor, and a PD-1 inhibitor, e.g., to treat a solid tumor or a hematological malignancy; or

(xix) a PD-1 inhibitor and a CXCR2 inhibitor, e.g., to treat a solid tumor, e.g., a colorectal cancer (e.g., a microsatellite stable colorectal cancer (MSS CRC)), a lung cancer (e.g., a non-small cell lung cancer (NSCLC)) or a breast cancer (e.g., a TNBC).

In some embodiments, a method of treating a subject, e.g., a subject having a cancer described herein, with a combination described herein, comprises administration of a combination as part of a therapeutic regimen. In an embodiment, a therapeutic regimen comprises one or more, e.g., two, three, or four combinations described herein. In some embodiments, the therapeutic regimen is administered to the subject in at least one phase, and optionally two phases, e.g., a first phase and a second phase. In some embodiments, the first phase comprises a dose escalation phase. In some embodiments, the first phase comprises one or more dose escalation phases, e.g., a first, second, or third dose escalation phase. In some embodiments, the dose escalation phase comprises administration of a combination comprising two, three, four, or more therapeutic agents, e.g., as described herein. In some embodiments, the second phase comprises a dose expansion phase. In some embodiments, the dose expansion phase comprises administration of a combination comprising two, three, four, or more therapeutic agents, e.g., as described herein. In some embodiments, the dose expansion phase comprises the same two, three, four, or more therapeutic agents as the dose escalation phase.

In some embodiments, the first dose escalation phase comprises administration of a combination comprising two therapeutic agents, e.g., two therapeutic agents described herein, wherein a maximum tolerated dose (MTD) or recommended dose for expansion (RDE) for one or both of the therapeutic agents of is determined. In some embodiments, prior to the first dose escalation phase, the subject was administered with one of the therapeutic agents administered in the first dose escalation phase as a single agent.

In some embodiments, the second dose escalation phase comprises administration of a combination comprising three therapeutic agents, e.g., three therapeutic agents described herein, wherein a maximum tolerated dose (MTD) or recommended dose for expansion (RDE) for one, two, or all of the therapeutic agents is determined. In some embodiments, the second dose escalation phase starts after the first dose escalation phase ends. In some embodiments, the second dose escalation phase comprises administration of one or more of the therapeutic agents administered in the first dose escalation phase. In some embodiments, the second dose escalation phase is performed without performing the first dose escalation phase.

In some embodiments, the third dose escalation phase comprises administration of a combination comprising four therapeutic agents, e.g., four therapeutic agents described herein, wherein a maximum tolerated dose (MTD) or recommended dose for expansion (RDE) of one, two, three, or all of the therapeutic agents is determined. In some embodiments, the third dose escalation phase starts after the first or second dose escalation phase ends. In some embodiments, the third dose escalation phase comprises administration of one or more (e.g., all) of therapeutic agents administered in the second dose escalation phase. In some embodiments, the third dose escalation phase comprises administration of one or more of the therapeutic agents administered in the first dose escalation phase. In some embodiments, the third dose escalation phase is performed without performing the first, second, or both dose escalation phases.

For example, the first dose escalation phase comprises administration of a PD-1 inhibitor and a LAG-3 inhibitor (e.g., a PD-1 inhibitor and a LAG-3 inhibitor described herein), and the second dose escalationphase can further comprise administration of a GITR agonist, a TIM-3 inhibitor, an IL-1β inhibitor, a TGF-β inhibitor, a c-MET inhibitor, a CSF-1/1R binding agent (e.g., a GITR agonist, a TIM-3 inhibitor, an IL-1β inhibitor, a TGF-β inhibitor, a c-MET inhibitor, or a CSF-1/1R binding agent described herein).

As another example, the first dose escalation phase comprises administration of an A2aR antagonist (e.g., an A2aR antagonist described herein), and a GITR agonist, a TIM-3 inhibitor, an IL-1β inhibitor, a TGF-β inhibitor, a c-MET inhibitor, or a CSF-1/1R binding agent (e.g., a GITR agonist, a TIM-3 inhibitor, an IL-1β inhibitor, a TGF-β inhibitor, a c-MET inhibitor, or a CSF-1/1R binding agent described herein).

As yet another example, the first dose escalation phase comprises administration of a PD-1 inhibitor, a LAG-3 inhibitor, and a GITR agonist (e.g., a PD-1 inhibitor, a LAG-3 inhibitor and a GITR agonist described herein), and the second dose escalationphase can further comprise administration of a TIM-3 inhibitor, an IL-1β inhibitor, a TGF-β inhibitor, a c-MET inhibitor, or a CSF-1/1R binding agent (e.g., a GITR agonist, a TIM-3 inhibitor, an IL-1β inhibitor, a TGF-β inhibitor, a c-MET inhibitor, or a CSF-1/1R binding agent described herein).

As a further example, the dose escalation phase, e.g., the first dose escalation phase, comprises administration of a PD-1 inhibitor and a CXCR2 inhibitor (e.g., a PD-1 inhibitor and a CXCR2 inhibitor described herein).

In some embodiments, a method of treating a subject, e.g., a subject having a cancer described herein (e.g., a breast cancer (e.g., a triple negative breast cancer (TNBC)), a lung cancer (e.g., an NSCLC), or a colorectal cancer (CRC) (e.g., a microsatellite stable colorectal cancer (MSS-CRC)), comprises administering to the subject in need thereof a PD-1 inhibitor (e.g., PDR001) and a CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof).

In some embodiments, the dose expansion phase starts after the first, second or third dose escalation phase ends. In some embodiments, the dose expansion phase comprises administration of a combination administered in the dose escalation phase, e.g., the first, second, or third dose escalation phase. In an embodiment, a biopsy is obtained from a subject in the dose expansion phase. In an embodiment, the subject is treated for a breast cancer, e.g., a triple-negative breast cancer (TNBC), e.g., advanced or metastatic TNBC.

Without wishing to be bound by theory, it is believed that in some embodiments, a therapeutic regimen comprising a dose escalation phase and a dose expansion phase allows for entry of new agents or regiments for combination, rapid generation of combinations, and/or assessment of safety and activity of tolerable combinations.

Additional features or embodiments of the methods, compositions, dosage formulations, and kits described herein include one or more of the following.

### Combination Therapies

### Combination Targeting PD-1, ER and CDK4/6

In an embodiment, the combination comprises a PD-1 inhibitor (e.g., a PD-1 inhibitor described herein), a SERD (e.g., a SERD described herein), and a CDK4/6 inhibitor (e.g., a CDK4/6 inhibitor described herein).

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. In some embodiments, the PD-1 inhibitor is administered at a dose of about 300-400 mg. In embodiments, the PD-1 inhibitor is administered once every 3 weeks. In embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the SERD is chosen from LSZ102, fulvestrant, brilanestrant, or elacestrant. In some embodiments, the SERD is LSZ102.

In some embodiments, the CDK4/6 inhibitor is chosen from ribociclib, abemaciclib (Eli Lilly), or palbociclib. In some embodiments, the CDK 4/6 inhibitor is ribociclib. In some embodiments, the CDK4/6 inhibitor, e.g., ribociclib, is administered once daily at a dose of about 200-600 mg. In one embodiment, the CDK4/6 inhibitor is administered once daily at a dose of about 200, 300, 400, 500, or 600 mg, or about 200-300, 300-400, 400-500, or 500-600 mg. In other embodiments, the CDK4/6 inhibitor (e.g., ribociclib) is administered once daily at a dose of 600 mg per day for e.g., three weeks, e.g., 21 days. In some embodiments, this treatment is followed by one week of no treatment. In some embodiments, the CDK4/6 inhibitor (e.g., ribociclib) is administered in repeated dosing cycles of 3 weeks on and 1 week off, e.g., the compound is administered once daily for 3 weeks (e.g., 21 days), followed by no administration for 1 week (e.g., 7 days), after which the cycle is repeated, e.g., the compound is administered daily for 3 weeks followed by no administration for 1 week. In some embodiments, the CDK4/6 inhibitor (e.g., ribociclib) is administered orally.

In some embodiments, the combination comprises PDR001, a SERD described herein, and a CDK4/6 inhibitor described herein. In some embodiments, the combination comprises a PD-1 inhibitor described herein, LSZ102, and a CDK4/6 inhibitor described herein. In some embodiments, the combination comprises a PD-1 inhibitor described herein, a SERD described herein, and ribociclib. In some embodiments, the combination comprises PDR001, LSZ102, and a CDK4/6 inhibitor described herein. In some embodiments, the combination comprises PDR001, a SERD described herein, and ribociclib. In some embodiments, the combination comprises a PD-1 inhibitor described herein, LSZ102, and ribociclib. In some embodiments, the combination comprises PDR001), LSZ102, and ribociclib.

In certain embodiments, the combination further comprises a fourth therapeutic agent, e.g., a therapeutic agent described herein.

In other embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject or cancer is identified as having a biomarker described herein. In some embodiments, the cancer is a cancer expressing estrogen receptor (ER+) or a breast cancer, e.g., an ER+ breast cancer.

### Combination Targeting PD-1, CXCR2 and CSF-1/1R

In an embodiment, the combination comprises a PD-1 inhibitor (e.g., a PD-1 inhibitor described herein), a CXCR2 inhibitor (e.g., a CXCR2 inhibitor described herein), and a CSF-1/1R binding agent (e.g., a CSF-1/1R binding agent disclosed herein).

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. In some embodiments, the PD-1 inhibitor is administered at a dose of about 300-400 mg. In embodiments, the PD-1 inhibitor is administered once every 3 weeks. In embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide choline salt, danirixin, reparixin, or navarixin. In some embodiments, the CXCR2 inhibitor is 6-chloro-3-((3,4-dioxo-2-(pentan-3-ylamino)cyclobut-1-en-1-yl)amino)-2-hydroxy-N-methoxy-N-methylbenzenesulfonamide choline salt.

In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R binding agent is BLZ945. In some embodiments, the CSF-1/1R binding agent is MCS110.

In some embodiments, the combination comprises PDR001, a CXCR2 inhibitor described herein, and a CSF-1/1R binding agent disclosed herein. In some embodiments, the combination comprises a PD-1 inhibitor described herein, a CXCR2 inhibitor described herein, and BLZ945. In some embodiments, the combination comprises PDR001, a CXCR2 inhibitor described herein, and BLZ945.

In some embodiments, the combination comprises PDR001, a CXCR2 inhibitor described herein, and a CSF-1/1R binding agent disclosed herein. In some embodiments, the combination comprises a PD-1 inhibitor described herein, a CXCR2 inhibitor described herein, and MCS110. In some embodiments, the combination comprises PDR001, a CXCR2 inhibitor described herein, and MCS110.

In some embodiments, the combination further comprises a TIM-3 inhibitor, e.g., a TIM-3 inhibitor disclosed herein.

In some embodiments, the TIM-3 inhibitor is MBG453 (Novartis) or TSR-022 (Tesaro). In some embodiments, the TIM-3 inhibitor is MBG453. In some embodiments, the combination comprises PDR001, a CXCR2 inhibitor described herein, a CSF-1/1R binding agent disclosed herein, and MBG453. In some embodiments, the combination comprises a PD-1 inhibitor described herein, a CXCR2 inhibitor described herein, BLZ945, and MBG453. In some embodiments, the combination comprises a PDR001, a CXCR2 inhibitor described herein, BLZ945, and MBG453. In some embodiments, the combination comprises a PD-1 inhibitor described herein, a CXCR2 inhibitor described herein, MCS110, and MBG453. In some embodiments, the combination comprises a PDR001, a CXCR2 inhibitor described herein, MCS110, and MBG453.

In some embodiments, the combination further comprises a c-MET inhibitor, e.g., a c-MET inhibitor disclosed herein. In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280). In some embodiments, the c-MET inhibitor (e.g., INC280) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., INC280) is administered twice a day at a dose of about 400 mg. In an embodiment, the c-MET inhibitor (e.g., INC280) is administered twice a day at a dose of about 600 mg.

In some embodiments, the combination comprises PDR001, a CXCR2 inhibitor described herein, a CSF-1/1R binding agent disclosed herein, and capmatinib (INC280). In some embodiments, the combination comprises a PD-1 inhibitor described herein, a CXCR2 inhibitor described herein, BLZ945, and capmatinib (INC280). In some embodiments, the combination comprises a PDR001, a CXCR2 inhibitor described herein, BLZ945, and capmatinib (INC280). In some embodiments, the combination comprises a PD-1 inhibitor described herein, a CXCR2 inhibitor described herein, MCS110, and capmatinib (INC280). In some embodiments, the combination comprises a PDR001, a CXCR2 inhibitor described herein, MCS110, and capmatinib (INC280).

In some embodiments, the combination further comprises an A2aR antagonist, e.g., an A2aR inhibitor disclosed herein. In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178).

In some embodiments, the combination comprises PDR001, a CXCR2 inhibitor described herein, a CSF-1/1R binding agent disclosed herein, and PBF509 (NIR178). In some embodiments, the combination comprises a PD-1 inhibitor described herein, a CXCR2 inhibitor described herein, BLZ945, and PBF509 (NIR178). In some embodiments, the combination comprises a PDR001, a CXCR2 inhibitor described herein, BLZ945, and PBF509 (NIR178).

In some embodiments, the combination comprises PDR001, a CXCR2 inhibitor described herein, a CSF-1/1R binding agent disclosed herein, and PBF509 (NIR178). In some embodiments, the combination comprises a PD-1 inhibitor described herein, a CXCR2 inhibitor described herein, MCS110, and PBF509 (NIR178). In some embodiments, the combination comprises a PDR001, a CXCR2 inhibitor described herein, MCS110, and PBF509 (NIR178).

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject or cancer is identified as having a biomarker described herein. In some embodiments, the cancer is a solid tumor, e.g., a pancreatic cancer or a colorectal cancer (CRC).

### Combination Targeting PD-1 and CXCR2

In an embodiment, the combination comprises a PD-1 inhibitor (e.g., a PD-1 inhibitor described herein), a CXCR2 inhibitor (e.g., a CXCR2 inhibitor described herein), and a third therapeutic agent.

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide choline salt, danirixin, reparixin, or navarixin.

In some embodiments, the CXCR2 inhibitor is 6-chloro-3-((3,4-dioxo-2-(pentan-3-ylamino)cyclobut-1-en-1-yl)amino)-2-hydroxy-N-methoxy-N-methylbenzenesulfonamide choline salt. In some embodiments, the CXCR2 inhibitor is 2-Hydroxy-N,N,N-trimethylethan-1-aminium 3-chloro-6-({3,4-dioxo-2-[(pentan-3-yl)amino]cyclobut-1-en-1-yl}amino)-2-(N-methoxy-N-methylsulfamoyl)phenolate (i.e., 6-chloro-3-((3,4-dioxo-2-(pentan-3-ylamino)cyclobut-1-en-1-yl)amino)-2-hydroxy-N-methoxy-N-methylbenzenesulfonamide choline salt). In some embodiments, the CXCR2 inhibitor is administered at a dose of about 50-1000 mg (e.g., about 50-400 mg, 50-300 mg, 50-200 mg, 50-100 mg, 150-900 mg, 150-600 mg, 200-800 mg, 300-600 mg, 400-500 mg, 300-500 mg, 200-500 mg, 100-500 mg, 100-400 mg, 200-300 mg, 100-200 mg, 250-350 mg, or about 75 mg, 150 mg, 300 mg, 450 mg, or 600 mg). In some embodiments, the CXCR2 inhibitor is administered daily, e.g., twice daily. In some embodiments, the CXCR2 inhibitor is administered for the first two weeks (e.g., 14 days) in a 4 week cycle (e.g., 28 day cycle). In some embodiments, the CXCR2 inhibitor is administered daily, e.g., twice daily at a total dose of about 50-1000 mg (e.g., about 50-400 mg, 50-300 mg, 50-200 mg, 50-100 mg, 150-900 mg, 150-600 mg, 200-800 mg, 300-600 mg, 400-500 mg, 300-500 mg, 200-500 mg, 100-500 mg, 100-400 mg, 200-300 mg, 100-200 mg, 250-350 mg, or about 75 mg, 150 mg, 300 mg, 450 mg, or 600 mg). In some embodiments, the CXCR2 inhibitor is administered twice daily and each dose, e.g., the first and second dose, is in the same amount. In some embodiments, the CXCR2 inhibitor is administered twice daily and each dose, e.g., the first and second dose, comprises about 25-400 mg (e.g., 25-100 mg, 50-200 mg, 75-150, or 100-400 mg) of the CXCR2 inhibitor. In some embodiments, the CXCR2 inhibitor is administered orally twice daily at a dose of 75 mg for two weeks (e.g., 14 days) in a 4 week cycle (e.g., 28 day cycle). In some embodiments, the CXCR2 inhibitor is administered orally twice daily at a does of 150 mg for two weeks (e.g., 14 days) in a 4 week cycle (e.g., 28 day cycle). In some embodiments, the CXCR2 inhibitor is administered orally twice daily for 2 weeks in a 4 week cycle, e.g., 2 weeks of treatment with the CXCR2 inhibitor and 2 weeks of no treatment in a 4 week cycle.

In some embodiments, the combination comprises PDR001 and a CXCR2 inhibitor described herein (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt). In some embodiments, the combination comprises a PD-1 inhibitor (e.g., PDR001) and 6-chloro-3-((3,4-dioxo-2-(pentan-3-ylamino)cyclobut-1-en-1-yl)amino)-2-hydroxy-N-methoxy-N-methylbenzenesulfonamide choline salt. In some embodiments, the combination comprises PDR001 and 6-chloro-3-((3,4-dioxo-2-(pentan-3-ylamino)cyclobut-1-en-1-yl)amino)-2-hydroxy-N-methoxy-N-methylbenzenesulfonamide choline salt.

In some embodiments, the combination comprises PDR001, a CXCR2 inhibitor described herein (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt), and a third therapeutic agent (e.g., a therapeutic agent described herein).

In some embodiments, the third therapeutic agent comprises a TIM-3 inhibitor, e.g., a TIM-3 inhibitor disclosed herein. In some embodiments, the TIM-3 inhibitor is MBG453 (Novartis) or TSR-022 (Tesaro). In some embodiments, the TIM-3 inhibitor is MBG453. In some embodiments, the combination comprises PDR001, a CXCR2 inhibitor described herein, and MBG453.

In some embodiments, third therapeutic agent comprises a c-MET inhibitor, e.g., a c-MET inhibitor disclosed herein. In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280). In some embodiments, the combination comprises PDR001, a CXCR2 inhibitor described herein, and capmatinib (INC280). In some embodiments, the c-MET inhibitor (e.g., INC280) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., INC280) is administered twice a day at a dose of about 400 mg. In an embodiment, the c-MET inhibitor (e.g., INC280) is administered twice a day at a dose of about 600 mg.

In some embodiments, the third therapeutic agent comprises an A2aR antagonist, e.g., an A2Ar inhibitor disclosed herein. In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178). In some embodiments, the combination comprises PDR001, a CXCR2 inhibitor described herein, and PBF509 (NIR178).

In some embodiments, the combination comprises a PD-1 inhibitor (e.g., a PD-1 inhibitor described herein), a CXCR2 inhibitor (e.g., a CXCR2 inhibitor described herein), and one or more (e.g., two or all) of a TIM-3 inhibitor (e.g., a TIM-3 inhibitor described herein), a c-MET inhibitor (e.g., a c-MET inhibitor described herein), or an A2aR antagonist (e.g., an A2aR antagonist described herein).

In certain embodiments, the combination further comprises a fourth therapeutic agent, e.g., a therapeutic agent described herein.

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject or cancer is identified as having a biomarker described herein. In some embodiments, the cancer is, e.g., a solid tumor, e.g., a pancreatic cancer, a breast cancer (e.g., a triple negative breast cancer (TNBC)), a lung cancer (e.g., an NSCLC), or a colorectal cancer (CRC) (e.g., a microsatellite stable colorectal cancer (MSS-CRC). In some embodiments, the subject in need of the combination (e.g., PD-1 inhibitor described herein, and a CXCR2 inhibitor described herein) is not a patient requiring medications that are strong inducers or strong inhibitors of CYP3A4. In some embodiments, the subject in need of the combination (e.g., PD-1 inhibitor described herein, and a CXCR2 inhibitor described herein) is not a patient requiring medications with narrow therapeutic index CYP3A4 substrates. In some embodiments, the subject in need of the combination (e.g., PD-1 inhibitor described herein, and a CXCR2 inhibitor described herein) is not a patient using any form of hormonal contraception (e.g., oral, injected, implanted, or transdermal).

### Combination Targeting PD-1 and GITR

In an embodiment, the combination comprises a PD-1 inhibitor (e.g., a PD-1 inhibitor described herein), a GITR agonist (e.g., a GITR agonist described herein), and a third therapeutic agent.

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the GITR agonist is chosen from GWN323, BMS-986156, MK-4166, MK-1248, TRX518, INCAGN1876, AMG 228, or INBRX-110. In some embodiments, the GITR agonist is GWN323.

In some embodiments, the combination comprises PDR001 and GWN323.

In some embodiments, the third therapeutic agent comprises a TGF-β inhibitor, e.g., a TGF-β inhibitor disclosed herein. In some embodiments, the TGF-β inhibitor is fresolimumab or XOMA 089.

In some embodiments, the TGF-β inhibitor is XOMA 089. In some embodiments, the combination comprises PDR001, a GITR agonist described herein, and XOMA 089. In some embodiments, the combination comprises a PD-1 inhibitor described herein, GWN323, and XOMA 089. In some embodiments, the combination comprises PDR001, GWN323, and XOMA 089.

In some embodiments, the third therapeutic agent comprises an A2aR antagonist, e.g., an A2Ar inhibitor disclosed herein. In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178).

In some embodiments, the combination comprises PDR001, a GITR agonist described herein, and PBF509 (NIR178). In some embodiments, the combination comprises a PD-1 inhibitor described herein, GWN323, and PBF509 (NIR178). In some embodiments, the combination comprises PDR001, GWN323, and PBF509 (NIR178).

In some embodiments, the third therapeutic agent comprises a c-MET inhibitor, e.g., a c-MET inhibitor disclosed herein. In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280). In some embodiments, the combination comprises PDR001, a GITR agonist described herein, and capmatinib (INC280). In some embodiments, the combination comprises a PD-1 inhibitor described herein, GWN323, and capmatinib (INC280). In some embodiments, the combination comprises PDR001, GWN323, and capmatinib (INC280). In some embodiments, the c-MET inhibitor (e.g., INC280) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., INC280) is administered twice a day at a dose of about 400 mg. In an embodiment, the c-MET inhibitor (e.g., INC280) is administered twice a day at a dose of about 600 mg.

In some embodiments, the third therapeutic agent comprises a TIM-3 inhibitor, e.g., a TIM-3 inhibitor disclosed herein. In some embodiments, the TIM-3 inhibitor is chosen from MBG453 or TSR-022. In some embodiments, the TIM-3 inhibitor is MBG453. In some embodiments, the combination comprises PDR001, a GITR agonist described herein, and MBG453. In some embodiments, the combination comprises a PD-1 inhibitor described herein, GWN323, and MBG453. In some embodiments, the combination comprises PDR001, GWN323, and MBG453.

In some embodiments, the third therapeutic agent comprises a LAG-3 inhibitor, e.g., a LAG-3 inhibitor disclosed herein. In some embodiments, the LAG-3 inhibitor is chosen from LAG525, BMS-986016, or TSR-033. In some embodiments, the LAG-3 inhibitor is LAG525.

In some embodiments, the LAG-3 inhibitor is administered at a dose of about 300 to about 500 mg, about 400 mg to about 800 mg, or about 700 to about 900 mg. In some embodiments, the LAG-3 inhibitor is administered once every 3 weeks. In some embodiments, the LAG-3 inhibitor is administered once every 4 weeks. In other embodiments, the LAG-3 inhibitor is administered at a dose of about 300 mg to about 500 mg (e.g., about 400 mg) once every 3 weeks. In other embodiments, the LAG-3 inhibitor is administered at a dose of about 400 mg to about 800 mg (e.g., about 600 mg) once every 4 weeks. In yet other embodiments, the LAG-3 inhibitor is administered at a dose of about 700 mg to about 900 mg (e.g., about 800 mg) once every 4 weeks.

In some embodiments, the combination comprises PDR001, a GITR agonist described herein, and LAG525. In some embodiments, the combination comprises a PD-1 inhibitor described herein, GWN323, and LAG525. In some embodiments, the combination comprises PDR001, GWN323, and LAG525.

In some embodiments, the GITR agonist, e.g., GWN323 is administered at a dose of about 2 mg to about 10 mg, about 5 mg to about 20 mg, about 20 mg to about 40 mg, about 50 mg to about 100 mg, about 100 mg to about 200 mg, about 200 mg to about 400 mg, or about 400 mg to about 600 mg, once every week, once every three weeks, or once every six weeks.

In some embodiments, the combination comprises a PD-1 inhibitor (e.g., a PD-1 inhibitor described herein), a GITR agonist (e.g., a GITR agonist described herein), and one or more (e.g., two or all) of a TGF-β inhibitor (e.g., a TGF-β inhibitor described herein), a c-MET inhibitor (e.g., a c-MET inhibitor described herein), an A2aR antagonist (e.g., an A2aR antagonist described herein), a TIM-3 inhibitor (e.g., a TIM-3 inhibitor described herein), or a LAG-3 inhibitor (e.g., a LAG-3 inhibitor described herein).

In certain embodiments, the combination further comprises a fourth therapeutic agent, e.g., a therapeutic agent described herein.

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has or cancer is identified as having a biomarker described herein. In some embodiments, the cancer is a solid tumor, e.g., a pancreatic cancer, a colorectal cancer (CRC), or a melanoma (e.g., a refractory melanoma).

### Combination Targeting PD-1 and LAG-3

In an embodiment, the combination comprises a PD-1 inhibitor, (e.g., a PD-1 inhibitor described herein), and a LAG-3 inhibitor (e.g., a LAG-3 inhibitor described herein).

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the LAG-3 inhibitor is chosen from LAG525 (Novartis), BMS-986016 (Bristol-Myers Squibb), or TSR-033 (Tesaro). In some embodiments, the LAG-3 inhibitor is LAG525. In some embodiments, the LAG-3 inhibitor is administered at a dose of about 300 to about 500 mg, about 400 mg to about 800 mg, or about 700 to about 900 mg. In some embodiments, the LAG-3 inhibitor is administered once every 3 weeks. In some embodiments, the LAG-3 inhibitor is administered once every 4 weeks. In other embodiments, the LAG-3 inhibitor is administered at a dose of about 300 mg to about 500 mg (e.g., about 400 mg) once every 3 weeks. In other embodiments, the LAG-3 inhibitor is administered at a dose of about 400 mg to about 800 mg (e.g., about 600 mg) once every 4 weeks. In yet other embodiments, the LAG-3 inhibitor is administered at a dose of about 700 mg to about 900 mg (e.g., about 800 mg) once every 4 weeks.

In some embodiments, the composition comprises a combination of a PD-1 inhibitor, e.g., PDR001, and a LAG-3 inhibitor, e.g., LAG525. In some embodiments, the combination comprises PDR001, and a LAG-3 inhibitor described herein. In some embodiments, the combination comprises a PD-1 inhibitor described herein and LAG525. In some embodiments, the combination comprises PDR001 and LAG525.

In some embodiments, the LAG-3 inhibitor, e.g., LAG525, is administered, e.g., infused, prior to administration, e.g., infusion, of the PD-1 inhibitor, e.g., PDR001. In some embodiments, the PD-1 inhibitor, e.g., PDR001, is administered, e.g., infused, after administration, e.g., infusion, of the LAG-3 inhibitor, e.g., LAG525. In some embodiments, both the PD-1 inhibitor, e.g., PDR001, and the LAG-3 inhibitor, e.g., LAG525, are administered, e.g., infused at the same site of administration, e.g., infusion site.

In some embodiments, the combination further comprises a TGF-β inhibitor, e.g., a TGF-β inhibitor disclosed herein. In some embodiments, the TGF-β inhibitor is fresolimumab or XOMA 089. In some embodiments, the TGF-β inhibitor is XOMA 089. In some embodiments, the combination comprises PDR001, a LAG-3 inhibitor described herein, and XOMA 089. In some embodiments, the combination comprises a PD-1 inhibitor described herein, LAG525 and XOMA 089. In some embodiments, the combination comprises PDR001, LAG525, and XOMA 089.

In some embodiments, the combination further comprises a TIM-3 inhibitor, e.g., a TIM-3 inhibitor described herein. In some embodiments, the TIM-3 inhibitor is chosen from MBG453 or TSR-022. In some embodiments, the TIM-3 inhibitor is MBG453. In some embodiments, the combination comprises PDR001, a LAG-3 inhibitor described herein, and MBG453. In some embodiments, the combination comprises a PD-1 inhibitor described herein, LAG525 and MBG453. In some embodiments, the combination comprises PDR001, LAG525, and MBG453.

In some embodiments, the combination further comprises a c-MET inhibitor, e.g., a c-MET inhibitor described herein. In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280). In some embodiments, the combination comprises PDR001, a LAG-3 inhibitor described herein, and capmatinib (INC280). In some embodiments, the combination comprises a PD-1 inhibitor described herein, LAG525 and capmatinib (INC280). In some embodiments, the combination comprises PDR001, LAG525, and capmatinib (INC280). In some embodiments, the c-MET inhibitor (e.g., capmatinib) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., capmatinib) is administered twice a day at a dose of about 400 mg. In an embodiment, the c-MET inhibitor (e.g., capmatinib) is administered twice a day at a dose of about 600 mg.

In some embodiments, the combination further comprises an IL-1β inhibitor, e.g., an IL-1β inhibitor described herein. In some embodiments, the IL-1β inhibitor is chosen from canakinumab, gevokizumab, Anakinra, or Rilonacept. In some embodiments, the combination comprises PDR001, a LAG-3 inhibitor described herein, and an IL-1β inhibitor described herein (e.g., canakinumab, gevokizumab, Anakinra, or Rilonacept). In some embodiments, the combination comprises a PD-1 inhibitor described herein, LAG525, and an IL-1β inhibitor described herein (e.g., canakinumab, gevokizumab, Anakinra, or Rilonacept). In some embodiments, the combination comprises PDR001, LAG525 and, an IL-1β inhibitor described herein (e.g., canakinumab, gevokizumab, Anakinra, or Rilonacept).

In some embodiments, the combination further comprises a MEK inhibitor, e.g., a MEK inhibitor described herein. In some embodiments, the MEK inhibitor is chosen from Trametinib, selumetinib, AS703026, BIX 02189, BIX 02188, CI-1040, PD0325901, PD98059, U0126, XL-518, G-38963, or G02443714. In some embodiments, the MEK inhibitor is Trametinib. In some embodiments, the combination comprises PDR001, a LAG-3 inhibitor described herein, and Trametinib. In some embodiments, the combination comprises a PD-1 inhibitor described herein, LAG525 and Trametinib. In some embodiments, the combination comprises PDR001, LAG525, and Trametinib.

In some embodiments, the combination further comprises a GITR agonist, e.g., a GITR agonist described herein. In some embodiments, the GITR agonist is chosen from GWN323, BMS-986156, MK-4166, MK-1248, TRX518, INCAGN1876, AMG 228, or INBRX-110. In some embodiments, the GITR agonist is GWN323. In some embodiments, the combination comprises PDR001, a LAG-3 inhibitor described herein, and GWN323. In some embodiments, the combination comprises a PD-1 inhibitor described herein, LAG525 and GWN323. In some embodiments, the combination comprises PDR001, LAG525, and GWN323.

In some embodiments, the combination further comprises a CSF-1/1R binding agent, e.g., a CSF-1/1R binding agent described herein. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110. In some embodiments, the combination comprises PDR001, a LAG-3 inhibitor described herein, and MCS110. In some embodiments, the combination comprises a PD-1 inhibitor described herein, LAG525 and MCS110. In some embodiments, the combination comprises PDR001, LAG525, and MCS110. In some embodiments, the combination comprises PDR001, a LAG-3 inhibitor described herein, and BLZ945. In some embodiments, the combination comprises a PD-1 inhibitor described herein, LAG525 and BLZ945. In some embodiments, the combination comprises PDR001, LAG525, and BLZ945.

In some embodiments, the combination further comprises an A2aR antagonist, e.g., an A2aR antagonist described herein. In some embodiments, the A2aR antagonist is chosen from: PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178). In some embodiments, the combination comprises PDR001, a LAG-3 inhibitor described herein, and PBF509 (NIR178). In some embodiments, the combination comprises PDR001, LAG525, and PBF509 (NIR178). In some embodiments, the combination comprises PDR001, LAG525, and PBF509 (NIR178).

In some embodiments, the combination further comprises a MEK inhibitor, e.g., trametinib or cobimetinib, paclitaxel, and a PD-L1 inhibitor, e.g., Atezolizumab. In some embodiments, the combination comprises a PD-1 inhibitor described herein, a MEK inhibitor, e.g., trametinib or cobimetinib and paclitaxel. In some embodiments, the combination comprises PDR001, cobimetinib and paclitaxel. In some embodiments, the combination comprises a PD-1 inhibitor described herein, a MEK inhibitor described herein, e.g., trametinib or cobimetinib, paclitaxel and Atezolizumab. In some embodiments, the combination comprises PDR001, cobimetinib, paclitaxel and Atezolizumab.

In some embodiments, the combination comprises a PD-1 inhibitor (e.g., a PD-1 inhibitor described herein), a LAG-3 inhibitor (e.g., a LAG-3 inhibitor described herein), and one or more (e.g., two or all) of a TGF-β inhibitor (e.g., a TGF-β inhibitor described herein), a TIM-3 inhibitor (e.g., a TIM-3 inhibitor described herein), a c-MET inhibitor (e.g., a c-MET inhibitor described herein), an IL-1β inhibitor (e.g., an IL-1β inhibitor described herein) a MEK inhibitor (e.g., a MEK inhibitor described herein) a GITR agonist (e.g., a GITR agonist described herein), an A2aR antagonist (e.g., an A2aR antagonist described herein), or a CSF-1/1R binding agent (e.g., a CSF-1/1R binding agent described herein).

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a breast cancer, e.g., a triple negative breast cancer (TNBC). In some embodiments, the cancer is a TNBC, e.g., an advanced or metastatic TNBC.

### Combination Targeting PD-1 and CSF-1/1R

In an embodiment, the combination comprises a PD-1 inhibitor, (e.g., a PD-1 inhibitor described herein), and a CSF-1/1R binding agent (e.g., a CSF-1/1R binding agent disclosed herein).

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110.

In some embodiments, the composition comprises a combination of a PD-1 inhibitor, e.g., PDR001, and a CSF-1/1R binding agent, e.g., BLZ945. In some embodiments, the combination comprises PDR001, and a CSF-1/1R binding agent described herein, e.g., MCS110 or BLZ945. In some embodiments, the combination comprises a PD-1 inhibitor described herein and BLZ945. In some embodiments, the combination comprises PDR001 and BLZ945.

In some embodiments, the composition comprises a combination of a PD-1 inhibitor, e.g., PDR001, and a CSF-1/1R binding agent, e.g., MCS110. In some embodiments, the combination comprises PDR001, and a CSF-1/1R binding agent described herein. In some embodiments, the combination comprises a PD-1 inhibitor described herein and MCS110. In some embodiments, the combination comprises PDR001 and MCS110.

In some embodiments, the combination further comprises a TGF-β inhibitor, e.g., a TGF-β inhibitor disclosed herein. In some embodiments, the TGF-β inhibitor is fresolimumab or XOMA 089. In some embodiments, the TGF-β inhibitor is XOMA 089. In some embodiments, the combination comprises PDR001, a CSF-1/1R binding agent described herein, and XOMA 089. In some embodiments, the combination comprises a PD-1 inhibitor described herein, BLZ945 and XOMA 089. In some embodiments, the combination comprises PDR001, BLZ945, and XOMA 089.

In some embodiments, the combination further comprises a TIM-3 inhibitor, e.g., a TIM-3 inhibitor described herein. In some embodiments, the TIM-3 inhibitor is chosen from MBG453 or TSR-022. In some embodiments, the TIM-3 inhibitor is MBG453. In some embodiments, the combination comprises PDR001, a CSF-1/1R binding agent described herein, and MBG453. In some embodiments, the combination comprises a PD-1 inhibitor described herein, BLZ945 and MBG453. In some embodiments, the combination comprises PDR001, BLZ945, and MBG453.

In some embodiments, the combination further comprises a c-MET inhibitor, e.g., a c-MET inhibitor described herein. In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280). In some embodiments, the combination comprises PDR001, a CSF-1/1R binding agent described herein, and capmatinib (INC280). In some embodiments, the combination comprises a PD-1 inhibitor described herein, BLZ945 and capmatinib (INC280). In some embodiments, the combination comprises PDR001, BLZ945, and capmatinib (INC280). In some embodiments, the c-MET inhibitor (e.g., INC280) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., INC280) is administered twice a day at a dose of about 400 mg. In an embodiment, the c-MET inhibitor (e.g., INC280) is administered twice a day at a dose of about 600 mg.

In some embodiments, the combination further comprises an IL-1β inhibitor, e.g., an IL-1β inhibitor described herein. In some embodiments, the IL-1β inhibitor is chosen from canakinumab, gevokizumab, Anakinra, or Rilonacept. In some embodiments, the combination comprises PDR001, a CSF-1/1R binding agent described herein, and an IL-1β inhibitor described herein (e.g., canakinumab, gevokizumab, Anakinra or Rilonacept). In some embodiments, the combination comprises a PD-1 inhibitor described herein, BLZ945 and, an IL-1β inhibitor described herein (e.g., canakinumab, gevokizumab, Anakinra or Rilonacept). In some embodiments, the combination comprises PDR001, BLZ945, and an IL-1β inhibitor described herein (e.g., canakinumab, gevokizumab, Anakinra or Rilonacept).

In some embodiments, the combination further comprises Eribulin, also known as E7389 and ER-086526. In some embodiments, the combination comprises PDR001, a CSF-1/1R binding agent described herein, e.g., BLZ945 or pexidartinib, and Eribulin. In some embodiments, the combination comprises PDR001, BLZ945, and Eribulin. IN some embodiments, the combination comprises PDR001, pexidartinib, and Eribulin.

In some embodiments, the combination comprises a PD-1 inhibitor (e.g., a PD-1 inhibitor described herein), a CSF-1/1R binding agent (e.g., a CSF-1/1R binding agent described herein), and one or more (e.g., two or all) of a TGF-β inhibitor (e.g., a TGF-β inhibitor described herein), a TIM-3 inhibitor (e.g., a TIM-3 inhibitor described herein), a c-MET inhibitor (e.g., a c-MET inhibitor described herein), and an IL-1beta inhibitor (e.g., an IL-1beta inhibitor described herein).

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a breast cancer, a colorectal cancer (CRC), a gastroesophageal cancer or a pancreatic cancer. In some embodiments, the breast cancer is a triple negative breast cancer (TNBC). In some embodiments, the CRC is a microsatellite stable CRC (MSS CRC).

### Combination Targeting PD-1 and A2aR

In an embodiment, the combination comprises a PD-1 inhibitor, (e.g., a PD-1 inhibitor described herein), and an A2aR antagonist (e.g., an A2aR antagonist described herein).

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178).

In some embodiments, the composition comprises a combination of a PD-1 inhibitor, e.g., PDR001, and an A2aR antagonist, e.g., PBF509 (NIR178). In some embodiments, the combination comprises PDR001 and an A2aR antagonist described herein. In some embodiments, the combination comprises a PD-1 inhibitor described herein and PBF509 (NIR178). In some embodiments, the combination comprises PDR001 and PBF509 (NIR178).

In some embodiments, the combination further comprises a TGF-β inhibitor, e.g., a TGF-β inhibitor disclosed herein. In some embodiments, the TGF-β inhibitor is fresolimumab or XOMA 089. In some embodiments, the TGF-β inhibitor is XOMA 089. In some embodiments, the combination comprises PDR001, an A2aR antagonist described herein, and XOMA 089. In some embodiments, the combination comprises a PD-1 inhibitor described herein, PBF509 (NIR178) and XOMA 089. In some embodiments, the combination comprises PDR001, PBF509 (NIR178), and XOMA 089.

In some embodiments, the combination further comprises a TIM-3 inhibitor, e.g., a TIM-3 inhibitor described herein. In some embodiments, the TIM-3 inhibitor is chosen from MBG453 or TSR-022. In some embodiments, the TIM-3 inhibitor is MBG453. In some embodiments, the combination comprises PDR001, an A2aR antagonist described herein, and MBG453. In some embodiments, the combination comprises a PD-1 inhibitor described herein, PBF509 (NIR178) and MBG453. In some embodiments, the combination comprises PDR001, PBF509 (NIR178), and MBG453.

In some embodiments, the combination further comprises a c-MET inhibitor, e.g., a c-MET inhibitor described herein. In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280). In some embodiments, the combination comprises PDR001, an A2aR antagonist described herein, and capmatinib (INC280). In some embodiments, the combination comprises a PD-1 inhibitor described herein, PBF509 (NIR178) and capmatinib (INC280). In some embodiments, the combination comprises PDR001, PBF509 (NIR178), and capmatinib (INC280). In some embodiments, the c-MET inhibitor (e.g., INC280) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., INC280) is administered twice a day at a dose of about 400 mg. In an embodiment, the c-MET inhibitor (e.g., INC280) is administered twice a day at a dose of about 600 mg.

In some embodiments, the combination further comprises an IL-1β inhibitor, e.g., an IL-1β inhibitor described herein. In some embodiments, the IL-1β inhibitor is chosen from canakinumab, gevokizumab, Anakinra, or Rilonacept. In some embodiments, the combination comprises PDR001, an A2aR antagonist described herein, and an IL-1β inhibitor described herein (e.g., canakinumab, gevokizumab, Anakinra, or Rilonacept). In some embodiments, the combination comprises a PD-1 inhibitor described herein, PBF509 (NIR178), an IL-1β inhibitor described herein (e.g., canakinumab, gevokizumab, Anakinra, or Rilonacept). In some embodiments, the combination comprises PDR001, PBF509 (NIR178), and an IL-1β inhibitor described herein (e.g., canakinumab, gevokizumab, Anakinra, or Rilonacept).

In some embodiments, the combination further comprises an IL-15/IL-15Ra complex, e.g., an IL-15/IL-15Ra complex described herein. In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. In some embodiments, the combination comprises PDR001, an A2aR antagonist described herein, and NIZ985. In some embodiments, the combination comprises a PD-1 inhibitor described herein, PBF509 (NIR178), and NIZ985. In some embodiments, the combination comprises PDR001, PBF509 (NIR178), and NIZ985.

In some embodiments, the combination further comprises a CSF-1/1R binding agent. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110.

In some embodiments, the combination comprises PDR001, an A2aR antagonist described herein, and BLZ945. In some embodiments, the combination comprises a PD-1 inhibitor described herein, PBF509 (NIR178) and BLZ945. In some embodiments, the combination comprises PDR001, PBF509 (NIR178), and BLZ945.

In some embodiments, the combination comprises PDR001, an A2aR antagonist described herein, and MCS110. In some embodiments, the combination comprises a PD-1 inhibitor described herein, PBF509 (NIR178), and MCS110. In some embodiments, the combination comprises PDR001, PBF509 (NIR178), and MCS110.

In some embodiments, the combination comprises a PD-1 inhibitor (e.g., a PD-1 inhibitor described herein), an A2aR antagonist (e.g., an A2aR antagonist described herein), and one or more (e.g., two, three, four, or all) of a TGF-β inhibitor (e.g., a TGF-β inhibitor described herein), a TIM-3 inhibitor (e.g., a TIM-3 inhibitor described herein), a c-MET inhibitor (e.g., a c-MET inhibitor described herein), an IL-1beta inhibitor (e.g., an IL-1beta inhibitor described herein), an IL-15/IL-15RA complex (e.g., an IL-15/IL-15RA complex described herein), or a CSF-1/1R binding agent (e.g., a CSF-1/1R binding agent described herein).

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a breast cancer, a colorectal cancer (CRC), a gastroesophageal cancer or a pancreatic cancer. In some embodiments, the breast cancer is a triple negative breast cancer (TNBC). In some embodiments, the CRC is a microsatellite stable CRC (MSS CRC).

### Combination Targeting PD-1 and IL-1 Beta Inhibitor

In an embodiment, the combination comprises a PD-1 inhibitor, (e.g., a PD-1 inhibitor described herein), and an IL-1beta inhibitor (e.g., an IL-1beta inhibitor described herein).

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the IL-1β inhibitor is chosen from canakinumab, gevokizumab, Anakinra, or Rilonacept

In some embodiments, the composition comprises a combination of a PD-1 inhibitor, e.g., PDR001, and an IL-1β inhibitor, e.g., canakinumab, gevokizumab, Anakinra, or Rilonacept. In some embodiments, the combination comprises PDR001 and an IL-1β inhibitor described herein. In some embodiments, the combination comprises a PD-1 inhibitor described herein and, canakinumab, gevokizumab, Anakinra, or Rilonacept. In some embodiments, the combination comprises PDR001 and, canakinumab, gevokizumab, Anakinra, or Rilonacept.

In some embodiments, the combination further comprises a TGFb inhibitor, e.g., a TGFb inhibitor disclosed herein. In some embodiments, the TGFb inhibitor is fresolimumab or XOMA 089. In some embodiments, the TGFb inhibitor is XOMA 089. In some embodiments, the combination comprises PDR001, an IL-1β inhibitor described herein (e.g., canakinumab, gevokizumab, Anakinra, or Rilonacept), and XOMA 089.

In some embodiments, the combination further comprises an IL-15/IL-15Ra complex, e.g., an IL-15/IL-15Ra complex described herein. In some embodiments, the IL-15/IL-15Ra complex is chosen from NIZ985 (Novartis), ATL-803 (Altor), or CYP0150 (Cytune). In some embodiments, the IL-15/IL-15RA complex is NIZ985. In some embodiments, the combination comprises PDR001, an IL-1β inhibitor described herein (e.g., canakinumab, gevokizumab, Anakinra, or Rilonacept), and NIZ985.

In some embodiments, the combination further comprises a CSF-1/1R binding agent. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110.

In some embodiments, the combination comprises PDR001, an IL-1β inhibitor described herein (e.g., canakinumab, gevokizumab, Anakinra, or Rilonacept), and MCS110. In some embodiments, the combination comprises a PD-1 inhibitor described herein, an IL-1β inhibitor described herein (e.g., canakinumab, gevokizumab, Anakinra, or Rilonacept), and MCS110.

In some embodiments, the combination comprises PDR001, an IL-1β inhibitor described herein (e.g., canakinumab, gevokizumab, Anakinra, or Rilonacept), and BLZ945. In some embodiments, the combination comprises a PD-1 inhibitor described herein, an IL-1β inhibitor described herein (e.g., canakinumab, gevokizumab, Anakinra, or Rilonacept) and BLZ945.

In some embodiments, the combination further comprises a TIM-3 inhibitor, e.g., a TIM-3 inhibitor described herein. In some embodiments, the TIM-3 inhibitor is chosen from MBG453 or TSR-022. In some embodiments, the TIM-3 inhibitor is MBG453. In some embodiments, the combination comprises PDR001, an IL-1β inhibitor described herein (e.g., canakinumab, gevokizumab, Anakinra, or Rilonacept), and MBG453. In some embodiments, the combination comprises a PD-1 inhibitor described herein, an IL-1β inhibitor described herein (e.g., canakinumab, gevokizumab, Anakinra, or Rilonacept), and MBG453.

In some embodiments, the combination further comprises a c-MET inhibitor, e.g., a c-MET inhibitor described herein. In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280). In some embodiments, the combination comprises PDR001, an IL-1β inhibitor described herein (e.g., canakinumab, gevokizumab, Anakinra, or Rilonacept), and capmatinib (INC280). In some embodiments, the combination comprises a PD-1 inhibitor described herein, an IL-1β inhibitor described herein (e.g., canakinumab, gevokizumab, Anakinra, or Rilonacept), and capmatinib (INC280). In some embodiments, the c-MET inhibitor (e.g., INC280) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., INC280) is administered twice a day at a dose of about 400 mg. In an embodiment, the c-MET inhibitor (e.g., INC280) is administered twice a day at a dose of about 600 mg.

In some embodiments, the combination comprises a PD-1 inhibitor (e.g., a PD-1 inhibitor described herein), an IL-1β inhibitor (e.g., an IL-1β inhibitor described herein), and one or more (e.g., two, three, four, or all) of a TGF-β inhibitor (e.g., a TGF-β inhibitor described herein), a TIM-3 inhibitor (e.g., a TIM-3 inhibitor described herein), a c-MET inhibitor (e.g., a c-MET inhibitor described herein), an IL-15/IL-15RA complex (e.g., an IL-15/IL-15RA complex described herein), or a CSF-1/1R binding agent (e.g., a CSF-1/1R binding agent described herein).

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a colorectal cancer (CRC), a gastroesophageal cancer or a pancreatic cancer. In some embodiments, the CRC is a microsatellite stable CRC (MSS CRC).

### Combination Targeting PD-1 and MEK Inhibitor

In an embodiment, the combination comprises a PD-1 inhibitor, (e.g., a PD-1 inhibitor described herein), and a MEK inhibitor (e.g., a MEK inhibitor described herein).

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the MEK inhibitor is chosen from Trametinib, binimetinib, selumetinib, AS703026, BIX 02189, BIX 02188, CI-1040, PD0325901, PD98059, U0126, XL-518, G-38963, or G02443714. In some embodiments, the MEK inhibitor is Trametinib. In some embodiments, the MEK inhibitor or trametinib is administered at a dose between 0.1 mg and 4 mg (e.g., between 0.5 mg and 3 mg, e.g., at a dose of 0.5 mg), e.g., once a day. In some embodiments, the MEK inhibitor or trametinib is administered at a dose of 0.5 mg, e.g., once a day. In some embodiments, the MEK inhibitor or trametinib is administered orally.

In some embodiments, the composition comprises a combination of a PD-1 inhibitor, e.g., PDR001, and a MEK inhibitor, e.g., trametinib. In some embodiments, the combination comprises PDR001 and a MEK inhibitor, e.g., trametinib. In some embodiments, the combination comprises a PD-1 inhibitor, e.g., PDR001, and trametinib. In some embodiments, the combination comprises PDR001 and trametinib.

In some embodiments, the combination comprises a PD-1 inhibitor, e.g., PDR001 and a MEK inhibitor, e.g., binimetinib. In some embodiments, the combination comprises PDR001 and binimetinib.

In some embodiments, the combination further comprises a TGF-β inhibitor, e.g., a TGF-β inhibitor disclosed herein. In some embodiments, the TGF-β inhibitor is fresolimumab or XOMA 089. In some embodiments, the TGF-β inhibitor is XOMA 089. In some embodiments, the combination comprises PDR001, a MEK inhibitor described herein, and XOMA 089. In some embodiments, the combination comprises a PD-1 inhibitor described herein, trametinib and XOMA 089. In some embodiments, the combination comprises PDR001, trametinib, and XOMA 089.

In some embodiments, the combination further comprises an IL-15/IL-15Ra complex, e.g., an IL-15/IL-15Ra complex described herein. In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. In some embodiments, the combination comprises PDR001, a MEK inhibitor described herein, and NIZ985. In some embodiments, the combination comprises a PD-1 inhibitor described herein, trametinib, and NIZ985. In some embodiments, the combination comprises PDR001, trametinib, and NIZ985.

In some embodiments, the combination further comprises a CSF-1/1R binding agent. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110.

In some embodiments, the combination comprises PDR001, a MEK inhibitor described herein, and MCS110. In some embodiments, the combination comprises a PD-1 inhibitor described herein, trametinib and MCS110. In some embodiments, the combination comprises PDR001, trametinib, and MCS110.

In some embodiments, the combination comprises PDR001, a MEK inhibitor described herein, and BLZ945. In some embodiments, the combination comprises a PD-1 inhibitor described herein, trametinib and BLZ945. In some embodiments, the combination comprises PDR001, trametinib, and BLZ945.

In some embodiments, the combination further comprises a TIM-3 inhibitor, e.g., a TIM-3 inhibitor described herein. In some embodiments, the TIM-3 inhibitor is chosen from MBG453 or TSR-022. In some embodiments, the TIM-3 inhibitor is MBG453. In some embodiments, the combination comprises PDR001, a MEK inhibitor described herein, and MBG453. In some embodiments, the combination comprises a PD-1 inhibitor described herein, trametinib and MBG453. In some embodiments, the combination comprises PDR001, trametinib, and MBG453.

In some embodiments, the combination further comprises a c-MET inhibitor, e.g., a c-MET inhibitor described herein. In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280). In some embodiments, the combination comprises PDR001, a MEK inhibitor described herein, and capmatinib (INC280). In some embodiments, the combination comprises a PD-1 inhibitor described herein, trametinib, and capmatinib (INC280). In some embodiments, the combination comprises PDR001, trametinib, and capmatinib (INC280). In some embodiments, the c-MET inhibitor (e.g., INC280) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., INC280) is administered twice a day at a dose of about 400 mg. In an embodiment, the c-MET inhibitor (e.g., INC280) is administered twice a day at a dose of about 600 mg.

In some embodiments, the combination comprises a PD-1 inhibitor (e.g., a PD-1 inhibitor described herein), a MEK inhibitor (e.g., a MEK inhibitor described herein), and one or more (e.g., two, three, four, or all) of a TGF-β inhibitor (e.g., a TGF-β inhibitor described herein), a TIM-3 inhibitor (e.g., a TIM-3 inhibitor described herein), a c-MET inhibitor (e.g., a c-MET inhibitor described herein), an IL-15/IL-15RA complex (e.g., an IL-15/IL-15RA complex described herein), or a CSF-1/1R binding agent (e.g., a CSF-1/1R binding agent described herein).

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a colorectal cancer (CRC), a gastroesophageal cancer or a pancreatic cancer. In some embodiments, the CRC is a microsatellite stable CRC (MSS CRC).

### Combination Targeting PD-1, LAG-3 and GITR

In an embodiment, the combination comprises a PD-1 inhibitor, (e.g., a PD-1 inhibitor described herein), a LAG-3 inhibitor (e.g., a LAG-3 inhibitor described herein), and a GITR agonist (e.g., a GITR agonist described herein).

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the LAG-3 inhibitor is chosen from LAG525 (Novartis), BMS-986016 (Bristol-Myers Squibb), or TSR-033 (Tesaro). In some embodiments, the LAG-3 inhibitor is LAG525.

In some embodiments, the GITR agonist is chosen from GWN323, BMS-986156, MK-4166, MK-1248, TRX518, INCAGN1876, AMG 228 or INBRX-110. In some embodiments, the GITR agonist is GWN323.

In some embodiments, the composition comprises a combination of a PD-1 inhibitor, e.g., PDR001, a GITR agonist, e.g., GWN323, and a LAG-3 inhibitor, e.g., LAG525.

In some embodiments, the combination comprises PDR001, a LAG-3 inhibitor described herein, and a GITR agonist described herein. In some embodiments, the combination comprises a PD-1 inhibitor described herein, LAG525, and a GITR agonist described herein. In some embodiments, the combination comprises a PD-1 inhibitor described herein, a LAG-3 inhibitor described herein, and GWN323. In some embodiments, the combination comprises PDR001, LAG525, and a GITR agonist described herein. In some embodiments, the combination comprises PDR001, a LAG-3 inhibitor described herein, and GWN323. In some embodiments, the combination comprises a PD-1 inhibitor described herein, LAG525, and GWN323. In some embodiments, the combination comprises PDR001, LAG525, and GWN323.

In some embodiments, the combination further comprises a TGF-β inhibitor, e.g., a TGF-β inhibitor disclosed herein. In some embodiments, the TGF-β inhibitor is fresolimumab or XOMA 089. In some embodiments, the TGF-β inhibitor is XOMA 089. In some embodiments, the combination comprises PDR001, a LAG-3 inhibitor described herein, a GITR agonist and XOMA 089. In some embodiments, the combination comprises a PD-1 inhibitor described herein, LAG525, a GITR agonist and XOMA 089. In some embodiments, the combination comprises a PD-1 inhibitor described herein, a LAG-3 inhibitor described herein, GWN323, and XOMA 809. In some embodiments, the combination comprises PDR001, LAG525, GWN323 and XOMA 089.

In some embodiments, the combination further comprises an A2aR antagonist, e.g., an A2aR antagonist disclosed herein. In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178).

In some embodiments, the combination comprises PDR001, a LAG-3 inhibitor described herein, a GITR agonist and PBF509 (NIR178). In some embodiments, the combination comprises a PD-1 inhibitor described herein, LAG525, a GITR agonist and PBF509 (NIR178). In some embodiments, the combination comprises a PD-1 inhibitor described herein, a LAG-3 inhibitor described herein, GWN323, and PBF509 (NIR178). In some embodiments, the combination comprises PDR001, LAG525, GWN323 and PBF509 (NIR178).

In some embodiments, the combination further comprises a c-MET inhibitor, e.g., a c-MET inhibitor disclosed herein. In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280). In some embodiments, the c-MET inhibitor (e.g., INC280) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., INC280) is administered twice a day at a dose of about 400 mg. In an embodiment, the c-MET inhibitor (e.g., INC280) is administered twice a day at a dose of about 600 mg.

In some embodiments, the combination comprises PDR001, a LAG-3 inhibitor described herein, a GITR agonist and capmatinib (INC280). In some embodiments, the combination comprises a PD-1 inhibitor described herein, LAG525, a GITR agonist and capmatinib (INC280). In some embodiments, the combination comprises a PD-1 inhibitor described herein, a LAG-3 inhibitor described herein, GWN323, and capmatinib (INC280). In some embodiments, the combination comprises PDR001, LAG525, GWN323 and capmatinib (INC280).

In some embodiments, the combination comprises a PD-1 inhibitor (e.g., a PD-1 inhibitor described herein), a LAG-3 inhibitor (e.g., a LAG-3 inhibitor described herein), a GITR agonist (e.g., a GITR agonist described herein), and one or more (e.g., two or all) of a TGF-β inhibitor (e.g., a TGF-β inhibitor described herein), a c-MET inhibitor (e.g., a c-MET inhibitor described herein), or an A2aR antagonist (e.g., an A2aR antagonist described herein).

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a pancreatic cancer, a colorectal cancer (CRC) or a melanoma (e.g., a refractory melanoma).

### Combination Targeting PD-1 and A2aR

In an embodiment, the combination comprises a PD-1 inhibitor (e.g., a PD-1 inhibitor described herein), an A2aR antagonist (e.g., an A2aR antagonist described herein), and a third therapeutic agent. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178).

In some embodiments, the combination comprises PDR001 and PBF509 (NIR178).

In some embodiments, the third therapeutic agent comprises a TGF-β inhibitor, e.g., a TGF-β inhibitor disclosed herein. In some embodiments, the TGF-β inhibitor is fresolimumab or XOMA 089. In some embodiments, the TGF-β inhibitor is XOMA 089. In some embodiments, the combination comprises PDR001, an A2aR antagonist described herein, and XOMA 089. In some embodiments, the combination comprises a PD-1 inhibitor described herein, PBF509 (NIR178), and XOMA 089. In some embodiments, the combination comprises PDR001, PBF509 (NIR178), and XOMA 089.

In some embodiments, the third therapeutic agent comprises a CSF-1/1R binding agent, e.g., a CSF-1/1R binding agent disclosed herein. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK)(e.g., pexidartinib), or an antibody targeting CSF1R (e.g., emactuzumab or FPA008). In some embodiments, the CSF-1/1R binding agent is BLZ945. In some embodiments, the CSF-1/1R binding agent is MCS110.

In some embodiments, the combination comprises PDR001, an A2aR antagonist described herein, and BLZ945. In some embodiments, the combination comprises a PD-1 inhibitor described herein, PBF509 (NIR178), and BLZ945. In some embodiments, the combination comprises a PDR001, PBF509 (NIR178), and BLZ945.

In some embodiments, the combination comprises PDR001, an A2aR antagonist described herein, and MCS110. In some embodiments, the combination comprises a PD-1 inhibitor described herein, PBF509 (NIR178), and MCS110. In some embodiments, the combination comprises a PDR001, PBF509 (NIR178), and MCS110.

In some embodiments, the combination comprises a PD-1 inhibitor (e.g., a PD-1 inhibitor described herein), an A2aR antagonist (e.g., an A2aR antagonist described herein), and one or both of a TGF-β inhibitor (e.g., a TGF-β inhibitor described herein) or a CSF-1/1R binding agent (e.g., a CSF-1/1R binding agent described herein).

In certain embodiments, the combination further comprises a fourth therapeutic agent, e.g., a therapeutic agent described herein.

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a pancreatic cancer, a colorectal cancer (CRC) or a melanoma (e.g., a refractory melanoma).

Combination Targeting PD-1 and c-MET

In an embodiment, the combination comprises a PD-1 inhibitor (e.g., a PD-1 inhibitor described herein), a c-MET inhibitor (e.g., a c-MET inhibitor described herein), and a third therapeutic agent.

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280).

In some embodiments, the combination comprises PDR001 and capmatinib (INC280).

In some embodiments, the therapeutic agent comprises a TGF-β inhibitor, e.g., a TGF-β inhibitor disclosed herein. In some embodiments, the TGF-β inhibitor is fresolimumab or XOMA 089. In some embodiments, the TGF-β inhibitor is XOMA 089. In some embodiments, the combination comprises PDR001, a c-MET inhibitor described herein, and XOMA 089. In some embodiments, the combination comprises a PD-1 inhibitor described herein, capmatinib (INC280), and XOMA 089. In some embodiments, the combination comprises PDR001, capmatinib (INC280), and XOMA 089.

In some embodiments, the third therapeutic agent comprises an A2aR antagonist, e.g., an A2Ar inhibitor disclosed herein. In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178).

In some embodiments, the combination comprises PDR001, a c-MET inhibitor described herein, and PBF509 (NIR178). In some embodiments, the combination comprises a PD-1 inhibitor described herein, capmatinib (INC280), and PBF509 (NIR178). In some embodiments, the combination comprises PDR001, capmatinib (INC280), and PBF509 (NIR178).

In some embodiments, the third therapeutic agent comprises a CSF-1/1R binding agent, e.g., a CSF-1/1R binding agent disclosed herein. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK)(e.g., pexidartinib), or an antibody targeting CSF-1R (e.g., emactuzumab or FPA008). In some embodiments, the CSF-1/1R binding agent is BLZ945. In some embodiments, the CSF-1/1R binding agent is MCS110.

In some embodiments, the combination comprises PDR001, a c-MET inhibitor described herein, and BLZ945. In some embodiments, the combination comprises a PD-1 inhibitor described herein, capmatinib (INC280), and BLZ945. In some embodiments, the combination comprises PDR001, capmatinib (INC280), and BLZ945.

In some embodiments, the combination comprises PDR001, a c-MET inhibitor described herein, and MCS110. In some embodiments, the combination comprises a PD-1 inhibitor described herein, capmatinib (INC280), and MCS110. In some embodiments, the combination comprises PDR001, capmatinib (INC280), and MCS110.

In certain embodiments, the combination further comprises a fourth therapeutic agent, e.g., a therapeutic agent described herein.

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a pancreatic cancer, a colorectal cancer (CRC) a gastric cancer, or a melanoma, e.g., a refractory melanoma.

### Combination Targeting PD-1 and IDO

In an embodiment, the combination comprises a PD-1 inhibitor (e.g., a PD-1 inhibitor described herein), an IDO inhibitor (e.g., an IDO inhibitor described herein), and a third therapeutic agent.

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the IDO inhibitor is chosen from epacadostat (also known as INCB24360), indoximod (NLG8189), NLG919, or BMS-986205 (formerly F001287). In some embodiments, the combination comprises PDR001 and an IDO inhibitor described herein. In some embodiments, the IDO inhibitor is epacadostat.

In some embodiments, the combination further comprises a TGF-β inhibitor, e.g., a TGF-β inhibitor disclosed herein. In some embodiments, the TGF-β inhibitor is fresolimumab or XOMA 089. In some embodiments, the TGF-β inhibitor comprises XOMA 089. In some embodiments, the combination comprises PDR001, an IDO inhibitor described herein, and a TGF-β inhibitor described herein. In some embodiments, the combination comprises a PD-1 inhibitor described herein, an IDO inhibitor described herein, and XOMA 089. In some embodiments, the combination comprises PDR001, an IDO inhibitor described herein, and XOMA 089.

In some embodiments, the third therapeutic agent comprises an A2aR antagonist, e.g., an A2Ar inhibitor disclosed herein. In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178).

In some embodiments, the combination comprises PDR001, an IDO inhibitor described herein, and an A2aR antagonist described herein. In some embodiments, the combination comprises a PD-1 inhibitor described herein, an IDO inhibitor described herein, and PBF509 (NIR178). In some embodiments, the combination comprises PDR001, an IDO inhibitor described herein, and PBF509 (NIR178).

In some embodiments, the third therapeutic agent comprises a CSF-1/1R binding agent, e.g., a CSF-1/1R binding agent disclosed herein. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK)(e.g., pexidartinib), or an antibody targeting CSF1R (e.g., emactuzumab or FPA008). In some embodiments, the CSF-1/1R binding agent comprises BLZ945. In some embodiments, the CSF-1/1R binding agent comprises MCS110.

In some embodiments, the combination comprises PDR001, an IDO inhibitor described herein, and a CSF-1/1R binding agent described herein. In some embodiments, the combination comprises a PD-1 inhibitor described herein, an IDO inhibitor described herein, and BLZ945. In some embodiments, the combination comprises PDR001, an IDO inhibitor described herein, and BLZ945.

In some embodiments, the combination comprises PDR001, an IDO inhibitor described herein, and a CSF-1/1R binding agent described herein. In some embodiments, the combination comprises a PD-1 inhibitor described herein, an IDO inhibitor described herein, and MCS110. In some embodiments, the combination comprises PDR001, an IDO inhibitor described herein, and MCS110.

In some embodiments, a composition further comprises a c-MET inhibitor, e.g., a c-MET inhibitor disclosed herein. In some embodiments, the c-MET inhibitor is chosen from capmatinib (INC280), JNJ-3887605, AMG 337, LY2801653, MSC2156119J, crizotinib, tivantinib or golvatinib. In some embodiments, the c-MET inhibitor comprises capmatinib (INC280). In some embodiments, the combination comprises PDR001, an IDO inhibitor described herein, and a c-MET inhibitor described herein. In some embodiments, the combination comprises a PD-1 inhibitor described herein, an IDO inhibitor described herein, and capmatinib (INC280). In some embodiments, the combination comprises PDR001, an IDO inhibitor described herein, and capmatinib (INC280). In some embodiments, the c-MET inhibitor (e.g., INC280) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., INC280) is administered twice a day at a dose of about 400 mg. In an embodiment, the c-MET inhibitor (e.g., INC280) is administered twice a day at a dose of about 600 mg.

In some embodiments, the combination further comprises a GITR agonist, e.g., a GITR agonist disclosed herein. In some embodiments, the GITR agonist is chosen from GWN323, BMS-986156, MK-4166, MK-1248, TRX518, INCAGN1876, AMG 228 or INBRX-110. In some embodiments, the GITR agonist is GWN323. In some embodiments, the combination comprises PDR001, an IDO inhibitor described herein, and a GITR agonist described herein. In some embodiments, the combination comprises a PD-1 inhibitor described herein, an IDO inhibitor described herein, and GWN323. In some embodiments, the combination comprises PDR001, an IDO inhibitor described herein, and GWN323.

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a pancreatic cancer, a colorectal cancer (CRC), a gastric cancer, or a melanoma, e.g., a refractory melanoma.

### Combination Targeting PD-1 and TIM-3

In an embodiment, the combination comprises a PD-1 inhibitor, (e.g., a PD-1 inhibitor described herein), a TIM-3 inhibitor (e.g., a TIM-3 inhibitor described herein), and a third therapeutic agent.

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the TIM-3 inhibitor is chosen from MBG453 or TSR-022. In some embodiments, the TIM-3 inhibitor is MBG453.

In some embodiments, the composition comprises PDR001, and a TIM-3 inhibitor described herein, and a third therapeutic agent (e.g., a third therapeutic agent described herein). In some embodiments, the combination comprises a PD-1 inhibitor described herein, MBG453, and a third therapeutic agent (e.g., a third therapeutic agent described herein). In some embodiments, the composition comprises PDR001, MBG453, and a third therapeutic agent (e.g., a third therapeutic agent described herein).

In some embodiments, the third therapeutic agent comprises a CSF-1/1R binding agent, e.g., a CSF-1/1R binding agent disclosed herein. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK)(e.g., pexidartinib), or an antibody targeting CSF1R (e.g., emactuzumab or FPA008). In some embodiments, the CSF-1/1R binding agent comprises BLZ945). In some embodiments, the CSF-1/1R binding agent comprises MCS110.

In some embodiments, the combination comprises PDR001, a TIM-3 inhibitor described herein, and a CSF-1/1R binding agent described herein. In some embodiments, the combination comprises a PD-1 inhibitor described herein, a TIM-3 inhibitor described herein, and BLZ945. In some embodiments, the combination comprises a PD-1 inhibitor described herein, MBG453, and a CSF-1/1R binding agent described herein. In some embodiments, the combination comprises PDR001, MBG453, and a CSF-1/1R binding agent described herein. In some embodiments, the combination comprises PDR001, a TIM-3 inhibitor described herein, and BLZ945. In some embodiments, the combination comprises a PD-1 inhibitor described herein, MBG453, and BLZ945. In some embodiments, the combination comprises PDR001, MBG453, and BLZ945.

In some embodiments, the combination comprises PDR001, a TIM-3 inhibitor described herein, and a CSF-1/1R binding agent described herein. In some embodiments, the combination comprises a PD-1 inhibitor described herein, a TIM-3 inhibitor described herein, and MCS110. In some embodiments, the combination comprises a PD-1 inhibitor described herein, MBG453, and a CSF-1/1R binding agent described herein. In some embodiments, the combination comprises PDR001, MBG453, and a CSF-1/1R binding agent described herein. In some embodiments, the combination comprises PDR001, a TIM-3 inhibitor described herein, and MCS110. In some embodiments, the combination comprises a PD-1 inhibitor described herein, MBG453, and MCS110. In some embodiments, the combination comprises PDR001, MBG453, and MCS110.

In some embodiments, the third therapeutic agent comprises a STING agonist, e.g., a STING agonist described herein. In some embodiments, the STING agonist comprises, MK-1454. In some embodiments, the combination comprises PDR001, a TIM-3 inhibitor described herein, and a STING agonist described herein. In some embodiments, the combination comprises a PD-1 inhibitor described herein, a TIM-3 inhibitor described herein, and MK-1454. In some embodiments, the combination comprises a PD-1 inhibitor described herein, MBG453, and a STING agonist described herein. In some embodiments, the combination comprises PDR001, MBG453, and MK-1454.

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject or cancer is identified as having a biomarker described herein. In some embodiments, the cancer is a solid tumor, e.g., a pancreatic cancer, or a colon cancer.

### Combination Targeting PD-1, TIM-3 and A2aR

In an embodiment, the combination comprises a PD-1 inhibitor, (e.g., a PD-1 inhibitor described herein), a TIM-3 inhibitor (e.g., a TIM-3 inhibitor described herein), and an A2ar antagonist (e.g., an A2aR antagonist described herein).

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the TIM-3 inhibitor is chosen from MBG453 or TSR-022. In some embodiments, the TIM-3 inhibitor is MBG453.

In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178).

In some embodiments, the composition comprises a combination of a PD-1 inhibitor, e.g., PDR001, a TIM-3 inhibitor, e.g., MBG453, and an A2aR antagonist, e.g., PBF509 (NIR178).

In some embodiments, the composition comprises PDR001, a TIM-3 inhibitor described herein, and an A2aR antagonist described herein. In some embodiments, the combination comprises a PD-1 inhibitor described herein, MBG453, and an A2aR antagonist described herein. In some embodiments, the combination comprises a PD-1 inhibitor described herein, a TIM-3 inhibitor described herein, and PBF509 (NIR178). In some embodiments, the composition comprises PDR001, MBG453, and an A2aR antagonist described herein. In some embodiments, the composition comprises PDR001, a TIM-3 inhibitor described herein, and PBF509 (NIR178). In some embodiments, the composition comprises a PD-1 inhibitor described herein, MBG453 and PBF509 (NIR178). In some embodiments, the composition comprises PDR001, MBG453, and PBF509 (NIR178).

In some embodiments, the combination further comprises a CSF-1/1R binding agent, e.g., a CSF-1/1R binding agent disclosed herein. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK)(e.g., pexidartinib), or an antibody targeting CSF1R (e.g., emactuzumab or FPA008). In some embodiments, the CSF-1/1R binding agent comprises BLZ945. In some embodiments, the CSF-1/1R binding agent comprises MCS110.

In some embodiments, the combination comprises PDR001, a TIM-3 inhibitor described herein, an A2aR antagonist described herein, and BLZ945. In some embodiments, the combination comprises a PD-1 inhibitor described herein, MBG453, an A2aR antagonist described herein, and BLZ945. In some embodiments, the combination comprises a PD-1 inhibitor described herein, a TIM-3 inhibitor described herein, PBF509 (NIR178), and BLZ945. In some embodiments, the combination comprises PDR001, MBG453, an A2aR antagonist described herein, and BLZ945. In some embodiments, the combination comprises PDR001, a TIM-3 inhibitor described herein, PBF509 (NIR178), and BLZ945. In some embodiments, the combination comprises a PD-1 inhibitor, MBG453, PBF509 (NIR178), and BLZ945. In some embodiments, the combination comprises PDR001, MBG453, PBF509 (NIR178), and BLZ945.

In some embodiments, the combination comprises PDR001, a TIM-3 inhibitor described herein, an A2aR antagonist described herein, and MCS110. In some embodiments, the combination comprises a PD-1 inhibitor described herein, MBG453, an A2aR antagonist described herein, and MCS110. In some embodiments, the combination comprises a PD-1 inhibitor described herein, a TIM-3 inhibitor described herein, PBF509 (NIR178), and MCS110. In some embodiments, the combination comprises PDR001, MBG453, an A2aR antagonist described herein, and MCS110. In some embodiments, the combination comprises PDR001, a TIM-3 inhibitor described herein, PBF509 (NIR178), and MCS110. In some embodiments, the combination comprises a PD-1 inhibitor, MBG453, PBF509 (NIR178), and MCS110. In some embodiments, the combination comprises PDR001, MBG453, PBF509 (NIR178), and MCS110.

In some embodiments, the combination further comprises a TGF-β inhibitor, e.g., a TGF-β inhibitor disclosed herein. In some embodiments, the TGF-β inhibitor is fresolimumab or XOMA 089. In some embodiments, the TGF-β inhibitor is XOMA 089.

In some embodiments, the combination comprises PDR001, a TIM-3 inhibitor described herein, an A2aR antagonist described herein, and XOMA 089. In some embodiments, the combination comprises a PD-1 inhibitor described herein, MBG453, an A2aR antagonist described herein, and XOMA 089. In some embodiments, the combination comprises a PD-1 inhibitor described herein, a TIM-3 inhibitor described herein, PBF509 (NIR178), and XOMA 089.

In some embodiments, the combination comprises PDR001, MBG453, an A2aR antagonist described herein, and XOMA 089. In some embodiments, the combination comprises PDR001, a TIM-3 inhibitor described herein, PBF509 (NIR178), and XOMA 089. In some embodiments, the combination comprises a PD-1 inhibitor, MBG453, PBF509 (NIR178), and XOMA 089. In some embodiments, the combination comprises PDR001, MBG453, PBF509 (NIR178), and XOMA 089.

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a pancreatic cancer, or a colon cancer.

### Combination Targeting IL-1/3 and A2aR

In an embodiment, the combination comprises an IL-1β inhibitor (e.g., an IL-1β inhibitor described herein), and an A2aR antagonist (e.g., an A2aR antagonist described herein). In an embodiment, the combination further comprises and an additional therapeutic agent, e.g., one or more additional therapeutic agents (e.g., a third therapeutic agent, or a third and a fourth therapeutic agent).

In some embodiments, the IL-1β inhibitor is chosen from canakinumab, gevokizumab, Anakinra, or Rilonacept.

In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178).

In some embodiments, the combination comprises an IL-1β inhibitor, e.g., canakinumab, gevokizumab, Anakinra, or Rilonacept, and an A2aR antagonist, e.g., PBF509 (NIR178). In some embodiments, the combination comprises an IL-1b inhibitor, e.g., canakinumab, gevokizumab, Anakinra, or Rilonacept, and PBF509 (NIR178).

In some embodiments, the combination comprises a third therapeutic agent. In some embodiments, the third therapeutic agent comprises an IL-15/IL-15Ra complex, e.g., an IL-15/IL-15Ra complex described herein. In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. In some embodiments, the combination comprises an IL-1b inhibitor, e.g., canakinumab, gevokizumab, Anakinra, or Rilonacept, an A2aR antagonist, e.g., PBF509 (NIR178), and an IL-15/IL-15Ra complex, e.g., NIZ985. In some embodiments, the combination comprises an IL-1β inhibitor, e.g., canakinumab, gevokizumab, Anakinra, or Rilonacept, PBF509 (NIR178), and an IL-15/IL-15Ra complex, e.g., NIZ985. In some embodiments, the combination comprises an IL-1b inhibitor, e.g., canakinumab, gevokizumab, Anakinra, or Rilonacept, PBF509 (NIR178), and NIZ985.

In some embodiments, the combination further comprises a fourth therapeutic agent. In some embodiments, the fourth therapeutic agent comprises a TGF-β inhibitor, e.g., a TGF-β inhibitor described herein. In some embodiments, the TGF-β inhibitor is fresolimumab or XOMA 089. In some embodiments, the TGF-β inhibitor is XOMA 089. In some embodiments, the combination comprises PDR001, a LAG-3 inhibitor described herein, and XOMA 089. In some embodiments, the combination comprises a PD-1 inhibitor described herein, LAG525 and XOMA 089. In some embodiments, the combination comprises PDR001, LAG525, and XOMA 089.

In some embodiments, the combination comprises an IL-1b inhibitor, e.g., canakinumab, gevokizumab, Anakinra, or Rilonacept, an A2aR antagonist, e.g., PBF509 (NIR178), an IL-15/IL-15Ra complex, e.g., NIZ985, and a TGF-β inhibitor, e.g., XOMA 089. In some embodiments, the combination comprises an IL-1β inhibitor, e.g., canakinumab, gevokizumab, Anakinra, or Rilonacept, PBF509 (NIR178), an IL-15/IL-15Ra complex, e.g., NIZ985, and a TGF-β inhibitor, e.g., XOMA 089. In some embodiments, the combination comprises an IL-1β inhibitor, e.g., canakinumab, gevokizumab, Anakinra, or Rilonacept, PBF509 (NIR178), NIZ985, and a TGF-β inhibitor, e.g., XOMA 089. In some embodiments, the combination comprises an IL-1β inhibitor, e.g., canakinumab, gevokizumab, Anakinra, or Rilonacept, PBF509 (NIR178), NIZ985, and XOMA 089.

In some embodiments, the combination comprises an IL-1β inhibitor (e.g., an IL-1β inhibitor described herein), an A2aR antagonist (e.g., an A2aR antagonist described herein), and one or both of an IL-15/IL-15Ra complex (e.g., and IL-15/IL-15Ra complex described herein) or a TGF-β inhibitor (e.g., a TGF-β inhibitor described herein).

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a colorectal cancer (CRC), a gastroesophageal cancer or a pancreatic cancer. In some embodiments, the CRC is a microsatellite stable CRC (MSS CRC).

### Combination Targeting IL-15/IL15Ra and TGF-β

In an embodiment, the combination comprises an IL-15/IL-15Ra complex, e.g., an IL-15/IL-15Ra complex described herein, and a TGF-β inhibitor, e.g., a TGF-β inhibitor described herein. In an embodiment, the combination further comprises additional therapeutic agents, e.g., one or two additional therapeutic agents, e.g., therapeutic agents described herein.

In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. In some embodiments, the combination comprises an IL-1b inhibitor, e.g., canakinumab, gevokizumab, Anakinra, or Rilonacept, an A2aR antagonist, e.g., PBF509 (NIR178), and an IL-15/IL-15Ra complex, e.g., NIZ985.

In some embodiments, the TGF-β inhibitor is fresolimumab or XOMA 089. In some embodiments, the TGF-β inhibitor is XOMA 089. In some embodiments, the combination comprises PDR001, a LAG-3 inhibitor described herein, and XOMA 089. In some embodiments, the combination comprises a PD-1 inhibitor described herein, LAG525 and XOMA 089. In some embodiments, the combination comprises PDR001, LAG525, and XOMA 089.

In some embodiments, the combination comprises an IL-15/IL-15Ra complex (e.g., NIZ985), and a TGF-β inhibitor (e.g., XOMA 089). In some embodiments, the combination comprises NIZ985, and a TGF-β inhibitor (e.g., XOMA 089). In some embodiments, the combination comprises NIZ985, and XOMA 089.

In some embodiments, the combination comprising an IL-15/IL-15Ra complex, e.g., an IL-15/IL-15Ra complex described herein, and a TGF-β inhibitor, e.g., a TGF-β inhibitor described herein, further comprises one or more, e.g., two, therapeutic agents. In some embodiments, the combination comprises an IL-1b inhibitor, e.g., an IL-1b inhibitor described herein, and a CSF-1/1R binding agent, e.g., a CSF-1/1R binding agent described herein.

In some embodiments, the IL-1b inhibitor is chosen from canakinumab, gevokizumab, Anakinra, or Rilonacept.

In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110.

In some embodiments, the combination comprises an IL-15/IL-15Ra complex (e.g., NIZ985), a TGF-β inhibitor (e.g., XOMA 089), an IL-1b inhibitor (e.g., canakinumab, gevokizumab, Anakinra, or Rilonacept), and a CSF-1/1R binding agent (e.g., MCS110 or BLZ495). In some embodiments, the combination comprises NIZ985, a TGF-β inhibitor (e.g., XOMA 089), an IL-1b inhibitor (e.g., canakinumab, gevokizumab, Anakinra, or Rilonacept), and a CSF-1/1R binding agent (e.g., MCS110 or BLZ495). In some embodiments, the combination comprises NIZ985, XOMA 089, an IL-1b inhibitor (e.g., canakinumab, gevokizumab, Anakinra, or Rilonacept), and a CSF-1/1R binding agent (e.g., MCS110 or BLZ495). In some embodiments, the combination comprises NIZ985, XOMA 089, an IL-1b inhibitor (e.g., canakinumab, gevokizumab, Anakinra, or Rilonacept), and MCS110. In some embodiments, the combination comprises NIZ985, XOMA 089, an IL-1b inhibitor (e.g., canakinumab, gevokizumab, Anakinra, or Rilonacept), and BLZ495.

In some embodiments, the combination comprising an IL-15/IL-15Ra complex, e.g., an IL-15/IL-15Ra complex described herein, and a TGF-β inhibitor, e.g., a TGF-β inhibitor described herein, further comprises one or more, e.g., two, therapeutic agents. In some embodiments, the combination comprises an A2aR antagonist, e.g., A2aR antagonist described herein, and a c-MET inhibitor, e.g., a c-MET inhibitor described herein.

In some embodiments, the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814. In some embodiments, the A2aR antagonist is PBF509 (NIR178).

In some embodiments, the c-MET inhibitor is chosen from capmatinib (INC280), JNJ-3887605, AMG 337, LY2801653, MSC2156119J, crizotinib, tivantinib, or golvatinib. In some embodiments, the c-MET inhibitor is capmatinib (INC280).

In some embodiments, the combination comprises an IL-15/IL-15Ra complex (e.g., NIZ985), a TGF-β inhibitor (e.g., XOMA 089), an A2aR antagonist (e.g., PBF509 (NIR178)), and a c-MET inhibitor (e.g., capmatinib). In some embodiments, the combination comprises NIZ985, a TGF-β inhibitor (e.g., XOMA 089), an A2aR antagonist (e.g., PBF509 (NIR178)), and a c-MET inhibitor (e.g., capmatinib). In some embodiments, the combination comprises NIZ985, XOMA 089, an A2aR antagonist (e.g., PBF509 (NIR178)), and a c-MET inhibitor (e.g., capmatinib). In some embodiments, the combination comprises NIZ985, XOMA 089, PBF509 (NIR178), and a c-MET inhibitor (e.g., capmatinib). In some embodiments, the combination comprises NIZ985, XOMA 089, an A2aR antagonist (e.g., PBF509 (NIR178)), and capmatinib. In some embodiments, the combination comprises NIZ985, XOMA 089, PBF509 (NIR178), and capmatinib.

In some embodiments, the combination comprises an IL-15/IL-15Ra complex (e.g., and IL-15/IL-15Ra complex described herein), a TGF-β inhibitor (e.g. a TGF-β inhibitor described herein), and one or more of (e.g., two, three, or more) of an IL-1β inhibitor (e.g., an IL-1β inhibitor described herein), a CSF-1/1R binding agent (e.g., a CSF-1/1R binding agent described herein), a c-MET inhibitor (e.g., a c-MET inhibitor described herein), or an A2aR antagonist (e.g., an A2aR antagonist described herein).

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor, e.g., a colorectal cancer (CRC), a gastroesophageal cancer or a pancreatic cancer. In some embodiments, the CRC is a microsatellite stable CRC (MSS CRC).

### Combination Targeting Galectin and Other Molecules

In some embodiments, the combination comprises a Galectin (e.g., Galectin-1 or Galectin-3) inhibitor, e.g., a Galectin (e.g., Galectin-1 or Galectin-3) inhibitor described herein. In some embodiments, the combination comprises a Galectin (e.g., Galectin-1 or Galectin-3) inhibitor, e.g., a Galectin (e.g., Galectin-1 or Galectin-3) inhibitor described herein, and an additional therapeutic agent, e.g., one or more therapeutic agents described herein. In some embodiments, the combination comprises a Galectin (e.g., Galectin-1 or Galectin-3) inhibitor, e.g., a Galectin (e.g., Galectin-1 or Galectin-3) inhibitor described herein, and a PD-1 inhibitor, e.g., a PD-1 inhibitor described herein.

In some embodiments, the combination comprises a Galectin-1 inhibitor (e.g., an anti-Galectin-1 antibody molecule) and a Galectin-3 inhibitor (e.g., an anti-Galectin-3 antibody molecule). The combination of antibody molecules can be administered separately, e.g., as separate antibody molecules, or linked, e.g., as a multispecific (e.g., bispecific) antibody molecule. In one embodiment, a bispecific antibody molecule that comprises an anti-Galectin-1 antibody molecule and an anti-Galectin-3 antibody molecule is administered. In some embodiments, the bispecific antibody molecule comprises an antigen-binding fragment of an anti-Galectin-1 antibody and an antigen-binding fragment of an anti-Galectin-3 antibody. In certain embodiments, the combination is used to treat a cancer, e.g., a cancer as described herein (e.g., a solid tumor or a hematologic malignancy).

In some embodiments, the Galectin, e.g., Galectin-1 or Galectin-3, inhibitor is chosen from an anti-Galectin (e.g., anti-Galectin-1 or anti-Galectin-3) antibody molecule, GR-MD-02, Galectin-3C, Anginex, or OTX-008. In some embodiments, the Galectin inhibitor is an anti Galectin (e.g., anti-Galectin-1 or anti-Galectin-3) antibody molecule, e.g., a monospecific or multispecific (e.g., bispecific) antibody molecule. In an embodiment, the Galectin inhibitor is a monospecific antibody molecule. In some embodiments, the Galectin inhibitor is an anti-Galectin-1 antibody, e.g., a monospecific antibody against Galectin-1. In some embodiments, the Galectin inhibitor is an anti-Galectin-3 antibody, e.g., a monospecific antibody against Galectin-3.

In some embodiments, the composition comprises a combination of a Galectin inhibitor, e.g., an anti-Galectin-1 monospecific antibody molecule, and an additional Galectin inhibitor, e.g., an anti-Galectin-3 monospecific antibody molecule.

In some embodiments, the combination comprises a Galectin (e.g., Galectin-1 or Galectin-3) inhibitor, e.g., a Galectin (e.g., Galectin-1 or Galectin-3) monospecific antibody molecule, and a PD-1 inhibitor, e.g., a PD-1 inhibitor described herein.

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In embodiments, the PD-1 inhibitor is administered once every 3 weeks. In embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the composition comprises a combination of a Galectin inhibitor, e.g., an anti-Galectin-1 monospecific antibody molecule, and a PD-1 inhibitor, e.g., PDR001. In some embodiments, the composition comprises a combination of a Galectin inhibitor, e.g., an anti-Galectin-3 monospecific antibody molecule, and a PD-1 inhibitor, e.g., PDR001.

In some embodiments, the combination comprises a Galectin (e.g., Galectin-1 or Galectin-3) inhibitor, e.g., a Galectin (e.g., Galectin-1 or Galectin-3) bispecific antibody molecule, and a PD-1 inhibitor, e.g., a PD-1 inhibitor described herein.

In an embodiment, the Galectin inhibitor is a bispecific antibody molecule. In an embodiment, the first epitope of the anti-Galectin bispecific antibody molecule is located on Galectin-1, and the second epitope of the anti-Galectin bispecific antibody molecule is located on Galectin-3.

In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is administered at a dose of about 300-400 mg. In some embodiments, the PD-1 inhibitor is PDR001. In some embodiments, the PD-1 inhibitor is administered once every 3 weeks. In some embodiments, the PD-1 inhibitor is administered once every 4 weeks. In other embodiments, the PD-1 inhibitor is administered at a dose of about 300 mg once every 3 weeks. In yet other embodiments, the PD-1 inhibitor is administered at a dose of about 400 mg once every 4 weeks.

In some embodiments, the composition comprises a combination of a Galectin inhibitor, e.g., an anti-Galectin-1 and anti-Galectin-3 bispecific antibody molecule, and a PD-1 inhibitor, e.g., PDR001.

In some embodiments, the combination is administered or used in a therapeutically effective amount (e.g., in accordance with a dosage regimen described herein) to treat a disorder (e.g., a cancer, e.g., a cancer described herein) in a subject in need thereof. In some embodiments, the subject has cancer or is identified as having a biomarker described herein. In some embodiments, the cancer is a solid tumor or a hematological malignancy.

### Uses of the Combination Therapies

The combinations disclosed herein can result in one or more of: an increase in antigen presentation, an increase in effector cell function (e.g., one or more of T cell proliferation, IFN-γ secretion or cytolytic function), inhibition of regulatory T cell function, an effect on the activity of multiple cell types (e.g., regulatory T cell, effector T cells and NK cells), an increase in tumor infiltrating lymphocytes, an increase in T-cell receptor mediated proliferation, a decrease in immune evasion by cancerous cells, and a decrease in oncogenic activity (e.g., overexpression of an oncogene). In one embodiment, the use of a PD-1 inhibitor in the combinations inhibits, reduces or neutralizes one or more activities of PD-1, resulting in blockade or reduction of an immune checkpoint. Thus, such combinations can be used to treat or prevent disorders where enhancing an immune response in a subject is desired.

Accordingly, in another aspect, a method of modulating an immune response in a subject is provided. The method comprises administering to the subject a combination disclosed herein (e.g., a combination comprising a therapeutically effective amount of a PD-1 inhibitor described herein), alone or in combination with one or more agents or procedures, such that the immune response in the subject is modulated. In one embodiment, the antibody molecule enhances, stimulates, restores, or increases the immune response in the subject. The subject can be a mammal, e.g., a primate, preferably a higher primate, e.g., a human (e.g., a patient having, or at risk of having, a disorder described herein). In one embodiment, the subject is in need of enhancing an immune response. In one embodiment, the subject has, or is at risk of, having a disorder described herein, e.g., a cancer or an infectious disorder as described herein. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

In one aspect, a method of treating (e.g., one or more of reducing, inhibiting, or delaying progression) a cancer or a tumor in a subject is provided. The method comprises administering to the subject a combination disclosed herein (e.g., e.g., a combination comprising a therapeutically effective amount of a PD-1 inhibitor described herein).

In certain embodiments, the cancer treated with the combination, includes but is not limited to, a solid tumor, a hematological cancer (e.g., leukemia, lymphoma, myeloma, e.g., multiple myeloma), and a metastatic lesion. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal cancer (e.g., renal-cell carcinoma (clear cell or non-clear cell renal cell carcinoma), liver cancer, lung cancer (e.g., non-small cell lung cancer (squamous or non-squamous non-small cell lung cancer)), cancer of the small intestine and cancer of the esophagus. The cancer may be at an early, intermediate, late stage or metastatic cancer.

In some embodiments, the cancer is chosen from a breast cancer, a pancreatic cancer, a colorectal cancer, a skin cancer, or a gastric cancer. In some embodiments, the cancer is an ER+ cancer (e.g., an ER+ breast cancer). In some embodiments, the cancer is a breast cancer. In some embodiments, the cancer is a pancreatic cancer. In some embodiments, the cancer is a colorectal cancer. In some embodiments, the cancer is a skin cancer (e.g., a melanoma, e.g., a refractory melanoma). In some embodiments, the cancer is a gastric cancer.

In some embodiments, the cancer is an advanced cancer. In some embodiments, the cancer is a metastatic cancer. In some embodiments, the cancer is a relapsed cancer. In some embodiments, the cancer is a refractory cancer. In some embodiments, the cancer is a recurrent cancer. In some embodiments, the cancer is an unresectable cancer.

In some embodiments, the cancer is a microsatellite instability-high (MSI-H) cancer. In some embodiments, the cancer is a mismatch repair deficient (dMMR) cancer.

In some embodiments, the cancer (e.g., cancer cells, cancer microenvironment, or both) has an elevated level of PD-L1 expression. Alternatively, or in combination, the cancer (e.g., cancer cells, cancer microenvironment, or both) can have increased IFNγ and/or CD8 expression.

In some embodiments, the subject has, or is identified as having, a cancer that has one or more of high PD-L1 level or expression, or as being tumor infiltrating lymphocyte (TIL)+ (e.g., as having an increased number of TILs), or both. In certain embodiments, the subject has, or is identified as having, a cancer that has high PD-L1 level or expression and that is TIL+. In some embodiments, the method described herein further includes identifying a subject based on having a cancer that has one or more of high PD-L1 level or expression, or as being TIL+, or both. In certain embodiments, the method described herein further includes identifying a subject based on having a cancer that has high PD-L1 level or expression and as being TIL+. In some embodiments, a cancer that is TIL+ is positive for CD8 and IFNγ. In some embodiments, the subject has, or is identified as having, a high percentage of cells that are positive for one, two or more of PD-L1, CD8, or IFNγ. In certain embodiments, the subject has, or is identified as having, a high percentage of cells that are positive for all of PD-L1, CD8, and IFNγ.

In some embodiments, the methods described herein further includes identifying a subject based on having a high percentage of cells that are positive for one, two or more of PD-L1, CD8, and/or IFNγ. In certain embodiments, the methods described herein further includes identifying a subject based on having a high percentage of cells that are positive for all of PD-L1, CD8, and IFNγ. In some embodiments, the subject has, or is identified as having, one, two or more of PD-L1, CD8, and/or IFNγ, and one or more of a breast cancer, a pancreatic cancer, a colorectal cancer, a skin cancer, a gastric cancer, or an ER+ cancer. In certain embodiments, the method described herein further includes identifying a subject based on having one, two or more of PD-L1, CD8, and/or IFNγ, and one or more of a breast cancer, a pancreatic cancer, a colorectal cancer, a skin cancer, a gastric cancer, or an ER+ cancer).

In some embodiments, the subject has, or is identified as having, a cancer that expresses one or more (e.g., two, three, four, or more) of PD-1, LAG-3, TIM-3, GITR, estrogen receptor (ER), CDK4, CDK6, CXCR2, CSF1, CSF1R, c-MET, TGF-β, A2Ar, IDO, STING or Galectin, e.g., Galectin-1 or Galectin-3.

Methods and compositions disclosed herein are useful for treating metastatic lesions associated with the aforementioned cancers.

In a further aspect, the invention provides a method of treating an infectious disease in a subject, comprising administering to a subject a combination as described herein, e.g., a combination comprising a therapeutically effective amount of a PD-1 inhibitor described herein. In one embodiment, the infection disease is chosen from hepatitis (e.g., hepatitis C infection), or sepsis.

Still further, the invention provides a method of enhancing an immune response to an antigen in a subject, comprising administering to the subject: (i) the antigen; and (ii) a combination as described herein, e.g., a combination comprising a therapeutically effective amount of a PD-1 inhibitor described herein, such that an immune response to the antigen in the subject is enhanced. The antigen can be, for example, a tumor antigen, a viral antigen, a bacterial antigen or an antigen from a pathogen.

The combinations as described herein can be administered to the subject systemically (e.g., orally, parenterally, subcutaneously, intravenously, rectally, intramuscularly, intraperitoneally, intranasally, transdermally, or by inhalation or intracavitary installation), topically, or by application to mucous membranes, such as the nose, throat and bronchial tubes.

Dosages and therapeutic regimens of the therapeutic agents disclosed herein can be determined. In some embodiments, the PD-1 inhibitor is administered by injection (e.g., subcutaneously or intravenously) at a dose (e.g., a flat dose) of about 100 mg to 600 mg, e.g., about 200 mg to 500 mg, e.g., about 250 mg to 450 mg, about 300 mg to 400 mg, about 250 mg to 350 mg, about 350 mg to 450 mg, or about 100 mg, about 200 mg, about 300 mg, or about 400 mg. The dosing schedule (e.g., flat dosing schedule) can vary from e.g., once a week to once every 2, 3, 4, 5, or 6 weeks. In one embodiment, the PD-1 inhibitor is administered at a dose from about 300 mg to 400 mg once every three weeks or once every four weeks. In one embodiment, the PD-1 inhibitor is administered at a dose from about 300 mg once every three weeks. In one embodiment, the PD-1 inhibitor is administered at a dose from about 400 mg once every four weeks. In one embodiment, the PD-1 inhibitor is administered at a dose from about 300 mg once every four weeks. In one embodiment, the PD-1 inhibitor is administered at a dose from about 400 mg once every three weeks.

In certain embodiments, the PD-1 inhibitor is administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks. In one embodiment, the PD-1 inhibitor is administered at a dose from about 10 to 20 mg/kg every other week.

### Biomarkers

In certain embodiments, any of the methods disclosed herein further includes evaluating or monitoring the effectiveness of a therapy (e.g., a monotherapy or a combination therapy) described herein, in a subject (e.g., a subject having a cancer, e.g., a cancer described herein). The method includes acquiring a value of effectiveness to the therapy, wherein said value is indicative of the effectiveness of the therapy.

In embodiments, the value of effectiveness to the therapy comprises a measure of one, two, three, four, five, six, seven, eight, nine or more (e.g., all) of the following:

(i) a parameter of a tumor infiltrating lymphocyte (TIL) phenotype;

(ii) a parameter of a myeloid cell population;

(iii) a parameter of a surface expression marker;

(iv) a parameter of a biomarker of an immunologic response;

(v) a parameter of a systemic cytokine modulation;

(vi) a parameter of circulating free DNA (cfDNA);

(vii) a parameter of systemic immune-modulation;

(viii) a parameter of microbiome;

(ix) a parameter of a marker of activation in a circulating immune cell; or

(x) a parameter of a circulating cytokine.

In some embodiments, the parameter of a TIL phenotype comprises the level or activity of one, two, three, four or more (e.g., all) of Hematoxylin and eosin (H&E) staining for TIL counts, CD8, FOXP3, CD4, or CD3, in the subject, e.g., in a sample from the subject (e.g., a tumor sample).

In some embodiments, the parameter of a myeloid cell population comprises the level or activity of one or both of CD68 or CD163, in the subject, e.g., in a sample from the subject (e.g., a tumor sample).

In some embodiments, the parameter of a surface expression marker comprises the level or activity of one or more (e.g., two, three, four, or all) of PD-1, PD-L1, LAG-3, TIM-3, or GITR, in the subject, e.g., in a sample from the subject (e.g., a tumor sample). In certain embodiments, the level of PD-1, PD-L1, LAG-3, TIM-3, or GITR is determined by immunohistochemistry (IHC).

In some embodiments, the parameter of a biomarker of an immunologic response comprises the level or sequence of one or more nucleic acid-based markers, in the subject, e.g., in a sample from the subject (e.g., a tumor sample).

In some embodiments, the parameter of systemic cytokine modulation comprises the level or activity of one, two, three, four, five, six, seven, eight, or more (e.g., all) of IL-18, IFN-γ, ITAC (CXCL11), IL-6, IL-10, IL-4, IL-17, IL-15, or TGF-beta, in the subject, e.g., in a sample from the subject (e.g., a blood sample, e.g., a plasma sample).

In some embodiments, the parameter of cfDNA comprises the sequence or level of one or more circulating tumor DNA (cfDNA) molecules (e.g., tumor mutation burden), in the subject, e.g., in a sample from the subject (e.g., a blood sample, e.g., a plasma sample).

In some embodiments, the parameter of systemic immune-modulation comprises phenotypic characterization of an activated immune cell, e.g., a CD3-expressing cell, a CD8-expressing cell, or both, in the subject, e.g., in a sample from the subject (e.g., a blood sample, e.g., a PBMC sample).

In some embodiments, the parameter of microbiome comprises the sequence or expression level of one or more genes in the microbiome, in the subject, e.g., in a sample from the subject (e.g., a stool sample).

In some embodiments, the parameter of a marker of activation in a circulating immune cell comprises the level or activity of one, two, three, four, five or more (e.g., all) of circulating CD8+, HLA-DR+Ki67+, T cells, IFN-γ, IL-18, or CXCL11 (IFN-γ induced CCK) expressing cells, in a sample (e.g., a blood sample, e.g., a plasma sample).

In some embodiments, the parameter of a circulating cytokine comprises the level or activity of IL-6, in the subject, e.g., in a sample from the subject (e.g., a blood sample, e.g., a plasma sample).

In some embodiments of any of the methods disclosed herein, the therapy comprises a combination described herein (e.g., a combination comprising a therapeutically effective amount of a PD-1 inhibitor described herein).

In some embodiments of any of the methods disclosed herein, the measure of one or more of (i)-(x) is obtained from a sample acquired from the subject. In some embodiments, the sample is chosen from a tumor sample, a blood sample (e.g., a plasma sample or a PBMC sample), or a stool sample.

In some embodiments of any of the methods disclosed herein, the subject is evaluated prior to receiving, during, or after receiving, the therapy.

In some embodiments of any of the methods disclosed herein, the measure of one or more of (i)-(x) evaluates a profile for one or more of gene expression, flow cytometry or protein expression.

In some embodiments of any of the methods disclosed herein, the presence of an increased level or activity of one, two, three, four, five, or more (e.g., all) of circulating CD8+, HLA-DR+Ki67+, T cells, IFN-γ, IL-18, or CXCL11 (IFN-γ induced CCK) expressing cells, and/or the presence of an decreased level or activity of IL-6, in the subject or sample, is a positive predictor of the effectiveness of the therapy.

Alternatively, or in combination with the methods disclosed herein, responsive to said value, performing one, two, three, four or more (e.g., all) of:

(i) administering to the subject the therapy;

(ii) administered an altered dosing of the therapy;

(iii) altering the schedule or time course of the therapy;

(iv) administering to the subject an additional agent (e.g., a therapeutic agent described herein) in combination with the therapy; or

(v) administering to the subject an alternative therapy.

### BRIEF DESCRIPTION OF DRAWINGS

FIG. 1 shows a Western blot of cell lysates from four MC38 cell lines (A-D) probed with an antibody against Galectin-3 or an antibody against Galectin-1. Sample A represents wild type MC38 cells; (B) represents Galectin-3 deleted MC38 cells, (C) represents Galectin-1 deleted MC38 cells, and (D) represents MC38 cells in which both Galectin-1 and Galectin-3 have been deleted.

FIG. 2 depicts flow cytometry analysis of tumors derived from MC38 derived cells lines A-D that were implanted in immunocompetent mice. Tumor cells were dissociated and stained with an anti-CD45 antibody.

FIG. 3 shows a graph of mean tumor volume of tumors generated from MC38 derived cells lines A-D in immunocompetent mice. The graph depicts mean tumor volume (y-axis) as a function of time post-implant in days (x-axis).

FIGS. 4A-4B depict graphs of IL-2 production from the SEB assay with samples from donor E411. FIG. 4A shows a graph of Group 1 parameters tested which include a fixed dose of PDR001 and/or LAG525 with titrations of GWN323. FIG. 4B shows a graph of Group 2 parameters tested which include a fixed dose of PDR001 and/or GWN323 with titrations of LAG525.

FIGS. 5A-5B depict graphs of IL-2 production from the SEB assay with samples from donor E490. FIG. 5A shows a graph of Group 1 parameters tested which include a fixed dose of PDR001 and/or LAG525 with titrations of GWN323. FIG. 5B shows a graph of Group 2 parameters tested which include a fixed dose of PDR001 and/or GWN323 with titrations of LAG525.

FIGS. 6A-6B depict graphs of IL-2 production from the SEB assay with samples from donor 1876. FIG. 6A shows a graph of Group 1 parameters tested which include a fixed dose of PDR001 and/or LAG525 with titrations of GWN323. FIG. 6B shows a graph of Group 2 parameters tested which include a fixed dose of PDR001 and/or GWN323 with titrations of LAG525.

FIG. 7 depicts PD-L1 expression in F480+ and F480− cells harvested from MC38 tumors implanted in mice treated with vehicle control, BLZ 945 and isotype control, vehicle and an anti-TIM3 antibody (5D12), or BLZ945 and an anti-TIM3 antibody (5D12).

FIGS. 8A-8B demonstrate TIM-3 expression in CD103+ dendritic cells from colon carcinoma infiltrates obtained from WT or TIM-3 KO mice. FIG. 8A shows dot-plots of TIM-3 expression in CD103+/− cells from TIM-3 WT mice, and TIM-3 expression in CD103+ cells from TIM-3 KO mice. FIG. 8B shows the quantity of infiltrated CD103+ cells per cm3 tumor in colon carcinomas harvested from TIM-3 WT or TIM-3 KO mice.

### DETAILED DESCRIPTION

### Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

By “combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In embodiments, the additional therapeutic agent is administered at a therapeutic or lower-than therapeutic dose. In certain embodiments, the concentration of the second therapeutic agent that is required to achieve inhibition, e.g., growth inhibition, is lower when the second therapeutic agent is administered in combination with the first therapeutic agent, e.g., the anti-PD-1 antibody molecule, than when the second therapeutic agent is administered individually. In certain embodiments, the concentration of the first therapeutic agent that is required to achieve inhibition, e.g., growth inhibition, is lower when the first therapeutic agent is administered in combination with the second therapeutic agent than when the first therapeutic agent is administered individually. In certain embodiments, in a combination therapy, the concentration of the second therapeutic agent that is required to achieve inhibition, e.g., growth inhibition, is lower than the therapeutic dose of the second therapeutic agent as a monotherapy, e.g., 10-20%, 20-30%, 30-40%, 40-50%, 50-60%, 60-70%, 70-80%, or 80-90% lower. In certain embodiments, in a combination therapy, the concentration of the first therapeutic agent that is required to achieve inhibition, e.g., growth inhibition, is lower than the therapeutic dose of the first therapeutic agent as a monotherapy, e.g., 10-20%, 20-30%, 30-40%, 40-50%, 50-60%, 60-70%, 70-80%, or 80-90% lower.

The term “inhibition,” “inhibitor,” or “antagonist” includes a reduction in a certain parameter, e.g., an activity, of a given molecule, e.g., an immune checkpoint inhibitor. For example, inhibition of an activity, e.g., an activity of a given molecule, e.g., an inhibitory molecule, of at least 5%, 10%, 20%, 30%, 40% or more is included by this term. Thus, inhibition need not be 100%.

A “fusion protein” and a “fusion polypeptide” refer to a polypeptide having at least two portions covalently linked together, where each of the portions is a polypeptide having a different property. The property may be a biological property, such as activity in vitro or in vivo. The property can also be simple chemical or physical property, such as binding to a target molecule, catalysis of a reaction, etc. The two portions can be linked directly by a single peptide bond or through a peptide linker, but are in reading frame with each other.

The term “activation,” “activator,” or “agonist” includes an increase in a certain parameter, e.g., an activity, of a given molecule, e.g., a costimulatory molecule. For example, increase of an activity, e.g., a costimulatory activity, of at least 5%, 10%, 25%, 50%, 75% or more is included by this term.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors. The term “cancer” as used herein includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

As used herein, the terms “treat,” “treatment” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The compositions and methods of the present invention encompass polypeptides and nucleic acids having the sequences specified, or sequences substantially identical or similar thereto, e.g., sequences at least 85%, 90%, 95%, 96%, 97%, 98%, 99% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### Antibody Molecules

In one embodiment, a combination described herein comprises a therapeutic agent which is an antibody molecule.

As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies of the present invention can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the PD-1 polypeptide, or an epitope thereof. With respect to proteins (or protein mimetics), the antigen-binding site typically includes one or more loops (of at least four amino acids or amino acid mimics) that form an interface that binds to the PD-1 polypeptide. Typically, the antigen-binding site of an antibody molecule includes at least one or two CDRs and/or hypervariable loops, or more typically at least three, four, five or six CDRs and/or hypervariable loops.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody molecule can be a polyclonal or a monoclonal antibody. In other embodiments, the antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to PD-1. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

An antibody molecules may be conjugated to another molecular entity, typically a label or a therapeutic (e.g., a cytotoxic or cytostatic) agent or moiety. Radioactive isotopes can be used in diagnostic or therapeutic applications. Radioactive isotopes that can be coupled to the anti-PSMA antibodies include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi ²¹³Bi), indium (¹¹¹In), technetium (⁹⁹ mTc), phosphorus (³²P), rhodium (¹⁸⁸Rh), sulfur (35S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or 213Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

The invention provides radiolabeled antibody molecules and methods of labeling the same. In one embodiment, a method of labeling an antibody molecule is disclosed. The method includes contacting an antibody molecule, with a chelating agent, to thereby produce a conjugated antibody. The conjugated antibody is radiolabeled with a radioisotope, e.g., 111Indium, 90Yttrium and 177Lutetium, to thereby produce a labeled antibody molecule.

As is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

### Multispecific Antibody Molecules

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, the Galectin inhibitor is a multispecific antibody molecule. In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope. In an embodiment, the Galectin inhibitor is a bispecific antibody molecule. In an embodiment, the first epitope is located on Galectin-1, and the second epitope is located on Galectin-3.

Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, U52003/211078A1, U52004/219643A1, U52004/220388A1, US2004/242847A1, US2005/003403A1, US2005/004352A1, US2005/069552A1, U52005/079170A1, U52005/100543A1, US2005/136049A1, U52005/136051A1, US2005/163782A1, US2005/266425A1, US2006/083747A1, U52006/120960A1, US2006/204493A1, US2006/263367A1, US2007/004909A1, U52007/087381A1, U52007/128150A1, US2007/141049A1, US2007/154901A1, US2007/274985A1, US2008/050370A1, US2008/069820A1, US2008/152645A1, U52008/171855A1, U52008/241884A1, U52008/254512A1, US2008/260738A1, US2009/130106A1, US2009/148905A1, US2009/155275A1, US2009/162359A1, US2009/162360A1, U52009/175851A1, US2009/175867A1, U52009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1. The contents of the above-referenced applications are incorporated herein by reference in their entireties.

In other embodiments, the anti-Galectin, e.g., anti-Galectin-1 or anti-Galectin-3, antibody molecule (e.g., a monospecific, bispecific, or multispecific antibody molecule) is covalently linked, e.g., fused, to another partner e.g., a protein, e.g., as a fusion molecule for example a fusion protein. In one embodiment, a bispecific antibody molecule has a first binding specificity to a first target (e.g., to Galectin-1), a second binding specificity to a second target (e.g., Galectin-3).

This invention provides an isolated nucleic acid molecule encoding the above antibody molecule, vectors and host cells thereof. The nucleic acid molecule includes but is not limited to RNA, genomic DNA and cDNA.

### Therapeutic Agents

### PD-1 Inhibitors

In certain embodiments, a combination described herein comprises a PD-1 inhibitor. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. PDR001 is also known as Spartalizumab.

Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In some embodiments, the anti-PD-1 antibody molecule is Spartalizumab (PDR001).

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of BAP049-Clone-E or BAP049-Clone-B disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In some embodiments, the CDRs are according to the combined CDR definitions of both Kabat and Chothia (e.g., as set out in Table 1). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 1.

In one embodiment, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In one embodiment, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In one embodiment, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence at least 85%, 90%, 95%, or 99% identical or higher to SEQ ID NO: 507. In one embodiment, the antibody molecule comprises a VL encoded by the nucleotide sequence of SEQ ID NO: 521 or 517, or a nucleotide sequence at least 85%, 90%, 95%, or 99% identical or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In one embodiment, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In one embodiment, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence at least 85%, 90%, 95%, or 99% identical or higher to SEQ ID NO: 509. In one embodiment, the antibody molecule comprises a light chain encoded by the nucleotide sequence of SEQ ID NO: 523 or 519, or a nucleotide sequence at least 85%, 90%, 95%, or 99% identical or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

TABLE 1
Amino acid and nucleotide sequences of exemplary anti-PD-1 antibody molecules

BAP049-Clone-B HC

| | | |
|---|---|---|
| SEQ ID NO: 501 (Kabat) | HCDR1 | TYWMH |
| SEQ ID NO: 502 (Kabat) | HCDR2 | NIYPGTGGSNFDEKFKN |
| SEQ ID NO: 503 (Kabat) | HCDR3 | WTTGTGAY |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQATGQG |
| | | LEWMGNIYPGTGGSNFDEKFKNRVTITADKSTSTAYMELSSLRSE |
| | | DTAVYYCTRWTTGTGAYWGQGTTVTVSS |
| SEQ ID NO: 507 | DNA VH | GAGGTGCAGCTGGTGCAGTCAGGCGCCGAAGTGAAGAAGCCCG |
| | | GCGAGTCACTGAGAATTAGCTGTAAAGGTTCAGGCTACACCTT |
| | | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQATGQG |
| | chain | LEWMGNIYPGTGGSNFDEKFKNRVTITADKSTSTAYMELSSLRSE |
| | | DTAVYYCTRWTTGTGAYWGQGTTVTVSSASTKGPSVFPLAPCSRS |
| | | TSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLG |
| SEQ ID NO: 509 | DNA | GAGGTGCAGCTGGTGCAGTCAGGCGCCGAAGTGAAGAAGCCCG |
| | heavy | GCGAGTCACTGAGAATTAGCTGTAAAGGTTCAGGCTACACCTT |
| | chain | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGCGCTAGCACTAAGGGCCCGTCCGTGTTCCCCCTGGCACC |
| | | TTGTAGCCGGAGCACTAGCGAATCCACCGCTGCCCTCGGCTGCC |
| | | TGGTCAAGGATTACTTCCCGGAGCCCGTGACCGTGTCCTGGAAC |
| | | AGCGGAGCCCTGACCTCCGGAGTGCACACCTTCCCCGCTGTGCT |
| | | GCAGAGCTCCGGGCTGTACTCGCTGTCGTCGGTGGTCACGGTGC |
| | | CTTCATCTAGCCTGGGTACCAAGACCTACACTTGCAACGTGGAC |
| | | CACAAGCCTTCCAACACTAAGGTGGACAAGCGCGTCGAATCGA |
| | | AGTACGGCCCACCGTGCCCGCCTTGTCCCGCGCCGGAGTTCCTC |
| | | GGCGGTCCCTCGGTCTTTCTGTTCCCACCGAAGCCCAAGGACAC |
| | | TTTGATGATTTCCCGCACCCCTGAAGTGACATGCGTGGTCGTGG |
| | | ACGTGTCACAGGAAGATCCGGAGGTGCAGTTCAATTGGTACGT |
| | | GGATGGCGTCGAGGTGCACAACGCCAAAACCAAGCCGAGGGA |
| | | GGAGCAGTTCAACTCCACTTACCGCGTCGTGTCCGTGCTGACGG |
| | | TGCTGCATCAGGACTGGCTGAACGGGAAGGAGTACAAGTGCAA |
| | | AGTGTCCAACAAGGGACTTCCTAGCTCAATCGAAAAGACCATC |
| | | TCGAAAGCCAAGGGACAGCCCCGGGAACCCCAAGTGTATACCC |
| | | TGCCACCGAGCCAGGAAGAAATGACTAAGAACCAAGTCTCATT |
| | | GACTTGCCTTGTGAAGGGCTTCTACCCATCGGATATCGCCGTGG |
| | | AATGGGAGTCCAACGGCCAGCCGGAAAACAACTACAAGACCA |
| | | CCCCTCCGGTGCTGGACTCAGACGGATCCTTCTTCCTCTACTCG |
| | | CGGCTGACCGTGGATAAGAGCAGATGGCAGGAGGGAAATGTGT |
| | | TCAGCTGTTCTGTGATGCATGAAGCCCTGCACAACCACTACACT |
| | | CAGAAGTCCCTGTCCCTCTCCCTGGGA |

BAP049-Clone-B LC

| | | |
|---|---|---|
| SEQ ID NO: 510 (Kabat) | LCDR1 | KSSQSLLDSGNQKNFLT |
| SEQ ID NO: 511 (Kabat) | LCDR2 | WASTRES |
| SEQ ID NO: 512 (Kabat) | LCDR3 | QNDYSYPYT |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 516 | VL | EIVLTQSPATLSLSPGERATLSCKSSQSLLDSGNQKNFLTWYQQKP |
| | | GKAPKLLIYWASTRESGVPSRFSGSGSGTDFTFTISSLQPEDIATYY |
| | | CQNDYSYPYTFGQGTKVEIK |
| SEQ ID NO: 517 | DNA VL | GAGATCGTCCTGACTCAGTCACCCGCTACCCTGAGCCTGAGCCC |
| | | TGGCGAGCGGGCTACACTGAGCTGTAAATCTAGTCAGTCACTG |
| | | CTGGATAGCGGTAATCAGAAGAACTTCCTGACCTGGTATCAGC |
| | | AGAAGCCCGGTAAAGCCCCTAAGCTGCTGATCTACTGGGCCTC |
| | | TACTAGAGAATCAGGCGTGCCCTCTAGGTTTAGCGGTAGCGGT |
| | | AGTGGCACCGACTTCACCTTCACTATCTCTAGCCTGCAGCCCGA |
| | | GGATATCGCTACCTACTACTGTCAGAACGACTATAGCTACCCCT |
| | | ACACCTTCGGTCAAGGCACTAAGGTCGAGATTAAG |
| SEQ ID NO: 518 | Light | EIVLTQSPATLSLSPGERATLSCKSSQSLLDSGNQKNFLTWYQQKP |
| | chain | GKAPKLLIYWASTRESGVPSRFSGSGSGTDFTFTISSLQPEDIATYY |
| | | CQNDYSYPYTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| SEQ ID NO: 519 | DNA | GAGATCGTCCTGACTCAGTCACCCGCTACCCTGAGCCTGAGCCC |
| | light | TGGCGAGCGGGCTACACTGAGCTGTAAATCTAGTCAGTCACTG |
| | chain | CTGGATAGCGGTAATCAGAAGAACTTCCTGACCTGGTATCAGC |
| | | AGAAGCCCGGTAAAGCCCCTAAGCTGCTGATCTACTGGGCCTC |
| | | TACTAGAGAATCAGGCGTGCCCTCTAGGTTTAGCGGTAGCGGT |
| | | AGTGGCACCGACTTCACCTTCACTATCTCTAGCCTGCAGCCCGA |
| | | GGATATCGCTACCTACTACTGTCAGAACGACTATAGCTACCCCT |
| | | ACACCTTCGGTCAAGGCACTAAGGTCGAGATTAAGCGTACGGT |
| | | GGCCGCTCCCAGCGTGTTCATCTTCCCCCCCAGCGACGAGCAGC |
| | | TGAAGAGCGGCACCGCCAGCGTGGTGTGCCTGCTGAACAACTT |
| | | CTACCCCCGGGAGGCCAAGGTGCAGTGGAAGGTGGACAACGCC |
| | | CTGCAGAGCGGCAACAGCCAGGAGAGCGTCACCGAGCAGGAC |
| | | AGCAAGGACTCCACCTACAGCCTGAGCAGCACCCTGACCCTGA |
| | | GCAAGGCCGACTACGAGAAGCATAAGGTGTACGCCTGCGAGGT |
| | | GACCCACCAGGGCCTGTCCAGCCCCGTGACCAAGAGCTTCAAC |
| | | AGGGGCGAGTGC |

BAP049-Clone-E HC

| | | |
|---|---|---|
| SEQ ID NO: 501 (Kabat) | HCDR1 | TYWMH |
| SEQ ID NO: 502 (Kabat) | HCDR2 | NIYPGTGGSNFDEKFKN |
| SEQ ID NO: 503 (Kabat) | HCDR3 | WTTGTGAY |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQATGQG |
| | | LEWMGNIYPGTGGSNFDEKFKNRVTITADKSTSTAYMELSSLRSE |
| | | DTAVYYCTRWTTGTGAYWGQGTTVTVSS |
| SEQ ID NO: 507 | DNA VH | GAGGTGCAGCTGGTGCAGTCAGGCGCCGAAGTGAAGAAGCCCG |
| | | GCGAGTCACTGAGAATTAGCTGTAAAGGTTCAGGCTACACCTT |
| | | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQATGQG |
| | chain | LEWMGNIYPGTGGSNFDEKFKNRVTITADKSTSTAYMELSSLRSE |
| | | DTAVYYCTRWTTGTGAYWGQGTTVTVSSASTKGPSVFPLAPCSRS |
| | | TSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLG |
| SEQ ID NO: 509 | DNA | GAGGTGCAGCTGGTGCAGTCAGGCGCCGAAGTGAAGAAGCCCG |
| | heavy | GCGAGTCACTGAGAATTAGCTGTAAAGGTTCAGGCTACACCTT |
| | chain | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGCGCTAGCACTAAGGGCCCGTCCGTGTTCCCCCTGGCACC |
| | | TTGTAGCCGGAGCACTAGCGAATCCACCGCTGCCCTCGGCTGCC |
| | | TGGTCAAGGATTACTTCCCGGAGCCCGTGACCGTGTCCTGGAAC |
| | | AGCGGAGCCCTGACCTCCGGAGTGCACACCTTCCCCGCTGTGCT |
| | | GCAGAGCTCCGGGCTGTACTCGCTGTCGTCGGTGGTCACGGTGC |
| | | CTTCATCTAGCCTGGGTACCAAGACCTACACTTGCAACGTGGAC |
| | | CACAAGCCTTCCAACACTAAGGTGGACAAGCGCGTCGAATCGA |
| | | AGTACGGCCCACCGTGCCCGCCTTGTCCCGCGCCGGAGTTCCTC |
| | | GGCGGTCCCTCGGTCTTTCTGTTCCCACCGAAGCCCAAGGACAC |
| | | TTTGATGATTTCCCGCACCCCTGAAGTGACATGCGTGGTCGTGG |
| | | ACGTGTCACAGGAAGATCCGGAGGTGCAGTTCAATTGGTACGT |
| | | GGATGGCGTCGAGGTGCACAACGCCAAAACCAAGCCGAGGGA |
| | | GGAGCAGTTCAACTCCACTTACCGCGTCGTGTCCGTGCTGACGG |
| | | TGCTGCATCAGGACTGGCTGAACGGGAAGGAGTACAAGTGCAA |
| | | AGTGTCCAACAAGGGACTTCCTAGCTCAATCGAAAAGACCATC |
| | | TCGAAAGCCAAGGGACAGCCCCGGGAACCCCAAGTGTATACCC |
| | | TGCCACCGAGCCAGGAAGAAATGACTAAGAACCAAGTCTCATT |
| | | GACTTGCCTTGTGAAGGGCTTCTACCCATCGGATATCGCCGTGG |
| | | AATGGGAGTCCAACGGCCAGCCGGAAAACAACTACAAGACCA |
| | | CCCCTCCGGTGCTGGACTCAGACGGATCCTTCTTCCTCTACTCG |
| | | CGGCTGACCGTGGATAAGAGCAGATGGCAGGAGGGAAATGTGT |
| | | TCAGCTGTTCTGTGATGCATGAAGCCCTGCACAACCACTACACT |
| | | CAGAAGTCCCTGTCCCTCTCCCTGGGA |

BAP049-Clone-E LC

| | | |
|---|---|---|
| SEQ ID NO: 510 (Kabat) | LCDR1 | KSSQSLLDSGNQKNFLT |
| SEQ ID NO: 511 (Kabat) | LCDR2 | WASTRES |
| SEQ ID NO: 512 (Kabat) | LCDR3 | QNDYSYPYT |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLDSGNQKNFLTWYQQKP |
| | | GQAPRLLIYWASTRESGVPSRFSGSGSGTDFTFTISSLEAEDAATYY |
| | | CQNDYSYPYTFGQGTKVEIK |
| SEQ ID NO: 521 | DNA VL | GAGATCGTCCTGACTCAGTCACCCGCTACCCTGAGCCTGAGCCC |
| | | TGGCGAGCGGGCTACACTGAGCTGTAAATCTAGTCAGTCACTG |
| | | CTGGATAGCGGTAATCAGAAGAACTTCCTGACCTGGTATCAGC |
| | | AGAAGCCCGGTCAAGCCCCTAGACTGCTGATCTACTGGGCCTCT |
| | | ACTAGAGAATCAGGCGTGCCCTCTAGGTTTAGCGGTAGCGGTA |
| | | GTGGCACCGACTTCACCTTCACTATCTCTAGCCTGGAAGCCGAG |
| | | GACGCCGCTACCTACTACTGTCAGAACGACTATAGCTACCCCTA |
| | | CACCTTCGGTCAAGGCACTAAGGTCGAGATTAAG |
| SEQ ID NO: 522 | Light | EIVLTQSPATLSLSPGERATLSCKSSQSLLDSGNQKNFLTWYQQKP |
| | chain | GQAPRLLIYWASTRESGVPSRFSGSGSGTDFTFTISSLEAEDAATYY |
| | | CQNDYSYPYTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| SEQ ID NO: 523 | DNA | GAGATCGTCCTGACTCAGTCACCCGCTACCCTGAGCCTGAGCCC |
| | light | TGGCGAGCGGGCTACACTGAGCTGTAAATCTAGTCAGTCACTG |
| | chain | CTGGATAGCGGTAATCAGAAGAACTTCCTGACCTGGTATCAGC |
| | | AGAAGCCCGGTCAAGCCCCTAGACTGCTGATCTACTGGGCCTCT |
| | | ACTAGAGAATCAGGCGTGCCCTCTAGGTTTAGCGGTAGCGGTA |
| | | GTGGCACCGACTTCACCTTCACTATCTCTAGCCTGGAAGCCGAG |
| | | GACGCCGCTACCTACTACTGTCAGAACGACTATAGCTACCCCTA |
| | | CACCTTCGGTCAAGGCACTAAGGTCGAGATTAAGCGTACGGTG |
| | | GCCGCTCCCAGCGTGTTCATCTTCCCCCCCAGCGACGAGCAGCT |
| | | GAAGAGCGGCACCGCCAGCGTGGTGTGCCTGCTGAACAACTTC |
| | | TACCCCCGGGAGGCCAAGGTGCAGTGGAAGGTGGACAACGCCC |
| | | TGCAGAGCGGCAACAGCCAGGAGAGCGTCACCGAGCAGGACA |
| | | GCAAGGACTCCACCTACAGCCTGAGCAGCACCCTGACCCTGAG |
| | | CAAGGCCGACTACGAGAAGCATAAGGTGTACGCCTGCGAGGTG |
| | | ACCCACCAGGGCCTGTCCAGCCCCGTGACCAAGAGCTTCAACA |
| | | GGGGCGAGTGC |

BAP049-Clone-B HC

| | | |
|---|---|---|
| SEQ ID NO: 524 (Kabat) | HCDR1 | ACCTACTGGATGCAC |
| SEQ ID NO: 525 (Kabat) | HCDR2 | AATATCTACCCCGGCACCGGCGGCTCTAACTTCGACGAGAAGT |
| | | TTAAGAAT |
| SEQ ID NO: 526 (Kabat) | HCDR3 | TGGACTACCGGCACAGGCGCCTAC |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |

BAP049-Clone-B LC

| | | |
|---|---|---|
| SEQ ID NO: 529 (Kabat) | LCDR1 | AAATCTAGTCAGTCACTGCTGGATAGCGGTAATCAGAAGAACT |
| | | TCCTGACC |
| SEQ ID NO: 530 (Kabat) | LCDR2 | TGGGCCTCTACTAGAGAATCA |
| SEQ ID NO: 531 (Kabat) | LCDR3 | CAGAACGACTATAGCTACCCCTACACC |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

BAP049-Clone-E HC

| | | |
|---|---|---|
| SEQ ID NO: 524 (Kabat) | HCDR1 | ACCTACTGGATGCAC |
| SEQ ID NO: 525 (Kabat) | HCDR2 | AATATCTACCCCGGCACCGGCGGCTCTAACTTCGACGAGAAGT |
| | | TTAAGAAT |
| SEQ ID NO: 526 (Kabat) | HCDR3 | TGGACTACCGGCACAGGCGCCTAC |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |

BAP049-Clone-E LC

| | | |
|---|---|---|
| SEQ ID NO: 529 (Kabat) | LCDR1 | AAATCTAGTCAGTCACTGCTGGATAGCGGTAATCAGAAGAACT |
| | | TCCTGACC |
| SEQ ID NO: 530 (Kabat) | LCDR2 | TGGGCCTCTACTAGAGAATCA |
| SEQ ID NO: 531 (Kabat) | LCDR3 | CAGAACGACTATAGCTACCCCTACACC |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

In some embodiments, the PD-1 inhibitor is administered at a dose of about 200 mg to about 500 mg (e.g., about 300 mg to about 400 mg). In some embodiments, the PD-1 inhibitor is administered once every 3 weeks. In some embodiments, the PD-1 inhibitor is administered once every 4 weeks. In other embodiments, the PD-1 inhibitor is administered at a dose of about 200 mg to about 400 mg (e.g., about 300 mg) once every 3 weeks. In yet other embodiments, the PD-1 inhibitor is administered at a dose of about 300 mg to about 500 mg (e.g., about 400 mg) once every 4 weeks.

In some embodiments, the combination comprises a PD-1 inhibitor, e.g., PDR001, and a TGF-β inhibitor, e.g., NIS793. In some embodiments, this combination is administered to a subject in a therapeutically effective amount to treat, e.g., a pancreatic cancer.

In some embodiments, the combination comprises a PD-1 inhibitor, e.g., PDR001, and a TLR7 agonist, e.g., LHC₁₋₆₅. In some embodiments, this combination is administered to a subject in a therapeutically effective amount to treat, e.g., a pancreatic cancer. In some embodiments, the TLR7 agonist, e.g., LHC₁₋₆₅ is administered via intra-tumoral injection.

In some embodiments, the combination comprises a PD-1 inhibitor, e.g., PDR001, and an adenosine receptor antagonist, e.g., PBF509 (NIR178). In some embodiments, this combination is administered to a subject in a therapeutically effective amount to treat, e.g., a pancreatic cancer.

In some embodiments, the combination comprises a PD-1 inhibitor, e.g., PDR001, and an inhibitor of Porcupine, e.g., WNT974. In some embodiments, this combination is administered to a subject in a therapeutically effective amount to treat, e.g., a pancreatic cancer.

In some embodiments, the combination comprises a PD-1 inhibitor, e.g., PDR001, and an A2aR antagonist, e.g., PBF509 (NIR178). In some embodiments, this combination is administered to a subject in a therapeutically effective amount to treat, e.g., a CRC or gastric cancer. Without wishing to be bound by theory, it is believed that a combination comprising a PD-1 inhibitor, e.g., PDR001, and an A2aR antagonist, e.g., PBF509 (NIR178), can result in increased efficacy of the anti-PD-1 inhibitor. In some embodiments, the combination of a PD-1 inhibitor, e.g., PDR001, and an A2aR antagonist, e.g., PBF509 (NIR178), results in regression of a CRC tumor.

In some embodiments, the combination comprises a PD-1 inhibitor, e.g., PDR001, and a PD-L1 inhibitor, e.g., FAZ053. In some embodiments, the combination is administered to a subject in a therapeutically effective amount to treat, e.g., a breast cancer, e.g., a triple negative breast cancer.

Other Exemplary PD-1 Inhibitors

In one embodiment, the anti-PD-1 antibody molecule is Nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 2.

In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 2.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 2.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

TABLE 2
Amino acid sequences of other exemplary anti-PD-1 antibody molecules

Nivolumab

| | | |
|---|---|---|
| SEQ ID NO: 535 | Heavy | QVQLVESGGGVVQPGRSLRLDCKASGITFSNSGMHWVRQAPGKGLE |
| | chain | WVAVIWYDGSKRYYADSVKGRFTISRDNSKNTLFLQMNSLRAEDTA |
| | | VYYCATNDDYWGQGTLVTVSSASTKGPSVFPLAPCSRSTSESTAALG |
| | | CLVKDYFPEPVTVSWNSGALTSGVHTFPAVLQSSGLYSLSSVVTVPSS |
| | | SLGTKTYTCNVDHKPSNTKVDKRVESKYGPPCPPCPAPEFLGGPSVFL |
| | | FPPKPKDTLMISRTPEVTCVVVDVSQEDPEVQFNWYVDGVEVHNAK |
| | | TKPREEQFNSTYRVVSVLTVLHQDWLNGKEYKCKVSNKGLPSSIEKTI |
| | | SKAKGQPREPQVYTLPPSQEEMTKNQVSLTCLVKGFYPSDIAVEWES |
| | | NGQPENNYKTTPPVLDSDGSFFLYSRLTVDKSRWQEGNVFSCSVMHE |
| | | ALHNHYTQKSLSLSLGK |
| SEQ ID NO: 536 | Light | EIVLTQSPATLSLSPGERATLSCRASQSVSSYLAWYQQKPGQAPRLLIY |
| | chain | DASNRATGIPARFSGSGSGTDFTLTISSLEPEDFAVYYCQQSSNWPRTF |
| | | GQGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVY |
| | | ACEVTHQGLSSPVTKSFNRGEC |

Pembrolizumab

| | | |
|---|---|---|
| SEQ ID NO: 537 | Heavy | QVQLVQSGVEVKKPGASVKVSCKASGYTFTNYYMYWVRQAPGQGL |
| | chain | EWMGGINPSNGGTNFNEKFKNRVTLTTDSSTTTAYMELKSLQFDDTA |
| | | VYYCARRDYRFDMGFDYWGQGTTVTVSSASTKGPSVFPLAPCSRSTS |
| | | ESTAALGCLVKDYFPEPVTVSWNSGALTSGVHTFPAVLQSSGLYSLSS |
| | | VVTVPSSSLGTKTYTCNVDHKPSNTKVDKRVESKYGPPCPPCPAPEFL |
| | | GGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGL |
| | | PSSIEKTISKAKGQPREPQVYTLPPSQEEMTKNQVSLTCLVKGFYPSDI |
| | | AVEWESNGQPENNYKTTPPVLDSDGSFFLYSRLTVDKSRWQEGNVFS |
| | | CSVMHEALHNHYTQKSLSLSLGK |
| SEQ ID NO: 538 | Light | EIVLTQSPATLSLSPGERATLSCRASKGVSTSGYSYLHWYQQKPGQAP |
| | chain | RLLIYLASYLESGVPARFSGSGSGTDFTLTISSLEPEDFAVYYCQHSRD |
| | | LPLTFGGGTKVEIKRTVAAPSVFIFPPSDEQLKSGTASVVCLLNNFYPR |
| | | EAKVQWKVDNALQSGNSQESVTEQDSKDSTYSLSSTLTLSKADYEK |
| | | HKVYACEVTHQGLSSPVTKSFNRGEC |

Pidilizumab

| | | |
|---|---|---|
| SEQ ID NO: 539 | Heavy | QVQLVQSGSELKKPGASVKISCKASGYTFTNYGMNWVRQAPGQGLQ |
| | chain | WMGWINTDSGESTYAEEFKGRFVFSLDTSVNTAYLQITSLTAEDTGM |
| | | YFCVRVGYDALDYWGQGTLVTVSSASTKGPSVFPLAPSSKSTSGGTA |
| | | ALGCLVKDYFPEPVTVSWNSGALTSGVHTFPAVLQSSGLYSLSSVVT |
| | | VPSSSLGTQTYICNVNHKPSNTKVDKRVEPKSCDKTHTCPPCPAPELL |
| | | GGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVF |
| | | SCSVMHEALHNHYTQKSLSLSPGK |
| SEQ ID NO: 540 | Light | EIVLTQSPSSLSASVGDRVTITCSARSSVSYMHWFQQKPGKAPKLWIY |
| | chain | RTSNLASGVPSRFSGSGSGTSYCLTINSLQPEDFATYYCQQRSSFPLTF |
| | | GGGTKLEIKRTVAAPSVFIFPPSDEQLKSGTASVVCLLNNFYPREAKV |
| | | QWKVDNALQSGNSQESVTEQDSKDSTYSLSSTLTLSKADYEKHKVY |
| | | ACEVTHQGLSSPVTKSFNRGEC |

Additional Combination Therapies

In an embodiment, the combination comprises a PD-1 inhibitor (e.g., PDR001), and an inhibitor of apoptosis (IAP) inhibitor (e.g., LCL161). In some embodiments, the combination comprises PDR001 and an IAP inhibitor. In some embodiments, the combination comprises PDR001 and LCL161.

In some embodiments, the IAP inhibitor comprises LCL161 or a compound disclosed in International Application Publication No. WO 2008/016893, which is hereby incorporated by reference in its entirety. In some embodiments, the IAP inhibitor (e.g., LCL161) is administered daily at a dose of 100-2000 mg, or 200-1500 mg, e.g., about 300-900 mg. In some embodiments, the IAP inhibitor (e.g., LCL161), is administered daily at a dose of about 300-900 mg. In some embodiments, the IAP inhibitor (e.g., LCL161) is administered once a week at a dose of 100-2000 mg, or 200-1500 mg, e.g., about 300-900 mg. In some embodiments, the IAP inhibitor (e.g., LCL161), is administered once a week at a dose of about 300-900 mg. In some embodiments, the IAP inhibitor (e.g., LCL161), is administered once a week at a dose of 300 mg. In some embodiments, the IAP inhibitor (e.g., LCL161), is administered once a week at a dose of 900 mg. In some embodiments, this combination is administered to a subject in a therapeutically effective amount to treat a cancer, e.g., a cancer described herein, e.g., a colorectal cancer.

In an embodiment, the combination comprises a PD-1 inhibitor (e.g., PDR001), and an mTOR inhibitor, e.g., RAD001 (also known as everolimus). In some embodiments, the combination comprises PDR001 and an mTOR inhibitor, e.g., RAD001. In some embodiments, the combination comprises PDR001 and RAD001. In some embodiments, the mTOR inhibitor, e.g., RAD001, is administered once weekly at a dose of at least 0.5 mg, 1 mg, 2 mg, 3 mg, 4 mg, 5 mg, 6 mg, 7 mg, 8 mg, 9 mg, or 10 mgs. In some embodiments, the mTOR inhibitor, e.g., RAD001, is administered once weekly at a dose of 10 mg. In some embodiments, the mTOR inhibitor, e.g., RAD001, is administered once weekly at a dose of 5 mg. In some embodiments, the mTOR inhibitor, e.g., RAD001, is administered once daily at a dose of at least 0.5 mg, 1 mg, 2 mg, 3 mg, 4 mg, 5 mg, 6 mg, 7 mg, 8 mg, 9 mg, or 10 mgs. In some embodiments, the mTOR inhibitor, e.g., RAD001, is administered once daily at a dose of 0.5 mg. In some embodiments, this combination is administered to a subject in a therapeutically effective amount to treat a cancer, e.g., a cancer described herein, e.g., a colorectal cancer.

In an embodiment, the combination comprises a PD-1 inhibitor (e.g., PDR001), and a HDAC inhibitor, e.g., LBH589. LBH589 is also known as panobinostat. In some embodiments, the combination comprises PDR001 and a HDAC inhibitor, e.g., LBH589. In some embodiments, the combination comprises PDR001 and LHB589. In some embodiments, the HDAC inhibitor, e.g., LBH589 is administered at a dose of at least 10, 15, 20, 25, 30, 40, 50, 60, 70, or 80 mg. In some embodiments, the HDAC inhibitor, e.g., LBH589 is administered at a dose of 20 mg. In some embodiments, the HDAC inhibitor, e.g., LBH589 is administered at a dose of 10 mg. In some embodiments, the HDAC inhibitor, e.g., LBH589 is administered at a dose of 10 mg or 20 mg once every other day, e.g., on days 1, 3, 5, 8, 10 and 12, of a dosing cycle, e.g., a dosing cycle consisting of 21 days. In some embodiments, the HDAC inhibitor, e.g., LBH589, is administered every other day, e.g., administered three times a week. In some embodiments, the HDAC inhibitor, e.g., LBH589, is administered for at least dosing 8 cycles, e.g., 1, 2, 3, 4, 5, 6, 7, or 8 cycles, wherein each dosing cycle consists of 21 days. In some embodiments, the HDAC inhibitor, e.g., LBH589, is administered at a dose of 10 mg or 20 mg on days 1, 3, 5, 8, 10 and 12 of a dosing cycle, for 8 dosing cycles. In some embodiments, this combination is administered to a subject in a therapeutically effective amount to treat a cancer, e.g., a cancer described herein, e.g., a colorectal cancer or multiple myeloma.

In an embodiment, the combination comprises a PD-1 inhibitor (e.g., PDR001), and an IL-17 inhibitor, e.g., CJM112. In some embodiments, the combination comprises PDR001 and an IL-17 inhibitor, e.g., CJM112. In some embodiments, the combination comprises PDR001 and CJM112. In some embodiments, this combination is administered to a subject in a therapeutically effective amount to treat a cancer, e.g., a cancer described herein, e.g., a colorectal cancer.

### LAG-3 Inhibitors

In certain embodiments, a combination described herein comprises a LAG-3 inhibitor. In some embodiments, the LAG-3 inhibitor is chosen from LAG525 (Novartis), BMS-986016 (Bristol-Myers Squibb), or TSR-033 (Tesaro).

Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of BAP050-Clone I or BAP050-Clone J disclosed in Table 5), or encoded by a nucleotide sequence shown in Table 5. In some embodiments, the CDRs are according to the Kabat definition (e.g., as set out in Table 5). In some embodiments, the CDRs are according to the Chothia definition (e.g., as set out in Table 5). In some embodiments, the CDRs are according to the combined CDR definitions of both Kabat and Chothia (e.g., as set out in Table 5). In one embodiment, the combination of Kabat and Chothia CDR of VH CDR1 comprises the amino acid sequence GFTLTNYGMN (SEQ ID NO: 766). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 5, or encoded by a nucleotide sequence shown in Table 5.

In one embodiment, the anti-LAG-3 antibody molecule comprises a heavy chain variable region (VH) comprising a VHCDR1 amino acid sequence of SEQ ID NO: 701, a VHCDR2 amino acid sequence of SEQ ID NO: 702, and a VHCDR3 amino acid sequence of SEQ ID NO: 703; and a light chain variable region (VL) comprising a VLCDR1 amino acid sequence of SEQ ID NO: 710, a VLCDR2 amino acid sequence of SEQ ID NO: 711, and a VLCDR3 amino acid sequence of SEQ ID NO: 712, each disclosed in Table 5.

In one embodiment, the anti-LAG-3 antibody molecule comprises a VH comprising a VHCDR1 encoded by the nucleotide sequence of SEQ ID NO: 736 or 737, a VHCDR2 encoded by the nucleotide sequence of SEQ ID NO: 738 or 739, and a VHCDR3 encoded by the nucleotide sequence of SEQ ID NO: 740 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 5. In one embodiment, the anti-LAG-3 antibody molecule comprises a VH comprising a VHCDR1 encoded by the nucleotide sequence of SEQ ID NO: 758 or 737, a VHCDR2 encoded by the nucleotide sequence of SEQ ID NO: 759 or 739, and a VHCDR3 encoded by the nucleotide sequence of SEQ ID NO: 760 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 5.

In one embodiment, the anti-LAG-3 antibody molecule comprises a VH comprising the amino acid sequence of SEQ ID NO: 706, or an amino acid sequence at least 85%, 90%, 95%, or 99% identical or higher to SEQ ID NO: 706. In one embodiment, the anti-LAG-3 antibody molecule comprises a VL comprising the amino acid sequence of SEQ ID NO: 718, or an amino acid sequence at least 85%, 90%, 95%, or 99% identical or higher to SEQ ID NO: 718. In one embodiment, the anti-LAG-3 antibody molecule comprises a VH comprising the amino acid sequence of SEQ ID NO: 724, or an amino acid sequence at least 85%, 90%, 95%, or 99% identical or higher to SEQ ID NO: 724. In one embodiment, the anti-LAG-3 antibody molecule comprises a VL comprising the amino acid sequence of SEQ ID NO: 730, or an amino acid sequence at least 85%, 90%, 95%, or 99% identical or higher to SEQ ID NO: 730. In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

In one embodiment, the antibody molecule comprises a VH encoded by the nucleotide sequence of SEQ ID NO: 707 or 708, or a nucleotide sequence at least 85%, 90%, 95%, or 99% identical or higher to SEQ ID NO: 707 or 708. In one embodiment, the antibody molecule comprises a VL encoded by the nucleotide sequence of SEQ ID NO: 719 or 720, or a nucleotide sequence at least 85%, 90%, 95%, or 99% identical or higher to SEQ ID NO: 719 or 720. In one embodiment, the antibody molecule comprises a VH encoded by the nucleotide sequence of SEQ ID NO: 725 or 726, or a nucleotide sequence at least 85%, 90%, 95%, or 99% identical or higher to SEQ ID NO: 725 or 726. In one embodiment, the antibody molecule comprises a VL encoded by the nucleotide sequence of SEQ ID NO: 731 or 732, or a nucleotide sequence at least 85%, 90%, 95%, or 99% identical or higher to SEQ ID NO: 731 or 732. In one embodiment, the antibody molecule comprises a VH encoded by the nucleotide sequence of SEQ ID NO: 707 or 708 and a VL encoded by the nucleotide sequence of SEQ ID NO: 719 or 720. In one embodiment, the antibody molecule comprises a VH encoded by the nucleotide sequence of SEQ ID NO: 725 or 726 and a VL encoded by the nucleotide sequence of SEQ ID NO: 731 or 732.

In one embodiment, the anti-LAG-3 antibody molecule comprises a heavy chain comprising the amino acid sequence of SEQ ID NO: 709, or an amino acid sequence at least 85%, 90%, 95%, or 99% identical or higher to SEQ ID NO: 709. In one embodiment, the anti-LAG-3 antibody molecule comprises a light chain comprising the amino acid sequence of SEQ ID NO: 721, or an amino acid sequence at least 85%, 90%, 95%, or 99% identical or higher to SEQ ID NO: 721. In one embodiment, the anti-LAG-3 antibody molecule comprises a heavy chain comprising the amino acid sequence of SEQ ID NO: 727, or an amino acid sequence at least 85%, 90%, 95%, or 99% identical or higher to SEQ ID NO: 727. In one embodiment, the anti-LAG-3 antibody molecule comprises a light chain comprising the amino acid sequence of SEQ ID NO: 733, or an amino acid sequence at least 85%, 90%, 95%, or 99% identical or higher to SEQ ID NO: 733. In one embodiment, the anti-LAG-3 antibody molecule comprises a heavy chain comprising the amino acid sequence of SEQ ID NO: 709 and a light chain comprising the amino acid sequence of SEQ ID NO: 721. In one embodiment, the anti-LAG-3 antibody molecule comprises a heavy chain comprising the amino acid sequence of SEQ ID NO: 727 and a light chain comprising the amino acid sequence of SEQ ID NO: 733.

In one embodiment, the antibody molecule comprises a heavy chain encoded by the nucleotide sequence of SEQ ID NO: 716 or 717, or a nucleotide sequence at least 85%, 90%, 95%, or 99% identical or higher to SEQ ID NO: 716 or 717. In one embodiment, the antibody molecule comprises a light chain encoded by the nucleotide sequence of SEQ ID NO: 722 or 723, or a nucleotide sequence at least 85%, 90%, 95%, or 99% identical or higher to SEQ ID NO: 722 or 723. In one embodiment, the antibody molecule comprises a heavy chain encoded by the nucleotide sequence of SEQ ID NO: 728 or 729, or a nucleotide sequence at least 85%, 90%, 95%, or 99% identical or higher to SEQ ID NO: 728 or 729. In one embodiment, the antibody molecule comprises a light chain encoded by the nucleotide sequence of SEQ ID NO: 734 or 735, or a nucleotide sequence at least 85%, 90%, 95%, or 99% identical or higher to SEQ ID NO: 734 or 735. In one embodiment, the antibody molecule comprises a heavy chain encoded by the nucleotide sequence of SEQ ID NO: 716 or 717 and a light chain encoded by the nucleotide sequence of SEQ ID NO: 722 or 723. In one embodiment, the antibody molecule comprises a heavy chain encoded by the nucleotide sequence of SEQ ID NO: 728 or 729 and a light chain encoded by the nucleotide sequence of SEQ ID NO: 734 or 735.

The antibody molecules described herein can be made by vectors, host cells, and methods described in US 2015/0259420, incorporated by reference in its entirety.

TABLE 5
Amino acid and nucleotide sequences of exemplary anti-LAG-3 antibody
molecules

BAP050-Clone I HC

| | | |
|---|---|---|
| SEQ ID NO: 701 (Kabat) | HCDR1 | NYGMN |
| SEQ ID NO: 702 (Kabat) | HCDR2 | WINTDTGEPTYADDFKG |
| SEQ ID NO: 703 (Kabat) | HCDR3 | NPPYYYGTNNAEAMDY |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 706 | VH | QVQLVQSGAEVKKPGASVKVSCKASGFTLTNYGMNWVRQ |
| | | ARGQRLEWIGWINTDTGEPTYADDFKGRFVFSLDTSVSTAY |
| | | LQISSLKAEDTAVYYCARNPPYYYGTNNAEAMDYWGQGTT |
| | | VTVSS |
| SEQ ID NO: 707 | DNA VH | CAAGTGCAGCTGGTGCAGTCGGGAGCCGAAGTGAAGAAG |
| | | CCTGGAGCCTCGGTGAAGGTGTCGTGCAAGGCATCCGGA |
| | | TTCACCCTCACCAATTACGGGATGAACTGGGTCAGACAG |
| | | GCCCGGGGTCAACGGCTGGAGTGGATCGGATGGATTAAC |
| | | ACCGACACCGGGGAGCCTACCTACGCGGACGATTTCAAG |
| | | GGACGGTTCGTGTTCTCCCTCGACACCTCCGTGTCCACCG |
| | | CCTACCTCCAAATCTCCTCACTGAAAGCGGAGGACACCG |
| | | CCGTGTACTATTGCGCGAGGAACCCGCCCTACTACTACGG |
| | | AACCAACAACGCCGAAGCCATGGACTACTGGGGCCAGGG |
| | | CACCACTGTGACTGTGTCCAGC |
| SEQ ID NO: 708 | DNA VH | CAGGTGCAGCTGGTGCAGTCTGGCGCCGAAGTGAAGAAA |
| | | CCTGGCGCCTCCGTGAAGGTGTCCTGCAAGGCCTCTGGCT |
| | | TCACCCTGACCAACTACGGCATGAACTGGGTGCGACAGG |
| | | CCAGGGGCCAGCGGCTGGAATGGATCGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCT |
| SEQ ID NO: 709 | Heavy | QVQLVQSGAEVKKPGASVKVSCKASGIFTLTNYGMNWVRQ |
| | chain | ARGQRLEWIGWINTDTGEPTYADDFKGRFVFSLDTSVSTAY |
| | | LQISSLKAEDTAVYYCARNPPYYYGTNNAEAMDYWGQGTT |
| | | VTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFLGGPSV |
| | | FLFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKNQV |
| | | SLTCLVKGFYPSDIAVEWESNGQPENNYKTTPPVLDSDGSFF |
| | | LYSRLTVDKSRWQEGNVFSCSVMHEALHNHYTQKSLSLSLG |
| SEQ ID NO: 716 | DNA | CAAGTGCAGCTGGTGCAGTCGGGAGCCGAAGTGAAGAAG |
| | heavy | CCTGGAGCCTCGGTGAAGGTGTCGTGCAAGGCATCCGGA |
| | chain | TTCACCCTCACCAATTACGGGATGAACTGGGTCAGACAG |
| | | GCCCGGGGTCAACGGCTGGAGTGGATCGGATGGATTAAC |
| | | ACCGACACCGGGGAGCCTACCTACGCGGACGATTTCAAG |
| | | GGACGGTTCGTGTTCTCCCTCGACACCTCCGTGTCCACCG |
| | | CCTACCTCCAAATCTCCTCACTGAAAGCGGAGGACACCG |
| | | CCGTGTACTATTGCGCGAGGAACCCGCCCTACTACTACGG |
| | | AACCAACAACGCCGAAGCCATGGACTACTGGGGCCAGGG |
| | | CACCACTGTGACTGTGTCCAGCGCGTCCACTAAGGGCCC |
| | | GTCCGTGTTCCCCCTGGCACCTTGTAGCCGGAGCACTAGC |
| | | GAATCCACCGCTGCCCTCGGCTGCCTGGTCAAGGATTACT |
| | | TCCCGGAGCCCGTGACCGTGTCCTGGAACAGCGGAGCCC |
| | | TGACCTCCGGAGTGCACACCTTCCCCGCTGTGCTGCAGAG |
| | | CTCCGGGCTGTACTCGCTGTCGTCGGTGGTCACGGTGCCT |
| | | TCATCTAGCCTGGGTACCAAGACCTACACTTGCAACGTGG |
| | | ACCACAAGCCTTCCAACACTAAGGTGGACAAGCGCGTCG |
| | | AATCGAAGTACGGCCCACCGTGCCCGCCTTGTCCCGCGCC |
| | | GGAGTTCCTCGGCGGTCCCTCGGTCTTTCTGTTCCCACCG |
| | | AAGCCCAAGGACACTTTGATGATTTCCCGCACCCCTGAA |
| | | GTGACATGCGTGGTCGTGGACGTGTCACAGGAAGATCCG |
| | | GAGGTGCAGTTCAATTGGTACGTGGATGGCGTCGAGGTG |
| | | CACAACGCCAAAACCAAGCCGAGGGAGGAGCAGTTCAA |
| | | CTCCACTTACCGCGTCGTGTCCGTGCTGACGGTGCTGCAT |
| | | CAGGACTGGCTGAACGGGAAGGAGTACAAGTGCAAAGT |
| | | GTCCAACAAGGGACTTCCTAGCTCAATCGAAAAGACCAT |
| | | CTCGAAAGCCAAGGGACAGCCCCGGGAACCCCAAGTGTA |
| | | TACCCTGCCACCGAGCCAGGAAGAAATGACTAAGAACCA |
| | | AGTCTCATTGACTTGCCTTGTGAAGGGCTTCTACCCATCG |
| | | GATATCGCCGTGGAATGGGAGTCCAACGGCCAGCCGGAA |
| | | AACAACTACAAGACCACCCCTCCGGTGCTGGACTCAGAC |
| | | GGATCCTTCTTCCTCTACTCGCGGCTGACCGTGGATAAGA |
| | | GCAGATGGCAGGAGGGAAATGTGTTCAGCTGTTCTGTGA |
| | | TGCATGAAGCCCTGCACAACCACTACACTCAGAAGTCCC |
| | | TGTCCCTCTCCCTGGGA |
| SEQ ID NO: 717 | DNA | CAGGTGCAGCTGGTGCAGTCTGGCGCCGAAGTGAAGAAA |
| | heavy | CCTGGCGCCTCCGTGAAGGTGTCCTGCAAGGCCTCTGGCT |
| | chain | TCACCCTGACCAACTACGGCATGAACTGGGTGCGACAGG |
| | | CCAGGGGCCAGCGGCTGGAATGGATCGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCTGCTTCTACCAAGGGGCCCA |
| | | GCGTGTTCCCCCTGGCCCCCTGCTCCAGAAGCACCAGCGA |
| | | GAGCACAGCCGCCCTGGGCTGCCTGGTGAAGGACTACTT |
| | | CCCCGAGCCCGTGACCGTGTCCTGGAACAGCGGAGCCCT |
| | | GACCAGCGGCGTGCACACCTTCCCCGCCGTGCTGCAGAG |
| | | CAGCGGCCTGTACAGCCTGAGCAGCGTGGTGACCGTGCC |
| | | CAGCAGCAGCCTGGGCACCAAGACCTACACCTGTAACGT |
| | | GGACCACAAGCCCAGCAACACCAAGGTGGACAAGAGGG |
| | | TGGAGAGCAAGTACGGCCCACCCTGCCCCCCCTGCCCAG |
| | | CCCCCGAGTTCCTGGGCGGACCCAGCGTGTTCCTGTTCCC |
| | | CCCCAAGCCCAAGGACACCCTGATGATCAGCAGAACCCC |
| | | CGAGGTGACCTGTGTGGTGGTGGACGTGTCCCAGGAGGA |
| | | CCCCGAGGTCCAGTTCAACTGGTACGTGGACGGCGTGGA |
| | | GGTGCACAACGCCAAGACCAAGCCCAGAGAGGAGCAGTT |
| | | TAACAGCACCTACCGGGTGGTGTCCGTGCTGACCGTGCTG |
| | | CACCAGGACTGGCTGAACGGCAAAGAGTACAAGTGTAAG |
| | | GTCTCCAACAAGGGCCTGCCAAGCAGCATCGAAAAGACC |
| | | ATCAGCAAGGCCAAGGGCCAGCCTAGAGAGCCCCAGGTC |
| | | TACACCCTGCCACCCAGCCAAGAGGAGATGACCAAGAAC |
| | | CAGGTGTCCCTGACCTGTCTGGTGAAGGGCTTCTACCCAA |
| | | GCGACATCGCCGTGGAGTGGGAGAGCAACGGCCAGCCCG |
| | | AGAACAACTACAAGACCACCCCCCCAGTGCTGGACAGCG |
| | | ACGGCAGCTTCTTCCTGTACAGCAGGCTGACCGTGGACA |
| | | AGTCCAGATGGCAGGAGGGCAACGTCTTTAGCTGCTCCG |
| | | TGATGCACGAGGCCCTGCACAACCACTACACCCAGAAGA |
| | | GCCTGAGCCTGTCCCTGGGC |

BAP050-Clone I LC

| | | |
|---|---|---|
| SEQ ID NO: 710 (Kabat) | LCDR1 | SSSQDISNYLN |
| SEQ ID NO: 711 (Kabat) | LCDR2 | YTSTLHL |
| SEQ ID NO: 712 (Kabat) | LCDR3 | QQYYNLPWT |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 718 | VL | DIQMTQSPSSLSASVGDRVTITCSSSQDISNYLNWYLQKPGQ |
| | | SPQLLIYYTSTLHLGVPSRFSGSGSGTEFTLTISSLQPDDFA |
| | | TYYCQQYYNLPWTFGQGTKVEIK |
| SEQ ID NO: 719 | DNA VL | GATATTCAGATGACTCAGTCACCTAGTAGCCTGAGCGCTA |
| | | GTGTGGGCGATAGAGTGACTATCACCTGTAGCTCTAGTCA |
| | | GGATATCTCTAACTACCTGAACTGGTATCTGCAGAAGCCC |
| | | GGTCAATCACCTCAGCTGCTGATCTACTACACTAGCACCC |
| | | TGCACCTGGGCGTGCCCTCTAGGTTTAGCGGTAGCGGTAG |
| | | TGGCACCGAGTTCACCCTGACTATCTCTAGCCTGCAGCCC |
| | | GACGACTTCGCTACCTACTACTGTCAGCAGTACTATAACC |
| | | TGCCCTGGACCTTCGGTCAAGGCACTAAGGTCGAGATTAAG |
| SEQ ID NO: 720 | DNA VL | GACATCCAGATGACCCAGTCCCCCTCCAGCCTGTCTGCTT |
| | | CCGTGGGCGACAGAGTGACCATCACCTGTTCCTCCAGCC |
| | | AGGACATCTCCAACTACCTGAACTGGTATCTGCAGAAGC |
| | | CCGGCCAGTCCCCTCAGCTGCTGATCTACTACACCTCCAC |
| | | CCTGCACCTGGGCGTGCCCTCCAGATTTTCCGGCTCTGGC |
| | | TCTGGCACCGAGTTTACCCTGACCATCAGCTCCCTGCAGC |
| | | CCGACGACTTCGCCACCTACTACTGCCAGCAGTACTACAA |
| | | CCTGCCCTGGACCTTCGGCCAGGGCACCAAGGTGGAAAT |
| | | CAAG |
| SEQ ID NO: 721 | Light | DIQMTQSPSSLSASVGDRVTITCSSSQDISNYLNWYLQKPGQ |
| | chain | SPQLLIYYTSTLHLGVPSRFSGSGSGTEFTLTISSLQPDDFAT |
| | | YYCQQYYNLPWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| SEQ ID NO: 722 | DNA light | GATATTCAGATGACTCAGTCACCTAGTAGCCTGAGCGCTA |
| | chain | GTGTGGGCGATAGAGTGACTATCACCTGTAGCTCTAGTCA |
| | | GGATATCTCTAACTACCTGAACTGGTATCTGCAGAAGCCC |
| | | GGTCAATCACCTCAGCTGCTGATCTACTACACTAGCACCC |
| | | TGCACCTGGGCGTGCCCTCTAGGTTTAGCGGTAGCGGTAG |
| | | TGGCACCGAGTTCACCCTGACTATCTCTAGCCTGCAGCCC |
| | | GACGACTTCGCTACCTACTACTGTCAGCAGTACTATAACC |
| | | TGCCCTGGACCTTCGGTCAAGGCACTAAGGTCGAGATTA |
| | | AGCGTACGGTGGCCGCTCCCAGCGTGTTCATCTTCCCCCC |
| | | CAGCGACGAGCAGCTGAAGAGCGGCACCGCCAGCGTGGT |
| | | GTGCCTGCTGAACAACTTCTACCCCCGGGAGGCCAAGGT |
| | | GCAGTGGAAGGTGGACAACGCCCTGCAGAGCGGCAACA |
| | | GCCAGGAGAGCGTCACCGAGCAGGACAGCAAGGACTCC |
| | | ACCTACAGCCTGAGCAGCACCCTGACCCTGAGCAAGGCC |
| | | GACTACGAGAAGCATAAGGTGTACGCCTGCGAGGTGACC |
| | | CACCAGGGCCTGTCCAGCCCCGTGACCAAGAGCTTCAAC |
| | | AGGGGCGAGTGC |
| SEQ ID NO: 723 | DNA light | GACATCCAGATGACCCAGTCCCCCTCCAGCCTGTCTGCTT |
| | chain | CCGTGGGCGACAGAGTGACCATCACCTGTTCCTCCAGCC |
| | | AGGACATCTCCAACTACCTGAACTGGTATCTGCAGAAGC |
| | | CCGGCCAGTCCCCTCAGCTGCTGATCTACTACACCTCCAC |
| | | CCTGCACCTGGGCGTGCCCTCCAGATTTTCCGGCTCTGGC |
| | | TCTGGCACCGAGTTTACCCTGACCATCAGCTCCCTGCAGC |
| | | CCGACGACTTCGCCACCTACTACTGCCAGCAGTACTACAA |
| | | CCTGCCCTGGACCTTCGGCCAGGGCACCAAGGTGGAAAT |
| | | CAAGCGTACGGTGGCCGCTCCCAGCGTGTTCATCTTCCCC |
| | | CCAAGCGACGAGCAGCTGAAGAGCGGCACCGCCAGCGTG |
| | | GTGTGTCTGCTGAACAACTTCTACCCCAGGGAGGCCAAG |
| | | GTGCAGTGGAAGGTGGACAACGCCCTGCAGAGCGGCAAC |
| | | AGCCAGGAGAGCGTCACCGAGCAGGACAGCAAGGACTC |
| | | CACCTACAGCCTGAGCAGCACCCTGACCCTGAGCAAGGC |
| | | CGACTACGAGAAGCACAAGGTGTACGCCTGTGAGGTGAC |
| | | CCACCAGGGCCTGTCCAGCCCCGTGACCAAGAGCTTCAA |
| | | CAGGGGCGAGTGC |

BAP050-Clone J HC

| | | |
|---|---|---|
| SEQ ID NO: 701 (Kabat) | HCDR1 | NYGMN |
| SEQ ID NO: 702 (Kabat) | HCDR2 | WINTDTGEPTYADDFKG |
| SEQ ID NO: 703 (Kabat) | HCDR3 | NPPYYYGTNNAEAMDY |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 724 | VH | QVQLVQSGAEVKKPGASVKVSCKASGFTLTNYGMNWVRQ |
| | | APGQGLEWMGWINTDTGEPTYADDFKGRFVFSLDTSVSTA |
| | | YLQISSLKAEDTAVYYCARNPPYYYGTNNAEAMDYWGQG |
| | | TTVTVSS |
| SEQ ID NO: 725 | DNA VH | CAGGTGCAGCTGGTGCAGTCAGGCGCCGAAGTGAAGAAA |
| | | CCCGGCGCTAGTGTGAAAGTCAGCTGTAAAGCTAGTGGC |
| | | TTCACCCTGACTAACTACGGGATGAACTGGGTCCGCCAG |
| | | GCCCCAGGTCAAGGCCTCGAGTGGATGGGCTGGATTAAC |
| | | ACCGACACCGGCGAGCCTACCTACGCCGACGACTTTAAG |
| | | GGCAGATTCGTGTTTAGCCTGGACACTAGTGTGTCTACCG |
| | | CCTACCTGCAGATCTCTAGCCTGAAGGCCGAGGACACCG |
| | | CCGTCTACTACTGCGCTAGAAACCCCCCCTACTACTACGG |
| | | CACTAACAACGCCGAGGCTATGGACTACTGGGGTCAAGG |
| | | CACTACCGTGACCGTGTCTAGC |
| SEQ ID NO: 726 | DNA VH | CAGGTGCAGCTGGTGCAGTCTGGCGCCGAAGTGAAGAAA |
| | | CCTGGCGCCTCCGTGAAGGTGTCCTGCAAGGCCTCTGGCT |
| | | TCACCCTGACCAACTACGGCATGAACTGGGTGCGACAGG |
| | | CCCCTGGACAGGGCCTGGAATGGATGGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCT |
| SEQ ID NO: 727 | Heavy | QVQLVQSGAEVKKPGASVKVSCKASGFTLTNYGMNWVRQ |
| | chain | APGQGLEWMGWINTDTGEPTYADDFKGRFVFSLDTSVSTA |
| | | YLQISSLKAEDTAVYYCARNPPYYYGTNNAEAMDYWGQG |
| | | TTVTVSSASTKGPSVFPLAPCSRSTSESTAALGCLVKDYFPEP |
| | | VTVSWNSGALTSGVHTFPAVLQSSGLYSLSSVVTVPSSSLGT |
| | | KTYTCNVDHKPSNTKVDKRVESKYGPPCPPCPAPEFLGGPS |
| | | VFLFPPKPKDTLMISRTPEVTCVVVDVSQEDPEVQFNWYVD |
| | | GVEVHNAKTKPREEQFNSTYRVVSVLTVLHQDWLNGKEYK |
| | | CKVSNKGLPSSIEKTISKAKGQPREPQVYTLPPSQEEMTKNQ |
| | | VSLTCLVKGFYPSDIAVEWESNGQPENNYKTTPPVLDSDGS |
| | | FFLYSRLTVDKSRWQEGNVFSCSVMHEALHNHYTQKSLSLSLG |
| SEQ ID NO: 728 | DNA | CAGGTGCAGCTGGTGCAGTCAGGCGCCGAAGTGAAGAAA |
| | heavy | CCCGGCGCTAGTGTGAAAGTCAGCTGTAAAGCTAGTGGC |
| | chain | TTCACCCTGACTAACTACGGGATGAACTGGGTCCGCCAG |
| | | GCCCCAGGTCAAGGCCTCGAGTGGATGGGCTGGATTAAC |
| | | ACCGACACCGGCGAGCCTACCTACGCCGACGACTTTAAG |
| | | GGCAGATTCGTGTTTAGCCTGGACACTAGTGTGTCTACCG |
| | | CCTACCTGCAGATCTCTAGCCTGAAGGCCGAGGACACCG |
| | | CCGTCTACTACTGCGCTAGAAACCCCCCCTACTACTACGG |
| | | CACTAACAACGCCGAGGCTATGGACTACTGGGGTCAAGG |
| | | CACTACCGTGACCGTGTCTAGCGCTAGCACTAAGGGCCC |
| | | GTCCGTGTTCCCCCTGGCACCTTGTAGCCGGAGCACTAGC |
| | | GAATCCACCGCTGCCCTCGGCTGCCTGGTCAAGGATTACT |
| | | TCCCGGAGCCCGTGACCGTGTCCTGGAACAGCGGAGCCC |
| | | TGACCTCCGGAGTGCACACCTTCCCCGCTGTGCTGCAGAG |
| | | CTCCGGGCTGTACTCGCTGTCGTCGGTGGTCACGGTGCCT |
| | | TCATCTAGCCTGGGTACCAAGACCTACACTTGCAACGTGG |
| | | ACCACAAGCCTTCCAACACTAAGGTGGACAAGCGCGTCG |
| | | AATCGAAGTACGGCCCACCGTGCCCGCCTTGTCCCGCGCC |
| | | GGAGTTCCTCGGCGGTCCCTCGGTCTTTCTGTTCCCACCG |
| | | AAGCCCAAGGACACTTTGATGATTTCCCGCACCCCTGAA |
| | | GTGACATGCGTGGTCGTGGACGTGTCACAGGAAGATCCG |
| | | GAGGTGCAGTTCAATTGGTACGTGGATGGCGTCGAGGTG |
| | | CACAACGCCAAAACCAAGCCGAGGGAGGAGCAGTTCAA |
| | | CTCCACTTACCGCGTCGTGTCCGTGCTGACGGTGCTGCAT |
| | | CAGGACTGGCTGAACGGGAAGGAGTACAAGTGCAAAGT |
| | | GTCCAACAAGGGACTTCCTAGCTCAATCGAAAAGACCAT |
| | | CTCGAAAGCCAAGGGACAGCCCCGGGAACCCCAAGTGTA |
| | | TACCCTGCCACCGAGCCAGGAAGAAATGACTAAGAACCA |
| | | AGTCTCATTGACTTGCCTTGTGAAGGGCTTCTACCCATCG |
| | | GATATCGCCGTGGAATGGGAGTCCAACGGCCAGCCGGAA |
| | | AACAACTACAAGACCACCCCTCCGGTGCTGGACTCAGAC |
| | | GGATCCTTCTTCCTCTACTCGCGGCTGACCGTGGATAAGA |
| | | GCAGATGGCAGGAGGGAAATGTGTTCAGCTGTTCTGTGA |
| | | TGCATGAAGCCCTGCACAACCACTACACTCAGAAGTCCC |
| | | TGTCCCTCTCCCTGGGA |
| SEQ ID NO: 729 | DNA | CAGGTGCAGCTGGTGCAGTCTGGCGCCGAAGTGAAGAAA |
| | heavy | CCTGGCGCCTCCGTGAAGGTGTCCTGCAAGGCCTCTGGCT |
| | chain | TCACCCTGACCAACTACGGCATGAACTGGGTGCGACAGG |
| | | CCCCTGGACAGGGCCTGGAATGGATGGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCTGCTTCTACCAAGGGGCCCA |
| | | GCGTGTTCCCCCTGGCCCCCTGCTCCAGAAGCACCAGCGA |
| | | GAGCACAGCCGCCCTGGGCTGCCTGGTGAAGGACTACTT |
| | | CCCCGAGCCCGTGACCGTGTCCTGGAACAGCGGAGCCCT |
| | | GACCAGCGGCGTGCACACCTTCCCCGCCGTGCTGCAGAG |
| | | CAGCGGCCTGTACAGCCTGAGCAGCGTGGTGACCGTGCC |
| | | CAGCAGCAGCCTGGGCACCAAGACCTACACCTGTAACGT |
| | | GGACCACAAGCCCAGCAACACCAAGGTGGACAAGAGGG |
| | | TGGAGAGCAAGTACGGCCCACCCTGCCCCCCCTGCCCAG |
| | | CCCCCGAGTTCCTGGGCGGACCCAGCGTGTTCCTGTTCCC |
| | | CCCCAAGCCCAAGGACACCCTGATGATCAGCAGAACCCC |
| | | CGAGGTGACCTGTGTGGTGGTGGACGTGTCCCAGGAGGA |
| | | CCCCGAGGTCCAGTTCAACTGGTACGTGGACGGCGTGGA |
| | | GGTGCACAACGCCAAGACCAAGCCCAGAGAGGAGCAGTT |
| | | TAACAGCACCTACCGGGTGGTGTCCGTGCTGACCGTGCTG |
| | | CACCAGGACTGGCTGAACGGCAAAGAGTACAAGTGTAAG |
| | | GTCTCCAACAAGGGCCTGCCAAGCAGCATCGAAAAGACC |
| | | ATCAGCAAGGCCAAGGGCCAGCCTAGAGAGCCCCAGGTC |
| | | TACACCCTGCCACCCAGCCAAGAGGAGATGACCAAGAAC |
| | | CAGGTGTCCCTGACCTGTCTGGTGAAGGGCTTCTACCCAA |
| | | GCGACATCGCCGTGGAGTGGGAGAGCAACGGCCAGCCCG |
| | | AGAACAACTACAAGACCACCCCCCCAGTGCTGGACAGCG |
| | | ACGGCAGCTTCTTCCTGTACAGCAGGCTGACCGTGGACA |
| | | AGTCCAGATGGCAGGAGGGCAACGTCTTTAGCTGCTCCG |
| | | TGATGCACGAGGCCCTGCACAACCACTACACCCAGAAGA |
| | | GCCTGAGCCTGTCCCTGGGC |

BAP050-Clone J LC

| | | |
|---|---|---|
| SEQ ID NO: 710 (Kabat) | LCDR1 | SSSQDISNYLN |
| SEQ ID NO: 711 (Kabat) | LCDR2 | YTSTLHL |
| SEQ ID NO: 712 (Kabat) | LCDR3 | QQYYNLPWT |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 730 | VL | DIQMTQSPSSLSASVGDRVTITCSSSQDISNYLNWYQQKPGK |
| | | APKLLIYYTSTLHLGIPPRFSGSGYGTDFTLTINNIESEDAA |
| | | YYFCQQYYNLPWTFGQGTKVEIK |
| SEQ ID NO: 731 | DNA VL | GATATTCAGATGACTCAGTCACCTAGTAGCCTGAGCGCTA |
| | | GTGTGGGCGATAGAGTGACTATCACCTGTAGCTCTAGTCA |
| | | GGATATCTCTAACTACCTGAACTGGTATCAGCAGAAGCC |
| | | CGGTAAAGCCCCTAAGCTGCTGATCTACTACACTAGCACC |
| | | CTGCACCTGGGAATCCCCCCTAGGTTTAGCGGTAGCGGCT |
| | | ACGGCACCGACTTCACCCTGACTATTAACAATATCGAGTC |
| | | AGAGGACGCCGCCTACTACTTCTGTCAGCAGTACTATAAC |
| | | CTGCCCTGGACCTTCGGTCAAGGCACTAAGGTCGAGATT |
| | | AAG |
| SEQ ID NO: 732 | DNA VL | GACATCCAGATGACCCAGTCCCCCTCCAGCCTGTCTGCTT |
| | | CCGTGGGCGACAGAGTGACCATCACCTGTTCCTCCAGCC |
| | | AGGACATCTCCAACTACCTGAACTGGTATCAGCAGAAGC |
| | | CCGGCAAGGCCCCCAAGCTGCTGATCTACTACACCTCCAC |
| | | CCTGCACCTGGGCATCCCCCCTAGATTCTCCGGCTCTGGC |
| | | TACGGCACCGACTTCACCCTGACCATCAACAACATCGAG |
| | | TCCGAGGACGCCGCCTACTACTTCTGCCAGCAGTACTACA |
| | | ACCTGCCCTGGACCTTCGGCCAGGGCACCAAGGTGGAAA |
| | | TCAAG |
| SEQ ID NO: 733 | Light | DIQMTQSPSSLSASVGDRVTITCSSSQDISNYLNWYQQKPGK |
| | chain | APKLLIYYTSTLHLGIPPRFSGSGYGTDFTLTINNIESEDAA |
| | | YYFCQQYYNLPWTFGQGTKVEIKRTVAAPSVFIFPPSDEQLK |
| | | SGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| SEQ ID NO: 734 | DNA light | GATATTCAGATGACTCAGTCACCTAGTAGCCTGAGCGCTA |
| | chain | GTGTGGGCGATAGAGTGACTATCACCTGTAGCTCTAGTCA |
| | | GGATATCTCTAACTACCTGAACTGGTATCAGCAGAAGCC |
| | | CGGTAAAGCCCCTAAGCTGCTGATCTACTACACTAGCACC |
| | | CTGCACCTGGGAATCCCCCCTAGGTTTAGCGGTAGCGGCT |
| | | ACGGCACCGACTTCACCCTGACTATTAACAATATCGAGTC |
| | | AGAGGACGCCGCCTACTACTTCTGTCAGCAGTACTATAAC |
| | | CTGCCCTGGACCTTCGGTCAAGGCACTAAGGTCGAGATT |
| | | AAGCGTACGGTGGCCGCTCCCAGCGTGTTCATCTTCCCCC |
| | | CCAGCGACGAGCAGCTGAAGAGCGGCACCGCCAGCGTGG |
| | | TGTGCCTGCTGAACAACTTCTACCCCCGGGAGGCCAAGG |
| | | TGCAGTGGAAGGTGGACAACGCCCTGCAGAGCGGCAACA |
| | | GCCAGGAGAGCGTCACCGAGCAGGACAGCAAGGACTCC |
| | | ACCTACAGCCTGAGCAGCACCCTGACCCTGAGCAAGGCC |
| | | GACTACGAGAAGCATAAGGTGTACGCCTGCGAGGTGACC |
| | | CACCAGGGCCTGTCCAGCCCCGTGACCAAGAGCTTCAAC |
| | | AGGGGCGAGTGC |
| SEQ ID NO: 735 | DNA light | GACATCCAGATGACCCAGTCCCCCTCCAGCCTGTCTGCTT |
| | chain | CCGTGGGCGACAGAGTGACCATCACCTGTTCCTCCAGCC |
| | | AGGACATCTCCAACTACCTGAACTGGTATCAGCAGAAGC |
| | | CCGGCAAGGCCCCCAAGCTGCTGATCTACTACACCTCCAC |
| | | CCTGCACCTGGGCATCCCCCCTAGATTCTCCGGCTCTGGC |
| | | TACGGCACCGACTTCACCCTGACCATCAACAACATCGAG |
| | | TCCGAGGACGCCGCCTACTACTTCTGCCAGCAGTACTACA |
| | | ACCTGCCCTGGACCTTCGGCCAGGGCACCAAGGTGGAAA |
| | | TCAAGCGTACGGTGGCCGCTCCCAGCGTGTTCATCTTCCC |
| | | CCCAAGCGACGAGCAGCTGAAGAGCGGCACCGCCAGCGT |
| | | GGTGTGTCTGCTGAACAACTTCTACCCCAGGGAGGCCAA |
| | | GGTGCAGTGGAAGGTGGACAACGCCCTGCAGAGCGGCAA |
| | | CAGCCAGGAGAGCGTCACCGAGCAGGACAGCAAGGACT |
| | | CCACCTACAGCCTGAGCAGCACCCTGACCCTGAGCAAGG |
| | | CCGACTACGAGAAGCACAAGGTGTACGCCTGTGAGGTGA |
| | | CCCACCAGGGCCTGTCCAGCCCCGTGACCAAGAGCTTCA |
| | | ACAGGGGCGAGTGC |

BAP050-Clone I HC

| | | |
|---|---|---|
| SEQ ID NO: 736 (Kabat) | HCDR1 | AATTACGGGATGAAC |
| SEQ ID NO: 737 (Kabat) | HCDR1 | AACTACGGCATGAAC |
| SEQ ID NO: 738 (Kabat) | HCDR2 | TGGATTAACACCGACACCGGGGAGCCTACCTACGCGGAC |
| | | GATTTCAAGGGA |
| SEQ ID NO: 739 (Kabat) | HCDR2 | TGGATCAACACCGACACCGGCGAGCCTACCTACGCCGAC |
| | | GACTTCAAGGGC |
| SEQ ID NO: 740 (Kabat) | HCDR3 | AACCCGCCCTACTACTACGGAACCAACAACGCCGAAGCC |
| | | ATGGACTAC |
| SEQ ID NO: 741 (Kabat) | HCDR3 | AACCCCCCTTACTACTACGGCACCAACAACGCCGAGGCC |
| | | ATGGACTAT |
| SEQ ID NO: 742 | HCDR1 | GGATTCACCCTCACCAATTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 740 | HCDR3 | AACCCGCCCTACTACTACGGAACCAACAACGCCGAAGCC |
| (Chothia) | | ATGGACTAC |
| SEQ ID NO: 741 | HCDR3 | AACCCCCCTTACTACTACGGCACCAACAACGCCGAGGCC |
| (Chothia) | | ATGGACTAT |

BAP050-Clone I LC

| | | |
|---|---|---|
| SEQ ID NO: 746 (Kabat) | LCDR1 | AGCTCTAGTCAGGATATCTCTAACTACCTGAAC |
| SEQ ID NO: 747 (Kabat) | LCDR1 | TCCTCCAGCCAGGACATCTCCAACTACCTGAAC |
| SEQ ID NO: 748 (Kabat) | LCDR2 | TACACTAGCACCCTGCACCTG |
| SEQ ID NO: 749 (Kabat) | LCDR2 | TACACCTCCACCCTGCACCTG |
| SEQ ID NO: 750 (Kabat) | LCDR3 | CAGCAGTACTATAACCTGCCCTGGACC |
| SEQ ID NO: 751 (Kabat) | LCDR3 | CAGCAGTACTACAACCTGCCCTGGACC |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |

BAP050-Clone J HC

| | | |
|---|---|---|
| SEQ ID NO: 758 (Kabat) | HCDR1 | AACTACGGGATGAAC |
| SEQ ID NO: 737 (Kabat) | HCDR1 | AACTACGGCATGAAC |
| SEQ ID NO: 759 (Kabat) | HCDR2 | TGGATTAACACCGACACCGGCGAGCCTACCTACGCCGAC |
| | | GACTTTAAGGGC |
| SEQ ID NO: 739 (Kabat) | HCDR2 | TGGATCAACACCGACACCGGCGAGCCTACCTACGCCGAC |
| | | GACTTCAAGGGC |
| SEQ ID NO: 760 (Kabat) | HCDR3 | AACCCCCCCTACTACTACGGCACTAACAACGCCGAGGCT |
| | | ATGGACTAC |
| SEQ ID NO: 741 (Kabat) | HCDR3 | AACCCCCCTTACTACTACGGCACCAACAACGCCGAGGCC |
| | | ATGGACTAT |
| SEQ ID NO: 761 | HCDR1 | GGCTTCACCCTGACTAACTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 760 | HCDR3 | AACCCCCCCTACTACTACGGCACTAACAACGCCGAGGCT |
| (Chothia) | | ATGGACTAC |
| SEQ ID NO: 741 | HCDR3 | AACCCCCCTTACTACTACGGCACCAACAACGCCGAGGCC |
| (Chothia) | | ATGGACTAT |

BAP050-Clone J LC

| | | |
|---|---|---|
| SEQ ID NO: 746 (Kabat) | LCDR1 | AGCTCTAGTCAGGATATCTCTAACTACCTGAAC |
| SEQ ID NO: 747 (Kabat) | LCDR1 | TCCTCCAGCCAGGACATCTCCAACTACCTGAAC |
| SEQ ID NO: 748 (Kabat) | LCDR2 | TACACTAGCACCCTGCACCTG |
| SEQ ID NO: 749 (Kabat) | LCDR2 | TACACCTCCACCCTGCACCTG |
| SEQ ID NO: 750 (Kabat) | LCDR3 | CAGCAGTACTATAACCTGCCCTGGACC |
| SEQ ID NO: 751 (Kabat) | LCDR3 | CAGCAGTACTACAACCTGCCCTGGACC |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |

In some embodiments, the LAG-3 inhibitor (e.g., an anti-LAG-3 antibody molecule described herein) is administered at a dose of about 300-1000 mg, e.g., about 300 mg to about 500 mg, about 400 mg to about 800 mg, or about 700 mg to about 900 mg. In embodiments, the LAG-3 inhibitor is administered once every week, once every two weeks, once every three weeks, once every four weeks, once every five weeks or once every six weeks. In embodiments, the LAG-3 inhibitor is administered once every 3 weeks. In embodiments, the LAG-3 inhibitor is administered once every 4 weeks. In other embodiments, the LAG-3 inhibitor is administered at a dose of about 300 mg to about 500 mg (e.g., about 400 mg) once every 3 weeks. In yet other embodiments, the PD-1 inhibitor is administered at a dose of about 700 mg to about 900 mg (e.g., about 800 mg) once every 4 weeks. In yet other embodiments, the LAG-3 inhibitor is administered at a dose of about 400 mg to about 800 mg (e.g., about 600 mg) once every 4 weeks.

In some embodiments, a composition comprises a LAG-3 inhibitor, e.g., a LAG-3 inhibitor described herein, and a PD-1 inhibitor, e.g., a PD-1 inhibitor described herein. In some embodiments, the combination of a LAG-3 inhibitor and a PD-1 inhibitor is administered in a therapeutically effective amount to a subject with a solid tumor, e.g., a breast cancer, e.g., a triple negative breast cancer. Without wishing to be bound by theory, it is believed that a combination comprising a LAG-3 inhibitor and a PD-1 inhibitor has increased activity compared to administration of a PD-1 inhibitor alone.

In some embodiments, a composition comprises a LAG-3 inhibitor, e.g., a LAG-3 inhibitor described herein, a GITR agonist, e.g., a GITR agonist described herein, and a PD-1 inhibitor, e.g., a PD-1 inhibitor described herein. In some embodiments, the combination of a LAG-3 inhibitor, a GITR agonist, and a PD-1 inhibitor is administered in a therapeutically effective amount to a subject with a solid tumor, e.g., a breast cancer, e.g., a triple negative breast cancer. In some embodiments, a combination comprising a LAG-3 inhibitor, a GITR agonist, and a PD-1 inhibitor can result in increased IL-2 production.

Other Exemplary LAG-3 Inhibitors

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 6.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 6. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 6
Amino acid
sequences of other exemplary anti-LAG-3 antibody molecules

| | | |
|---|---|---|
| BMS-986016 | | |
| SEQ ID NO: 762 | Heavy chain | QVQLQQWGAGLLKPSETLSLTCAVYGGSFSDYYWNWIRQPPGK |
| | | GLEWIGEINHRGSTNSNPSLKSRVTLSLDTSKNQFSLKLRSVTAA |
| | | DTAVYYCAFGYSDYEYNWFDPWGQGTLVTVSSASTKGPSVFPL |
| | | APCSRSTSESTAALGCLVKDYFPEPVTVSWNSGALTSGVHTFPA |
| | | VLQSSGLYSLSSVVTVPSSSLGTKTYTCNVDHKPSNTKVDKRVE |
| | | SKYGPPCPPCPAPEFLGGPSVFLFPPKPKDTLMISRTPEVTCVVVD |
| | | VSQEDPEVQFNWYVDGVEVHNAKTKPREEQFNSTYRVVSVLTV |
| | | LHQDWLNGKEYKCKVSNKGLPSSIEKTISKAKGQPREPQVYTLP |
| | | PSQEEMTKNQVSLTCLVKGFYPSDIAVEWESNGQPENNYKTTPP |
| | | VLDSDGSFFLYSRLTVDKSRWQEGNVFSCSVMHEALHNHYTQK |
| | | SLSLSLGK |
| SEQ ID NO: 763 | Light chain | EIVLTQSPATLSLSPGERATLSCRASQSISSYLAWYQQKPGQAPR |
| | | LLIYDASNRATGIPARFSGSGSGTDFTLTISSLEPEDFAVYYCQQR |
| | | SNWPLTFGQGTNLEIKRTVAAPSVFIFPPSDEQLKSGTASVVCLL |
| | | NNFYPREAKVQWKVDNALQSGNSQESVTEQDSKDSTYSLSSTL |
| | | TLSKADYEKHKVYACEVTHQGLSSPVTKSFNRGEC |
| IMP731 | | |
| SEQ ID NO: 764 | Heavy chain | QVQLKESGPGLVAPSQSLSITCTVSGFSLTAYGVNWVRQPPGKG |
| | | LEWLGMIWDDGSTDYNSALKSRLSISKDNSKSQVFLKMNSLQT |
| | | DDTARYYCAREGDVAFDYWGQGTTLTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSRDELTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| SEQ ID NO: 765 | Light chain | DIVMTQSPSSLAVSVGQKVTMSCKSSQSLLNGSNQKNYLAWYQ |
| | | QKPGQSPKLLVYFASTRDSGVPDRFIGSGSGTDFTLTISSVQAED |
| | | LADYFCLQHFGTPPTFGGGTKLEIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |

### TIM-3 Inhibitors

In certain embodiments, a combination described herein comprises a TIM-3 inhibitor.

Without wishing to be bound by theory, it is believed that TIM-3 correlates with tumor myeloid signature in The Cancer Genome Atlas (TCGA) database and the most abundant TIM-3 on normal peripheral blood mononuclear cells (PBMCs) is on myeloid cells. TIM-3 is expressed on multiple myeloid subsets in human PBMCs, including, but not limited to, monocytes, macrophages and dendritic cells.

Tumor purity estimates are negatively correlated with TIM-3 expression in a number of TCGA tumor samples (including, e.g., adrenocortical carcinoma (ACC), bladder urothelial carcinoma (BLCA), breast invasive carcinoma (BRCA), cervical squamous cell carcinoma and endocervical adenocarcinoma (CESC), colon adenocarcinoma (COAD), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), brain low grade glioma (LGG), liver hepatocellular carcinoma (LIHC), lung adenocarcinoma (LUAD), lung squamous cell carcinoma (LUSC), ovarian serous cystadenocarcinoma (OV), prostate adenocarcinoma (PRAD), rectum adenocarcinoma (READ), skin cutaneous melanoma (SKCM), thyroid carcinoma (THCA), uterine corpus endometrial carcinoma (UCEC), and uterine carcinosarcoma (UCS)), suggesting TIM-3 expression in tumor samples is from tumor infiltrates.

In certain embodiments, the combination is used to treat a kidney cancer (e.g., a kidney renal clear cell carcinoma (KIRC) or a kidney renal papillary cell carcinoma (KIRP)). In other embodiments, the combination is used to treat a brain tumor (e.g., a brain low grade glioma (LGG) or a glioblastoma multiforme (GBM)). In some embodiments, the combination is used to treat a mesothelioma (MESO). In some embodiments, the combination is used to treat a sarcoma (SARC), a lung adenocarcinoma (LUAD), a pancreatic adenocarcinoma (PAAD), or a lung squamous cell carcinoma (LUSC).

Without wishing to be bound by theory, it is believed that in some embodiments, by clustering indications by immune signatures, cancers that can be effectively treated by a combination described herein can be identified, e.g., by determining the fraction of patients in each indication above 75^{th} percentile across TCGA.

In some embodiments, a T cell gene signature comprises expression of one or more (e.g., all) of: CD2, CD247, CD3D, CD3E, CD3G, CD8A, CD8B, CXCR6, GZMK, PYHIN1, SH2D1A, SIRPG or TRAT1.

In some embodiments, a Myeloid gene signature comprises expression of one or more (e.g., all) of SIGLEC1, MSR1, LILRB4, ITGAM or CD163.

In some embodiments, a TIM-3 gene signature comprises expression of one or more (e.g., all) of HAVCR2, ADGRG1, PIK3AP1, CCL3, CCL4, PRF1, CD8A, NKG7, or KLRK1.

Without wishing to be bound by theory, it is believed that in some embodiments, a TIM-3 inhibitor, e.g., MBG453, synergizes with a PD-1 inhibitor, e.g., PDR001, in a mixed lymphocyte reaction (MLR) assay. In some embodiments, inhibition of PD-L1 and TIM-3 results in tumor reduction and survival in mouse models of cancer. In some embodiments, inhibition of PD-L1 and LAG-3 results in tumor reduction and survival in mouse models of cancer.

In some embodiments, the combination is used to treat a cancer having high levels of expression of TIM-3 and one or more of myeloid signature genes (e.g., one or more genes expressed in macrophages). In some embodiments, the cancer having high levels of expression of TIM-3 and myeloid signature genes is chosen from a sarcoma (SARC), a mesothelioma (MESO), a brain tumor (e.g., a glioblastoma (GBM), or a kidney cancer (e.g., a kidney renal papillary cell carcinoma (KIRP)). In other embodiments, the combination is used to treat a cancer having high levels of expression of TIM-3 and one or more of T cell signature genes (e.g., one or more genes expressed in dendritic cells and/or T cells). In some embodiments, the cancer having high levels of expression of TIM-3 and T cell signature genes is chosen from a kidney cancer (e.g., a kidney renal clear cell carcinoma (KIRC)), a lung cancer (e.g., a lung adenocarcinoma (LUAD)), a pancreatic adenocarcinoma (PAAD), or a testicular cancer (e.g., a testicular germ cell tumor (TGCT)).

Without wishing to be bound by theory, it is believed that in some embodiments, by clustering indications by immune signatures, cancers that can be effectively treated by a combination targeting two, three, or more targets described herein can be identified, e.g., by determining the fraction of patients above 75^{th} percentile in both or all of the targets.

In some embodiments, the combination comprises a TIM-3 inhibitor (e.g., a TIM-3 inhibitor described herein) and a PD-1 inhibitor (e.g., a PD-1 inhibitor described herein), e.g., to treat cancer chosen from a kidney cancer (e.g., a kidney renal papillary cell carcinoma (KIRC) or a kidney renal papillary cell carcinoma (KIRP)), a mesothelioma (MESO), a lung cancer (e.g., a lung adenocarcinoma (LUAD) or a lung squamous cell carcinoma (LUSC)), a sarcoma (SARC), a testicular cancer (e.g., a testicular germ cell tumor (TGCT)), a pancreatic cancer (e.g., a pancreatic adenocarcinoma (PAAD)), a cervical cancer (e.g., cervical squamous cell carcinoma and endocervical adenocarcinoma (CESC)), a head and neck cancer (e.g., a head and neck squamous cell carcinoma (HNSC)), a bladder cancer (e.g., bladder urothelial carcinoma (BLCA), a stomach cancer (e.g., stomach adenocarcinoma (STAD)), a skin cancer (e.g., skin cutaneous melanoma (SKCM)), a breast cancer (e.g., breast invasive carcinoma (BRCA)), or a cholangiocarcinoma (CHOL).

In some embodiments, the combination comprises a TIM-3 inhibitor (e.g., a TIM-3 inhibitor described herein) and a LAG-3 inhibitor (e.g., a LAG-3 inhibitor described herein), e.g., to treat cancer chosen from a kidney cancer (e.g., a kidney renal papillary cell carcinoma (KIRC)), a mesothelioma (MESO), a lung cancer (e.g., a lung adenocarcinoma (LUAD) or a lung squamous cell carcinoma (LUSC)), a sarcoma (SARC), a testicular cancer (e.g., a testicular germ cell tumor (TGCT)), a cervical cancer (e.g., cervical squamous cell carcinoma and endocervical adenocarcinoma (CESC)), an ovarian cancer (OV), a head and neck cancer (e.g., a head and neck squamous cell carcinoma (HNSC)), a stomach cancer (e.g., stomach adenocarcinoma (STAD)), a bladder cancer (e.g., bladder urothelial carcinoma (BLCA), a breast cancer (e.g., breast invasive carcinoma (BRCA)), or a skin cancer (e.g., skin cutaneous melanoma (SKCM)).

In some embodiments, the combination comprises a TIM-3 inhibitor (e.g., a TIM-3 inhibitor described herein), a PD-1 inhibitor (e.g., a PD-1 inhibitor described herein), and a LAG-3 inhibitor (e.g., a LAG-3 inhibitor described herein), e.g., to treat a cancer chosen from a kidney cancer (e.g., a kidney renal papillary cell carcinoma (KIRC)), a lung cancer (e.g., a lung adenocarcinoma (LUAD) or a lung squamous cell carcinoma (LUSC)), a mesothelioma (MESO), a testicular cancer (e.g., a testicular germ cell tumor (TGCT)), a sarcoma (SARC), a cervical cancer (e.g., cervical squamous cell carcinoma and endocervical adenocarcinoma (CESC)), a head and neck cancer (e.g., a head and neck squamous cell carcinoma (HNSC)), a stomach cancer (e.g., stomach adenocarcinoma (STAD)), an ovarian cancer (OV), a bladder cancer (e.g., bladder urothelial carcinoma (BLCA), a breast cancer (e.g., breast invasive carcinoma (BRCA)), or a skin cancer (e.g., skin cutaneous melanoma (SKCM)).

In some embodiments, the combination comprises a TIM-3 inhibitor (e.g., a TIM-3 inhibitor described herein), a PD-1 inhibitor (e.g., a PD-1 inhibitor described herein), and a c-MET inhibitor (e.g., a c-MET inhibitor described herein), e.g., to treat a cancer chosen from a kidney cancer (e.g., a kidney renal papillary cell carcinoma (KIRC)), a lung cancer (e.g., a lung adenocarcinoma (LUAD), or a mesothelioma (MESO).

In some embodiments, the TIM-3 inhibitor is MBG453 (Novartis) or TSR-022 (Tesaro). In some embodiments, the TIM-3 inhibitor is MBG453.

Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 7 (e.g., from the heavy and light chain variable region sequences of ABTIM3-hum11 or ABTIM3-hum03 disclosed in Table 7), or encoded by a nucleotide sequence shown in Table 7. In some embodiments, the CDRs are according to the Kabat definition (e.g., as set out in Table 7). In some embodiments, the CDRs are according to the Chothia definition (e.g., as set out in Table 7). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 7, or encoded by a nucleotide sequence shown in Table 7.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 7. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 7.

In one embodiment, the anti-TIM-3 antibody molecule comprises a VH comprising the amino acid sequence of SEQ ID NO: 806, or an amino acid sequence at least 85%, 90%, 95%, or 99% identical or higher to SEQ ID NO: 806. In one embodiment, the anti-TIM-3 antibody molecule comprises a VL comprising the amino acid sequence of SEQ ID NO: 816, or an amino acid sequence at least 85%, 90%, 95%, or 99% identical or higher to SEQ ID NO: 816. In one embodiment, the anti-TIM-3 antibody molecule comprises a VH comprising the amino acid sequence of SEQ ID NO: 822, or an amino acid sequence at least 85%, 90%, 95%, or 99% identical or higher to SEQ ID NO: 822. In one embodiment, the anti-TIM-3 antibody molecule comprises a VL comprising the amino acid sequence of SEQ ID NO: 826, or an amino acid sequence at least 85%, 90%, 95%, or 99% identical or higher to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence at least 85%, 90%, 95%, or 99% identical or higher to SEQ ID NO: 807. In one embodiment, the antibody molecule comprises a VL encoded by the nucleotide sequence of SEQ ID NO: 817, or a nucleotide sequence at least 85%, 90%, 95%, or 99% identical or higher to SEQ ID NO: 817. In one embodiment, the antibody molecule comprises a VH encoded by the nucleotide sequence of SEQ ID NO: 823, or a nucleotide sequence at least 85%, 90%, 95%, or 99% identical or higher to SEQ ID NO: 823. In one embodiment, the antibody molecule comprises a VL encoded by the nucleotide sequence of SEQ ID NO: 827, or a nucleotide sequence at least 85%, 90%, 95%, or 99% identical or higher to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence at least 85%, 90%, 95%, or 99% identical or higher to SEQ ID NO: 808. In one embodiment, the anti-TIM-3 antibody molecule comprises a light chain comprising the amino acid sequence of SEQ ID NO: 818, or an amino acid sequence at least 85%, 90%, 95%, or 99% identical or higher to SEQ ID NO: 818. In one embodiment, the anti-TIM-3 antibody molecule comprises a heavy chain comprising the amino acid sequence of SEQ ID NO: 824, or an amino acid sequence at least 85%, 90%, 95%, or 99% identical or higher to SEQ ID NO: 824. In one embodiment, the anti-TIM-3 antibody molecule comprises a light chain comprising the amino acid sequence of SEQ ID NO: 828, or an amino acid sequence at least 85%, 90%, 95%, or 99% identical or higher to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence at least 85%, 90%, 95%, or 99% identical or higher to SEQ ID NO: 809. In one embodiment, the antibody molecule comprises a light chain encoded by the nucleotide sequence of SEQ ID NO: 819, or a nucleotide sequence at least 85%, 90%, 95%, or 99% identical or higher to SEQ ID NO: 819. In one embodiment, the antibody molecule comprises a heavy chain encoded by the nucleotide sequence of SEQ ID NO: 825, or a nucleotide sequence at least 85%, 90%, 95%, or 99% identical or higher to SEQ ID NO: 825. In one embodiment, the antibody molecule comprises a light chain encoded by the nucleotide sequence of SEQ ID NO: 829, or a nucleotide sequence at least 85%, 90%, 95%, or 99% identical or higher to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

The antibody molecules described herein can be made by vectors, host cells, and methods described in US 2015/0218274, incorporated by reference in its entirety.

TABLE 7
Amino acid and nucleotide sequences of exemplary
anti-TIM-3 antibody molecules

| | | |
|---|---|---|
| ABTIM3-hum11 | | |
| SEQ ID NO: 801 (Kabat) | HCDR1 | SYNMH |
| SEQ ID NO: 802 (Kabat) | HCDR2 | DIYPGNGDTSYNQKFKG |
| SEQ ID NO: 803 (Kabat) | HCDR3 | VGGAFPMDY |
| SEQ ID NO: 804 (Chothia) | HCDR1 | GYTFTSY |
| SEQ ID NO: 805 (Chothia) | HCDR2 | YPGNGD |
| SEQ ID NO: 803 (Chothia) | HCDR3 | VGGAFPMDY |
| SEQ ID NO: 806 | VH | QVQLVQSGAEVKKPGSSVKVSCKASGYTFTSYNMHWV |
| | | RQAPGQGLEWMGDIYPGNGDTSYNQKFKGRVTITADKS |
| | | TSTVYMELSSLRSEDTAVYYCARVGGAFPMDYWGQGTT |
| | | VTVSS |
| SEQ ID NO: 807 | DNA VH | CAGGTGCAGCTGGTGCAGTCAGGCGCCGAAGTGAAGA |
| | | AACCCGGCTCTAGCGTGAAAGTTTCTTGTAAAGCTAGT |
| | | GGCTACACCTTCACTAGCTATAATATGCACTGGGTTCG |
| | | CCAGGCCCCAGGGCAAGGCCTCGAGTGGATGGGCGAT |
| | | ATCTACCCCGGGAACGGCGACACTAGTTATAATCAGA |
| | | AGTTTAAGGGTAGAGTCACTATCACCGCCGATAAGTCT |
| | | ACTAGCACCGTCTATATGGAACTGAGTTCCCTGAGGTC |
| | | TGAGGACACCGCCGTCTACTACTGCGCTAGAGTGGGC |
| | | GGAGCCTTCCCTATGGACTACTGGGGTCAAGGCACTA |
| | | CCGTGACCGTGTCTAGC |
| SEQ ID NO: 808 | Heavy | QVQLVQSGAEVKKPGSSVKVSCKASGYTFTSYNMHWV |
| | chain | RQAPGQGLEWMGDIYPGNGDTSYNQKFKGRVTITADKS |
| | | TSTVYMELSSLRSEDTAVYYCARVGGAFPMDYWGQGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEF |
| | | LGGPSVFLFPPKPKDTLMISRTPEVTCVVVDVSQEDPEVQ |
| | | FNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLG |
| SEQ ID NO: 809 | DNA | CAGGTGCAGCTGGTGCAGTCAGGCGCCGAAGTGAAGA |
| | heavy | AACCCGGCTCTAGCGTGAAAGTTTCTTGTAAAGCTAGT |
| | chain | GGCTACACCTTCACTAGCTATAATATGCACTGGGTTCG |
| | | CCAGGCCCCAGGGCAAGGCCTCGAGTGGATGGGCGAT |
| | | ATCTACCCCGGGAACGGCGACACTAGTTATAATCAGA |
| | | AGTTTAAGGGTAGAGTCACTATCACCGCCGATAAGTCT |
| | | ACTAGCACCGTCTATATGGAACTGAGTTCCCTGAGGTC |
| | | TGAGGACACCGCCGTCTACTACTGCGCTAGAGTGGGC |
| | | GGAGCCTTCCCTATGGACTACTGGGGTCAAGGCACTA |
| | | CCGTGACCGTGTCTAGCGCTAGCACTAAGGGCCCGTCC |
| | | GTGTTCCCCCTGGCACCTTGTAGCCGGAGCACTAGCGA |
| | | ATCCACCGCTGCCCTCGGCTGCCTGGTCAAGGATTACT |
| | | TCCCGGAGCCCGTGACCGTGTCCTGGAACAGCGGAGC |
| | | CCTGACCTCCGGAGTGCACACCTTCCCCGCTGTGCTGC |
| | | AGAGCTCCGGGCTGTACTCGCTGTCGTCGGTGGTCACG |
| | | GTGCCTTCATCTAGCCTGGGTACCAAGACCTACACTTG |
| | | CAACGTGGACCACAAGCCTTCCAACACTAAGGTGGAC |
| | | AAGCGCGTCGAATCGAAGTACGGCCCACCGTGCCCGC |
| | | CTTGTCCCGCGCCGGAGTTCCTCGGCGGTCCCTCGGTC |
| | | TTTCTGTTCCCACCGAAGCCCAAGGACACTTTGATGAT |
| | | TTCCCGCACCCCTGAAGTGACATGCGTGGTCGTGGACG |
| | | TGTCACAGGAAGATCCGGAGGTGCAGTTCAATTGGTA |
| | | CGTGGATGGCGTCGAGGTGCACAACGCCAAAACCAAG |
| | | CCGAGGGAGGAGCAGTTCAACTCCACTTACCGCGTCG |
| | | TGTCCGTGCTGACGGTGCTGCATCAGGACTGGCTGAAC |
| | | GGGAAGGAGTACAAGTGCAAAGTGTCCAACAAGGGA |
| | | CTTCCTAGCTCAATCGAAAAGACCATCTCGAAAGCCA |
| | | AGGGACAGCCCCGGGAACCCCAAGTGTATACCCTGCC |
| | | ACCGAGCCAGGAAGAAATGACTAAGAACCAAGTCTCA |
| | | TTGACTTGCCTTGTGAAGGGCTTCTACCCATCGGATAT |
| | | CGCCGTGGAATGGGAGTCCAACGGCCAGCCGGAAAAC |
| | | AACTACAAGACCACCCCTCCGGTGCTGGACTCAGACG |
| | | GATCCTTCTTCCTCTACTCGCGGCTGACCGTGGATAAG |
| | | AGCAGATGGCAGGAGGGAAATGTGTTCAGCTGTTCTG |
| | | TGATGCATGAAGCCCTGCACAACCACTACACTCAGAA |
| | | GTCCCTGTCCCTCTCCCTGGGA |
| SEQ ID NO: 810 (Kabat | LCDR1 | RASESVEYYGTSLMQ |
| SEQ ID NO: 811 (Kabat) | LCDR1 | AASNVES |
| SEQ ID NO: 812 (Kabat) | LCDR3 | QQSRKDPST |
| SEQ ID NO: 813 (Chothia) | LCDR1 | SESVEYYGTSL |
| SEQ ID NO: 814 (Chothia | LCDR2 | AAS |
| SEQ ID NO: 815 (Chothia) | LCDR3 | SRKDPS |
| SEQ ID NO: 816 | VL | AIQLTQSPSSLSASVGDRVTITCRASESVEYYGTSLMQWY |
| | | QQKPGKAPKLLIYAASNVESGVPSRFSGSGSGTDFTLTISS |
| | | LQPEDFATYFCQQSRKDPSTFGGGTKVEIK |
| SEQ ID NO: 817 | DNA VL | GCTATTCAGCTGACTCAGTCACCTAGTAGCCTGAGCGC |
| | | TAGTGTGGGCGATAGAGTGACTATCACCTGTAGAGCT |
| | | AGTGAATCAGTCGAGTACTACGGCACTAGCCTGATGC |
| | | AGTGGTATCAGCAGAAGCCCGGGAAAGCCCCTAAGCT |
| | | GCTGATCTACGCCGCCTCTAACGTGGAATCAGGCGTGC |
| | | CCTCTAGGTTTAGCGGTAGCGGTAGTGGCACCGACTTC |
| | | ACCCTGACTATCTCTAGCCTGCAGCCCGAGGACTTCGC |
| | | TACCTACTTCTGTCAGCAGTCTAGGAAGGACCCTAGCA |
| | | CCTTCGGCGGAGGCACTAAGGTCGAGATTAAG |
| SEQ ID NO: 818 | Light | AIQLTQSPSSLSASVGDRVTITCRASESVEYYGTSLMQWY |
| | chain | QQKPGKAPKLLIYAASNVESGVPSRFSGSGSGTDFTLTISS |
| | | LQPEDFATYFCQQSRKDPSTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| SEQ ID NO: 819 | DNA light | GCTATTCAGCTGACTCAGTCACCTAGTAGCCTGAGCGC |
| | chain | TAGTGTGGGCGATAGAGTGACTATCACCTGTAGAGCT |
| | | AGTGAATCAGTCGAGTACTACGGCACTAGCCTGATGC |
| | | AGTGGTATCAGCAGAAGCCCGGGAAAGCCCCTAAGCT |
| | | GCTGATCTACGCCGCCTCTAACGTGGAATCAGGCGTGC |
| | | CCTCTAGGTTTAGCGGTAGCGGTAGTGGCACCGACTTC |
| | | ACCCTGACTATCTCTAGCCTGCAGCCCGAGGACTTCGC |
| | | TACCTACTTCTGTCAGCAGTCTAGGAAGGACCCTAGCA |
| | | CCTTCGGCGGAGGCACTAAGGTCGAGATTAAGCGTAC |
| | | GGTGGCCGCTCCCAGCGTGTTCATCTTCCCCCCCAGCG |
| | | ACGAGCAGCTGAAGAGCGGCACCGCCAGCGTGGTGTG |
| | | CCTGCTGAACAACTTCTACCCCCGGGAGGCCAAGGTG |
| | | CAGTGGAAGGTGGACAACGCCCTGCAGAGCGGCAACA |
| | | GCCAGGAGAGCGTCACCGAGCAGGACAGCAAGGACTC |
| | | CACCTACAGCCTGAGCAGCACCCTGACCCTGAGCAAG |
| | | GCCGACTACGAGAAGCATAAGGTGTACGCCTGCGAGG |
| | | TGACCCACCAGGGCCTGTCCAGCCCCGTGACCAAGAG |
| | | CTTCAACAGGGGCGAGTGC |
| ABTIM3-hum03 | | |
| SEQ ID NO: 801 (Kabat) | HCDR1 | SYNMH |
| SEQ ID NO: 820 (Kabat) | HCDR2 | DIYPGQGDTSYNQKFKG |
| SEQ ID NO: 803 (Kabat) | HCDR3 | VGGAFPMDY |
| SEQ ID NO: 804 (Chothia) | HCDR1 | GYTFTSY |
| SEQ ID NO: 821 (Chothia) | HCDR2 | YPGQGD |
| SEQ ID NO: 803 (Chothia) | HCDR3 | VGGAFPMDY |
| SEQ ID NO: 822 | VH | QVQLVQSGAEVKKPGASVKVSCKASGYTFTSYNMHWV |
| | | RQAPGQGLEWIGDIYPGQGDTSYNQKFKGRATMTADKS |
| | | TSTVYMELSSLRSEDTAVYYCARVGGAFPMDYWGQGTL |
| | | VTVSS |
| SEQ ID NO: 823 | DNA VH | CAGGTGCAGCTGGTGCAGTCAGGCGCCGAAGTGAAGA |
| | | AACCCGGCGCTAGTGTGAAAGTTAGCTGTAAAGCTAG |
| | | TGGCTATACTTTCACTTCTTATAATATGCACTGGGTCC |
| | | GCCAGGCCCCAGGTCAAGGCCTCGAGTGGATCGGCGA |
| | | TATCTACCCCGGTCAAGGCGACACTTCCTATAATCAGA |
| | | AGTTTAAGGGTAGAGCTACTATGACCGCCGATAAGTC |
| | | TACTTCTACCGTCTATATGGAACTGAGTTCCCTGAGGT |
| | | CTGAGGACACCGCCGTCTACTACTGCGCTAGAGTGGG |
| | | CGGAGCCTTCCCAATGGACTACTGGGGTCAAGGCACC |
| | | CTGGTCACCGTGTCTAGC |
| SEQ ID NO: 824 | Heavy | QVQLVQSGAEVKKPGASVKVSCKASGYTFTSYNMHWV |
| | chain | RQAPGQGLEWIGDIYPGQGDTSYNQKFKGRATMTADKS |
| | | TSTVYMELSSLRSEDTAVYYCARVGGAFPMDYWGQGTL |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEF |
| | | LGGPSVFLFPPKPKDTLMISRTPEVTCVVVDVSQEDPEVQ |
| | | FNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLG |
| SEQ ID NO: 825 | DNA | CAGGTGCAGCTGGTGCAGTCAGGCGCCGAAGTGAAGA |
| | heavy | AACCCGGCGCTAGTGTGAAAGTTAGCTGTAAAGCTAG |
| | chain | TGGCTATACTTTCACTTCTTATAATATGCACTGGGTCC |
| | | GCCAGGCCCCAGGTCAAGGCCTCGAGTGGATCGGCGA |
| | | TATCTACCCCGGTCAAGGCGACACTTCCTATAATCAGA |
| | | AGTTTAAGGGTAGAGCTACTATGACCGCCGATAAGTC |
| | | TACTTCTACCGTCTATATGGAACTGAGTTCCCTGAGGT |
| | | CTGAGGACACCGCCGTCTACTACTGCGCTAGAGTGGG |
| | | CGGAGCCTTCCCAATGGACTACTGGGGTCAAGGCACC |
| | | CTGGTCACCGTGTCTAGCGCTAGCACTAAGGGCCCGTC |
| | | CGTGTTCCCCCTGGCACCTTGTAGCCGGAGCACTAGCG |
| | | AATCCACCGCTGCCCTCGGCTGCCTGGTCAAGGATTAC |
| | | TTCCCGGAGCCCGTGACCGTGTCCTGGAACAGCGGAG |
| | | CCCTGACCTCCGGAGTGCACACCTTCCCCGCTGTGCTG |
| | | CAGAGCTCCGGGCTGTACTCGCTGTCGTCGGTGGTCAC |
| | | GGTGCCTTCATCTAGCCTGGGTACCAAGACCTACACTT |
| | | GCAACGTGGACCACAAGCCTTCCAACACTAAGGTGGA |
| | | CAAGCGCGTCGAATCGAAGTACGGCCCACCGTGCCCG |
| | | CCTTGTCCCGCGCCGGAGTTCCTCGGCGGTCCCTCGGT |
| | | CTTTCTGTTCCCACCGAAGCCCAAGGACACTTTGATGA |
| | | TTTCCCGCACCCCTGAAGTGACATGCGTGGTCGTGGAC |
| | | GTGTCACAGGAAGATCCGGAGGTGCAGTTCAATTGGT |
| | | ACGTGGATGGCGTCGAGGTGCACAACGCCAAAACCAA |
| | | GCCGAGGGAGGAGCAGTTCAACTCCACTTACCGCGTC |
| | | GTGTCCGTGCTGACGGTGCTGCATCAGGACTGGCTGA |
| | | ACGGGAAGGAGTACAAGTGCAAAGTGTCCAACAAGG |
| | | GACTTCCTAGCTCAATCGAAAAGACCATCTCGAAAGC |
| | | CAAGGGACAGCCCCGGGAACCCCAAGTGTATACCCTG |
| | | CCACCGAGCCAGGAAGAAATGACTAAGAACCAAGTCT |
| | | CATTGACTTGCCTTGTGAAGGGCTTCTACCCATCGGAT |
| | | ATCGCCGTGGAATGGGAGTCCAACGGCCAGCCGGAAA |
| | | ACAACTACAAGACCACCCCTCCGGTGCTGGACTCAGA |
| | | CGGATCCTTCTTCCTCTACTCGCGGCTGACCGTGGATA |
| | | AGAGCAGATGGCAGGAGGGAAATGTGTTCAGCTGTTC |
| | | TGTGATGCATGAAGCCCTGCACAACCACTACACTCAG |
| | | AAGTCCCTGTCCCTCTCCCTGGGA |
| SEQ ID NO: 810 (Kabat) | LCDR1 | RASESVEYYGTSLMQ |
| SEQ ID NO: 811 (Kabat) | LCDR2 | AASNVES |
| SEQ ID NO: 812 (Kabat) | LCDR3 | QQSRKDPST |
| SEQ ID NO: 813 (Chothia) | LCDR1 | SESVEYYGTSL |
| SEQ ID NO: 814 (Chothia | LCDR2 | AAS |
| SEQ ID NO: 815 (Chothia) | LCDR3 | SRKDPS |
| SEQ ID NO: 826 | VL | DIVLTQSPDSLAVSLGERATINCRASESVEYYGTSLMQW |
| | | YQQKPGQPPKLLIYAASNVESGVPDRFSGSGSGTDFTLTI |
| | | SSLQAEDVAVYYCQQSRKDPSTFGGGTKVEIK |
| SEQ ID NO: 827 | DNA VL | GATATCGTCCTGACTCAGTCACCCGATAGCCTGGCCGT |
| | | CAGCCTGGGCGAGCGGGCTACTATTAACTGTAGAGCT |
| | | AGTGAATCAGTCGAGTACTACGGCACTAGCCTGATGC |
| | | AGTGGTATCAGCAGAAGCCCGGTCAACCCCCTAAGCT |
| | | GCTGATCTACGCCGCCTCTAACGTGGAATCAGGCGTGC |
| | | CCGATAGGTTTAGCGGTAGCGGTAGTGGCACCGACTT |
| | | CACCCTGACTATTAGTAGCCTGCAGGCCGAGGACGTG |
| | | GCCGTCTACTACTGTCAGCAGTCTAGGAAGGACCCTA |
| | | GCACCTTCGGCGGAGGCACTAAGGTCGAGATTAAG |
| SEQ ID NO: 828 | Light | DIVLTQSPDSLAVSLGERATINCRASESVEYYGTSLMQW |
| | chain | YQQKPGQPPKLLIYAASNVESGVPDRFSGSGSGTDFTLTI |
| | | SSLQAEDVAVYYCQQSRKDPSTFGGGTKVEIKRTVAAPS |
| | | VFIFPPSDEQLKSGTASVVCLLNNFYPREAKVQWKVDNA |
| | | LQSGNSQESVTEQDSKDSTYSLSSTLTLSKADYEKHKVY |
| | | ACEVTHQGLSSPVTKSFNRGEC |
| SEQ ID NO: 829 | DNA light | GATATCGTCCTGACTCAGTCACCCGATAGCCTGGCCGT |
| | chain | CAGCCTGGGCGAGCGGGCTACTATTAACTGTAGAGCT |
| | | AGTGAATCAGTCGAGTACTACGGCACTAGCCTGATGC |
| | | AGTGGTATCAGCAGAAGCCCGGTCAACCCCCTAAGCT |
| | | GCTGATCTACGCCGCCTCTAACGTGGAATCAGGCGTGC |
| | | CCGATAGGTTTAGCGGTAGCGGTAGTGGCACCGACTT |
| | | CACCCTGACTATTAGTAGCCTGCAGGCCGAGGACGTG |
| | | GCCGTCTACTACTGTCAGCAGTCTAGGAAGGACCCTA |
| | | GCACCTTCGGCGGAGGCACTAAGGTCGAGATTAAGCG |
| | | TACGGTGGCCGCTCCCAGCGTGTTCATCTTCCCCCCCA |
| | | GCGACGAGCAGCTGAAGAGCGGCACCGCCAGCGTGGT |
| | | GTGCCTGCTGAACAACTTCTACCCCCGGGAGGCCAAG |
| | | GTGCAGTGGAAGGTGGACAACGCCCTGCAGAGCGGCA |
| | | ACAGCCAGGAGAGCGTCACCGAGCAGGACAGCAAGG |
| | | ACTCCACCTACAGCCTGAGCAGCACCCTGACCCTGAG |
| | | CAAGGCCGACTACGAGAAGCATAAGGTGTACGCCTGC |
| | | GAGGTGACCCACCAGGGCCTGTCCAGCCCCGTGACCA |
| | | AGAGCTTCAACAGGGGCGAGTGC |

In some embodiments, the TIM-3 inhibitor is administered at a dose of about 50 mg to about 100 mg, about 200 mg to about 250 mg, about 500 mg to about 1000 mg, or about 1000 mg to about 1500 mg. In embodiments, the TIM-3 inhibitor is administered once every 4 weeks. In other embodiments, the TIM-3 inhibitor is administered at a dose of about 50 mg to about 100 mg once every four weeks. In other embodiments, the TIM-3 inhibitor is administered at a dose of about 200 mg to about 250 mg once every four weeks. In other embodiments, the TIM-3 inhibitor is administered at a dose of about 500 mg to about 1000 mg once every four weeks. In other embodiments, the TIM-3 inhibitor is administered at a dose of about 1000 mg to about 1500 mg once every four weeks.

Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 8. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 8
Amino acid sequences of other exemplary
anti-TIM-3 antibody molecules

| | | |
|---|---|---|
| APE5137 | | |
| SEQ ID NO: 830 | VH | EVQLLESGGGLVQPGGSLRLSCAAASGFTFSSYDMSWVRQAPGKGL |
| | | DWVSTISGGGTYTYYQDSVKGRFTISRDNSKNTLYLQMNSLRAEDTA |
| | | VYYCASMDYWGQGTTVTVSSA |
| SEQ ID NO: 831 | VL | DIQMTQSPSSLSASVGDRVTITCRASQSIRRYLNWYHQKPGKAPKLLI |
| | | YGASTLQSGVPSRFSGSGSGTDFTLTISSLQPEDFAVYYCQQSHSAPLT |
| | | FGGGTKVEIKR |
| APE5121 | | |
| SEQ ID NO: 832 | VH | EVQVLESGGGLVQPGGSLRLYCVASGFTFSGSYAMSWVRQAPGKGL |
| | | EWVSAISGSGGSTYYADSVKGRFTISRDNSKNTLYLQMNSLRAEDTA |
| | | VYYCAKKYYVGPADYWGQGTLVTVSSG |
| SEQ ID NO: 833 | VL | DIVMTQSPDSLAVSLGERATINCKSSQSVLYSSNNKNYLAWYQHKPG |
| | | QPPKLLIYWASTRESGVPDRFSGSGSGTDFTLTISSLQAEDVAVYYCQ |
| | | QYYSSPLTFGGGTKIEVK |

### GITR Agonists

In certain embodiments, a combination described herein comprises a GITR agonist. In some embodiments, the GITR agonist is chosen from GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

Exemplary GITR Agonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 9 (e.g., from the heavy and light chain variable region sequences of MAB7 disclosed in Table 9), or encoded by a nucleotide sequence shown in Table 9. In some embodiments, the CDRs are according to the Kabat definition (e.g., as set out in Table 9). In some embodiments, the CDRs are according to the Chothia definition (e.g., as set out in Table 9). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 9, or encoded by a nucleotide sequence shown in Table 9.

In one embodiment, the anti-GITR antibody molecule comprises a heavy chain variable region (VH) comprising a VHCDR1 amino acid sequence of SEQ ID NO: 909, a VHCDR2 amino acid sequence of SEQ ID NO: 911, and a VHCDR3 amino acid sequence of SEQ ID NO: 913; and a light chain variable region (VL) comprising a VLCDR1 amino acid sequence of SEQ ID NO: 914, a VLCDR2 amino acid sequence of SEQ ID NO: 916, and a VLCDR3 amino acid sequence of SEQ ID NO: 918, each disclosed in Table 9.

In one embodiment, the anti-GITR antibody molecule comprises a VH comprising the amino acid sequence of SEQ ID NO: 901, or an amino acid sequence at least 85%, 90%, 95%, or 99% identical or higher to SEQ ID NO: 901. In one embodiment, the anti-GITR antibody molecule comprises a VL comprising the amino acid sequence of SEQ ID NO: 902, or an amino acid sequence at least 85%, 90%, 95%, or 99% identical or higher to SEQ ID NO: 902. In one embodiment, the anti-GITR antibody molecule comprises a VH comprising the amino acid sequence of SEQ ID NO: 901 and a VL comprising the amino acid sequence of SEQ ID NO: 902.

In one embodiment, the antibody molecule comprises a VH encoded by the nucleotide sequence of SEQ ID NO: 905, or a nucleotide sequence at least 85%, 90%, 95%, or 99% identical or higher to SEQ ID NO: 905. In one embodiment, the antibody molecule comprises a VL encoded by the nucleotide sequence of SEQ ID NO: 906, or a nucleotide sequence at least 85%, 90%, 95%, or 99% identical or higher to SEQ ID NO: 906. In one embodiment, the antibody molecule comprises a VH encoded by the nucleotide sequence of SEQ ID NO: 905 and a VL encoded by the nucleotide sequence of SEQ ID NO: 906.

In one embodiment, the anti-GITR antibody molecule comprises a heavy chain comprising the amino acid sequence of SEQ ID NO: 903, or an amino acid sequence at least 85%, 90%, 95%, or 99% identical or higher to SEQ ID NO: 903. In one embodiment, the anti-GITR antibody molecule comprises a light chain comprising the amino acid sequence of SEQ ID NO: 904, or an amino acid sequence at least 85%, 90%, 95%, or 99% identical or higher to SEQ ID NO: 904. In one embodiment, the anti-GITR antibody molecule comprises a heavy chain comprising the amino acid sequence of SEQ ID NO: 903 and a light chain comprising the amino acid sequence of SEQ ID NO: 904.

In one embodiment, the antibody molecule comprises a heavy chain encoded by the nucleotide sequence of SEQ ID NO: 907, or a nucleotide sequence at least 85%, 90%, 95%, or 99% identical or higher to SEQ ID NO: 907. In one embodiment, the antibody molecule comprises a light chain encoded by the nucleotide sequence of SEQ ID NO: 908, or a nucleotide sequence at least 85%, 90%, 95%, or 99% identical or higher to SEQ ID NO: 908. In one embodiment, the antibody molecule comprises a heavy chain encoded by the nucleotide sequence of SEQ ID NO: 907 and a light chain encoded by the nucleotide sequence of SEQ ID NO: 908.

The antibody molecules described herein can be made by vectors, host cells, and methods described in WO 2016/057846, incorporated by reference in its entirety.

TABLE 9
Amino acid and nucleotide
sequences of exemplary anti-GITR antibody molecule

| MAB7 | | |
|---|---|---|
| SEQ ID NO: 901 | VH | EVQLVESGGGLVQSGGSLRLSCAASGFSLSSYGVDWVRQ |
| | | APGKGLEWVGVIWGGGGTYYASSLMGRFTISRDNSKNTL |
| | | YLQMNSLRAEDTAVYYCARHAYGHDGGFAMDYWGQGT |
| | | LVTVSS |
| SEQ ID NO: 902 | VL | EIVMTQSPATLSVSPGERATLSCRASESVSSNVAWYQQRP |
| | | GQAPRLLIYGASNRATGIPARFSGSGSGTDFTLTISRLEPED |
| | | FAVYYCGQSYSYPFTFGQGTKLEIK |
| SEQ ID NO: 903 | Heavy | EVQLVESGGGLVQSGGSLRLSCAASGFSLSSYGVDWVRQ |
| | Chain | APGKGLEWVGVIWGGGGTYYASSLMGRFTISRDNSKNTL |
| | | YLQMNSLRAEDTAVYYCARHAYGHDGGFAMDYWGQGT |
| | | LVTVSSASTKGPSVFPLAPSSKSTSGGTAALGCLVKDYFPE |
| | | PVTVSWNSGALTSGVHTFPAVLQSSGLYSLSSVVTVPSSSL |
| | | GTQTYICNVNHKPSNTKVDKRVEPKSCDKTHTCPPCPAPE |
| | | LLGGPSVFLFPPKPKDTLMISRTPEVTCVVVDVSHEDPEVK |
| | | FNWYVDGVEVHNAKTKPREEQYNSTYRVVSVLTVLHQD |
| | | WLNGKEYKCKVSNKALPAPIEKTISKAKGQPREPQVYTLP |
| | | PSREEMTKNQVSLTCLVKGFYPSDIAVEWESNGQPENNY |
| | | KTTPPVLDSDGSFFLYSKLTVDKSRWQQGNVFSCSVMHE |
| | | ALHNHYTQKSLSLSPGK |
| SEQ ID NO: 904 | Light | EIVMTQSPATLSVSPGERATLSCRASESVSSNVAWYQQRP |
| | Chain | GQAPRLLIYGASNRATGIPARFSGSGSGTDFTLTISRLEPED |
| | | FAVYYCGQSYSYPFTFGQGTKLEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| SEQ ID NO: 905 | DNA VH | GAGGTGCAGCTGGTGGAATCTGGCGGCGGACTGGTGCA |
| | | GTCCGGCGGCTCTCTGAGACTGTCTTGCGCTGCCTCCGG |
| | | CTTCTCCCTGTCCTCTTACGGCGTGGACTGGGTGCGACA |
| | | GGCCCCTGGCAAGGGCCTGGAATGGGTGGGAGTGATCT |
| | | GGGGCGGAGGCGGCACCTACTACGCCTCTTCCCTGATG |
| | | GGCCGGTTCACCATCTCCCGGGACAACTCCAAGAACAC |
| | | CCTGTACCTGCAGATGAACTCCCTGCGGGCCGAGGACA |
| | | CCGCCGTGTACTACTGCGCCAGACACGCCTACGGCCAC |
| | | GACGGCGGCTTCGCCATGGATTATTGGGGCCAGGGCAC |
| | | CCTGGTGACAGTGTCCTCC |
| SEQ ID NO: 906 | DNA VL | GAGATCGTGATGACCCAGTCCCCCGCCACCCTGTCTGT |
| | | GTCTCCCGGCGAGAGAGCCACCCTGAGCTGCAGAGCCT |
| | | CCGAGTCCGTGTCCTCCAACGTGGCCTGGTATCAGCAG |
| | | AGACCTGGTCAGGCCCCTCGGCTGCTGATCTACGGCGC |
| | | CTCTAACCGGGCCACCGGCATCCCTGCCAGATTCTCCG |
| | | GCTCCGGCAGCGGCACCGACTTCACCCTGACCATCTCC |
| | | CGGCTGGAACCCGAGGACTTCGCCGTGTACTACTGCGG |
| | | CCAGTCCTACTCATACCCCTTCACCTTCGGCCAGGGCAC |
| | | CAAGCTGGAAATCAAG |
| SEQ ID NO: 907 | DNA | GAGGTGCAGCTGGTGGAATCTGGCGGCGGACTGGTGCA |
| | Heavy | GTCCGGCGGCTCTCTGAGACTGTCTTGCGCTGCCTCCGG |
| | Chain | CTTCTCCCTGTCCTCTTACGGCGTGGACTGGGTGCGACA |
| | | GGCCCCTGGCAAGGGCCTGGAATGGGTGGGAGTGATCT |
| | | GGGGCGGAGGCGGCACCTACTACGCCTCTTCCCTGATG |
| | | GGCCGGTTCACCATCTCCCGGGACAACTCCAAGAACAC |
| | | CCTGTACCTGCAGATGAACTCCCTGCGGGCCGAGGACA |
| | | CCGCCGTGTACTACTGCGCCAGACACGCCTACGGCCAC |
| | | GACGGCGGCTTCGCCATGGATTATTGGGGCCAGGGCAC |
| | | CCTGGTGACAGTGTCCTCCGCTAGCACCAAGGGCCCAA |
| | | GTGTGTTTCCCCTGGCCCCCAGCAGCAAGTCTACTTCCG |
| | | GCGGAACTGCTGCCCTGGGTTGCCTGGTGAAGGACTAC |
| | | TTCCCCGAGCCCGTGACAGTGTCCTGGAACTCTGGGGC |
| | | TCTGACTTCCGGCGTGCACACCTTCCCCGCCGTGCTGCA |
| | | GAGCAGCGGCCTGTACAGCCTGAGCAGCGTGGTGACAG |
| | | TGCCCTCCAGCTCTCTGGGAACCCAGACCTATATCTGCA |
| | | ACGTGAACCACAAGCCCAGCAACACCAAGGTGGACAA |
| | | GAGAGTGGAGCCCAAGAGCTGCGACAAGACCCACACC |
| | | TGCCCCCCCTGCCCAGCTCCAGAACTGCTGGGAGGGCC |
| | | TTCCGTGTTCCTGTTCCCCCCCAAGCCCAAGGACACCCT |
| | | GATGATCAGCAGGACCCCCGAGGTGACCTGCGTGGTGG |
| | | TGGACGTGTCCCACGAGGACCCAGAGGTGAAGTTCAAC |
| | | TGGTACGTGGACGGCGTGGAGGTGCACAACGCCAAGA |
| | | CCAAGCCCAGAGAGGAGCAGTACAACAGCACCTACAG |
| | | GGTGGTGTCCGTGCTGACCGTGCTGCACCAGGACTGGC |
| | | TGAACGGCAAAGAATACAAGTGCAAAGTCTCCAACAA |
| | | GGCCCTGCCAGCCCCAATCGAAAAGACAATCAGCAAG |
| | | GCCAAGGGCCAGCCACGGGAGCCCCAGGTGTACACCCT |
| | | GCCCCCCAGCCGGGAGGAGATGACCAAGAACCAGGTG |
| | | TCCCTGACCTGTCTGGTGAAGGGCTTCTACCCCAGCGAT |
| | | ATCGCCGTGGAGTGGGAGAGCAACGGCCAGCCCGAGA |
| | | ACAACTACAAGACCACCCCCCCAGTGCTGGACAGCGAC |
| | | GGCAGCTTCTTCCTGTACAGCAAGCTGACCGTGGACAA |
| | | GTCCAGGTGGCAGCAGGGCAACGTGTTCAGCTGCAGCG |
| | | TGATGCACGAGGCCCTGCACAACCACTACACCCAGAAG |
| | | TCCCTGAGCCTGAGCCCCGGCAAG |
| SEQ ID NO: 908 | DNA | GAGATCGTGATGACCCAGTCCCCCGCCACCCTGTCTGT |
| | Light | GTCTCCCGGCGAGAGAGCCACCCTGAGCTGCAGAGCCT |
| | Chain | CCGAGTCCGTGTCCTCCAACGTGGCCTGGTATCAGCAG |
| | | AGACCTGGTCAGGCCCCTCGGCTGCTGATCTACGGCGC |
| | | CTCTAACCGGGCCACCGGCATCCCTGCCAGATTCTCCG |
| | | GCTCCGGCAGCGGCACCGACTTCACCCTGACCATCTCC |
| | | CGGCTGGAACCCGAGGACTTCGCCGTGTACTACTGCGG |
| | | CCAGTCCTACTCATACCCCTTCACCTTCGGCCAGGGCAC |
| | | CAAGCTGGAAATCAAGCGTACGGTGGCCGCTCCCAGCG |
| | | TGTTCATCTTCCCCCCCAGCGACGAGCAGCTGAAGAGC |
| | | GGCACCGCCAGCGTGGTGTGCCTGCTGAACAACTTCTA |
| | | CCCCCGGGAGGCCAAGGTGCAGTGGAAGGTGGACAAC |
| | | GCCCTGCAGAGCGGCAACAGCCAGGAGAGCGTCACCG |
| | | AGCAGGACAGCAAGGACTCCACCTACAGCCTGAGCAG |
| | | CACCCTGACCCTGAGCAAGGCCGACTACGAGAAGCATA |
| | | AGGTGTACGCCTGCGAGGTGACCCACCAGGGCCTGTCC |
| | | AGCCCCGTGACCAAGAGCTTCAACAGGGGCGAGTGC |
| SEQ ID NO: 909 (KABAT) | HCDR1 | SYGVD |
| SEQ ID NO: 910 (CHOTHIA) | HCDR1 | GFSLSSY |
| SEQ ID NO: 911 (KABAT) | HCDR2 | VIWGGGGTYYASSLMG |
| SEQ ID NO: 912 (CHOTHIA) | HCDR2 | WGGGG |
| SEQ ID NO: 913 (KABAT) | HCDR3 | HAYGHDGGFAMDY |
| SEQ ID NO: 913 (CHOTHIA) | HCDR3 | HAYGHDGGFAMDY |
| SEQ ID NO: 914 (KABAT) | LCDR1 | RASESVSSNVA |
| SEQ ID NO: 915 (CHOTHIA) | LCDR1 | SESVSSN |
| SEQ ID NO: 916 (KABAT) | LCDR2 | GASNRAT |
| SEQ ID NO: 917 (CHOTHIA) | LCDR2 | GAS |
| SEQ ID NO: 918 (KABAT) | LCDR3 | GQSYSYPFT |
| SEQ ID NO: 919 (CHOTHIA) | LCDR3 | SYSYPF |

In some embodiments, the GITR agonist is administered at a dose of about 2 mg to about 600 mg (e.g., about 5 mg to about 500 mg). In some embodiments, the GITR agonist is administered once every week. In other embodiments, the GITR agonist is administered once every three weeks. In other embodiments, the GITR agonist is administered once every six weeks.

In some embodiments, the GITR agonist is administered at a dose of about 2 mg to about 10 mg (e.g., about 5 mg), about 5 mg to about 20 mg (e.g., about 10 mg), about 20 mg to about 40 mg (e.g., about 30 mg), about 50 mg to about 100 mg (e.g., about 60 mg), about 100 mg to about 200 mg (e.g., about 150 mg), about 200 mg to about 400 mg (e.g., about 300 mg), or about 400 mg to about 600 mg (e.g., about 500 mg), once every week.

In some embodiments, the GITR agonist is administered at a dose of about 2 mg to about 10 mg (e.g., about 5 mg), about 5 mg to about 20 mg (e.g., about 10 mg), about 20 mg to about 40 mg (e.g., about 30 mg), about 50 mg to about 100 mg (e.g., about 60 mg), about 100 mg to about 200 mg (e.g., about 150 mg), about 200 mg to about 400 mg (e.g., about 300 mg), or about 400 mg to about 600 mg (e.g., about 500 mg), once every three weeks.

In some embodiments, the GITR agonist is administered at a dose of about 2 mg to about 10 mg (e.g., about 5 mg), about 5 mg to about 20 mg (e.g., about 10 mg), about 20 mg to about 40 mg (e.g., about 30 mg), about 50 mg to about 100 mg (e.g., about 60 mg), about 100 mg to about 200 mg (e.g., about 150 mg), about 200 mg to about 400 mg (e.g., about 300 mg), or about 400 mg to about 600 mg (e.g., about 500 mg), once every six weeks.

In some embodiments, three doses of the GITR agonist are administered over a period of three weeks followed by a nine-week pause. In some embodiments, four doses of the GITR agonist are administered over a period of twelve weeks followed by a nine-week pause. In some embodiments, four doses of the GITR agonists are administered over a period of twenty-one or twenty-four weeks followed by a nine-week pause.

Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 10.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

In one embodiment, the anti-GITR antibody molecule is an anti-GITR antibody molecule disclosed in WO 2013/039954, herein incorporated by reference in its entirety. In an embodiment, the anti-GITR antibody molecule is an anti-GITR antibody molecule disclosed in US 2014/0072566, herein incorporated by reference in its entirety.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 10
Amino acid sequence of other exemplary
anti-GITR antibody molecules

| BMS-986156 | | |
|---|---|---|
| SEQ ID NO: 920 | VH | QVQLVESGGGVVQPGRSLRLSCAASGFTFSSYGMHWVRQAPG |
| | | KGLEWVAVIWYEGSNKYYADSVKGRFTISRDNSKNTLYLQMN |
| | | SLRAEDTAVYYCARGGSMVRGDYYYGMDVWGQGTTVTVSS |
| SEQ ID NO: 921 | VL | AIQLTQSPSSLSASVGDRVTITCRASQGISSALAWYQQKPGKAP |
| | | KLLIYDASSLESGVPSRFSGSGSGTDFTLTISSLQPEDFATYYCQ |
| | | QFNSYPYTFGQGTKLEIK |

### Estrogen Receptor Antagonists

In certain embodiments, a combination described herein comprises an estrogen receptor (ER) antagonist. In some embodiments, the estrogen receptor antagonist is used in combination with a PD-1 inhibitor, a CDK4/6 inhibitor, or both. In some embodiments, the combination is used to treat an ER positive (ER+) cancer or a breast cancer (e.g., an ER+ breast cancer).

In some embodiments, the estrogen receptor antagonist is a selective estrogen receptor degrader (SERD). SERDs are estrogen receptor antagonists which bind to the receptor and result in e.g., degradation or down-regulation of the receptor (Boer K. et al., (2017) Therapeutic Advances in Medical Oncology 9(7): 465-479). ER is a hormone-activated transcription factor important for e.g., the growth, development and physiology of the human reproductive system. ER is activated by, e.g., the hormone estrogen (17beta estradiol). ER expression and signaling is implicated in cancers (e.g., breast cancer), e.g., ER positive (ER+) breast cancer. In some embodiments, the SERD is chosen from LSZ102, fulvestrant, brilanestrant, or elacestrant.

Exemplary Estrogen Receptor Antagonists

In some embodiments, the SERD comprises a compound disclosed in International Application Publication No. WO 2014/130310, which is hereby incorporated by reference in its entirety.

In some embodiments, the SERD comprises a compound of formula I:

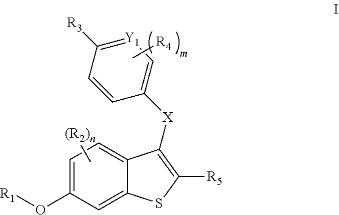

in which:

n is selected from 0, 1 and 2;

m is selected from 0, 1 and 2;

X is selected from O and NR₆; wherein R₆ is C₁₋₄alkyl;

Y₁ is selected from N and CR₇; wherein R₇ is selected from hydrogen and C₁₋₄alkyl;

R₁ is hydrogen;

R₂ is selected from hydrogen and halo;

R₃ is selected from —CH₂CH₂R_{8b} and —CR₈ₐ═CR₈ₐR_{8b}; wherein each R₈ₐ is independently selected from hydrogen, fluoro and C₁₋₄alkyl; and R_{8b} is selected from —C(O)OR₉ₐ, —C(O)NR₉ₐR_{9b}, —C(O)NHOR₉ₐ, —C(O)X₂R₉ₐ and a 5-6 member heteroaryl selected from:

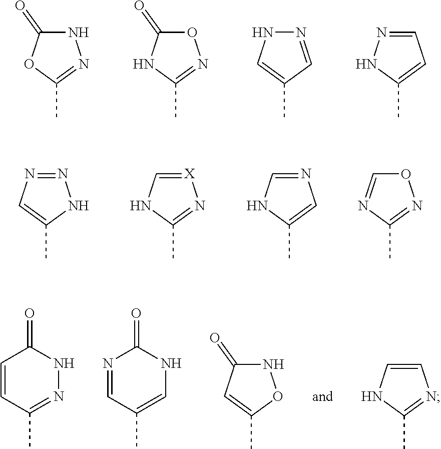

wherein the dotted line indicates the point of attachment with —CH₂CH₂ or —CR₈ₐ═R₈ₐ of R₃;

wherein X₂ is C₁₋₄alkylene; R₉ₐ and R_{9b} are independently selected from hydrogen, C₁₋₄alkyl, hydroxy-substituted-C₁₋₄alkyl and halo-substituted-C₁₋₄alkyl; wherein said heteroaryl of R_{8b} is unsubstituted or substituted with a group selected from C₁₋₄alkyl and C₃₋₈cycloalkyl;

R₄ is selected from hydrogen, C₁₋₄alkyl, halo and C₁₋₃alkoxy;

R₅ is selected from C₆₋₁₀aryl and a 5-6 member heteroaryl selected from:

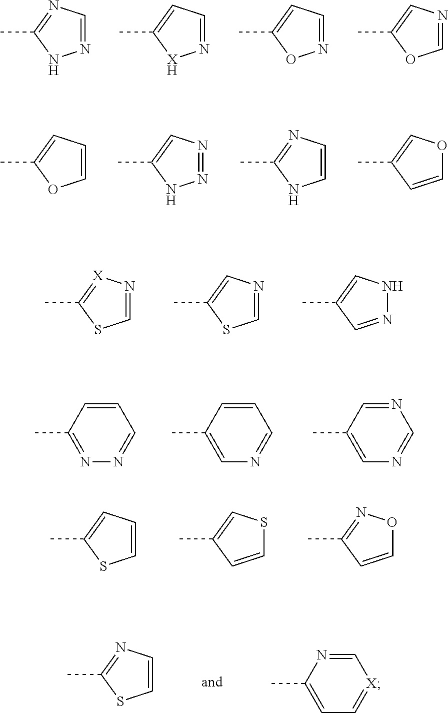

wherein the dotted line indicates the point of attachment with the benzothiophene core; wherein said C₆₋₁₀aryl or heteroaryl of R₅ is substituted with 1 to 3 R₅ₐ groups independently selected from hydroxy, amino, C₁₋₄alkyl, halo, nitro, cyano, halo-substituted-C₁₋₄alkyl, cyano-substituted-C₁₋₄alkyl, hydroxy-substituted-C₁₋₄alkyl, halo-substituted-C₁₋₄alkoxy, C₁₋₄alkoxy, —SF₅, —NR₁₁ₐR_{11b}, —C(O)R₁₁ₐ and a 4-7 member saturated ring containing one other heteroatom or group selected from O, NH, and S(O)₀₋₂; wherein R₁₁ₐ and R_{11b} are independently selected from hydrogen and C₁₋₄alkyl; or R₁₁ₐ and R_{11b} together with the nitrogen to which they are both attached form a 4 to 7 member saturated ring containing one other heteroatom or group selected from O, NH, and S(O)₀₋₂; wherein said 4-7 member ring of R₅ₐ can be unsubstituted or substituted with C₁₋₄alkyl; or a pharmaceutically acceptable salt thereof.

In some embodiments, the SERD comprises LSZ102. LSZ102 has the chemical name: (E)-3-(4-((2-(2-(1,1-difluoroethyl)-4-fluorophenyl)-6-hydroxybenzo[b]thiophen-3-yl)oxy)phenyl)acrylic acid.

Other Exemplary Estrogen Receptor Antagonists

In some embodiments, the SERD comprises fulvestrant (CAS Registry Number: 129453-61-8), or a compound disclosed in International Application Publication No. WO 2001/051056, which is hereby incorporated by reference in its entirety.

Fulvestrant is also known as ICI 182780, ZM 182780, FASLODEX®, or (7α,17β)-7-{9-[(4,4,5,5,5-pentafluoropentyl)sulfinyl]nonyl}estra-1,3,5(10)-triene-3,17-diol. Fulvestrant is a high affinity estrogen receptor antagonist with an IC50 of 0.29 nM. In some embodiments, fulvestrant is administered at a dose of about 250 mg to about 500 mg. In some embodiments, fulvestrant is administered at a dose of about 500 mg via intramuscular injection every 14 days for three administrations, e.g., a dose of about 500 mg is administered on days 1, 15 and 29. In other embodiments, a dose of about 500 mg of fulvestrant is administered once a month, e.g., once every 28-31 days.

In some embodiments, the SERD comprises elacestrant (CAS Registry Number: 722533-56-4), or a compound disclosed in U.S. Pat. No. 7,612,114, which is incorporated by reference in its entirety.

Elacestrant is also known as RAD1901, ER-306323 or (6R)-6-{2-[Ethyl({4-[2-(ethylamino)ethyl]phenyl}methyl)amino]-4-methoxyphenyl}-5,6,7,8-tetrahydronaphthalen-2-ol. Elacestrant is an orally bioavailable, non-steroidal combined selective estrogens receptor modulator (SERM) and a SERD. Elacestrant is also disclosed, e.g., in Garner F et al., (2015) Anticancer Drugs 26(9):948-56.

In some embodiments, the SERD is brilanestrant (CAS Registry Number: 1365888-06-7), or a compound disclosed in International Application Publication No. WO 2015/136017, which is incorporated by reference in its entirety.

Brilanestrant is also known as GDC-0810, ARN810, RG-6046, RO-7056118 or (2E)-3-{4-[(1E)-2-(2-chloro-4-fluorophenyl)-1-(1H-indazol-5-yl)but-1-en-1-yl]phenyl}prop-2-enoic acid. Brilanestrant is a next-generation, orally bioavailable selective SERD with an IC50 of 0.7 nM. Brilanestrant is also disclosed, e.g., in Lai A. et al. (2015) Journal of Medicinal Chemistry 58 (12): 4888-4904.

In some embodiments, the SERD is chosen from RU 58668, GW7604, AZD9496, bazedoxifene, pipendoxifene, arzoxifene, OP-1074, or acolbifene, e.g., as disclosed in McDonell et al. (2015) Journal of Medicinal Chemistry 58(12) 4883-4887.

Other exemplary estrogen receptor antagonists are disclosed, e.g., in WO 2011/156518, WO 2011/159769, WO 2012/037410, WO 2012/037411, and US 2012/0071535, all of which are hereby incorporated by reference in their entirety.

### CDK4/6 Inhibitors

In certain embodiments, a combination described herein comprises an inhibitor of Cyclin-Dependent Kinases 4 or 6 (CDK4/6). In some embodiments, the CDK4/6 inhibitor is used in combination with a PD-1 inhibitor, an estrogen receptor (ER) antagonist, or both. In some embodiments, the combination is used to treat an ER positive (ER+) cancer or a breast cancer (e.g., an ER+ breast cancer). In some embodiments, the CDK4/6 inhibitor is chosen from ribociclib, abemaciclib (Eli Lilly), or palbociclib.

Exemplary CDK4/6 Inhibitors

In some embodiments, the CDK4/6 inhibitor comprises ribociclib (CAS Registry Number: 1211441-98-3), or a compound disclosed in U.S. Pat. Nos. 8,415,355 and 8,685,980, which are incorporated by reference in their entirety.

In some embodiments, the CDK4/6 inhibitor comprises a compound disclosed in International Application Publication No. WO 2010/020675 and U.S. Pat. Nos. 8,415,355 and 8,685,980, which are incorporated by reference in their entirety.

In some embodiments, the CDK4/6 inhibitor comprises a compound of formula I:

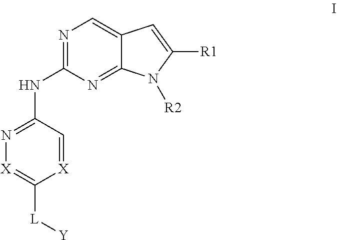

or a pharmaceutically acceptable salt thereof, wherein

X is CR⁹;

R¹ is CONR⁵R⁶, and R⁵ and R⁶ are C₁₋₈alkyl;

R² is C₃₋₁₄cycloalkyl;

L is a bond, C₁₋₈alkylene, C(O), or C(O)NH, and wherein L may be substituted or unsubstituted;

Y is H, R¹¹, NR¹²R¹³, OH, or Y is part of the following group

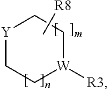

where Y is CR⁹ or N;

where 0-3 R⁸ may be present, and R⁸ is C₁₋₈alkyl, oxo, halogen, or two or more R⁸ may form a bridged alkyl group;

W is CR⁹, or N;

R³ is H, C₁₋₈alkyl, C₁₋₈alkylR¹⁴, C₃₋₁₄cycloalkyl, C(O)C₁₋₈ alkyl, C₁₋₈haloalkyl, C₁₋₈alkylOH, C(O)NR¹⁴R¹⁵, C₁₋₈cyanoalkyl, C(O)R¹⁴, C₀₋₈alkylC(O)C₀₋₈alkylNR¹⁴R¹⁵, C₀₋₈alkylC(O)OR¹⁴, NR¹⁴R¹⁵, SO₂C₁₋₈alkyl, C₁₋₈alkylC₃₋₁₄cycloalkyl, C(O)C₁₋₈alkylC₃₋₁₄cycloalkyl, C₁₋₈alkoxy, or OH which may be substituted or unsubstituted when R³ is not H;

R⁹ is H or halogen;

R¹¹, R¹², R¹³, R¹⁴, and R¹⁵ are each independently selected from H, C₁₋₈alkyl, C₃₋₁₄ cycloalkyl, a 3-14 membered cycloheteroalkyl group, a C₆₋₁₄ aryl group, a 5-14 membered heteroaryl group, alkoxy, C(O)H, C(NH)OH, C(NH)OCH₃, C(O)C₁₋₃alkyl, C₁₋₈alkylNH₂, and C₁₋₆alkylOH, and wherein R¹¹, R¹², and R¹³, R¹⁴, and R¹⁵ when not H may be substituted or unsubstituted;

m and n are independently 0-2; and

wherein L, R³, R¹¹, R¹², and R¹³, R¹⁴, and R¹⁵ may be substituted with one or more of C₁₋₈alkyl, C₂₋₈alkenyl, C₂₋₈alkynyl, C₃₋₁₄cycloalkyl, 5-14 membered heteroaryl group, C₆₋₁₄aryl group, a 3-14 membered cycloheteroalkyl group, OH, (O), CN, alkoxy, halogen, or NH₂.

In some embodiments, the CDK4/6 inhibitor comprises a compound chosen from:
7-Cyclopentyl-2-{5-[4-(2-fluoro-ethyl)-piperazin-1-yl]-pyridin-2-ylamino}-7H-pyrrolo[2,3-d]pyrimidine-6-carboxylic acid dimethylamide;
7-Cyclopentyl-2-(4-dimethylamino-3,4,5,6-tetrahydro-2H-[1,3′]bipyridinyl-6′-ylamino)-7H-pyrrolo[2,3-d]pyrimidine-6-carboxylic acid dimethylamide;
2-[5-(4-Carbamoylmethyl-piperazin-1-yl)-pyridin-2-ylamino]-7-cyclopentyl-7H-pyrrolo[2,3-d]pyrimidine-6-carboxylic acid dimethylamide;
2-{5-[4-(2-Amino-acetyl)-piperazin-1-yl]-pyridin-2-ylamino}-7-cyclopentyl-7H-pyrrolo[2,3-d]pyrimidine-6-carboxylic acid dimethylamide;
2-[5-(3-Amino-pyrrolidin-1-yl)-pyridin-2-ylamino]-7-cyclopentyl-7H-pyrrolo[2,3-d]pyrimidine-6-carboxylic acid dimethylamide;
7-Cyclopentyl-2-{5-[4-(2-methoxy-ethyl)-piperazin-1-yl]-pyridin-2-ylamino}-7H-pyrrolo[2,3-d]pyrimidine-6-carboxylic acid dimethylamide;
7-Cyclopentyl-2-[4-(2-hydroxyethyl)-3,4,5,6-tetrahydro-2H-[1,2′]bipyrazinyl-5′-ylamino]-7H-pyrrolo[2,3-d]pyrimidine-6-carboxylic acid dimethylamide;
7-Cyclopentyl-2-[5-((R)-3-methyl-piperazin-1-yl)-pyridin-2-ylamino]-7H-pyrrolo[2,3-d]pyrimidine-6-carboxylic acid dimethylamide;
7-Cyclopentyl-2-[5-((S)-3-methylpiperazin-1-yl)-pyridin-2-ylamino]-7H-pyrrolo[2,3-d]pyrimidine-6-carboxylic acid dimethylamide;
7-Cyclopentyl-2-[5-(3-methylpiperazin-1-yl)-pyridin-2-ylamino]-7H-pyrrolo[2,3-d]pyrimidine-6-carboxylic acid dimethylamide;
7-Cyclopentyl-2-{5-[4-(3-hydroxypropyl)-piperazin-1-yl]-pyridin-2-ylamino}-7H-pyrrolo[2,3-d]pyrimidine-6-carboxylic acid dimethylamide;
7-Cyclopentyl-2-{5-[4-(pyrrolidine-1-carbonyl)-piperazin-1-yl]-pyridin-2-ylamino}-7H-pyrrolo[2,3-d]pyrimidine-6-carboxylic acid dimethylamide;
7-Cyclopentyl-2-{5-[4-(2-hydroxy-ethyl)-piperazin-1-yl]-pyridin-2-ylamino}-7H-pyrrolo[2,3-d]pyrimidine-6-carboxylic acid dimethylamide;
7-Cyclopentyl-2-{5-[4-((S)-2,3-dihydroxypropyl)-piperazin-1-yl]-pyridin-2-ylamino}-7H-pyrrolo[2,3-d]pyrimidine-6-carboxylic acid dimethylamide;
7-Cyclopentyl-2-(5-{4-[2-(2-hydroxyethoxy)-ethyl]-piperazin-1-yl}-pyridin-2-ylamino)-7H-pyrrolo[2,3-d]pyrimidine-6-carboxylic acid dimethylamide;
7-Cyclopentyl-2-{5-[4-(2-hydroxy-1-methylethyl)-piperazin-1-yl]-pyridin-2-ylamino}-7H-pyrrolo[2,3-d]pyrimidine-6-carboxylic acid dimethylamide;
7-Cyclopentyl-2-{6-[4-(2-hydroxyethyl)-piperazin-1-yl]-pyridazin-3-ylamino}-7H-pyrrolo[2,3-d]pyrimidine-6-carboxylic acid dimethylamide;
7-Cyclopentyl-2-{5-[4-(2,3-dihydroxypropyl)-piperazin-1-yl]-pyridin-2-ylamino}-7H-pyrrolo[2,3-d]pyrimidine-6-carboxylic acid dimethylamide;
7-Cyclopentyl-2-{5-[4-((R)-2,3-dihydroxypropyl)-piperazin-1-yl]-pyridin-2-ylamino}-7H-pyrrolo[2,3-d]pyrimidine-6-carboxylic acid dimethylamide;
7-Cyclopentyl-2-(3,4,5,6-tetrahydro-2H-[1,2′]bipyrazinyl-5′-ylamino)-7H-pyrrolo[2,3-d]pyrimidine-6-carboxylic acid dimethylamide;
7-Cyclopentyl-2-[5-(piperazine-1-carbonyl)-pyridin-2-ylamino]-7H-pyrrolo[2,3d]pyrimidine-6-carboxylic acid dimethylamide;
7-Cyclopentyl-2-[5-(4-dimethylaminopiperidine-1-carbonyl)-pyridin-2-ylamino]-7H-pyrrolo[2,3-d]pyrimidine-6-carboxylic acid dimethylamide;
7-Cyclopentyl-2-(1′,2′,3′,4′,5′,6′-hexahydro-[3,4′]bipyridinyl-6-ylamino)-7H-pyrrolo[2,3d]pyrimidine-6-carboxylic acid dimethylamide;
7-Cyclopentyl-2-[5-((S)-3-methylpiperazin-1-ylmethyl)-pyridin-2-ylamino]-7Hpyrrolo[2,3-d]pyrimidine-6-carboxylic acid dimethylamide;
7-Cyclopentyl-2-{5-[4-((S)-2-hydroxypropyl)-piperazin-1-yl]-pyridin-2-ylamino}-7H-pyrrolo[2,3-d]pyrimidine-6-carboxylic acid dimethylamide;
7-Cyclopentyl-2-{5-[4-((R)-2-hydroxypropyl)-piperazin-1-yl]-pyridin-2-ylamino}-7H-pyrrolo[2,3-d]pyrimidine-6-carboxylic acid dimethylamide;
7-Cyclopentyl-2-[5-(4-isopropyl-piperazin-1-yl)-pyridin-2-ylamino]-7H-pyrrolo[2,3d]pyrimidine-6-carboxylic acid dimethylamide;
7-Cyclopentyl-2-[5-(4-isopropyl-piperazine-1-carbonyl)-pyridin-2-ylamino-1-7H-pyrrolo[2,3-d]pyrimidine-6-carboxylic acid dimethylamide;
7-Cyclopentyl-2-{5-[4-(4-methyl-pentyl)-piperazin-1-yl]-pyridin-2-ylamino}-7H-pyrrolo[2,3-d]pyrimidine-6-carboxylic acid dimethylamide;
7-Cyclopentyl-2-{5-[4-(2-hydroxy-2methylpropyl)-piperazin-1-yl]-pyridin-2-ylamino}-7H-pyrrolo[2,3-d]pyrimidine-6-carboxylic acid dimethylamide;
7-Cyclopentyl-2-[5-(3,3-dimethyl-piperazin-1-yl)-pyridin-2-ylamino]-7H-pyrrolo[2,3-d]pyrimidine-6-carboxylic acid dimethylamide;
7-Cyclopentyl-2-[5-(3,8-diaza-bicyclo[3.2.4]oct-3-ylmethyl)-pyridin-2-ylamino]-7H-pyrrolo[2,3-d]pyrimidine-6-carboxylic acid dimethylamide;
7-Cyclopentyl-2-[5-(4-ethyl-piperazin-1-yl)-pyridin-2-ylamino]-7H-pyrrolo[2,3-d]pyrimidine-6-carboxylic acid dimethylamide;
7-Cyclopentyl-2-[5-(4-cyclopentyl-piperazin-1-yl)-pyridin-2-ylamino]-7H-pyrrolo[2,3-d]pyrimidine-6-carboxylic acid dimethylamide;
7-Cyclopentyl-2-(1′-isopropyl-1′,2′,3′,4′,5′,6′-hexahydro-[3,4′]bipyridinyl-6-ylamino)-7H-pyrrolo[2,3-d]pyrimidine-6-carboxylic acid dimethylamide;
7-Cyclopentyl-2-{5-[(R)-4-(2-hydroxyethyl)-3-methyl-piperazin-1-yl]-pyridin-2-ylamino}-7H-pyrrolo[2,3-d]pyrimidine-6-carboxylic acid dimethylamide;
7-Cyclopentyl-2-{5-[(S)-4-(2-hydroxyethyl)-3-methyl-piperazin-1-yl]-pyridin-2-ylamino}-7H-pyrrolo[2,3-d]pyrimidine-6-carboxylic acid dimethylamide;
7-Cyclopentyl-2-{5-[4-(2-hydroxyethyl)-piperazin-1-ylmethyl]-pyridin-2-ylamino}-7H-pyrrolo[2,3-d]pyrimidine-6-carboxylic acid dimethylamide;
7-Cyclopentyl-2-{5-[4-(2-dimethylaminoacetyl)-piperazin-1-yl]-pyridin-2-ylamino}-7H-pyrrolo[2,3-d]pyrimidine-6-carboxylic acid dimethylamide;
7-Cyclopentyl-2-{5-[4-(2-ethyl-butyl)piperazin-1-yl]-pyridin-2-ylamino}-7H-pyrrolo[2,3-d]pyrimidine-6-carboxylic acid dimethylamide;
2-{5-[4-(2-Cyclohexyl-acetyl)piperazin-1-yl]-pyridin-2-ylamino}-7-cyclopentyl-7H-pyrrolo[2,3-d]pyrimidine-6-carboxylic acid dimethylamide;
7-Cyclopentyl-2-{5-[4-(3-cyclopentyl-propionyl)-piperazin-1-yl]-pyridin-2-ylamino}7H-pyrrolo[2,3-d]pyrimidine-6-carboxylic acid dimethylamide;
7-Cyclopentyl-2-{5-(4-isobutylpiperazin-1-yl)-pyridin-2-ylamino}-7H-pyrrolo[2,3d]pyrimidine-6-carboxylic acid dimethylamide;
7-Cyclopentyl-2-{5-[4-(2-isopropoxyethyl)-piperazin-1-yl]-pyridin-2-ylamino}-7H-pyrrolo[2,3-d]pyrimidine-6-carboxylic acid dimethylamide;
7-Cyclopentyl-2-{5-[4-(2-methyl-butyl)piperazin-1-yl]-pyridin-2-ylamino}-7H-pyrrolo[2,3-d]pyrimidine-6-carboxylic acid dimethylamide; or
7-Cyclopentyl-2-[1′-(2-hydroxy-ethyl)-1′,2′,3′,4′,5′,6′-hexahydro-[3,4′]bipyridinyl-6-ylamino]-7H-pyrrolo[2,3-d]pyrimidine-6-carboxylic acid dimethylamide;
P or a pharmaceutically acceptable salt thereof.

In some embodiments, the CDK4/6 inhibitor comprises ribociclib (CAS Registry Number: 1211441-98-3). Ribociclib is also known as LEE011, KISQALI®, or 7-cyclopentyl-N,N-dimethyl-2-((5-(piperazin-1-yl)pyridin-2-yl)amino)-7H-pyrrolo[2,3-d]pyrimidine-6-carboxamide.

In some embodiments, the CDK4/6 inhibitor comprises a compound 7-Cyclopentyl-2-(5-piperazin-1-yl-pyridin-2-ylamino)-7H-pyrrolo[2,3-d]pyrimidine-6-carboxylic acid dimethylamide of the formula

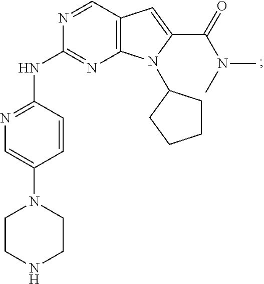 [SMILES: CN(C)C(=O)c1cc2cnc(Nc3ccc(N4CCNCC4)cn3)nc2n1C1CCCC1]

or a pharmaceutically acceptable salt thereof.

In some embodiments, the CDK4/6 inhibitor (e.g., ribociclib) is administered once daily at a dose of about 200 to about 600 mg, e.g., per day. In one embodiment, the CDK4/6 inhibitor (e.g., ribociclib) is administered once daily at a dose of about 200 mg, about 300 mg, about 400 mg, about 500 mg, or about 600 mg, or about 200 mg to about 300 mg, about 300 mg to about 400 mg, about 400 mg to about 500 mg, or about 500 mg to about 600 mg. In other embodiments, the CDK4/6 inhibitor (e.g., ribociclib) is administered once daily at a dose of 600 mg per day for e.g., three weeks, e.g., 21 days. In some embodiments, this treatment is followed by one week of no treatment. In some embodiments, the CDK4/6 inhibitor (e.g., ribociclib) is administered in repeated dosing cycles of 3 weeks on and 1 week off, e.g., the compound is administered daily for 3 weeks, e.g., 21 days, followed by no administration for 1 week (e.g., 7 days), after which the cycle is repeated, e.g., the compound is administered daily for 3 weeks followed by no administration for 1 week. In some embodiments, the CDK4/6 inhibitor (e.g., ribociclib) is administered orally.

Other Exemplary CDK4/6 Inhibitors

In some embodiments, the CDK4/6 inhibitor comprises abemaciclib (CAS Registry Number: 1231929-97-7). Abemaciclib is also known as LY835219 or N-[5-[(4-Ethyl-1-piperazinyl)methyl]-2-pyridinyl]-5-fluoro-4-[4-fluoro-2-methyl-1-(1-methylethyl)-1H-benzimidazol-6-yl]-2-pyrimidinamine Abemaciclib is a CDK inhibitor selective for CDK4 and CDK6 and is disclosed, e.g., in Torres-Guzman R et al. (2017) Oncotarget 10.18632/oncotarget.17778.

In some embodiments, the CDK4/6 inhibitor comprises palbociclib (CAS Registry Number: 571190-30-2). Palbociclib is also known as PD-0332991, IBRANCE® or 6-Acetyl-8-cyclopentyl-5-methyl-2-{[5-(1-piperazinyl)-2-pyridinyl]amino}pyrido[2,3-d]pyrimidin-7(8H)-one. Palbociclib inhibits CDK4 with an IC50 of 11 nM, and inhibits CDK6 with an IC50 of 16 nM, and is disclosed, e.g., in Finn et al. (2009) Breast Cancer Research 11(5):R77.

In some embodiments, the CDK4/6 inhibitor (e.g., palbociclib) is administered at a dose of about 125 mg per day for e.g., three weeks. In some embodiments, this treatment is followed by one week of no treatment. In some embodiments, the CDK4/6 inhibitor (e.g., palbociclib) is administered in repeated dosing cycles of 3 weeks on and 1 week off, e.g., the compound is administered daily for 3 weeks followed by no administration for 1 week, after which the cycle is repeated, e.g., the compound is administered daily for 3 weeks followed by no administration for 1 week.

### CXCR2 Inhibitors

In certain embodiments, a combination described herein comprises an inhibitor of chemokine (C—X—C motif) receptor 2 (CXCR2). In some embodiments, the CXCR2 inhibitor is used in combination with a PD-1 inhibitor, and one or more (e.g., two, three, or all) of a CSF-1/1R binding agent, a TIM-3 inhibitor, a c-MET inhibitor, or an A2aR antagonist. In some embodiments, the combination is used to treat a pancreatic cancer or a colorectal cancer. In some embodiments, the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide, danirixin, reparixin, or navarixin.

Exemplary CXCR2 Inhibitors

In some embodiments, the CXCR2 inhibitor comprises a compound disclosed in U.S. Pat. Nos. 7,989,497, 8,288,588, 8,329,754, 8,722,925, 9,115,087, U.S. Application Publication Nos. US 2010/0152205, US 2011/0251205 and US 2011/0251206, and International Application Publication Nos. WO 2008/061740, WO 2008/061741, WO 2008/062026, WO 2009/106539, WO2010/063802, WO 2012/062713, WO 2013/168108, WO 2010/015613 and WO 2013/030803. In some embodiments, the CXCR2 inhibitor comprises 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof. In some embodiments, the CXCR2 inhibitor comprises 6-chloro-3-((3,4-dioxo-2-(pentan-3-ylamino)cyclobut-1-en-1-yl)amino)-2-hydroxy-N-methoxy-N-methylbenzenesulfonamide choline salt. In some embodiments, the CXCR2 inhibitor is 2-Hydroxy-N,N,N-trimethylethan-1-aminium 3-chloro-6-({3,4-dioxo-2-[(pentan-3-yl)amino]cyclobut-1-en-1-yl}amino)-2-(N-methoxy-N-methylsulfamoyl)phenolate (i.e., 6-chloro-3-((3,4-dioxo-2-(pentan-3-ylamino)cyclobut-1-en-1-yl)amino)-2-hydroxy-N-methoxy-N-methylbenzenesulfonamide choline salt) and has the following chemical structure.

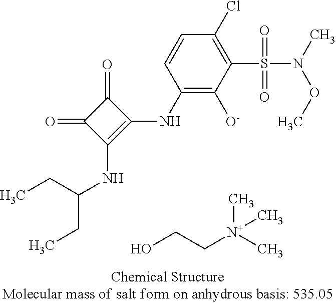

In some embodiments, the CXCR2 inhibitor is administered at a dose of about 50-1000 mg (e.g., about 50-400 mg, 50-300 mg, 50-200 mg, 50-100 mg, 150-900 mg, 150-600 mg, 200-800 mg, 300-600 mg, 400-500 mg, 300-500 mg, 200-500 mg, 100-500 mg, 100-400 mg, 200-300 mg, 100-200 mg, 250-350 mg, or about 75 mg, 150 mg, 300 mg, 450 mg, or 600 mg). In some embodiments, the CXCR2 inhibitor is administered daily, e.g., once daily or twice daily. In some embodiments, the CXCR2 inhibitor is administered for two weeks (e.g., 14 days) in a 4 week cycle (e.g., 28 day cycle). In some embodiments, the CXCR2 inhibitor is administered for the first two weeks (e.g., 14 days) in a 4 week cycle (e.g., 28 day cycle). In some embodiments, the CXCR2 inhibitor is administered for 2 weeks in a 4 week cycle, e.g., 2 weeks of treatment with the CXCR2 inhibitor and 2 weeks of no treatment in a 4 week cycle. In some embodiments, the CXCR2 inhibitor is administered daily, e.g., twice daily, for the first two weeks (e.g., 14 days) in a 4 week cycle (e.g., 28 day cycle). In some embodiments, the CXCR2 inhibitor is administered daily, e.g., twice daily, for two weeks (e.g., 14 days) in a 4 week cycle (e.g., 28 day cycle). In some embodiments, the CXCR2 inhibitor is administered daily, e.g., twice daily, for 2 weeks in a 4 week cycle, e.g., 2 weeks of treatment with the CXCR2 inhibitor and 2 weeks of no treatment in a 4 week cycle. In some embodiments, the CXCR2 inhibitor is administered daily, e.g., once daily or twice daily at a total dose of about 50-1000 mg (e.g., about 50-400 mg, 50-300 mg, 50-200 mg, 50-100 mg, 150-900 mg, 150-600 mg, 200-800 mg, 300-600 mg, 400-500 mg, 300-500 mg, 200-500 mg, 100-500 mg, 100-400 mg, 200-300 mg, 100-200 mg, 250-350 mg, or about 75 mg, 150 mg, 300 mg, 450 mg, or 600 mg). In some embodiments, the CXCR2 is administered once daily. In other embodiments, the CXCR2 inhibitor is administered twice daily. In some embodiments, the CXCR2 inhibitor is administered twice daily and each dose, e.g., the first and second dose, comprises about 25-400 mg (e.g., 25-100 mg, 50-200 mg, 75-150, or 100-400 mg) of the CXCR2 inhibitor. In some embodiments, the CXCR2 inhibitor is administered once daily and the dose comprises about 50-600 mg (e.g., 50-150 mg, 100-400 mg, 200-300, or 300-500 mg) of the CXCR2 inhibitor. In some embodiments, the CXCR2 inhibitor is administered orally. In some embodiments, the CXCR2 inhibitor is administered orally twice daily at a dose of 75 mg for two weeks (e.g., 14 days) in a 4 week cycle (e.g., 28 day cycle). In some embodiments, the CXCR2 inhibitor is administered orally twice daily at a does of 150 mg for two weeks (e.g., 14 days) in a 4 week cycle (e.g., 28 day cycle).

In some embodiments, the CXCR2 inhibitor is administered twice daily, e.g., about 12 hours apart. In some embodiments, the CXCR2 inhibitor is administered on an empty stomach at least e.g., 0.5, 1, 1.5, or 2 hours before a meal. In some embodiments, the CXCR2 inhibitor is administered at the same time daily. In some embodiments, if a subject misses a dose of the CXCR2 inhibitor, the subject will be administered the missed dose of the CXCR2 inhibitor within, e.g., 1, 2, 3 or 4 hours of the missed dose.

In some embodiments, the dose provides >60% inhibition of whole blood neutrophil shape change (e.g., over 24 h) in humans, e.g., a dose of 100 mg once daily or 50 mg twice daily. In other embodiments, the dose provides >80% inhibition of whole blood neutrophil shape change (e.g., over 24 h) in humans, e.g., a dose of 150 mg twice daily. In other embodiments, the dose provides >90% inhibition of whole blood neutrophil shape change (e.g., over 24 h) in humans, e.g., a dose of 500 mg once daily. Methods for determining whole blood neutrophil shape change are described, e.g., in Bryan et al. Am J Respir Crit Care Med. 2002; 165(12): 1602-9. Without wishing to be bound by theory, it is believed that in some embodiments, blockade of CXCR2 by a CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt) inhibits myeloid cell or neutrophil migration. In some embodiments, myeloid cell infiltration, e.g., neutrophils and myeloid derived suppressor cells (MDSC), in tumors is a prognostic marker and is associated with adverse clinical outcomes. In some embodiments, inhibition of myeloid cell migration into tumors in combination with PD-1 blockade, e.g., by PDR001, can enhance the activity of cyotoxic T cells.

Without wishing to be bound by theory, it is believed that in some embodiments, the immunosuppressive effects of a CXCR2 inhibitor, e.g., 6-chloro-3-((3,4-dioxo-2-(pentan-3-ylamino)cyclobut-1-en-1-yl)amino)-2-hydroxy-N-methoxy-N-methylbenzenesulfonamide choline salt, on neutrophils or MDSCs can enhance anti-tumor activity induced by a PD-1 inhibitor, e.g., PDR001.

In some embodiments, the CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt) is administered substantively simultaneously with or immediately after administration of a PD-1 inhibitor, e.g., PDR001. For example, the CXCR2 inhibitor is administered immediately after completion of the PDR001 infusion during a clinic visit. In other embodiments, the CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt) is administered prior to administration of a PD-1 inhibitor, e.g., PDR001. For example, the CXCR2 inhibitor is administered immediately before administration of a PD-1 inhibitor, e.g., PDR001. For example, the CXCR2 inhibitor is administered about 1-14 days (e.g., 7 or 14 days) before administration of a PD-1 inhibitor, e.g., PDR001. In any of the embodiments described herein, the CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt) is administered orally twice daily at a dose of 25-300 mg (e.g., 25-100 mg, 50-200 mg, 75-150 mg, 50 mg, 75 mg, 100 mg, or 150 mg) for (i) two weeks (e.g., 14 days) in a 4-week cycle (e.g., 28 day cycle), i.e., 2 weeks on/2 weeks off, or (ii) for one week (e.g., 7 days) in a 3-week or 21-day cycle, i.e., 1 week on/2 weeks off. For example, the CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt) is administered orally twice daily at a dose of 75 mg 2 weeks on/2 weeks off or 1 week on/2 weeks off. As another example, the CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt) is administered orally twice daily at a dose of 150 mg 2 weeks on/2 weeks off or 1 week on/2 weeks off.

Without wishing to be bound by theory, it is believed that in some embodiments, neutrophils can promote aspects of tumorigenesis including neo angiogenesis and can also inhibit an effective immune anti-tumor response (see e.g., Raccosta L. et al., (2013) J. Exp. Med. p. 1711-1728). In some embodiments, the CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt) binds to the CXCR2 receptor expressed on neutrophils and other myeloid cells, and e.g., inhibits neutrophil shape change, promotes T cell infiltration of tumor and enhances response to PD-1 inhibitors (Steele C. et al., (2016) Cancer Cell 29:6 p. 832-845). In other embodiments, the CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzene sulfonamide choline salt) reduces neutrophil counts in, e.g., blood and sputum.

In some embodiments, the CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt) inhibits both GROα and IL-8-stimulated [³⁵S]G-TPgS binding in membranes prepared from CHO cells expressing the human CXCR2 receptor. In some embodiments, the CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt) resultes in dose-dependent inhibition of neutrophil shape change induced by rhGROα in whole human blood. In other embodiments, the CXCR2 inhibitor (e.g., 6-chloro-3-((3,4-dioxo-2-(pentan-3-ylamino)cyclobut-1-en-1-yl)amino)-2-hydroxy-N-methoxy-N-methylbenzenesulfonamide choline salt) resultes in dose-dependent inhibition of rhGROα-induced neutrophil migration.

Other Exemplary CXCR2 Inhibitors

In some embodiments, the CXCR2 inhibitor comprises danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl)urea. Danirixin is disclosed, e.g., in Miller et al. Eur J Drug Metab Pharmacokinet (2014) 39:173-181; and Miller et al. BMC Pharmacology and Toxicology (2015), 16:18.

In some embodiments, the CXCR2 inhibitor comprises reparixin (CAS Registry Number: 266359-83-5). Reparixin is also known as repertaxin or (2R)-2-[4-(2-methylpropyl)phenyl]-N-methylsulfonylpropanamide. Reparixin is a non-competitive allosteric inhibitor of CXCR1/2. Reparixin is disclosed, e.g., in Zarbock et al. Br J Pharmacol. 2008; 155(3):357-64.

In some embodiments, the CXCR2 inhibitor comprises navarixin. Navarixin is also known as MK-7123, SCH 527123, PS291822, or 2-hydroxy-N,N-dimethyl-3-[[2-[[(1R)-1-(5-methylfuran-2-yl)propyl]amino]-3,4-dioxocyclobuten-1-yl]amino]benzamide. Navarixin is disclosed, e.g., in Ning et al. Mol Cancer Ther. 2012; 11(6):1353-64.

### CSF-1/1R Binding Agents

In certain embodiments, a combination described herein comprises a CSF-1/1R binding agent. In some embodiments, the CSF-1/1R binding agent is used in combination with a PD-1 inhibitor, and one or more (e.g., two, three, four, or all) of CXCR2 inhibitor, a TIM-3 inhibitor, a c-MET inhibitor, an A2aR antagonist, or an IDO inhibitor. In some embodiments, the combination is used to treat a pancreatic cancer, colorectal cancer, a gastric cancer, or a melanoma (e.g., a refractory melanoma).

In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110. In other embodiments, the CSF-1/1R binding agent is pexidartinib.

Exemplary CSF-1 Binding Agents

In some embodiments, the CSF-1/1R binding agent comprises an inhibitor of macrophage colony-stimulating factor (M-CSF). M-CSF is also sometimes known as CSF-1. In certain embodiments, the CSF-1/1R binding agent is an antibody to CSF-1 (e.g., MCS110). In other embodiments, the CSF-1/1R binding agent is an inhibitor of CSF-1R (e.g., BLZ945).

In some embodiments, the CSF-1/1R binding agent comprises a monoclonal antibody or Fab to M-CSF (e.g., MCS110/H-RX1), or a binding agent to CSF-1 disclosed in International Application Publication Nos. WO 2004/045532 and WO 2005/068503, including H-RX1 or 5H4 (e.g., an antibody molecule or Fab fragment against M-CSF) and U.S. Pat. No. 9,079,956, which applications and patent are incorporated by reference in their entirety.

In some embodiments, the CSF-1/1R binding agent, e.g., an M-CSF inhibitor, a monoclonal antibody or Fab to M-CSF (e.g., MCS110), or a compound disclosed in PCT Publication No. WO 2004/045532 and WO 2005/068503 and U.S. Pat. No. 9,079,956 (e.g., an antibody molecule or Fab fragment against M-CSF), is administered at an average dose of about 10 mg/kg.

TABLE 19
Amino acid and nucleotide sequences of an exemplary anti-M-CSF
antibody molecule (MCS110)

| | |
|---|---|
| (H-RX1) HC | QVQLQESGPGLVKPSQTLSLTCTVSDYSITSDYAWNWIRQFPGKGLEWMGYISY |
| | SGSTSYNPSLKSRITISRDTSKNQFSLQLNSVTAADTAVYYCASFDYAHAMDYW |
| | GQGTTVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKRVEP |
| | KSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVS |
| | NKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALH |
| | NHYTQKSLSLSPGK (SEQ ID NO: 1007) |
| (H-RX1) LC | DIVLTQSPAFLSVTPGEKVTFTCQASQSIGTSIHWYQQKTDQAPKLLIKYASESIS |
| | GIPSRFSGSGSGTDFTLTISSVEAEDAADYYCQQINSWPTTFGGGTKLEIKRTVAA |
| | PSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| | (SEQ ID NO: 1008) |
| Heavy Chain | SDYAWN (SEQ ID NO: 1009) |
| CDR1 (Kabat) | |
| Heavy Chain | YISYSGSTSYNPSLKS (SEQ ID NO: 1010) |
| CDR2 (Kabat) | |
| Heavy Chain | FDYAHAMDY (SEQ ID NO: 1011) |
| CDR3 (Kabat) | |
| Light Chain | QASQSIGTSIH (SEQ ID NO: 1012) |
| CDR1 (Kabat) | |
| Light Chain | YASESIS (SEQ ID NO: 1013) |
| CDR2 (Kabat) | |
| Light Chain | QQINSWPTT (SEQ ID NO: 1014) |
| CDR3 (Kabat) | |

In another embodiment, the CSF-1/1R binding agent comprises a CSF-1R tyrosine kinase inhibitor, 4-((2-(((1R,2R)-2-hydroxycyclohexyl)amino)benzo[d]thiazol-6-yl)oxy)-N-methylpicolinamide (BLZ945), or a compound disclosed in International Application Publication No. WO 2007/121484, and U.S. Pat. Nos. 7,553,854, 8,173,689, and 8,710,048, which are incorporated by reference in their entirety.

In some embodiments, the CSF-1/1R binding agent comprises a compound, stereoisomer, tautomer, solvate, oxide, ester, or prodrug of Formula (I) or a pharmaceutically acceptable salt thereof

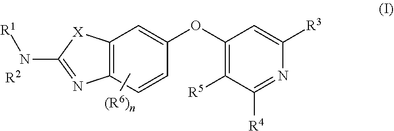

wherein X is O, S, or S(O);

R¹ and R² are independently selected from the group consisting of hydrogen, alkyl, substituted alkyl, acyl, cycloalkyl, substituted cycloalkyl, cycloalkenyl, substituted cycloalkenyl, aryl, substituted aryl, heterocyclyl, substituted heterocyclyl, heteroaryl, and substituted heteroaryl; or R and R are taken together to form a group selected from heterocyclyl, substituted heterocyclyl, heteroaryl, or substituted heteroaryl;

R³ is selected from the group consisting of hydrogen, halo, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclyl, substituted heterocyclyl, amino, substituted amino, acyl, acylamino, alkoxy, substituted alkoxy, carboxyl, carboxyl ester, substituted sulfonyl, aminosulfonyl, and aminocarbonyl; each R⁶ is independently alkyl, substituted alkyl, alkoxy, substituted alkoxy, amino, substituted amino, or halo; n is 0, 1, or 2; and when X is O, R⁴ is hydrogen, substituted alkyl, alkenyl, substituted alkenyl, alkynyl, or substituted alkynyl, and R⁵ is hydrogen, alkyl, substituted alkyl, alkenyl, substituted alkenyl, alkynyl, substituted alkynyl, aminocarbonyl, halo, heteroaryl, substituted heteroaryl, cycloalkyl, or substituted cycloalkyl, or R⁴ and R⁵ are taken together to form a group selected from heterocyclyl, substituted heterocyclyl, cycloalkyl, substituted cycloalkyl, aryl, substituted aryl, heteroaryl, and substituted heteroaryl; and when X is S or S(O), R⁴ is hydrogen, substituted alkyl, alkenyl, substituted alkenyl, alkynyl, or substituted alkynyl, and R⁵ is hydrogen, alkyl, substituted alkyl, alkenyl, substituted alkenyl, alkynyl, substituted alkynyl, aminocarbonyl, halo, heteroaryl, substituted heteroaryl, cycloalkyl, or substituted cycloalkyl.

In some embodiments, the CSF-1/1R binding agent comprises a compound of the formula:

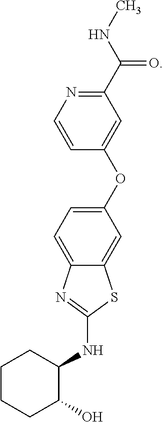 [SMILES: CNC(=O)c1cc(Oc2ccc3nc(N[C@@H]4CCCC[C@H]4O)sc3c2)ccn1]

In some embodiments, the CSF-1/1R binding agent (e.g., BLZ945) is administered at a dose between 50 mg and 1500 mg, e.g., between 75 mg and 1000 mg, between 100 mg and 900 mg, between 200 mg and 800 mg, between 300 mg and 700 mg, between 400 mg and 600 mg, between 100 mg and 700 mg, between 100 mg and 500 mg, between 100 mg and 300 mg, between 700 mg and 900 mg, between 500 mg and 900 mg, between 300 mg and 900 mg, between 75 mg and 150 mg, between 100 mg and 200 mg, between 200 mg and 400 mg, between 500 mg and 700 mg, or between 800 mg and 1000 mg, e.g., at a dose of 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg. In certain embodiments, the CSF-1/1R binding agent (e.g., BLZ945) is administered daily, e.g., according to a 7 days on/7 days off schedule. In other embodiments, the CSF-1/1R binding agent (e.g., BLZ945) is administered twice a week, once a week, once every two weeks, once every three weeks, or once every four weeks.

In some embodiments, the CSF-1/1R binding agent (e.g., BLZ945) is administered at a dose of between 50 mg and 150 mg, e.g., about 100 mg, e.g., daily, e.g., according to a 7 days on/7 days off schedule. In other embodiments, the CSF-1/1R binding agent (e.g., BLZ945) is administered at a dose of between 100 mg and 200 mg, e.g., about 150 mg, e.g., daily, e.g., according to a 7 days on/7 days off schedule. In other embodiments, the CSF-1/1R binding agent (e.g., BLZ945) is administered at a dose of between 200 mg and 400 mg, e.g., about 300 mg, e.g., daily, e.g., according to a 7 days on/7 days off schedule. In other embodiments, the CSF-1/1R binding agent (e.g., BLZ945) is administered at a dose of between 500 mg and 700 mg, e.g., about 600 mg, e.g., daily, e.g., according to a 7 days on/7 days off schedule. In other embodiments, the CSF-1/1R binding agent (e.g., BLZ945) is administered at a dose of between 800 mg and 1000 mg, e.g., about 900 mg, e.g., daily, e.g., according to a 7 days on/7 days off schedule.

In some embodiments, the CSF-1/1R binding agent (e.g., BLZ945) is administered at a dose of between 50 mg and 150 mg, e.g., about 100 mg, once a week. In other embodiments, the CSF-1/1R binding agent (e.g., BLZ945) is administered at a dose of between 100 mg and 200 mg, e.g., about 150 mg, once a week. In other embodiments, the CSF-1/1R binding agent (e.g., BLZ945) is administered at a dose of between 200 mg and 400 mg, e.g., about 300 mg, once a week. In other embodiments, the CSF-1/1R binding agent (e.g., BLZ945) is administered at a dose of between 500 mg and 700 mg, e.g., about 600 mg, once a week. In other embodiments, the CSF-1/1R binding agent (e.g., BLZ945) is administered at a dose of between 800 mg and 1000 mg, e.g., about 900 mg, once a week.

In some embodiments, the CSF-1/1R binding agent (e.g., BLZ945) is administered orally.

In some embodiments, the CSF-1/1R binding agent (e.g., BLZ945) is administered in combination with a PD-1 inhibitor (e.g., an anti-PD-1 antibody molecule). In one embodiment, the CSF-1/1R binding agent (e.g., BLZ945) is administered at a dose between 50 mg and 150 mg (e.g., about 100 mg), e.g., daily (e.g., according to a 7 days on/7 days off schedule) or once a week, e.g., orally, and the PD-1 inhibitor (e.g., the anti-PD-1 antibody molecule) is administered at a dose between 300 mg and 500 mg (e.g., at a dose of 400 mg), e.g., once every 4 weeks, or at a dose between 200 mg and 400 mg (e.g., at a dose of 300 mg), e.g., once every 3 weeks, e.g., by intravenous infusion. In another embodiment, the CSF-1/1R binding agent (e.g., BLZ945) is administered at a dose between 100 mg and 200 mg (e.g., about 150 mg), e.g., daily (e.g., according to a 7 days on/7 days off schedule) or once a week, e.g., orally, and the PD-1 inhibitor (e.g., the anti-PD-1 antibody molecule) is administered at a dose between 300 mg and 500 mg (e.g., at a dose of 400 mg), e.g., once every 4 weeks, or at a dose between 200 mg and 400 mg (e.g., at a dose of 300 mg), e.g., once every 3 weeks, e.g., by intravenous infusion. In another embodiment, the CSF-1/1R binding agent (e.g., BLZ945) is administered at a dose between 200 mg and 400 mg (e.g., about 300 mg), e.g., daily (e.g., according to a 7 days on/7 days off schedule) or once a week, e.g., orally, and the PD-1 inhibitor (e.g., the anti-PD-1 antibody molecule) is administered at a dose between 300 mg and 500 mg (e.g., at a dose of 400 mg), e.g., once every 4 weeks, or at a dose between 200 mg and 400 mg (e.g., at a dose of 300 mg), e.g., once every 3 weeks, e.g., by intravenous infusion. In another embodiment, the CSF-1/1R binding agent (e.g., BLZ945) is administered at a dose between 500 mg and 700 mg (e.g., about 600 mg), e.g., daily (e.g., according to a 7 days on/7 days off schedule) or once a week, e.g., orally, and the PD-1 inhibitor (e.g., the anti-PD-1 antibody molecule) is administered at a dose between 300 mg and 500 mg (e.g., at a dose of 400 mg), e.g., once every 4 weeks, or at a dose between 200 mg and 400 mg (e.g., at a dose of 300 mg), e.g., once every 3 weeks, e.g., by intravenous infusion. In another embodiment, the CSF-1/1R binding agent (e.g., BLZ945) is administered at a dose between 800 mg and 1000 mg (e.g., about 900 mg), e.g., daily (e.g., according to a 7 days on/7 days off schedule) or once a week, e.g., orally, and the PD-1 inhibitor (e.g., the anti-PD-1 antibody molecule) is administered at a dose between 300 mg and 500 mg (e.g., at a dose of 400 mg), e.g., once every 4 weeks, or at a dose between 200 mg and 400 mg (e.g., at a dose of 300 mg), e.g., once every 3 weeks, e.g., by intravenous infusion.

In some embodiments, the CSF-1/1R binding agent (e.g., MCS110) is administered at a dose of between 1-20 mg/kg, e.g., about 2-4 mg/kg, 4-6 mg/kg or 6-10 mg/kg, e.g., about 3 mg/kg, 5 mg/kg or 7.5 mg/kg. In some embodiments, the CSF-1/1R binding agent (e.g., MCS110) is administered at a dose of about 5 mg/kg. In other embodiments, the CSF-1/1R binding agent (e.g., MCS110) is administered twice a week, once a week, once every two weeks, once every three weeks, or once every four weeks. In some embodiments, the CSF-1/1R binding agent (e.g., MCS110) is administered at a dose of between 1-20 mg/kg, e.g., about 2-4 mg/kg, 4-6 mg/kg or 6-10 mg/kg, e.g., about 3 mg/kg, 5 mg/kg or 7.5 mg/kg twice a week, once a week, once every two weeks, once every three weeks, or once every four weeks. In some embodiments, the CSF-1/1R binding agent (e.g., MCS110) is administered at a dose of about 4-6 mg/kg, e.g., 5 mg/kg, once every four weeks.

In some embodiments, the CSF-1/1R binding agent (e.g., MCS110) is administered intravenously, e.g., by intravenous infusion.

In some embodiments, the CSF-1/1R binding agent (e.g., MCS110) is administered in combination with a PD-1 inhibitor (e.g., an anti-PD-1 antibody molecule). In one embodiment, the CSF-1/1R binding agent (e.g., MCS110) is administered at a dose of between 1-20 mg/kg, e.g., about 2-4 mg/kg, 4-6 mg/kg or 6-10 mg/kg, e.g., about 3 mg/kg, 5 mg/kg or 7.5 mg/kg (e.g., about 4-6 mg/kg, e.g., 5 mg/kg) once every four weeks, e.g., intravenously, e.g., by intravenous infusion, and the PD-1 inhibitor (e.g., the anti-PD-1 antibody molecule) is administered at a dose between 300 mg and 500 mg (e.g., at a dose of 400 mg), e.g., once every 4 weeks, or at a dose between 200 mg and 400 mg (e.g., at a dose of 300 mg), e.g., once every 3 weeks, e.g., by intravenous infusion.

In some embodiments, the CSF-1/1R binding agent (e.g., MCS110) is administered in combination with a PD-1 inhibitor (e.g., an anti-PD-1 antibody molecule) and a LAG-3 inhibitor (e.g., an anti-LAG3 antibody molecule). In one embodiment, the CSF-1/1R binding agent (e.g., MCS110) is administered at a dose of between 1-20 mg/kg, e.g., about 2-4 mg/kg, 4-6 mg/kg or 6-10 mg/kg, e.g., about 3 mg/kg, 5 mg/kg or 7.5 mg/kg (e.g., about 4-6 mg/kg, e.g., 5 mg/kg) once every four weeks, e.g., intravenously, e.g., by intravenous infusion, the PD-1 inhibitor (e.g., the anti-PD-1 antibody molecule) is administered at a dose between 300 mg and 500 mg (e.g., at a dose of 400 mg), e.g., once every 4 weeks, or at a dose between 200 mg and 400 mg (e.g., at a dose of 300 mg), e.g., once every 3 weeks, e.g., by intravenous infusion, and the LAG-3 inhibitor (e.g., the anti-LAG-3 antibody molecule) is administered at a dose of about 400 mg to about 800 mg (e.g., about 600 mg) once every 4 weeks.

In some embodiments, the combination comprising a CSF-1/1R binding agent, e.g., a CSF-1-1R binding agent described herein, is administered in a therapeutically effective amount to a subject with a solid tumor, e.g., a breast cancer (e.g., a triple negative breast cancer (TNBC)), a pancreatic cancer, a gastroesophageal cancer or a CRC (e.g., a MSS CRC). Without wishing to be bound by theory, it is believed that in some embodiments, CSF-1 regulates macrophage proliferation and recruitment to tumors. In some embodiments, the tumor associated macrophages can contribute to an immunosuppressive microenvironment (e.g., as described in Wiliams et al., (2016) Breast Cancer). In some embodiments, the combination comprising a CSF-1/1R binding agent, e.g., BLZ945 or MCS110, has an improved efficacy compared to either agent alone in a CRC mouse model.

In some embodiments, TNBCs have a low T cell:myeloid cell ratio which presents as a poor prognostic factor, e.g., worse prognosis. In some embodiments, bone marrow cells express more CSF-1R which can contribute to a pro-tumorigenic environment in TNBC.

In some embodiments, the combination comprising a CSF-1/1R binding agent, e.g., a CSF-1-1R binding agent described herein, and a PD-1 inhibitor, e.g., PDR001 is administered in a therapeutically effective amount to a subject with a solid tumor, e.g., a breast cancer (e.g., a triple negative breast cancer (TNBC). Without wishing to be bound by theory, it is believed that in some embodiments, a combination comprising a CSF-1/1R binding agent, e.g., a CSF-1-1R binding agent described herein, and a PD-1 inhibitor, e.g., PDR001, can result in, e.g., anti-tumor activity and/or tumor regression. In some embodiments, the combination comprising a CSF-1/1R binding agent, e.g., a CSF-1-1R binding agent described herein, and a PD-1 inhibitor, e.g., PDR001, has improved activity compared to administration of, e.g., a PD-1 inhibitor alone.

In some embodiments, a pancreatic cancer or a gastric cancer has high CD68 expression and high or mid CSF-1R expression. In some embodiments, a combination comprising a CSF-1/1R binding agent, e.g., BLZ945 or MCS110, is administered in a therapeutically effective amount to a subject with a pancreatic cancer or a gastric cancer with high CD68 expression and high or mid CSF-1R expression.

Other Exemplary CSF-1/1R Binding Agents

In some embodiments, the CSF-1/1R binding agent comprises pexidartinib (CAS Registry Number 1029044-16-3). Pexidrtinib is also known as PLX3397 or 5-((5-chloro-1H-pyrrolo[2,3-b]pyridin-3-yl)methyl)-N-((6-(trifluoromethyl)pyridin-3-yl)methyl)pyridin-2-amine Pexidartinib is a small-molecule receptor tyrosine kinase (RTK) inhibitor of KIT, CSF1R and FLT3. FLT3, CSF1R and FLT3 are overexpressed or mutated in many cancer cell types and play major roles in tumor cell proliferation and metastasis. PLX3397 can bind to and inhibit phosphorylation of stem cell factor receptor (KIT), colony-stimulating factor-1 receptor (CSF1R) and FMS-like tyrosine kinase 3 (FLT3), which may result in the inhibition of tumor cell proliferation and down-modulation of macrophages, osteoclasts and mast cells involved in the osteolytic metastatic disease.

In some embodiments, the CSF-1/1R binding agent is emactuzumab. Emactuzumab is also known as RG7155 or RO5509554. Emactuzumab is a humanized IgG1 mAb targeting CSF1R.

In some embodiments, the CSF-1/1R binding agent is FPA008. FPA008 is a humanized mAb that inhibits CSF1R.

c-MET Inhibitors

In certain embodiments, a combination described herein comprises a c-MET inhibitor. c-MET, a receptor tyrosine kinase overexpressed or mutated in many tumor cell types, plays key roles in tumor cell proliferation, survival, invasion, metastasis, and tumor angiogenesis. Inhibition of c-MET may induce cell death in tumor cells overexpressing c-MET protein or expressing constitutively activated c-MET protein.

In some embodiments, the c-MET inhibitor is used in combination with a PD-1 inhibitor, and one or more (e.g., two, three, four, five, six, or all) of CXCR2 inhibitor, a CSF-1/1R binding agent, a LAG-3 inhibitor, a GITR agonist, a TGF-β inhibitor, an A2aR antagonist, or an IDO inhibitor. In some embodiments, the combination is used to treat a pancreatic cancer, a colorectal cancer, a gastric cancer, or a melanoma (e.g., a refractory melanoma). In some embodiments, the c-MET inhibitor is chosen from capmatinib (INC280), JNJ-3887605, AMG 337, LY2801653, MSC2156119J, crizotinib, tivantinib, or golvatinib.

Exemplary c-MET Inhibitors

In some embodiments, the c-MET inhibitor comprises capmatinib (INC280), or a compound described in U.S. Pat. Nos. 7,767,675, and 8,461,330, which are incorporated by reference in their entirety.

In some embodiments, the c-MET inhibitor comprises a compound of Formula I:

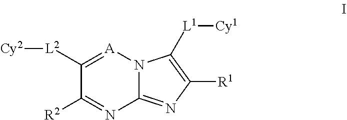

or pharmaceutically acceptable salt thereof, wherein:

A is N; and

Cy¹ is aryl, heteroaryl, cycloalkyl, or heterocycloalkyl, each optionally substituted by 1, 2, 3, 4, or 5-W-X-Y-Z groups;

Cy² is aryl, heteroaryl, cycloalkyl, or heterocycloalkyl, each optionally substituted by 1, 2, 3, 4, or 5-W′-X′-Y′-Z′ groups;

L¹ is CH₂, CH₂CH₂, cycloalkylene, (CR⁴R⁵)ₚO(CR⁴R⁵)_{q} or (CR⁴R⁵)ₚS(CR⁴R⁵)_{q} wherein said cycloalkylene is optionally substituted with 1, 2, or 3 substituents independently selected from Cy³, halo, C₁₋₆ alkyl, C₂₋₆ alkenyl, C₂₋₆ alkynyl, C₁₋₆haloalkyl, halosulfanyl, CN, NO₂, N₃, OR^{a}, SR^{a}, C(O)R^{b}, C(O)NR^{C}R^{d}, C(O)OR^{a}, OC(O)R^{a}, OC(O)NR^{C}R^{d}, NR^{C}R^{d}, NR^{C}C(O)R^{b}, NR^{C}C(O)NR^{C}R^{d}, NR^{C}C(O)OR^{a}, C(═NR^{g})NR^{C}R^{d}, NR^{C}C(═NR^{g})NR^{C}R^{d}, P(R^{f})₂, P(OR^{e})₂, P(O)R^{e}R^{f}, P(O)OR^{e}OR^{f}, S(O)R^{b}, S(O)NR^{C}R^{d}, S(O)₂R^{b}, NR^{C}S(O)₂R^{b}, and S(O)₂NR^{C}R^{d};

L² is (CR⁷R⁸)_{f}, (CR⁷R⁸)ₛ-(cycloalkylene)-(CR⁷R⁸)ₜ, (CR⁷R⁸)ₛ-(arylene)-(CR⁷R⁸)ₜ, (CR⁷R⁸)ₛ-(heterocycloalkylene)-(CR⁷R⁸)ₜ, (CR⁷R⁸)ₛ-(heteroarylene)-(CR⁷R⁸)ₜ, (CR⁷R⁸)ₛO(CR⁷R⁸)ₜ, (CR⁷R⁸)ₛS(CR⁷R⁸)ₜ, (CR⁷R⁸)ₛC(O)(CR⁷R⁸)ₜ, (CR⁷R⁸)ₛC(O)NR⁹(CR⁷R⁸)ₜ, (CR⁷R⁸)ₛC(O)O(CR⁷R⁸)ₜ, (CR⁷R⁸)ₛOC(O)(CR⁷R⁸)ₜ, (CR⁷R⁸)ₛOC(O)NR⁹(CR⁷R⁸)ₜ, (CR⁷R⁸)ₛNR⁹(CR⁷R⁸)ₜ, (CR⁷R⁸)ₛNR⁹C(O)NR⁹(CR⁷R⁸)ₜ, (CR⁷R⁸)ₛS(O)(CR⁷R⁸)ₜ, (CR⁷R⁸)ₛS(O)NR⁷(CR⁸R⁹)ₜ, (CR⁷R⁸)ₛS(O)₂(CR⁷R⁸)ₜ, or (CR⁷R⁸)ₛS(O)₂NR⁹(CR⁷R⁸)ₜ, wherein said cycloalkylene, arylene, heterocycloalkylene, or heteroarylene is optionally substituted with 1, 2, or 3 substituents independently selected from Cy⁴, halo, C₁₋₆ alkyl, C₂₋₆ alkenyl, C₂₋₆ alkynyl, C₁₋₆haloalkyl, halo sulfanyl, CN, NO₂, N₃, OR^{a1}, SR^{a1}, C(O)R^{b1}, C(O)NR^{c1}R^{d1}, C(O)OR^{a1}, OC(O)R^{b1}, OC(O)NR^{c1}R_{d1}, NR^{c1}R^{d1}, NR^{c1}C(O)R^{b1}, NR^{c1}C(O)NR^{c1}R^{d1}, NR^{c1}C(O)OR^{a1}, C(═NR^{g})NR^{c1}R^{d1}, NR^{c1}C(═NR^{g})NR^{c1}R^{d1}, P(R^{f1})₂, P(OR^{e1})₂, P(O)R^{e1}R^{f1}, P(O)OR^{e1}OR^{f1}, S(O)R^{b1}, S(O)NR^{c1}R^{d1}, S(O)₂R^{b1}, NR^{c1}S(O)₂R^{b1}, and S(O)₂NR^{c1}R^{d1};

R¹ is H or -W″-X″-Y″-Z″;

R² is H, halo, C₁₋₆ alkyl, C₂₋₆ alkenyl, C₂₋₆ alkynyl, C₁₋₆haloalkyl, CN, NO₂, OR^{A}, SR^{A}, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{B}, NR^{C}C(O)NR^{C}R^{D}, NR^{C}C(O)OR^{A}, S(O)R^{B}, S(O)NR^{C}R^{D}, S(O)₂R^{B}, NR^{C}S(O)₂R^{B}, or S(O)₂NR^{C}R^{D};

or R² and -L²-Cy² are linked together to form a group of formula:

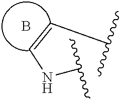

wherein ring B is a fused aryl or fused heteroaryl ring, each optionally substituted with 1, 2, or 3-W′-X′-Y′-Z′ groups;

R⁴ and R⁵ are independently selected from H, halo, OH, C₁₋₆alkyl, C₂₋₆ alkenyl, C₂₋₆ alkynyl, C₁₋₆ alkoxy, alkoxyalkyl, cyanoalkyl, heterocycloalkyl, cycloalkyl, C₁₋₆ haloalkyl, CN, and NO₂;

or R⁴ and R⁵ together with the C atom to which they are attached form a 3, 4, 5, 6, or 7-membered cycloalkyl or heterocycloalkyl ring, each optionally substituted by 1, 2, or 3 substituents independently selected from halo, OH, C₁₋₆ alkyl, C₂₋₆ alkenyl, C₂₋₆ alkynyl, C₁₋₆ alkoxy, alkoxyalkyl, cyanoalkyl, heterocycloalkyl, cycloalkyl, C₁₋₆haloalkyl, CN, and NO₂;

R⁷ and R⁸ are independently selected from H, halo, OH, C₁₋₆alkyl, C₂₋₆ alkenyl, C₂₋₆ alkynyl, C₁₋₆ alkoxy, C₁₋₆haloalkyl, CN, and NO₂;

or R⁷ and R⁸ together with the C atom to which they are attached form a 3, 4, 5, 6, or 7-membered cycloalkyl or heterocycloalkyl ring, each optionally substituted by 1, 2, or 3 substituent independently selected from halo, OH, C₁₋₆ alkyl, C₂₋₆ alkenyl, C₂₋₆ alkynyl, C₁₋₆ alkoxy, C₁₋₆haloalkyl, CN, and NO₂;

R⁹ is H, C₁₋₆ alkyl, C₂₋₆ alkenyl, or C₂₋₆ alkynyl;

W, W′, and W″ are independently absent or independently selected from C₁₋₆ alkylene, C₂₋₆ alkenylene, C₂₋₆ alkynylene, O, S, NR^{h}, CO, COO, CONR^{h}, SO, SO₂, SONR^{h} and NR^{h}CONRⁱ, wherein each of the C₁₋₆ alkylene, C₂₋₆ alkenylene, and C₂₋₆alkynylene is optionally substituted by 1, 2 or 3 substituents independently selected from halo, C₁₋₆ alkyl, C₁₋₆haloalkyl, OH, C₁₋₆ alkoxy, C₁₋₆ haloalkoxy, amino, C₁₋₆ alkylamino, and C₂₋₈dialkylamino;

X, X′, and X″ are independently absent or independently selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cyclo alkylene, heteroarylene, and heterocycloalkylene is optionally substituted by 1, 2 or 3 substituents independently selected from halo, CN, NO₂, OH, C₁₋₆ alkyl, C₁₋₆ haloalkyl, C₂₋₈alkoxyalkyl, C₁₋₆ alkoxy, C₁₋₆haloalkoxy, C₂₋₈ alkoxyalkoxy, cycloalkyl, heterocycloalkyl, C(O)OR^{j}, C(O)NR^{h}Rⁱ, amino, C₁₋₆alkylamino, and C₂₋₈ dialkylamino;

Y, Y′, and Y″ are independently absent or independently selected from C₁₋₆ alkylene, C₂₋₆ alkenylene, C₂₋₆ alkynylene, O, S, NR^{h}, CO, COO, CONR^{h}, SO, SO₂, SONR^{h}, and NR^{h}CONRⁱ, wherein each of the C₁₋₆ alkylene, C₂₋₆ alkenylene, and C₂₋₆ alkynylene is optionally substituted by 1, 2 or 3 substituents independently selected from halo, C₁₋₆ alkyl, C₁₋₆haloalkyl, OH, C₁₋₆ alkoxy, C₁₋₆ haloalkoxy, amino, C₁₋₆ alkylamino, and C₂₋₈ dialkylamino;

Z, Z′, and Z″ are independently selected from H, halo, C₁₋₆ alkyl, C₂₋₆ alkenyl, C₂₋₆ alkynyl, C₁₋₆haloalkyl, halosulfanyl, CN, NO₂, N₃, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, C(═NR^{g})NR^{c2}R^{d2}, NR^{c2}C(═NR^{g})NR^{c2}R^{d2}, P(R^{f2})₂, P(OR^{e2})₂, P(O)R^{e2}R^{f2}, P(O)OR^{e2}OR^{f2}, S(O)R^{b2}, S(O)NR^{c2}R^{d2}, S(O)₂R^{b2}, NR^{c2}S(O)₂R^{b2}, S(O)₂NR^{c2}R^{d2}, aryl, cycloalkyl, heteroaryl, and heterocycloalkyl, wherein said C₁₋₆alkyl, C₂₋₆ alkenyl, C₂₋₆ alkynyl, aryl, cycloalkyl, heteroaryl, and heterocycloalkyl are optionally substituted by 1, 2, 3, 4 or 5 substituents independently selected from halo, C₁₋₆ alkyl, C₂₋₆alkenyl, C₂₋₆ alkynyl, C₁₋₆haloalkyl, halosulfanyl, CN, NO₂, N₃, OR^{a2}, SR^{a3}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR′, C(═NR^{g})NR^{c2}R^{d2}, NR^{c2}C(═NR^{g})NR^{c2}R^{d2}, P(R^{f2})₂, P(OR^{e2})₂, P(O)R^{e2}R^{f2}, P(O)OR^{e2}OR^{f2}, S(O)R^{b2}, S(O)NR^{c2}R^{d2}, S(O)₂R^{b2}, NR^{c2}S(O)₂R^{b2}, and S(O)₂NR^{c2}R^{d2};

wherein two adjacent -W-X-Y-Z groups, together with the atoms to which they are attached, optionally form a fused 4-20 membered cycloalkyl ring or a fused 4-20 membered heterocycloalkyl ring, each optionally substituted by 1, 2, or 3 substituents independently selected from halo, C₁₋₆ alkyl, C₂₋₆ alkenyl, C₂₋₆alkynyl, C₁₋₆haloalkyl, halosulfanyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{g})NR^{c3}R^{d3}NR^{c3}C(═NR^{g})NR^{c3}R^{d3}S(O)NR^{b3}, S(O)NR^{c3}R^{d3}, S(O)₂R^{b3}, NR^{c3}S(O)₂R^{b3}, S(O)₂NR^{c3}R^{d3}, aryl, cycloalkyl, heteroaryl, and heterocycloalkyl;

wherein two adjacent -W′-X′-Y′-Z′ groups, together with the atoms to which they are attached, optionally form a fused 4-20 membered cycloalkyl ring or a fused 4-20 membered heterocycloalkyl ring, each optionally substituted by 1, 2, or 3 substituents independently selected from halo, C₁₋₆ alkyl, C₂₋₆alkenyl, C₂₋₆ alkynyl, C₁₋₆haloalkyl, halosulfanyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{g})NR^{c3}R^{d3}, NR^{c3}C(═NR^{g})NR^{c3}R^{d3}S(O)R^{b3}, S(O)NR^{c3}R^{d3}, S(O)₂R^{b3}, NR^{c3}S(O)₂R^{b3}, S(O)₂NR^{c3}R^{d3}, aryl, cycloalkyl, heteroaryl, and heterocycloalkyl;

Cy³ and Cy⁴ are independently selected from aryl, cycloalkyl, heteroaryl, and heterocycloalkyl, each optionally substituted by 1, 2, 3, 4, or 5 substituents independently selected from halo, C₁₋₆alkyl, C₂₋₆ alkenyl, C₂₋₆ alkynyl, C₁₋₆haloalkyl, halosulfanyl, CN, NO₂, N₃, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{g})NR^{c4}R^{d4}, NR^{c4}C(═NR^{g})NR^{c4}R^{d4}, P(R^{f4})₂, P(OR⁴)₂, P(O)R^{e4}R^{f4}, P(O)OR^{e4}OR^{f4}, S(O)R^{b4}, S(O)NR^{c4}R^{d4}, S(O)₂R^{b4}, —NR^{c4}S(O)₂R^{b4}, and S(O)₂NR^{c4}R^{d4};

R^{A} is H, C₁₋₄ alkyl, C₂₋₄ alkenyl, C₂₋₄ alkynyl, cycloalkyl, heterocycloalkyl, aryl, or heteroaryl wherein said C₁₋₄ alkyl, C₂₋₄alkenyl, C₂₋₄ alkynyl, cycloalkyl, heterocycloalkyl, aryl or heteroaryl is optionally substituted with 1, 2, or 3 substituents independently selected from OH, CN, amino, halo, and C₁₋₄ alkyl;

R^{B} is H, C₁₋₄ alkyl, C₂₋₄ alkenyl, C₂₋₄ alkynyl, cycloalkyl, heterocycloalkyl, aryl, or heteroaryl wherein said C₁₋₄ alkyl, C₂₋₄alkenyl, or C₂₋₄ alkynyl, cycloalkyl, heterocycloalkyl, aryl, or heteroaryl is optionally substituted with 1, 2, or 3 substituents independently selected from OH, CN, amino, halo, and C₁₋₄ alkyl;

R^{C} and R^{D} are independently selected from H, C₁₋₄ alkyl, C₂₋₄alkenyl, or C₂₋₄ alkynyl, wherein said C₁₋₄ alkyl, C₂₋₄ alkenyl, or C₂₋₄ alkynyl, is optionally substituted with 1, 2, or 3 substituents independently selected from OH, CN, amino, halo, and C₁₋₄ alkyl;

or R^{C} and R^{D} together with the N atom to which they are attached form a 4-, 5-, 6- or 7-membered heterocycloalkyl group or heteroaryl group, each optionally substituted with 1, 2, or 3 substituents independently selected from OH, CN, amino, halo, and C₁₋₄ alkyl;

R^{a}, R^{a1}, R^{a2}, R^{a3} and R^{a4} are independently selected from H, C₁₋₆ alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, halo, C₁₋₆ alkyl, C₁₋₆ alkoxy, C₁₋₆haloalkyl, and C₁₋₆haloalkoxy;

R^{b}, R^{b1}, R^{b2}, R^{b3}, and R^{b4} are independently selected from H, C₁₋₆ alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, CN, amino, halo, C₁₋₆ alkyl, C₁₋₆ alkoxy, C₁₋₆haloalkyl, and C₁₋₆haloalkoxy;

R^{c} and R^{d} are independently selected from H, C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆haloalkyl, C₂₋₆alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ alkoxy, C₁₋₆haloalkyl, and C₁₋₆ haloalkoxy;

or R^{c} and R^{d} together with the N atom to which they are attached form a 4-, 5-, 6- or 7-membered heterocycloalkyl group or heteroaryl group, each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

R^{c1} and R^{d1} are independently selected from H, C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ alkoxy, C₁₋₆haloalkyl, and C₁₋₆ haloalkoxy;

or R^{c1} and R^{d1} together with the N atom to which they are attached form a 4-, 5-, 6- or 7-membered heterocycloalkyl group or heteroaryl group, each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

R^{c2} and R^{d2} are independently selected from H, C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, and biheteroaryl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, heterocycloalkylalkyl, arylcycloalkyl, arylheterocycloalkyl, arylheteroaryl, biaryl, heteroarylcycloalkyl, heteroarylheterocycloalkyl, heteroarylaryl, and biheteroaryl are each optionally substituted with 1, 2, or 3 substituents independently selected from OH, CN, amino, halo, C₁₋₆ alkyl, C₁₋₆ alkoxy, C₁₋₆ haloalkyl, C₁₋₆ haloalkoxy, hydroxyalkyl, cyanoalkyl, aryl, heteroaryl, C(O)OR^{a4}, C(O)R^{b4}, S(O)₂R^{b3}, alkoxyalkyl, and alkoxyalkoxy;

or R^{c2} and R^{d2} together with the N atom to which they are attached form a 4-, 5-, 6- or 7-membered heterocycloalkyl group or heteroaryl group, each optionally substituted with 1, 2, or 3 substituents independently selected from OH, CN, amino, halo, C₁₋₆ alkyl, C₁₋₆ alkoxy, C₁₋₆ haloalkyl, C₁₋₆ haloalkoxy, hydroxyalkyl, cyanoalkyl, aryl, heteroaryl, C(O)OR^{a4}, C(O)R^{b4}, S(O)₂R^{b3}, alkoxyalkyl, and alkoxyalkoxy;

R^{c3} and R^{d3} are independently selected from H, C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ alkoxy, C₁₋₆haloalkyl, and C₁₋₆ haloalkoxy;

or R^{c3} and R^{d3} together with the N atom to which they are attached form a 4-, 5-, 6- or 7-membered heterocycloalkyl group or heteroaryl group, each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

R^{c4} and R^{d4} are independently selected from H, C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ alkoxy, C₁₋₆haloalkyl, and C₁₋₆ haloalkoxy;

or R^{c4} and R^{d4} together with the N atom to which they are attached form a 4-, 5-, 6- or 7-membered heterocycloalkyl group or heteroaryl group, each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

R^{e}, R^{e1}, R^{e2}, and R^{e4} are independently selected from H, C₁₋₆alkyl, C₁₋₆ haloalkyl, C₂₋₆ alkenyl, (C₁₋₆ alkoxy)-C₁₋₆ alkyl, C₂₋₆alkynyl, aryl, cycloalkyl, heteroaryl, heterocycloalkyl, arylalkyl, cycloalkylalkyl, heteroarylalkyl, and heterocycloalkylalkyl;

R^{f}, R^{f1}, R^{f2}, and R^{f4} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl;

R^{g} is H, CN, and NO₂;

R^{h} and Rⁱ are independently selected from H and C₁₋₆ alkyl;

R^{j} is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl;

p is 0, 1, 2, 3, or 4;

q is 0, 1, 2, 3, or 4;

r is 0, 1, 2, 3, 4, 5, or 6;

s is 0, 1, 2, 3, or 4; and

t is 0, 1, 2, 3, or 4.

In some embodiments, the c-MET inhibitor comprises a compound chosen from: 2-(4-Fluorophenyl)-7-(4-methoxybenzyl)imidazo[1,2-b][1,2,4]triazine; 2-(4-Fluorophenyl)-7-[1-(4-methoxyphenyl)-cyclopropyl]-imidazo[1,2-b][1,2,4]triazine; 6-(1-(2-(4-Fluorophenyl)imidazo[1,2-b][1,2,4]triazin-7-yl)cyclopropyl)quinoline; 2-Fluoro-N-methyl-4-[7-(quinolin-6-ylmethyl)imidazo[1,2-b][1,2,4]triazin-2-yl]benzamide; 2-(4-Bromo-3-fluorophenyl)-7-[(4-methoxyphenyl)thio]-imidazo[1,2-b][1,2,4]triazine; Methyl 2-fluoro-4-[7-(quinolin-6-ylthio)imidazo[1,2-b][1,2,4]triazin-2-yl]benzoate; 2-(4-Bromo-3-fluorophenyl)-7-(4-methoxyphenoxy)imidazo[1,2-b][1,2,4]triazine; 2-(4-fluorophenyl)-7-[(4-methoxyphenyl)thio]imidazo[1,2-b][1,2,4]triazine; 2-Fluoro-N-methyl-4-[7-(quinoxalin-6-ylmethyl)imidazo[1,2-b][1,2,4]triazin-2-yl]benzamide; N-Methyl-5-{4-[7-(1-quinolin-6-ylcyclopropyl)imidazo[1,2-b][1,2,4]triazin-2-yl]phenyl}pyridine-2-carboxamide; 6-{1-[2-(4-Pyrimidin-5-yl-phenyl)imidazo[1,2-b][1,2,4]triazin-7-yl]cyclopropyl}quinoline; 6-(1-{2-[4-(1-Acetyl-1,2,3,6-tetrahydropyridin-4-yl)phenyl]imidazo[1,2-b][1,2,4]triazin-7-yl}cyclopropyl)quinoline; 6-[1-(2-{4-[1-(Methylsulfonyl)-1,2,3,6-tetrahydropyridin-4-yl]phenyl}imidazo[1,2-b][1,2,4]triazin-7-yl)cyclopropyl]quinoline; N,N-Dimethyl-5-{4-[7-(1-quinolin-6-ylcyclopropyl)imidazo[1,2-b][1,2,4]triazin-2-yl]phenyl}pyridine-2-carboxamide; 6-(1-{2-[4-(1H-Imidazol-1-yl)phenyl]imidazo[1,2-b][1,2,4]triazin-7-yl}cyclopropyl)-quinoline; 2-Fluoro-N-(trans-4-hydroxycyclohexyl)-4-[7-(1-quinolin-6-ylcyclopropyl)imidazo[1,2-b][1,2,4]triazin-2-yl]benzamide; N-Cyclopropyl-2-fluoro-4-[7-(1-quinolin-6-ylcyclopropyl)imidazo[1,2-b][1,2,4]triazin-2-yl]benzamide; 2-Fluoro-N-methyl-4-[7-(1-quinolin-6-ylcyclopropyl)imidazo[1,2-b][1,2,4]triazin-2-yl]benzamide; 2-Fluoro-N-[1-(methoxymethyl)cyclopropyl]-4-[7-(1-quinolin-6-ylcyclopropyl)imidazo[1,2-b][1,2,4]triazin-2-yl]benzamide; 2-Fluoro-4-(7-(1-(quinolin-6-yl)cyclopropyl)imidazo[1,2-b][1,2,4]triazin-2-yl)benzamide; 4-[7-(1-Quinolin-6-ylcyclopropyl)imidazo[1,2-b][1,2,4]triazin-2-yl]-N-(tetrahydrofuran-2-ylmethyl)benzamide; N-(Pyridin-2-ylmethyl)-4-[7-(1-quinolin-6-ylcyclopropyl)imidazo[1,2-b][1,2,4]triazin-2-yl]benzamide; N-Cyclopropyl-4-[7-(1-quinolin-6-ylcyclopropyl)imidazo[1,2-b][1,2,4]triazin-2-yl]benzamide; N-Cyclobutyl-4-[7-(1-quinolin-6-ylcyclopropyl)imidazo[1,2-b][1,2,4]triazin-2-yl]benzamide; N-(1-Pyridin-2-ylcyclopropyl)-4-[7-(1-quinolin-6-ylcyclopropyl)imidazo[1,2-b][1,2,4]triazin-2-yl]benzamide; N-(2-Hydroxy-1,1-dimethylethyl)-4-[7-(1-quinolin-6-ylcyclopropyl)imidazo[1,2-b][1,2,4]triazin-2-yl]benzamide; N-[(1S)-1-Benzyl-2-hydroxyethyl]-4-[7-(1-quinolin-6-ylcyclopropyl)imidazo[1,2-b][1,2,4]triazin-2-yl]benzamide; (3R)-1-{4-[7-(1-quinolin-6-ylcyclopropyl)imidazo[1,2-b][1,2,4]triazin-2-yl]benzoyl}pyrrolidin-3-ol; 4-(7-(1-(Quinolin-6-yl)cyclopropyl)imidazo[1,2-b][1,2,4]triazin-2-yl)-N-(tetrahydro-2H-pyran-4-yl)benzamide; N-Cyclopropyl-N-methyl-4-[7-(1-quinolin-6-ylcyclopropyl)imidazo[1,2-b][1,2,4]triazin-2-yl]benzamide; N-[1-(Methoxymethyl)cyclopropyl]-4-[7-(1-quinolin-6-ylcyclopropyl)imidazo[1,2-b][1,2,4]triazin-2-yl]benzamide; N-[1-(Methoxymethyl)cyclobutyl]-4-[7-(1-quinolin-6-ylcyclopropyl)imidazo[1,2-b][1,2,4]triazin-2-yl]benzamide; N-[(1S)-1-(Methoxymethyl)-2-methylpropyl]-4-[7-(1-quinolin-6-ylcyclopropyl)imidazo[1,2-b][1,2,4]triazin-2-yl]benzamide; N-[4-(Methoxymethyl)tetrahydro-2H-pyran-4-yl]-4-[7-(1-quinolin-6-ylcyclopropyl)imidazo[1,2-b][1,2,4]triazin-2-yl]benzamide; 4-[7-(1-Quinolin-6-ylcyclopropyl)imidazo[1,2-b][1,2,4]triazin-2-yl]-N-1,3-thiazol-2-ylbenzamide; N-Pyrimidin-4-yl-4-[7-(1-quinolin-6-ylcyclopropyl)imidazo[1,2-b][1,2,4]triazin-2-yl]benzamide; N-[4-(Methoxymethyl)tetrahydro-2H-pyran-4-yl]-4-[7-(1-quinolin-6-ylcyclopropyl)imidazo[1,2-b][1,2,4]triazin-2-yl]benzamide; N-{(1R)-1-[(Dimethylamino)carbonyl]-2-methylpropyl}-4-[7-(1-quinolin-6-ylethyl)imidazo[1,2-b][1,2,4]triazin-2-yl]benzamide; N-Cyclopropyl-2-fluoro-4-[7-(quinolin-6-ylmethyl)imidazo[1,2-b][1,2,4]triazin-2-yl]benzamide; 2-Fluoro-N-[1-(methoxymethyl)cyclopropyl]-4-[7-(quinolin-6-ylmethyl)imidazo[1,2-b][1,2,4]triazin-2-yl]benzamide; 2-Fluoro-4-[7-(quinolin-6-ylmethyl)imidazo[1,2-b][1,2,4]triazin-2-yl]-N-(tetrahydro-2H-pyran-4-yl)benzamide; (3R)-1-{2-Fluoro-4-[7-(quinolin-6-ylmethyl)imidazo[1,2-b][1,2,4]triazin-2-yl]benzoyl}pyrrolidin-3-ol; 2-Fluoro-4-[7-(quinolin-6-ylmethyl)imidazo[1,2-b][1,2,4]triazin-2-yl]benzamide; 2-Fluoro-N-(trans-4-hydroxycyclohexyl)-4-[7-(quinolin-6-ylmethyl)imidazo[1,2-b][1,2,4]triazin-2-yl]benzamide; 6-{2-[3-Fluoro-4-(1H-imidazol-1-yl)phenyl]imidazo[1,2-b][1,2,4]triazin-7-ylmethyl}quinoline; 3-{2-Fluoro-4-[7-(quinolin-6-ylmethyl)imidazo[1,2-b][1,2,4]triazin-2-yl]phenyl}-1,3-oxazolidin-2-one; N-(1S)-2,2-Dimethyl-1-[(methylamino)carbonyl]propyl-2-fluoro-4-[7-(quinolin-6-ylmethyl)imidazo[1,2-b][1,2,4]triazin-2-yl]benzamide; N-(1S)-1-[(Dimethylamino)carbonyl]-2,2-dimethylpropyl-2-fluoro-4-[7-(quinolin-6-ylmethyl)imidazo[1,2-b][1,2,4]triazin-2-yl]benzamide; N-[(1S)-1-(Azetidin-1-ylcarbonyl)-2,2-dimethylpropyl]-2-fluoro-4-[7-(quinolin-6-ylmethyl)imidazo[1,2-b][1,2,4]triazin-2-yl]benzamide; N-{(1S)-1-[(Dimethylamino)carbonyl]-3-methylbutyl}-2-fluoro-4-[7-(quinolin-6-ylmethyl)imidazo[1,2-b][1,2,4]triazin-2-yl]benzamide; 2-Fluoro-N-{(1R)-3-methyl-1-[(methylamino)carbonyl]butyl}-4-[7-(quinolin-6-ylmethyl)imidazo[1,2-b][1,2,4]triazin-2-yl]benzamide; N-{(1R)-1-[(Dimethylamino)carbonyl]-3-methylbutyl}-2-fluoro-4-[7-(quinolin-6-ylmethyl)imidazo[1,2-b][1,2,4]triazin-2-yl]benzamide; N-[(1R)-1-(Azetidin-1-ylcarbonyl)-3-methylbutyl]-2-fluoro-4-[7-(quinolin-6-ylmethyl)imidazo[1,2-b][1,2,4]triazin-2-yl]benzamide; 3-{4-[7-(Quinolin-6-ylmethyl)imidazo[1,2-b][1,2,4]triazin-2-yl]-1H-pyrazol-1-yl}propanenitrile; 4-[7-(Quinolin-6-ylmethyl)imidazo[1,2-b][1,2,4]triazin-2-yl]-1H-pyrazol-1-ylacetonitrile; 2-{4-[7-(Quinolin-6-ylmethyl)imidazo[1,2-b][1,2,4]triazin-2-yl]-1H-pyrazol-1-yl}acetamide; Methyl 4-{4-[7-(quinolin-6-ylmethyl)imidazo[1,2-b][1,2,4]triazin-2-yl]-1H-pyrazol-1-yl}piperidine-1-carboxylate; 2-Fluoro-N-[(1S,2S)-2-hydroxycyclopentyl]-4-[7-(quinolin-6-ylmethyl)imidazo[1,2-b][1,2,4]triazin-2-yl]benzamide; 2-Fluoro-N-(2-hydroxyethyl)-4-[7-(quinolin-6-ylmethyl)imidazo[1,2-b][1,2,4]triazin-2-yl]benzamide; 2-Fluoro-N-[1-(methoxymethyl)cyclobutyl]-4-[7-(quinolin-6-ylmethyl)imidazo[1,2-b][1,2,4]triazin-2-yl]benzamide; 2-Fluoro-N-[4-(methoxymethyl)tetrahydro-2H-pyran-4-yl]-4-[7-(quinolin-6-ylmethyl)imidazo[1,2-b][1,2,4]triazin-2-yl]benzamide; N-(Cyclopropylmethyl)-2-fluoro-4-[7-(quinolin-6-ylmethyl)imidazo[1,2-b][1,2,4]triazin-2-yl]benzamide; 2-Fluoro-4-[7-(quinolin-6-ylmethyl)imidazo[1,2-b][1,2,4]triazin-2-yl]-N-(tetrahydro-2H-pyran-4-ylmethyl)benzamide; N-[2-(Dimethylamino)ethyl]-2-fluoro-4-[7-(quinolin-6-ylmethyl)imidazo[1,2-b][1,2,4]triazin-2-yl]benzamide; 2-Fluoro-N-(2-piperidin-1-ylethyl)-4-[7-(quinolin-6-ylmethyl)imidazo[1,2-b][1,2,4]triazin-2-yl]benzamide; 2-Fluoro-N-[2-(1-methylpyrrolidin-2-yl)ethyl]-4-[7-(quinolin-6-ylmethyl)imidazo[1,2-b][1,2,4]triazin-2-yl]benzamide; 2-Fluoro-N-(pyridin-2-ylmethyl)-4-[7-(quinolin-6-ylmethyl)imidazo[1,2-b][1,2,4]triazin-2-yl]benzamide; 2-Fluoro-N-(pyridin-3-ylmethyl)-4-[7-(quinolin-6-ylmethyl)imidazo[1,2-b][1,2,4]triazin-2-yl]benzamide; 2-Fluoro-N-(pyridin-4-ylmethyl)-4-[7-(quinolin-6-ylmethyl)imidazo[1,2-b][1,2,4]triazin-2-yl]benzamide; 2-Fluoro-N-(2-pyridin-2-ylethyl)-4-[7-(quinolin-6-ylmethyl)imidazo[1,2-b][1,2,4]triazin-2-yl]benzamide; 2-Fluoro-N-(1-pyridin-3-ylethyl)-4-[7-(quinolin-6-ylmethyl)imidazo[1,2-b][1,2,4]triazin-2-yl]benzamide; 2-Fluoro-N-(1-pyridin-4-ylethyl)-4-[7-(quinolin-6-ylmethyl)imidazo[1,2-b][1,2,4]triazin-2-yl]benzamide; 2-Fluoro-N-[(1S)-1-(hydroxymethyl)-2,2-dimethylpropyl]-4-[7-(quinolin-6-ylmethyl)imidazo[1,2-b][1,2,4]triazin-2-yl]benzamide; 2-Fluoro-N-[1-(hydroxymethyl)cyclopentyl]-4-[7-(quinolin-6-ylmethyl)imidazo[1,2-b][1,2,4]triazin-2-yl]benzamide; 2-Fluoro-N-(trans-4-hydroxycyclohexyl)-4-[7-(1-quinolin-6-ylethyl)imidazo[1,2-b][1,2,4]triazin-2-yl]benzamide; 2-Fluoro-N-methyl-4-[7-(1-quinolin-6-ylethyl)imidazo[1,2-b][1,2,4]triazin-2-yl]benzamide; N-Cyclopropyl-2-fluoro-4-[7-(1-quinolin-6-ylethyl)imidazo[1,2-b][1,2,4]triazin-2-yl]benzamide; 2-Fluoro-N-[1-(methoxymethyl)cyclopropyl]-4-[7-(1-quinolin-6-ylethyl)imidazo[1,2-b][1,2,4]triazin-2-yl]benzamide; N-(3-[2-(4-Bromo-3-fluorophenyl)imidazo[1,2-b][1,2,4]triazin-7-yl]methylphenyl)-N′-ethylurea; 2-(2,3-Dichlorophenyl)-7-(1-quinolin-6-ylcyclopropyl)imidazo[1,2-b][1,2,4]triazin-3-amine; 2-Fluoro-N-[(1-hydroxycyclopropyl)methyl]-4-[7-(quinolin-6-ylmethyl)imidazo[1,2-b][1,2,4]triazin-2-yl]benzamide; Methyl 4-(cyanomethyl)-4-{4-[7-(quinolin-6-ylmethyl)imidazo[1,2-b][1,2,4]triazin-2-yl]-1H-pyrazol-1-yl}piperidine-1-carboxylate; Ethyl 4-(cyanomethyl)-4-{4-[7-(quinolin-6-ylmethyl)imidazo[1,2-b][1,2,4]triazin-2-yl]-1H-pyrazol-1-yl}piperidine-1-carboxylate; (1-Acetyl-4-{4-[7-(quinolin-6-ylmethyl)imidazo[1,2-b][1,2,4]triazin-2-yl]-1H-pyrazol-1-yl}piperidin-4-yl)acetonitrile, or a pharmaceutically acceptable salt thereof.

In some embodiments, the c-MET inhibitor comprises 2-fluoro-N-methyl-4-[7-(quinolin-6-ylmethyl)imidazo[1,2-b][1,2,4]triazin-2-yl]benzamide dihydrochloric acid salt, or a hydrate or solvate thereof.

In some embodiments, the c-MET inhibitor comprises a compound of formula:

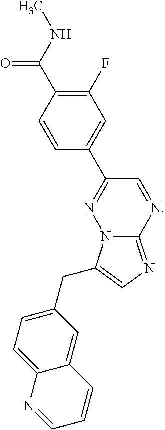 [SMILES: CNC(=O)c1ccc(-c2cnc3ncc(Cc4ccc5ncccc5c4)n3n2)cc1F]

In some embodiments, the combination comprises a PD-1 inhibitor (e.g., a PD-1 inhibitor described herein), a c-MET inhibitor (e.g., a c-MET inhibitor described herein) and one or more of a MEK inhibitor (e.g., a MEK inhibitor described herein), an IL-1b inhibitor (e.g., a IL-1b inhibitor described herein) or an A2aR antagonist (e.g., an A2aR antagonist described herein).

In some embodiments, a combination comprising a PD-1 inhibitor (e.g., a PD-1 inhibitor described herein), and a c-MET inhibitor (e.g., a c-MET inhibitor described herein), results in improved tumor control in an MC38 mouse model, compared to either agent alone.

In some embodiments, the c-MET inhibitor (e.g., capmatinib (INC280)) is administered twice a day at a dose of about 100-2000 mg, about 200-2000 mg, about 200-1000 mg, or about 200-800 mg, e.g., about 400 mg, about 500 mg, or about 600 mg. In an embodiment, the c-MET inhibitor (e.g., capmatinib (INC280)) is administered twice a day at a dose of about 400 mg. In an embodiment, the c-MET inhibitor (e.g., capmatinib (INC280)) is administered twice a day at a dose of about 600 mg. In an embodiment, the c-MET inhibitor (e.g., capmatinib (INC280) is administered twice a day at a dose of about 200 mg, e.g., 200 mg per dose.

In some embodiments, the c-MET inhibitor (e.g., capmatinib (INC280), is administered in combination with a PD-1 inhibitor (e.g., an anti-PD-1 antibody molecule) and a LAG-3 inhibitor (e.g., an anti-LAG3 antibody molecule). In one embodiment the c-MET inhibitor (e.g., capmatinib (INC280), is administered at a dose of about 200 mg twice a day, e.g., 200 mg per dose, the PD-1 inhibitor (e.g., the anti-PD-1 antibody molecule) is administered at a dose between 300 mg and 500 mg (e.g., at a dose of 400 mg), e.g., once every 4 weeks, or at a dose between 200 mg and 400 mg (e.g., at a dose of 300 mg), e.g., once every 3 weeks, e.g., by intravenous infusion, and the LAG-3 inhibitor (e.g., the anti-LAG-3 antibody molecule) is administered at a dose of about 400 mg to about 800 mg (e.g., about 600 mg) once every 4 weeks.

In some embodiments, the combination comprises a PD-1 inhibitor, e.g., PDR001, a LAG-3 inhibitor, e.g., LAG525, and a c-MET inhibitor (e.g., a c-MET inhibitor described herein). In some embodiments, this combination is administered to a subject in a therapeutically effective amount to treat, e.g., a TNBC. Without wishing to be bound by theory, it is believed that a combination comprising a PD-1 inhibitor, e.g., PDR001, a LAG-3 inhibitor, e.g., LAG525, and a c-MET inhibitor (e.g., a c-MET inhibitor described herein) is supported by the role of c-MET in tumorigenesis.

In some embodiments, the combination comprises a PD-1 inhibitor, e.g., PDR001, a LAG-3 inhibitor, e.g., LAG525, and a c-MET inhibitor (e.g., a c-MET inhibitor described herein). In some embodiments, LAG525 is administered, e.g., infused, e.g., prior to administration of PDR001. In some embodiments, PDR001 is administered, e.g., infused, after administration of LAG525. In some embodiments, both PDR001 and LAG525 are administered, e.g., infused at the same site. In some embodiments, the c-MET inhibitor administered is on the same day as the administration, e.g., infusion, of LAG525 and PDR001. In some embodiments, when the c-MET inhibitor is administered on the same day as the administration of LAG525 and PDR001, the c-MET inhibitor is administered prior to the administration, e.g., infusion of LAG525 and PDR001.

Other Exemplary c-MET Inhibitors

In some embodiments, the c-MET inhibitor comprises JNJ-38877605. JNJ-38877605 is an orally available, small molecule inhibitor of c-Met. JNJ-38877605 selectively binds to c-MET, thereby inhibiting c-MET phosphorylation and disrupting c-Met signal transduction pathways.

In some embodiments, the c-Met inhibitor is AMG 208. AMG 208 is a selective small-molecule inhibitor of c-MET. AMG 208 inhibits the ligand-dependent and ligand-independent activation of c-MET, inhibiting its tyrosine kinase activity, which may result in cell growth inhibition in tumors that overexpress c-Met.

In some embodiments, the c-Met inhibitor comprises AMG 337. AMG 337 is an orally bioavailable inhibitor of c-Met. AMG 337 selectively binds to c-MET, thereby disrupting c-MET signal transduction pathways.

In some embodiments, the c-Met inhibitor comprises LY2801653. LY2801653 is an orally available, small molecule inhibitor of c-Met. LY2801653 selectively binds to c-MET, thereby inhibiting c-MET phosphorylation and disrupting c-Met signal transduction pathways.

In some embodiments, c-Met inhibitor comprises MSC2156119J. MSC2156119J is an orally bioavailable inhibitor of c-Met. MSC2156119J selectively binds to c-MET, which inhibits c-MET phosphorylation and disrupts c-Met-mediated signal transduction pathways.

In some embodiments, the c-MET inhibitor is capmatinib. Capmatinib is also known as INCB028060. Capmatinib is an orally bioavailable inhibitor of c-MET. Capmatinib selectively binds to c-Met, thereby inhibiting c-Met phosphorylation and disrupting c-Met signal transduction pathways.

In some embodiments, the c-MET inhibitor comprises crizotinib. Crizotinib is also known as PF-02341066. Crizotinib is an orally available aminopyridine-based inhibitor of the receptor tyrosine kinase anaplastic lymphoma kinase (ALK) and the c-Met/hepatocyte growth factor receptor (HGFR). Crizotinib, in an ATP-competitive manner, binds to and inhibits ALK kinase and ALK fusion proteins. In addition, crizotinib inhibits c-Met kinase, and disrupts the c-Met signaling pathway. Altogether, this agent inhibits tumor cell growth.

In some embodiments, the c-MET inhibitor comprises golvatinib. Golvatinib is an orally bioavailable dual kinase inhibitor of c-MET and VEGFR-2 with potential antineoplastic activity. Golvatinib binds to and inhibits the activities of both c-MET and VEGFR-2, which may inhibit tumor cell growth and survival of tumor cells that overexpress these receptor tyrosine kinases.

In some embodiments, the c-MET inhibitor is tivantinib. Tivantinib is also known as ARQ 197. Tivantinib is an orally bioavailable small molecule inhibitor of c-MET. Tivantinib binds to the c-MET protein and disrupts c-Met signal transduction pathways, which may induce cell death in tumor cells overexpressing c-MET protein or expressing constitutively activated c-Met protein.

### TGF-β Inhibitors

In certain embodiments, a combination described herein comprises a transforming growth factor beta (also known as TGF-β TGFβ, TGFb, or TGF-beta, used interchangeably herein) inhibitor.

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β1, TGF-β2, or TGF-β3).

Under normal conditions, TGF-β maintains homeostasis and limits the growth of epithelial, endothelial, neuronal and hematopoietic cell lineages, e.g., through the induction of anti-proliferative and apoptotic responses. Canonical and non-canonical signaling pathways are involved in cellular responses to TGF-β. Activation of the TGF-β/Smad canonical pathway can mediate the anti-proliferative effects of TGF-β. The non-canonical TGF-β pathway can activate additional intra-cellular pathways, e.g., mitogen-activated protein kinases (MAPK), phosphatidylinositol 3 kinase/Protein Kinase B, Rho-like GTPases (Tian et al. Cell Signal. 2011; 23(6):951-62; Blobe et al. N Engl J Med. 2000; 342(18):1350-8), thus modulating epithelial to mesenchymal transition (EMT) and/or cell motility.

Alterations of TGF-β signaling pathway are associated with human diseases, e.g., cancers, cardio-vascular diseases, fibrosis, reproductive disorders, and wound healing. Without wishing to be bound by theory, it is believed that in some embodiments, the role of TGF-β in cancer is dependent on the disease setting (e.g., tumor stage and genetic alteration) and/or cellular context. For example, in late stages of cancer, TGF-β can modulate a cancer-related process, e.g., by promoting tumor growth (e.g., inducing EMT), blocking anti-tumor immune responses, increasing tumor-associated fibrosis, or enhancing angiogenesis (Wakefield and Hill Nat Rev Cancer. 2013; 13(5):328-41). In certain embodiments, a combination comprising a TGF-β inhibitor described herein is used to treat a cancer in a late stage, a metastatic cancer, or an advanced cancer.

Preclinical evidence indicates that TGF-β plays an important role in immune regulation (Wojtowicz-Praga Invest New Drugs. 2003; 21(1):21-32; Yang et al. Trends Immunol. 2010; 31(6):220-7). TGF-β can down-regulate the host-immune response via several mechanisms, e.g., shift of the T-helper balance toward Th2 immune phenotype; inhibition of anti-tumoral Th1 type response and M1-type macrophages; suppression of cytotoxic CD8+ T lymphocytes (CTL), NK lymphocytes and dendritic cell functions, generation of CD4+CD25+ T-regulatory cells; or promotion of M2-type macrophages with pro-tumoral activity mediated by secretion of immunosuppressive cytokines (e.g., IL10 or VEGF), pro-inflammatory cytokines (e.g., IL6, TNFα, or IL1) and generation of reactive oxygen species (ROS) with genotoxic activity (Yang et al. Trends Immunol. 2010; 31(6):220-7; Truty and Urrutia Pancreatology. 2007; 7(5-6):423-35; Achyut et al Gastroenterology. 2011; 141(4):1167-78).

In some embodiments, the TGF-β inhibitor is used in combination with a PD-1 inhibitor, and one or more (e.g., two, three, four, or all) of LAG-3 inhibitor, a GITR agonist, a c-MET inhibitor, an IDO inhibitor, or an A2aR antagonist. In some embodiments, the combination is used to treat a pancreatic cancer, a colorectal cancer, a gastric cancer, or a melanoma (e.g., a refractory melanoma). In some embodiments, the TGF-β inhibitor is chosen from fresolimumab or XOMA 089.

Exemplary TGF-β Inhibitors

In some embodiments, the TGF-β inhibitor comprises XOMA 089, or a compound disclosed in International Application Publication No. WO 2012/167143, which is incorporated by reference in its entirety.

XOMA 089 is also known as XPA.42.089. XOMA 089 is a fully human monoclonal antibody that specifically binds and neutralizes TGF-beta 1 and 2 ligands.

The heavy chain variable region of XOMA 089 has the amino acid sequence of: QVQLVQSGAEVKKPGSSVKVSCKASGGTFSSYAISWVRQAPGQGLEWMGGIIPIFGTANYAQKF QGRVTITADESTSTAYMELSSLRSEDTAVYYCARGLWEVRALPSVYWGQGTLVTVSS (SEQ ID NO: 240) (disclosed as SEQ ID NO: 6 in WO 2012/167143). The light chain variable region of XOMA 089 has the amino acid sequence of: SYELTQPPSVSVAPGQTARITCGANDIGSKSVHWYQQKAGQAPVLVVSEDIIRPSGIPERISGSNSG NTATLTISRVEAGDEADYYCQVWDRDSDQYVFGTGTKVTVLG (SEQ ID NO: 241) (disclosed as SEQ ID NO: 8 in WO 2012/167143).

XOMA 089 binds with high affinity to the human TGF-β isoforms. Generally, XOMA 089 binds with high affinity to TGF-β1 and TGF-β2, and to a lesser extent to TGF-β3. In Biacore assays, the K_{D} of XOMA 089 on human TGF-β is 14.6 pM for TGF-β1, 67.3 pM for TGF-β2, and 948 pM for TGF-β3. Given the high affinity binding to all three TGF-β isoforms, in certain embodiments, XOMA 089 is expected to bind to TGF-β1, 2 and 3 at a dose of XOMA 089 as described herein. XOMA 089 cross-reacts with rodent and cynomolgus monkey TGF-β and shows functional activity in vitro and in vivo, making rodent and cynomolgus monkey relevant species for toxicology studies.

Without wishing to be bound by theory, it is believed that in some embodiments, resistance to PD-1 immunotherapy is associated with the presence of a transcriptional signature which includes, e.g., genes connected to TGF-β signaling and TGF-β-dependent processes, e.g., wound healing or angiogenesis (Hugo et al. Cell. 2016; 165(1):35-44). In some embodiments, TGF-β blockade extends the therapeutic window of a therapy that inhibits the PD-1/PD-L1 axis. TGF-β inhibitors can affect the clinical benefits of PD-1 immunotherapy, e.g., by modulating tumor microenvironment, e.g., vasculogenesis, fibrosis, or factors that affect the recruitment of effector T cells (Yang et al. Trends Immunol. 2010; 31(6):220-7; Wakefield and Hill Nat Rev Cancer. 2013; 13(5):328-41; Truty and Urrutia Pancreatology. 2007; 7(5-6):423-35).

Without wishing to be bound by theory, it is also believed that in some embodiments, a number of elements of the anti-tumor immunity cycle express both PD-1 and TGF-β receptors, and PD-1 and TGF-β receptors are likely to propagate non-redundant cellular signals. For example, in mouse models of autochthonous prostate cancer, the use of either a dominant-negative form of TGFBRII, or abrogation of TGF-β production in T cells delays tumor growth (Donkor et al. Immunity. 2011; 35(1):123-34; Diener et al. Lab Invest. 2009; 89(2):142-51). Studies in the transgenic adenocarcinoma of the mouse prostate (TRAMP) mice showed that blocking TGF-β signaling in adoptively transferred T cells increases their persistence and antitumor activity (Chou et al. J Immunol. 2012; 189(8):3936-46). The antitumor activity of the transferred T cells may decrease over time, partially due to PD-1 upregulation in tumor-infiltrating lymphocytes, supporting a combination of PD-1 and TGF-β inhibition as described herein. The use of neutralizing antibodies against either PD-1 or TGF-β can also affect Tregs, given their high expression levels of PD-1 and their responsiveness to TGF-β stimulation (Riella et al. Am J Transplant. 2012; 12(10):2575-87), supporting a combination of PD-1 and TGF-β inhibition to treat cancer, e.g., by enhancing the modulation of Tregs differentiation and function.

Without wishing to be bound by theory, it is believed that cancers can use TGF-β to escape immune surveillance to facilitate tumor growth and metastatic progression. For example, in certain advanced cancers, high levels of TGF-β are associated with tumor aggressiveness and poor prognosis, and TGF-β pathway can promote one or more of cancer cell motility, invasion, EMT, or a stem cell phenotype. Immune regulation mediated by cancer cells and leukocyte populations (e.g., through a variety of cell-expressed or secreted molecules, e.g., IL-10 or TGF-β) may limit the response to checkpoint inhibitors as monotherapy in certain patients. In certain embodiments, a combined inhibition of TGF-β with a checkpoint inhibitor (e.g., an inhibitor of PD-1 described herein) is used to treat a cancer that does not respond, or responds poorly, to a checkpoint inhibitor (e.g., anti-PD-1) monotherapy, e.g., a pancreatic cancer or a colorectal cancer (e.g., a microsatellite stable colorectal cancer (MSS-CRC)). In other embodiments, a combined inhibition of TGF-β with a checkpoint inhibitor (e.g., an inhibitor of PD-1 described herein) is used to treat a cancer that shows a high level of effector T cell infiltration, e.g., a lung cancer (e.g., a non-small cell lung cancer), a breast cancer (e.g., a triple negative breast cancer), a liver cancer (e.g., a hepatocellular carcinoma), a prostate cancer, or a renal cancer (e.g., a clear cell renal cell carcinoma). In some embodiments, the combination of a TGF-β inhibitor and an inhibitor of PD-1 results in a synergistic effect.

In one embodiment, the TGF-β inhibitor (e.g., XOMA 089) is administered at a dose between 0.1 mg/kg and 20 mg/kg, e.g., between 0.1 mg/kg and 15 mg/kg, between 0.1 mg/kg and 12 mg/kg, between 0.3 mg/kg and 6 mg/kg, between 1 mg/kg and 3 mg/kg, between 0.1 mg/kg and 1 mg/kg, between 0.1 mg/kg and 0.5 mg/kg, between 0.1 mg/kg and 0.3 mg/kg, between 0.3 mg/kg and 3 mg/kg, between 0.3 mg/kg and 1 mg/kg, between 3 mg/kg and 6 mg/kg, or between 6 mg/kg and 12 mg/kg, e.g., at a dose of about 0.1 mg/kg, 0.3 mg/kg, 0.5 mg/kg, 1 mg/kg, 3 mg/kg, 6 mg/kg, 12 mg/kg, or 15 mg/kg, e.g., once every week, once every two weeks, once every three weeks, once every four weeks, or once every six weeks.

In one embodiment, the TGF-β inhibitor (e.g., XOMA 089) is administered at a dose between 0.1 mg/kg and 15 mg/kg (e.g., between 0.3 mg/kg and 12 mg/kg or between 1 mg/kg and 6 mg, e.g., about 0.1 mg/kg, 0.3 mg/kg, 1 mg/kg, 3 mg/kg, 6 mg/kg, 12 mg/kg, or 15 mg/kg), e.g., once every three weeks. For example, the TGF-β inhibitor (e.g., XOMA 089) can be administered at a dose between 0.1 mg/kg and 1 mg/kg (e.g., between 0.1 mg/kg and 1 mg/kg, e.g., 0.3 mg/kg), e.g., once every three weeks. In one embodiment, the TGF-β inhibitor (e.g., XOMA 089) is administered intravenously.

In some embodiments, the TGF-β inhibitor is administered in combination with a PD-1 inhibitor (e.g., an anti-PD-1 antibody molecule).

In one embodiment, the TGF-β inhibitor (e.g., XOMA 089) is administered at a dose between 0.1 mg/kg and 15 mg/kg (e.g., between 0.3 mg/kg and 12 mg/kg or between 1 mg/kg and 6 mg, e.g., about 0.1 mg/kg, 0.3 mg/kg, 1 mg/kg, 3 mg/kg, 6 mg/kg, 12 mg/kg, or 15 mg/kg), e.g., once every three weeks, e.g., intravenously, and the PD-1 inhibitor (e.g., the anti-PD-1 antibody molecule) is administered at a dose between 50 mg and 500 mg (e.g., between 100 mg and 400 mg, e.g., at a dose of about 100 mg, 200 mg, 300 mg, or 400 mg), e.g., once every 3 weeks or once every 4 weeks, e.g., by intravenous infusion. In some embodiments, the PD-1 inhibitor (e.g., the anti-PD-1 antibody molecule) is administered at a dose between 100 mg and 300 mg (e.g., at a dose of about 100 mg, 200 mg, or 300 mg), e.g., once every 3 weeks, e.g., by intravenous infusion.

In some embodiments, the TGF-β inhibitor (e.g., XOMA 089) is administered at a dose of about 0.1 mg/kg or 0.3 mg/kg, e.g., once every 3 weeks, e.g., by intravenous infusion, and the PD-1 inhibitor (e.g., the anti-PD-1 antibody molecule) is administered at a dose of about 100 mg, e.g., once every 3 weeks, e.g., by intravenous infusion. In some embodiments, the TGF-β inhibitor (e.g., XOMA 089) is administered at a dose of about 0.3 mg/kg, e.g., once every 3 weeks, e.g., by intravenous infusion, and the PD-1 inhibitor (e.g., the anti-PD-1 antibody molecule) is administered at a dose of about 100 mg or 300 mg, e.g., once every 3 weeks, e.g., by intravenous infusion. In some embodiments, the TGF-β inhibitor (e.g., XOMA 089) is administered at a dose of about 1 mg/kg, 3 mg/kg, 6 mg/kg, 12 mg/kg, or 15 mg/kg, e.g., once every 3 weeks, e.g., by intravenous infusion, and the PD-1 inhibitor (e.g., the anti-PD-1 antibody molecule) is administered at a dose of about 300 mg, e.g., once every 3 weeks, e.g., by intravenous infusion.

In some embodiments, the TGF-β inhibitor (e.g., XOMA 089) is administered at a dose between 0.1 mg and 0.2 mg (e.g., about 0.1 mg/kg), e.g., once every three weeks, e.g., by intravenous infusion, and the PD-1 inhibitor (e.g., the anti-PD-1 antibody molecule) is administered at a dose between 50 mg and 200 mg (e.g., about 100 mg), e.g., once every three weeks, e.g., by intravenous infusion.

In some embodiments, the TGF-β inhibitor (e.g., XOMA 089) is administered at a dose between 0.2 mg and 0.5 mg (e.g., about 0.3 mg/kg), e.g., once every three weeks, e.g., by intravenous infusion, and the PD-1 inhibitor (e.g., the anti-PD-1 antibody molecule) is administered at a dose between 50 mg and 200 mg (e.g., about 100 mg), e.g., once every three weeks, e.g., by intravenous infusion.

In some embodiments, the TGF-β inhibitor (e.g., XOMA 089) is administered at a dose between 0.2 mg and 0.5 mg (e.g., about 0.3 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 0.5 mg and 2 mg (e.g., about 1 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 2 mg and 5 mg (e.g., about 3 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 5 mg and 10 mg (e.g., about 6 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 10 mg and 15 mg (e.g., about 12 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 10 mg and 20 mg (e.g., about 15 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered before the PD-1 inhibitor (e.g., the anti-PD-1 antibody molecule) is administered. In other embodiments, the TGF-β inhibitor (e.g., XOMA 089) is administered after the PD-1 inhibitor (e.g., the anti-PD-1 antibody molecule) is administered. In certain embodiments, the TGF-β inhibitor (e.g., XOMA 089) and the PD-1 inhibitor (e.g., the anti-PD-1 antibody molecule), are administered separately with at least a 30-minute (e.g., at least 1, 1.5, or 2 hours) break between the two administrations.

In some embodiments, the combination comprises a PD-1 inhibitor (e.g., a PD-1 inhibitor described herein), a TGF-β inhibitor (e.g., a TGF-β inhibitor described herein) and one or more of a MEK inhibitor (e.g., a MEK inhibitor described herein), an IL-1β inhibitor (e.g., a IL-1b inhibitor described herein) or an A2aR antagonist (e.g., an A2aR antagonist described herein). Without wishing to be bound by theory, it is believe that in some embodiments TGFβ facilitates immunosuppression by Treg subsets in CRC and pancreatic cancer. In some embodiments, the combination comprising a PD-1 inhibitor, a TGF-β inhibitor, and one or more of a MEK inhibitor, an IL-1b inhibitor or an A2aR antagonist is administered in a therapeutically effective amount to a subject, e.g., with CRC or pancreatic cancer.

In some embodiments, a combination comprising a PD-1 inhibitor (e.g., a PD-1 inhibitor described herein), and a TGF-β inhibitor (e.g., a TGF-β inhibitor described herein) shows improved efficacy in controlling tumor growth in a murine MC38 CRC model compared to either single agent alone. Without wishing to be bound by theory, it is believed that in some embodiments a TGF-β inhibitor in combination with a PD-1 inhibitor improves, e.g., increases, the efficacy of the PD-1 inhibitor. In some embodiments, a combination comprising a PD-1 inhibitor (e.g., a PD-1 inhibitor described herein), and a TGF-β inhibitor (e.g., a TGF-β inhibitor described herein) administered to a subject with, e.g., a CRC, can result in an improved, e.g., increased, efficacy of the PD-1 inhibitor.

Other Exemplary TGF-β Inhibitors

In some embodiments, the TGF-β inhibitor comprises fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino acid sequence of:

(SEQ ID NO: 238)
QVQLVQSGAEVKKPGSSVKVSCKASGYTFSSNVISWVRQAPGQGLEWM
GGVIPIVDIANYAQRFKGRVTITADESTSTTYMELSSLRSEDTAVYYC
ASTLGLVLDAMDYWGQGTLVTVSSASTKGPSVFPLAPCSRSTSESTAA
LGCLVKDYFPEPVTVSWNSGALTSGVHTFPAVLQSSGLYSLSSVVTVP
SSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEAL
HNHYTQKSLSLSLGK.

The light chain of fresolimumab has the amino acid sequence of:

(SEQ ID NO: 239)
ETVLTQSPGTLSLSPGERATLSCRASQSLGSSYLAWYQQKPGQAPRLL
IYGASSRAPGIPDRFSGSGSGTDFTLTISRLEPEDFAVYYCQQYADSP
ITFGQGTRLEIKRTVAAPSVFIFPPSDEQLKSGTASVVCLLNNFYPRE
AKVQWKVDNALQSGNSQESVTEQDSKDSTYSLSSTLTLSKADYEKHKV
YACEVTHQGLSSPVTKSFNRGEC.

Fresolimumab is disclosed, e.g., in International Application Publication No. WO 2006/086469, and U.S. Pat. Nos. 8,383,780 and 8,591,901, which are incorporated by reference in their entirety.

### A2aR Antagonists

In certain embodiments, a combination described herein comprises an adenosine A2a receptor (A2aR) antagonist (e.g., an inhibitor of A2aR pathway, e.g., an adenosine inhibitor, e.g., an inhibitor of A2aR or CD-73). In some embodiments, the A2aR antagonist is used in combination with a PD-1 inhibitor, and one or more (e.g., two, three, four, five, or all) of a CXCR2 inhibitor, a CSF-1/1R binding agent, LAG-3 inhibitor, a GITR agonist, a c-MET inhibitor, or an IDO inhibitor. In some embodiments, the combination is used to treat a pancreatic cancer, a colorectal cancer, a gastric cancer, or a melanoma (e.g., a refractory melanoma). In some embodiments, the A2aR antagonist is chosen from PBF509 (NIR178) (Palobiofarma/Novartis), CPI444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), or Preladenant/SCH 420814 (Merck/Schering). Without wishing to be bound by theory, it is believed that in some embodiments, inhibition of A2aR leads to upregulation of IL-1b.

Exemplary A2aR Antagonists

In some embodiments, the A2aR antagonist comprises PBF509 (NIR178) or a compound disclosed in U.S. Pat. No. 8,796,284 or in International Application Publication No. WO 2017/025918, herein incorporated by reference in their entirety. PBF509 (NIR178) is also known as NIR178.

In some embodiments, the A2aR antagonist comprises a compound of formula (I):

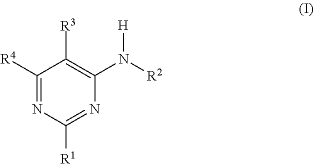

wherein

R¹ represents a five-membered heteroaryl ring selected from the group consisting of a pyrazole, a thiazole, and a triazole ring optionally substituted by one or two halogen atoms or by one or two methyl groups;

R² represents a hydrogen atom;

R³ represents bromine or chlorine atom;

R⁴ represents independently:

a) a five-membered heteroaryl group optionally substituted by one or more halogen atoms or by one or more groups selected from the group consisting of alkyl, cycloalkyl, alkoxy, alkylthio, amino, mono- or dialkylamino

b) a group —N(R⁵)(R⁶) in which R⁵ and R⁶ represent independently:

a hydrogen atom;

an alkyl or cycloalkyl group of 3 to 6 carbon atoms, linear or branched, optionally substituted by one or more halogen atoms or by one or more groups selected from the group consisting of cycloalkyl (3-8 carbon atoms), hydroxy, alkoxy, amino, mono- and dialkylamino (1-8 carbon atoms);

or R⁵ and R⁶ form together with the nitrogen atom to that they are attached a saturated heterocyclic group of 4 to 6 members in which further heteroatom may be inserted, which is optionally substituted by one or more halogen atoms or by one or more alkyl groups (1-8 carbon atoms), hydroxy, lower alkoxy, amino, mono- or dialkylamino, or

c) a group —OR⁷ or —SR⁷, where R⁷ represents independently:

an alkyl (1-8 carbon atoms) or cycloalkyl (3-8 carbon atoms) group, linear or branched, optionally substituted by one or more halogen atoms or by one or more groups selected from the group consisting of alkyl (1-8 carbon atoms), alkoxy (1-8 carbon atoms), amino, mono- or dialkylamino (1-8 carbon atoms); or

a Phenyl ring optionally substituted with one or more halogen atoms.

In certain embodiments, the A2aR antagonist comprises 5-bromo-2,6-di-(1H-pyrazol-1-yl)pyrimidin-4-amine.

In some embodiments, the A2aR antagonist (e.g., 5-bromo-2,6-di-(1H-pyrazol-1-yl)pyrimidin-4-amine) is administered at a daily dose of about 2 mg to about 2000 mg, about 2 mg to about 500 mg, about 50 mg to about 300 mg, e.g., about 50 mg to about 100 mg (e.g., about 80 mg), about 150 mg to about 200 mg (e.g., about 160 mg), or about 200 mg to about 250 mg (e.g., about 240 mg). In some embodiments, the A2aR antagonist (e.g., 5-bromo-2,6-di-(1H-pyrazol-1-yl)pyrimidin-4-amine) is administered orally once a day or twice a day, at a dose of about 1 to 30 mg/kg, e.g., about 1 to 25 mg/kg, about 1 to 20 mg/kg, or about 1 to 6 mg/kg. In one embodiment, the A2aR antagonist (e.g., 5-bromo-2,6-di-(1H-pyrazol-1-yl)pyrimidin-4-amine) is administered twice a day at a dose of about 80 mg, 160 mg, 320 mg or 640 mg, to a subject of about 50-70 kg. In one embodiment, the A2aR antagonist (e.g., 5-bromo-2,6-di-(1H-pyrazol-1-yl)pyrimidin-4-amine) is administered twice a day at a dose of about 80 mg per dose, e.g., a total dose of about 160 mg per day. In some embodiments, the A2aR antagonist (e.g., 5-bromo-2,6-di-(1H-pyrazol-1-yl)pyrimidin-4-amine) is administered orally.

In some embodiments, a combination described herein comprises a PD-1 inhibitor, e.g., PDR001, a LAG-3 inhibitor, e.g., LAG525, and an A2aR antagonist, e.g., PBF509 (NIR178). In some embodiments, this combination is administered to a subject in a therapeutically effective amount to treat, e.g., a TNBC. Without wishing to be bound by theory, it is believed that a combination comprising a PD-1 inhibitor, e.g., PDR001, a LAG-3 inhibitor, e.g., LAG525, and an A2aR antagonist, e.g., PBF509 (NIR178), can result in modulation of the tumor microenvironment, resulting in, e.g., an anti-tumor response.

In some embodiments, a combination described herein comprises a PD-1 inhibitor, e.g., PDR001, a LAG-3 inhibitor, e.g., LAG525, and an A2aR antagonist, e.g., PBF509 (NIR178) is administered according to a dosing regimen described herein. In some embodiments, the A2aR antagonist, e.g., PBF509 (NIR178) is administered on Day 1 of a cycle, e.g., a 28-day cycle. In some embodiments, the A2aR antagonist, e.g., PBF509 (NIR178) is administered twice a day at a dose of about 60-100 mg, e.g., about 80 mg, per dose, e.g., a total dose of about 120-200 mg, e.g., 160 mg, per day, e.g., orally, on day 1 of a 28-day cycle. In some embodiments, the A2aR antagonist, e.g., PBF509 (NIR178) is administered twice a day at a dose of about 60-100 mg, e.g., about 80 mg, per dose, e.g., orally, the PD-1 inhibitor (e.g., the anti-PD-1 antibody molecule) is administered at a dose between 300 mg and 500 mg (e.g., at a dose of 400 mg), e.g., once every 4 weeks, or at a dose between 200 mg and 400 mg (e.g., at a dose of 300 mg), e.g., once every 3 weeks, e.g., by intravenous infusion, and the LAG-3 inhibitor (e.g., the anti-LAG-3 antibody molecule) is administered at a dose of about 400 mg to about 800 mg (e.g., about 600 mg) once every 4 weeks.

In some embodiments, a combination described herein comprises an A2aR antagonist, e.g., PBF509 (NIR178), and a GITR agonist, e.g., a GITR agonist described herein, e.g., GWN323. In some embodiments the combination comprises PBF509 (NIR178) and GWN323. In some embodiments, the combination is administered to a subject in a therapeutically effective amount to treat, e.g., a breast cancer, e.g., a triple negative breast cancer.

In some embodiments, a combination described herein comprises an A2aR antagonist, e.g., PBF509 (NIR178), and a TIM-3 inhibitor, e.g., MBG453. In some embodiments, the combination comprises PBF509 (NIR178) and MBG453. In some embodiments, the combination is administered to a subject in a therapeutically effective amount to treat, e.g., a breast cancer, e.g., a triple negative breast cancer.

In some embodiments, a combination described herein comprises an A2aR antagonist, e.g., PBF509 (NIR178), and an IL-1b inhibitor, e.g., an IL-1b inhibitor described herein. In some embodiments, the combination is administered to a subject in a therapeutically effective amount to treat, e.g., a breast cancer, e.g., a triple negative breast cancer.

In some embodiments, a combination described herein comprises an A2aR antagonist, e.g., PBF509 (NIR178), and a TGF-β inhibitor, e.g., a TGF-β inhibitor described herein, e.g., NIS793. In some embodiments, the combination comprises PBF509 (NIR178) and NIS793. In some embodiments, the combination is administered to a subject in a therapeutically effective amount to treat, e.g., a breast cancer, e.g., a triple negative breast cancer.

In some embodiments, a combination described herein comprises an A2aR antagonist, e.g., PBF509 (NIR178), and a c-MET inhibitor, e.g., a c-MET inhibitor described herein, e.g., capmatinib (INC280). In some embodiments, the combination comprises PBF509 (NIR178) and capmatinib (INC280). In some embodiments, the combination is administered to a subject in a therapeutically effective amount to treat, e.g., a breast cancer, e.g., a triple negative breast cancer.

In some embodiments, a combination described herein comprises an A2aR antagonist, e.g., PBF509 (NIR178), and a CSF-1/1R binding agent, e.g., a CSF-1/1R binding agent described herein, e.g., BLZ945 or MCS110. In some embodiments, the combination comprises PBF509 (NIR178) and BLZ945. In some embodiments, the combination comprises PBF509 (NIR178) and MCS110. In some embodiments, the combination is administered to a subject in a therapeutically effective amount to treat, e.g., a breast cancer, e.g., a triple negative breast cancer.

Other Exemplary A2aR Antagonists

In certain embodiments, the A2AR antagonist comprises CPI444/V81444. CPI-444 and other A2aR antagonists are disclosed in International Application Publication No. WO 2009/156737, herein incorporated by reference in its entirety. In certain embodiments, the A2aR antagonist is (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine In certain embodiments, the A2aR antagonist is (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof. In certain embodiments, the A2aR antagonist is 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine In certain embodiments, the A2aR antagonist is AZD4635/HTL-1071. A2aR antagonists are disclosed in International Application Publication No. WO 2011/095625, herein incorporated by reference in its entirety. In certain embodiments, the A2aR antagonist is 6-(2-chloro-6-methylpyridin-4-yl)-5-(4-fluorophenyl)-1,2,4-triazin-3-amine.

In certain embodiments, the A2aR antagonist is ST-4206 (Leadiant Biosciences). In certain embodiments, the A2aR antagonist is an A2aR antagonist described in U.S. Pat. No. 9,133,197, herein incorporated by reference in its entirety.

In certain embodiments, the A2AR antagonist is an A2aR antagonist described in U.S. Pat. Nos. 8,114,845 and 9,029,393, U.S. Application Publication Nos. 2017/0015758 and 2016/0129108, herein incorporated by reference in their entirety.

In some embodiments, the A2aR antagonist is istradefylline (CAS Registry Number: 155270-99-8). Istradefylline is also known as KW-6002 or 8-[(E)-2-(3,4-dimethoxyphenyl)vinyl]-1,3-diethyl-7-methyl-3,7-dihydro-1H-purine-2,6-dione. Istradefylline is disclosed, e.g., in LeWitt et al. (2008) Annals of Neurology 63 (3): 295-302).

In some embodiments, the A2aR antagonist is tozadenant (Biotie). Tozadenant is also known as SYN115 or 4-hydroxy-N-(4-methoxy-7-morpholin-4-yl-1,3-benzothiazol-2-yl)-4-methylpiperidine-1-carboxamide. Tozadenant blocks the effect of endogenous adenosine at the A2a receptors, resulting in the potentiation of the effect of dopamine at the D2 receptor and inhibition of the effect of glutamate at the mGluR5 receptor. In some embodiments, the A2aR antagonist is preladenant (CAS Registry Number: 377727-87-2). Preladenant is also known as SCH 420814 or 2-(2-Furanyl)-7-[2-[4-[4-(2-methoxyethoxy)phenyl]-1-piperazinyl]ethyl]7H-pyrazolo[4,3-e][1,2,4]triazolo[1,5-c]pyrimidine-5-amine Preladenant was developed as a drug that acted as a potent and selective antagonist at the adenosine A2A receptor.

In some embodiments, the A2aR antagonist is vipadenan. Vipadenan is also known as BIIB014, V2006, or 3-[(4-amino-3-methylphenyl)methyl]-7-(furan-2-yl)triazolo[4,5-d]pyrimidin-5-amine.

Other exemplary A2aR antagonists include, e.g., ATL-444, MSX-3, SCH-58261, SCH-412,348, SCH-442,416, VER-6623, VER-6947, VER-7835, CGS-15943, or ZM-241,385.

In some embodiments, the A2aR antagonist is an A2aR pathway antagonist (e.g., a CD-73 inhibitor, e.g., an anti-CD73 antibody) is MEDI9447. MEDI9447 is a monoclonal antibody specific for CD73. Targeting the extracellular production of adenosine by CD73 may reduce the immunosuppressive effects of adenosine. MEDI9447 was reported to have a range of activities, e.g., inhibition of CD73 ectonucleotidase activity, relief from AMP-mediated lymphocyte suppression, and inhibition of syngeneic tumor growth. MEDI9447 can drive changes in both myeloid and lymphoid infiltrating leukocyte populations within the tumor microenvironment. These changes include, e.g., increases in CD8 effector cells and activated macrophages, as well as a reduction in the proportions of myeloid-derived suppressor cells (MDSC) and regulatory T lymphocytes.

### IDO Inhibitors

In certain embodiments, a combination described herein comprises an inhibitor of indoleamine 2,3-dioxygenase (IDO) and/or tryptophan 2,3-dioxygenase (TDO). In some embodiments, the IDO inhibitor is used in combination with a PD-1 inhibitor, and one or more (e.g., two, three, four, or all) of a TGF-β inhibitor, an A2aR antagonist, a CSF-1/1R binding agent, a c-MET inhibitor, or a GITR agonist. In some embodiments, the combination is used to treat a pancreatic cancer, a colorectal cancer, a gastric cancer, or a melanoma (e.g., a refractory melanoma). In some embodiments, the IDO inhibitor is chosen from (4E)-4-[(3-chloro-4-fluoroanilino)-nitrosomethylidene]-1,2,5-oxadiazol-3-amine (also known as epacadostat or INCB24360), indoximod (NLG889), (1-methyl-D-tryptophan), α-cyclohexyl-5H-Imidazo[5,1-a]isoindole-5-ethanol (also known as NLG919), indoximod, BMS-986205 (formerly F001287).

Exemplary IDO Inhibitors

In some embodiments, the IDO/TDO inhibitor is indoximod (New Link Genetics). Indoximod, the D isomer of 1-methyl-tryptophan, is an orally administered small-molecule indoleamine 2,3-dioxygenase (IDO) pathway inhibitor that disrupts the mechanisms by which tumors evade immune-mediated destruction.

In some embodiments, the IDO/TDO inhibitor is NLG919 (New Link Genetics). NLG919 is a potent IDO (indoleamine-(2,3)-dioxygenase) pathway inhibitor with Ki/EC50 of 7 nM/75 nM in cell-free assays.

In some embodiments, the IDO/TDO inhibitor is epacadostat (CAS Registry Number: 1204669-58-8). Epacadostat is also known as INCB24360 or INCB024360 (Incyte). Epacadostat is a potent and selective indoleamine 2,3-dioxygenase (IDO1) inhibitor with IC50 of 10 nM, highly selective over other related enzymes such as IDO2 or tryptophan 2,3-dioxygenase (TDO).

In some embodiments, the IDO/TDO inhibitor is F001287 (Flexus/BMS). F001287 is a small molecule inhibitor of indoleamine 2,3-dioxygenase 1 (IDO1).

### STING Agonists

In certain embodiments, a combination described herein comprises a STING agonist. In some embodiments, the STING agonist is cyclic dinucleotide, e.g., a cyclic dinucleotide comprising purine or pyrimidine nucleobases (e.g., adenosine, guanine, uracil, thymine, or cytosine nucleobases). In some embodiments, the nucleobases of the cyclic dinucleotide comprise the same nucleobase or different nucleobases.

In some embodiments, the STING agonist comprises an adenosine or a guanosine nucleobase. In some embodiments, the STING agonist comprises one adenosine nucleobase and one guanosine nucleobase. In some embodiments, the STING agonist comprises two adenosine nucleobases or two guanosine nucleobases.

In some embodiments, the STING agonist comprises a modified cyclic dinucleotide, e.g., comprising a modified nucleobase, a modified ribose, or a modified phosphate linkage In some embodiments, the modified cyclic dinucleotide comprises a modified phosphate linkage, e.g., a thiophosphate.

In some embodiments, the STING agonist comprises a cyclic dinucleotide (e.g., a modified cyclic dinucleotide) with 2′,5′ or 3′,5′ phosphate linkages. In some embodiments, the STING agonist comprises a cyclic dinucleotide (e.g., a modified cyclic dinucleotide) with Rp or Sp stereochemistry around the phosphate linkages.

In some embodiments, the STING agonist is MK-1454 (Merck). MK-1454 is a cyclic dinucleotide Stimulator of Interferon Genes (STING) agonist that activates the STING pathway. Exemplary STING agonist are disclosed, e.g., in PCT Publication No. WO 2017/027645.

### Galectin Inhibitors

Galectins are a family of proteins that bind to beta galactosidase sugars. The Galectin family of proteins comprises at least of Galectin-1, Galectin-2, Galectin-3, Galectin-4, Galectin-7, and Galectin-8. Galectins are also referred to as S-type lectins, and are soluble proteins with, e.g., intracellular and extracellular functions.

Galectin-1 and Galectin-3 are highly expressed in various tumor types. Galectin-1 and Galectin-3 can promote angiogenesis and/or reprogram myeloid cells toward a pro-tumor phenotype, e.g., enhance immunosuppression from myeloid cells. Soluble Galectin-3 can also bind to and/or inactivate infiltrating T cells. In some embodiments, a cancer described herein expresses a high level of Galectin-1 or Galectin-3, or both.

Without wishing to be bound by theory, it is believed that in some embodiments, reducing (e.g., inhibiting) one or more functions of Galectin-1 or Galectin-3, or both Galectin-1 and Galectin-3, with an inhibitor (e.g., an inhibitor described herein) can reduce the growth of tumors by reducing immunosuppression, e.g., promoting or restoring an anti-tumor immune response, in the tumor microenvironment. For example, an anti-tumor immune response can be promoted or restored by increasing the numbers of infiltrating T cells, activating infiltrating T cells, and/or reprogramming myeloid cells toward an anti-tumor phenotype. In some embodiments, inhibition of Galectin-1 or Galectin-3, or both, results in an increase of immune cell infiltration, e.g., T cell infiltration, e.g., in the tumor microenvironment. In some embodiments, inhibition of Galectin-1 or Galectin-3, or both, results in increased T cell (e.g., infiltrating T cell) activation, e.g., in the tumor microenvironment, leading to, e.g., a reduction in tumor growth or elimination of a tumor. In other embodiments, inhibition of Galectin-1 or Galectin-3 or both, results in a reprogramming of myeloid cells toward an anti-tumor phenotype. In certain embodiments, inhibition of Galectin-1 or Galectin-3, or both, reduces tumor growth and/or eliminate a tumor, e.g., by reversing or restoring immunosuppression.

In certain embodiments, a combination described herein comprises a Galectin, e.g., Galectin-1 or Galectin-3, inhibitor. In some embodiments, the combination comprises a Galectin-1 inhibitor and a Galectin-3 inhibitor. In some embodiments, the combination comprises a bispecific inhibitor (e.g., a bispecific antibody molecule) targeting both Galectin-1 and Galectin-3. In some embodiments, the Galectin inhibitor is used in combination with one or more therapeutic agents described herein. In some embodiments, the Galectin inhibitor is used in combination with a PD-1 inhibitor, e.g., a PD-1 inhibitor described herein (e.g., PDR001). In some embodiments, the Galectin inhibitor is used in combination with a PD-1 inhibitor, and one or more additional therapeutic agents described herein. In some embodiments, the Galectin inhibitor is chosen from an anti-Galectin antibody molecule, GR-MD-02 (Galectin Therapeutics), Galectin-3C (Mandal Med), Anginex, or OTX-008 (OncoEthix, Merck).

Exemplary Galectin Inhibitors

In some embodiments, a Galectin inhibitor is an antibody molecule. In an embodiment, an antibody molecule is a monospecific antibody molecule and binds a single epitope. E.g., a monospecific antibody molecule having a plurality of immunoglobulin variable domain sequences, each of which binds the same epitope. In an embodiment, the Galectin inhibitor is an anti-Galectin, e.g., anti-Galectin-1 or anti-Galectin-3, antibody molecule. In some embodiments, the Galectin inhibitor is an anti-Galectin-1 antibody molecule. In some embodiments, the Galectin inhibitor is an anti-Galectin-3 antibody molecule.

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain In an embodiment, a multispecific antibody molecule is a bispecific antibody molecule, a trispecific antibody molecule, or tetraspecific antibody molecule.

In an embodiment, the Galectin inhibitor is a multispecific antibody molecule. In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope. In an embodiment, the Galectin inhibitor is a bispecific antibody molecule. In an embodiment, the first epitope is located on Galectin-1, and the second epitope is located on Galectin-3.

Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, U.S. Pat. Nos. 6,239,259, 6,294,353, 6,333,396, 6,476,198, 6,511,663, 6,670,453, U.S. Pat. Nos. 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, U52003/211078A1, U52004/219643A1, U52004/220388A1, US2004/242847A1, US2005/003403A1, US2005/004352A1, US2005/069552A1, U52005/079170A1, U52005/100543A1, US2005/136049A1, U52005/136051A1, US2005/163782A1, US2005/266425A1, US2006/083747A1, U52006/120960A1, US2006/204493A1, US2006/263367A1, US2007/004909A1, U52007/087381A1, U52007/128150A1, US2007/141049A1, US2007/154901A1, US2007/274985A1, US2008/050370A1, US2008/069820A1, US2008/152645A1, U52008/171855A1, U52008/241884A1, U52008/254512A1, US2008/260738A1, US2009/130106A1, US2009/148905A1, US2009/155275A1, US2009/162359A1, US2009/162360A1, U52009/175851A1, US2009/175867A1, U52009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1. The contents of the above-referenced applications are incorporated herein by reference in their entireties.

In other embodiments, the anti-Galectin, e.g., anti-Galectin-1 or anti-Galectin-3, antibody molecule (e.g., a monospecific, bispecific, or multispecific antibody molecule) is covalently linked, e.g., fused, to another partner e.g., a protein, e.g., as a fusion molecule for example a fusion protein. In one embodiment, a bispecific antibody molecule has a first binding specificity to a first target (e.g., to Galectin-1), a second binding specificity to a second target (e.g., Galectin-3).

This invention provides an isolated nucleic acid molecule encoding the above antibody molecule, vectors and host cells thereof. The nucleic acid molecule includes but is not limited to RNA, genomic DNA and cDNA.

In some embodiments, a Galectin inhibitor is a peptide, e.g., protein, which can bind to, and inhibit Galectin, e.g., Galectin-1 or Galectin-3, function. In some embodiments, the Galectin inhibitor is a peptide which can bind to, and inhibit Galectin-3 function. In some embodiments, the Galectin inhibitor is the peptide Galectin-3C. In some embodiments, the Galectin inhibitor is a Galectin-3 inhibitor disclosed in U.S. Pat. No. 6,770,622, which is hereby incorporated by reference in its entirety.

Galectin-3C is an N-terminal truncated protein of Galectin-3, and functions, e.g., as a competitive inhibitor of Galectin-3. Galectin-3C prevents binding of endogenous Galectin-3 to e.g., laminin on the surface of, e.g., cancer cells, and other beta-galactosidase glycoconjugates in the extracellular matrix (ECM). Galectin-3C and other exemplary Galectin inhibiting peptides are disclosed in U.S. Pat. No. 6,770,622.

In some embodiments, Galectin-3C comprises the amino acid sequence of SEQ ID NO: 1000, or an amino acid substantially identical (e.g., 90, 95 or 99%) identical thereto.

(SEQ ID NO: 1000)
GAPAGPLIVPYNLPLPGGVVPRMLITILGTVKPNANRIALDFQRGNDV
AFHFNPRFNENNRRVIVCNTKLDNNWGREERQSVFPFESGKPFKIQVL
VEPDHFKVAVNDAHLLQYNHRVKKLNEISKLGISGDIDITSASYTMI.

In some embodiments, the Galectin inhibitor is a peptide which can bind to, and inhibit Galectin-1 function. In some embodiments, the Galectin inhibitor is the peptide Anginex: Anginex is an anti-angiongenic peptide that binds Galectin-1 (Salomonsson E, et al., (2011) Journal of Biological Chemistry, 286(16):13801-13804). Binding of Anginex to Galectin-1 can interfere with, e.g., the pro-angiongenic effects of Galectin-1.

In some embodiments, the Galectin, e.g., Galectin-1 or Galectin-3, inhibitor is a non-peptidic topomimetic molecule. In some embodiments, the non-peptidic topomimetic Galectin inhibitor is OTX-008 (OncoEthix). In some embodiments, the non-peptidic topomimetic is a non-peptidic topomimetic disclosed in U.S. Pat. No. 8,207,228, which is herein incorporated by reference in its entirety. OTX-008, also known as PTX-008 or Calixarene 0118, is a selective allosteric inhibitor of Galectin-1. OTX-008 has the chemical name: N[2-(dimethylamino)ethyl]-2-{[26,27,28-tris({[2-(dimethylamino)ethyl]carbamoyl}methoxy)pentacyclo[19.3.1.1,7.1,.15,]octacosa-1(25),3(28),4,6,9(27),1012,15,17,19(26),21,23-dodecaen-25-yl]oxy}acetamide.

In some embodiments, the Galectin, e.g., Galectin-1 or Galectin-3, inhibitor is a carbohydrate based compound. In some embodiments, the Galectin inhibitor is GR-MD-02 (Galectin Therapeutics). In some embodiments, GR-MD-02 is a Galectin-3 inhibitor. GR-MD-02 is a galactose-pronged polysaccharide also referred to as, e.g., a galactoarabino-rhamnogalaturonate. GR-MD-02 and other galactose-pronged polymers, e.g., galactoarabino-rhamnogalaturonates, are disclosed in U.S. Pat. No. 8,236,780 and U.S. Publication 2014/0086932, the entire contents of which are herein incorporated by reference in their entirety.

### MEK Inhibitors

In some embodiments, a combination described herein comprises a MEK inhibitor. In some embodiments, the MEK inhibitor is chosen from Trametinib, selumetinib, AS703026, BIX 02189, BIX 02188, CI-1040, PD0325901, PD98059, U0126, XL-518, G-38963, or G02443714. In some embodiments, the MEK inhibitor is Trametinib.

Exemplary MEK Inhibitors

In some embodiments, the MEK inhibitor is trametinib. Trametinib is also known as JTP-74057, TMT212, N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide, or Mekinist (CAS Number 871700-17-3). Without wishing to be bound by theory, it is believed that in some embodiments, trametinib is a reversible and highly selective allosteric inhibitor of MEK1 and MEK2. MEK proteins are critical components of the MAPK pathway which is commonly hyperactivated in tumor cells such as melanoma cells. Oncogenic mutations in both BRAF and RAS can signal through MEK1 or MEK2.

In some embodiments, the MEK inhibitor or trametinib is administered at a dose between 0.1 mg and 4 mg (e.g., between 0.5 mg and 3 mg, e.g., at a dose of 0.5 mg), e.g., once a day. In some embodiments, the MEK inhibitor or trametinib is administered at a dose of 0.5 mg, e.g., once a day. In some embodiments, the MEK inhibitor or trametinib is administered orally.

In certain embodiments, the combination includes PD-1 inhibitor (e.g., PDR001), and an inhibitor of MEK (e.g., trametinib). In some embodiments, the PD-1 inhibitor (e.g., PDR001) is administered at a dose between 300 mg and 500 mg (e.g., at a dose of 400 mg), e.g., once every four weeks, e.g., intravenously; and the MEK inhibitor (e.g., trametinib) is administered at a dose between 0.1 mg and 4 mg (e.g., between 0.5 mg and 3 mg, e.g., at a dose of 0.5 mg), e.g., once a day, e.g., orally.

Other Exemplary MEK Inhibitors

In some embodiments the MEK inhibitor comprises selumetinib which has the chemical name: (5-[(4-bromo-2-chlorophenyl)amino]-4-fluoro-N-(2-hydroxyethoxy)-1-methyl-1H-benzimidazole-6-carboxamide. Selumetinib is also known as AZD6244 or ARRY 142886, e.g., as described in PCT Publication No. WO2003077914.

In some embodiments, the MEK inhibitor comprises AS703026, BIX 02189 or BIX 02188.

In some embodiments, the MEK inhibitor comprises 2-[(2-Chloro-4-iodophenyl)amino]-N-(cyclopropylmethoxy)-3,4-difluoro-benzamide (also known as CI-1040 or PD184352), e.g., as described in PCT Publication No. WO2000035436).

In some embodiments, the MEK inhibitor comprises N-[(2R)-2,3-Dihydroxypropoxy]-3,4-difluoro-2-[(2-fluoro-4-iodophenyl)amino]-benzamide (also known as PD0325901), e.g., as described in PCT Publication No. WO2002006213).

In some embodiments, the MEK inhibitor comprises 2′-amino-3′-methoxyflavone (also known as PD98059) which is available from Biaffin GmbH & Co., KG, Germany.

In some embodiments, the MEK inhibitor comprises 2,3-bis[amino[(2-aminophenyl)thio]methylene]-butanedinitrile (also known as U0126), e.g., as described in U.S. Pat. No. 2,779,780).

In some embodiments, the MEK inhibitor comprises XL-518 (also known as GDC-0973) which has a Cas No. 1029872-29-4 and is available from ACC Corp.

In some embodiments, the MEK inhibitor comprises G-38963.

In some embodiments, the MEK inhibitor comprises G02443714 (also known as AS703206) Additional examples of MEK inhibitors are disclosed in WO 2013/019906, WO 03/077914, WO 2005/121142, WO 2007/04415, WO 2008/024725 and WO 2009/085983, the contents of which are incorporated herein by reference. Further examples of MEK inhibitors include, but are not limited to, 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

### IL-1β Inhibitors

The Interleukin-1 (IL-1) family of cytokines is a group of secreted pleotropic cytokines with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). The IL-1 family comprises, inter alia, IL-1 beta (IL-1β), and IL-1alpha (IL-1a). IL-1β is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-113, derived from the tumor microenvironment and by malignant cells, promotes tumor cell proliferation, increases invasiveness and dampens anti-tumor immune response, in part by recruiting inhibitory neutrophils (Apte et al. (2006) Cancer Metastasis Rev. p. 387-408; Miller et al. (2007) J. Immunol. p. 6933-42). Experimental data indicate that inhibition of IL-1β results in a decrease in tumor burden and metastasis (Voronov et al. (2003) Proc. Natl. Acad. Sci. U.S.A. p. 2645-50).

In one embodiment, a combination described herein includes an interleukin-1 beta (IL-1β) inhibitor. In some embodiments, the IL-1β inhibitor is chosen from canakinumab, gevokizumab, Anakinra, or Rilonacept. In some embodiments, the IL-1β inhibitor is canakinumab. In some embodiments, the IL-1β inhibitor is administered in combination with one or more compounds disclosed herein to a subject with a colorectal cancer (e.g., MSS CRC), a pancreatic cancer, a gastroesophageal cancer, or a breast cancer (e.g., a triple negative breast cancer (TNBC)).

Exemplary IL-1β Inhibitors

In some embodiments, the IL-1β inhibitor is canakinumab. Canakinumab is also known as ACZ885 or ILARIS®. Canakinumab is a human monoclonal IgG1/κ antibody that neutralizes the bioactivity of human IL-1β.

Canakinumab is disclosed, e.g., in WO 2002/16436, U.S. Pat. No. 7,446,175, and EP 1313769. The heavy chain variable region of canakinumab has the amino acid sequence of: MEFGLSWVFLVALLRGVQCQVQLVESGGGVVQPGRSLRLSCAASGFTFSVYGMNWVRQAPGK GLEWVAIIWYDGDNQYYADSVKGRFTISRDNSKNTLYLQMNGLRAEDTAVYYCARDLRTGPFD YWGQGTLVTVSS (SEQ ID NO: 2001) (disclosed as SEQ ID NO: 1 in U.S. Pat. No. 7,446,175). The light chain variable region of canakinumab has the amino acid sequence of:

(SEQ ID NO: 2002)
MLPSQLIGFLLLWVPASRGEIVLTQSPDFQSVTPKEKVTITCRASQSI
GSSLHWYQQKPDQSPKLLIKYASQSFSGVPSRFSGSGSGTDFTLTINS
LEAEDAAAYYCHQSSSLPFTFGPGTKVDIK (disclosed as SEQ
ID NO: 2 in U.S. Pat. No. 7,446,175).

Canakinumab has been used, e.g., for the treatment of Cryopyrin Associated Periodic Syndromes (CAPS), in adults and children, for the treatment of systemic juvenile idiopathic arthritis (SJIA), for the symptomatic treatment of acute gouty arthritis attacks in adults, and for other IL-1β driven inflammatory diseases. Without wishing to be bound by theory, it is believed that in some embodiments, IL-1β inhibitors, e.g., canakinumab, can increase anti-tumor immune response, e.g., by blocking one or more functions of IL-1β including, e.g., recruitment of immunosuppressive neutrophils to the tumor microenvironment, stimulation of tumor angiogenesis, and/or promotion of metastasis (Dinarello (2010) Eur. J. Immunol. p. 599-606).

In some embodiments, the combination described herein includes an IL-1β inhibitor, canakinumab, or a compound disclosed in WO 2002/16436, and an inhibitor of an immune checkpoint molecule, e.g., an inhibitor of PD-1 (e.g., an anti-PD-1 antibody molecule). IL-1 is a secreted pleotropic cytokine with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). IL-1β is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-1β, derived from the tumor microenvironment and by malignant cells, promotes tumor cell proliferation, increases invasiveness and dampens anti-tumor immune response, in part by recruiting inhibitory neutrophils (Apte et al. (2006) Cancer Metastasis Rev. p. 387-408; Miller et al. (2007) J. Immunol. p. 6933-42). Experimental data indicate that inhibition of IL-1β results in a decrease in tumor burden and metastasis (Voronov et al. (2003) Proc. Natl. Acad. Sci. U.S.A. p. 2645-50). Canakinumab can bind IL-1β and inhibit IL-1-mediated signaling. Accordingly, in certain embodiments, an IL-1β inhibitor, e.g., canakinumab, enhances, or is used to enhance, an immune-mediated anti-tumor effect of an inhibitor of PD-1 (e.g., an anti-PD-1 antibody molecule).

In some embodiments, the IL-1β inhibitor, canakinumab, or a compound disclosed in WO 2002/16436, and the inhibitor of an immune checkpoint molecule, e.g., an inhibitor of PD-1 (e.g., an anti-PD-1 antibody molecule), each is administered at a dose and/or on a time schedule, that in combination, achieves a desired anti-tumor activity.

In one embodiment, the IL-1β inhibitor, canakinumab, or a compound disclosed in WO 2002/16436, is administered at a dose between 25 mg and 1000 mg, e.g., between 50 mg and 900 mg, between 80 mg and 800 mg, between 100 mg and 700 mg, between 200 mg and 600 mg, between 250 mg and 500 mg, or between 300 mg and 400 mg, e.g., at a dose of about 100 mg, 150 mg, 200 mg, 250 mg, 260 mg, 270 mg, 280 mg, 290 mg, 300 mg, 350 mg, 400 mg, 450 mg, 500 mg, 550 mg or 600 mg, e.g., once every four weeks, once every six weeks, once every eight weeks, once every ten weeks, or once every twelve weeks. In some embodiments, the IL-1β inhibitor, canakinumab is administered subcutaneously. In one embodiment, the IL-1β inhibitor, canakinumab is administered at a dose of about 600 mg, once every eight weeks, e.g., subcutaneously.

In one embodiment, the IL-1β binding antibody is canakinumab, wherein canakinumab is administered to a patient in the range of about 100 mg to about 750 mg per treatment, alternatively 100 mg-600 mg, 100 mg to 450 mg, 100 mg to 300 mg, alternatively 150 mg-600 mg, 150 mg to 450 mg, 150 mg to 300 mg per treatment, alternatively about 200 mg to 400 mg, 200 mg to 300 mg, alternatively at least 150 mg, at least 200 mg, at least 250 mg, at least 300 mg per treatment. In one embodiment the patient with cancer receives each treatment every 2 weeks, every 3 weeks, every 4 weeks (monthly), every 6 weeks, bimonthly (every 2 months) or quarterly (every 3 months). In one embodiment the patient receives canakinumab monthly or every three weeks. In one embodiment the preferred dose range of canakinumab is 200 mg to 450 mg, further preferred 300 mg to 450 mg, further preferred 350 mg to 450 mg per treatment. In one embodiment the preferred dose range of canakinumab is 200 mg to 450 mg every 3 weeks or monthly. In one embodiment the preferred dose of canakinumab is 200 mg every 3 weeks. In one embodiment the preferred dose of canakinumab is 200 mg monthly. In one embodiment the patient with cancer receives canakinumab monthly or every three week. In one embodiment the patient with cancer receives canakinumab in the dose range of 200 mg to 450 mg monthly or every three week. In one embodiment the patient with cancer receives canakinumab at a dose of 200 mg monthly or every three weeks. When safety concerns arise, the dose can be down-titrated, preferably by increasing the dosing interval, preferably by doubling the dosing interval. For example 200 mg monthly or every 3 weeks regimen can be changed to every two months or every 6 weeks respectively. In an alternative embodiment the patient with cancer receives canakinumab at a dose of 200 mg every two month or every 6 weeks in the down-tiration phase or in the maintenance phase independent from any safety issue or throughout the treatment phase.

In one embodiment, the IL-1β inhibitor, canakinumab, or a compound disclosed in WO 2002/16436, is administered at a dose between 280 mg and 320 mg (e.g., at a dose of 300 mg), e.g., once every eight weeks. In some embodiments, the IL-1β inhibitor, canakinumab, or a compound disclosed in WO 2002/16436, is administered is administered subcutaneously, e.g., in the abdomen or thigh. In one embodiment, the IL-1β inhibitor, e.g., canakinumab, is administered at a dose between 280 mg and 320 mg (e.g., at a dose of 300 mg), e.g., once every eight weeks, e.g., by subcutaneous injection, and the PD-1 inhibitor (e.g., the anti-PD-1 antibody molecule) is administered at a dose between 300 mg and 500 mg (e.g., at a dose of 400 mg), e.g., once every 4 weeks, e.g., by intravenous infusion.

In some embodiments, the IL-1β inhibitor, e.g., canakinumab, is administered on day 1 of a cycle, e.g., a cycle of two 28-day periods. In some embodiments, the IL-1β inhibitor, e.g., canakinumab, is administered on day 1 of a cycle of two 28-day periods, e.g., on day 1 of every two 28-day cycles.

In some embodiments, the IL-1β inhibitor, e.g., canakinumab, is administered in combination with a PD-1 inhibitor (e.g., an anti-PD-1 antibody molecule) and a LAG-3 inhibitor (e.g., an anti-LAG3 antibody molecule). In one embodiment the IL-1β inhibitor, e.g., canakinumab, is administered at a dose of about 600 mg once every eight weeks, e.g., subcutaneously, the PD-1 inhibitor (e.g., the anti-PD-1 antibody molecule) is administered at a dose between 300 mg and 500 mg (e.g., at a dose of 400 mg), e.g., once every 4 weeks, or at a dose between 200 mg and 400 mg (e.g., at a dose of 300 mg), e.g., once every 3 weeks, e.g., by intravenous infusion, and the LAG-3 inhibitor (e.g., the anti-LAG-3 antibody molecule) is administered at a dose of about 400 mg to about 800 mg (e.g., about 600 mg) once every 4 weeks. In some embodiments, the combinationa comprising the IL-1β inhibitor, e.g., canakinumab, a PD-1 inhibitor (e.g., an anti-PD-1 antibody molecule) and a LAG-3 inhibitor (e.g., an anti-LAG3 antibody molecule) are administered on the same day. In some embodiments, when the combination comprising the IL-1β inhibitor, e.g., canakinumab, a PD-1 inhibitor (e.g., an anti-PD-1 antibody molecule) and a LAG-3 inhibitor (e.g., an anti-LAG3 antibody molecule) are administered on the same day, the IL-1β inhibitor, e.g., canakinumab, can be administered before or after the administration, e.g., infusions, of PD-1 inhibitor (e.g., an anti-PD-1 antibody molecule) and a LAG-3 inhibitor (e.g., an anti-LAG3 antibody molecule).

In some embodiments, the IL-1β inhibitor, canakinumab, or a compound disclosed in WO 2002/16436, is administered in combination with one or more compounds disclosed herein to a subject with a colorectal cancer (e.g., MSS CRC), a pancreatic cancer, a gastroesophageal cancer, or a breast cancer (e.g., a triple negative breast cancer (TNBC)).

In other embodiments, said IL-1β binding antibody is gevokizumab. Gevokizumab (XOMA-052) is a high-affinity, humanized monoclonal antibody of the IgG2 isotype to interleukin-1β, developed for the treatment of IL-1β driven inflammatory diseases. Gevokizumab modulates IL-1β binding to its signaling receptor. Gevokizumab is disclosed in WO2007/002261 which is hereby incorporated by reference in its entirety.

In one embodiment, the present invention comprises administering gevokizumab to a patient with cancer in the range of about 30 mg to about 450 mg per treatment, alternatively 90 mg-450 mg, 90 mg to 360 mg, 90 mg to 270 mg, 90 mg to 180 mg per treatment; alternatively 120 mg-450 mg, 120 mg to 360 mg, 120 mg to 270 mg, 120 mg to 180 mg per treatment, alternatively 150 mg-450 mg, 150 mg to 360 mg, 150 mg to 270 mg, 150 mg to 180 mg per treatment, alternatively 180 mg-450 mg, 180 mg to 360 mg, 180 mg to 270 mg per treatment; alternatively about 60 mg to about 360 mg, about 60 mg to 180 mg per treatment; alternatively at least 150 mg, at least 180 mg, at least 240 mg, at least 270 mg per treatment. In one embodiment the patient with cancer receives treatment every 2 weeks, every 3 weeks, monthly (every 4 weeks), every 6 weeks, bimonthly (every 2 months) or quarterly (every 3 months). In one embodiment the patient with cancer receives at least one, preferably one treatment per month. In one embodiment the preferred range of gevokizumab is 150 mg to 270 mg. In one embodiment the preferred range of gevokizumab is 60 mg to 180 mg, further preferred 60 mg to 90 mg. In one embodiment the preferred range of gevokizumab is 90 mg to 270 mg, further preferred 90 mg to 180 mg. In one embodiment the preferred schedule is every 3 weeks or monthly. In one embodiment the patient receives gevokizumab 60 mg to 90 mg every 3 weeks. In one embodiment the patient receives gevokizumab 60 mg to 90 mg monthly. In one embodiment the patient with cancer receives gevokizumab about 90 mg to about 360 mg, 90 mg to about 270 mg, 120 mg to 270 mg, 90 mg to 180 mg, 120 mg to 180 mg, 120 mg or 90 mg every 3 weeks. In one embodiment the patient with cancer receives gevokizumab about 90 mg to about 360 mg, 90 mg to about 270 mg, 120 mg to 270 mg, 90 mg to 180 mg, 120 mg to 180 mg, 120 mg or 90 mg monthly.

In one embodiment the patient with cancer receives gevokizumab about 120 mg every 3 weeks. In one embodiment the patient receives gevokizumab about 120 mg monthly. In one embodiment the patient with cancer receives gevokizumab about 90 mg every 3 weeks. In one embodiment the patient receives gevokizumab about 90 mg monthly. In one embodiment the patient with cancer receives gevokizumab about 180 mg every 3 weeks. In one embodiment the patient receives gevokizumab about 180 mg monthly. In one embodiment the patient with cancer receives gevokizumab about 200 mg every 3 weeks. In one embodiment the patient receives gevokizumab about 200 mg monthly.

When safety concern raises, the dose can be down-titrated, preferably by increasing the dosing interval, preferably by doubling the dosing interval. For example 120 mg monthly or every 3 weeks regimen can be changed to every two month or every 6 weeks respectively. In an alternative embodiment the patients with cancer receive gevokizumab at a dose of 120 mg every two month or every 6 weeks in the down-tiration phase or in the maintenance phase independent from any safety issue or throughout the treatment phase.

In one embodiment gevokizumab or a functional fragment thereof is administered intravenously. In one embodiment gevokizumab is administered subtutaneously.

In one embodiment gevokizumab is administered 20-120 mg, preferably 30-60 mg, 30-90 mg, 60-90 mg, preferably administered intravenously, preferably every 3 weeks. In one embodiment gevokizumab is administered 20-120 mg, preferably 30-60 mg, 30-90 mg, 60-90 mg, preferably administered intravenously, preferably every 4 weeks. In one embodiment gevokizumab is administered 30-180 mg, preferably 30-60 mg, 30-90 mg, or 60-90 mg, 90-120 mg, preferably administered subcutaneously, preferably every 3 weeks. In one embodiment gevokizumab is administered 30-180 mg, preferably 30-60 mg, 30-90 mg, or 60-90 mg, 90-120 mg, 120 mg-180 mg, preferably administered subcutaneously, preferably every 4 weeks. The dosing regimens disclosed herein are applicable in each and every gevokizumab related embodiment disclosed in this application, including but not limited to monotherpy or in combination with one or more combination partner, chemotherapeutic agent, different cancer indications, such as lung cancer, RCC, CRC, gastric cancer, melanoma, breast cancer, pancreatic cancer, used in adjuvant setting or in the first line, 2nd line or 3rd line treatment.

In one embodiment, the present invention comprises administering gevokizumab at a dose of 60 mg every 2 weeks, every 3 weeks or monthly.

In one embodiment, the present invention comprises administering gevokizumab at a dose of 90 mg every 2 weeks, every 3 weeks or monthly.

In one embodiment, the present invention comprises administering gevokizumab at a dose of 180 mg every 2 weeks, every 3 weeks (±3 days), monthly, every 6 weeks, bimonthly (every 2 months) or quarterly (every 3 months).

In one embodiment, the present invention comprises administering gevokizumab at a dose of 180 mg once per month (monthly). In one further embodiment, the present invention, while keeping the above described dosing schedules, envisages the second administration of gevokizumab at 180 mg is at most two weeks, preferably two weeks apart from the first administration.

Other Exemplary IL-1β Inhibitors

In some embodiments, the IL-1β inhibitor is Anakinra (Amgen), also known as Kineret. Anakinra is an IL-1Ra antagonist that competes with IL-1β for binding to the cell surface receptor.

In some embodiments, the IL-1β inhibitor is Rilonacept (Regeneron), also known as Arcalyst. Rilonacept is a fusion protein consisting of the ligand-binding domains of the extracellular portions of the human interleukin-1 receptor component (IL-1R1) and IL-1 receptor accessory protein (IL-1RAcP) linked to the fragment-crystallizable portion (Fc region) of human IgG1. Rilonacept is an IL-1β inhibitor which, e.g., binds and neturalizes IL-1.

In one embodiment said IL-1β binding antibody is LY-2189102, which is a humanised interleukin-1 beta (IL-1β) monoclonal antibody.

In one embodiment said IL-1β binding antibody or a functional fragment thereof is CDP-484 (Celltech), which is an antibody fragment blocking IL-1P.

In one embodiment said IL-1β binding antibody or a functional fragment thereof is IL-1 Affibody (SOBI 006, Z-FC (Swedish Orphan Biovitrum/Affibody)).

### IL-15/IL-15Ra Complexes

In certain embodiments, a combination described herein comprises an IL-15/IL-15Ra complex. In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. Without wishing to be bound by theory, it is believed that in some embodiments, IL-15 potentiates, e.g., enhances, Natural Killer cells to eliminate, e.g., kill, pancreatic cancer cells. In an embodiment, response, e.g., therapeutic response, to a combination described herein, e.g., a combination comprising an IL-15/IL15Ra complex, in, e.g., an animal model of colorectal cancer is associated with Natural Killer cell infiltration.

Exemplary IL-15/IL-15Ra Complexes

In one embodiment, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 1001 in Table 11 and the soluble form of human IL-15Ra comprises an amino acid sequence of SEQ ID NO:1002 in Table 11, as described in WO 2014/066527, incorporated by reference in its entirety. The molecules described herein can be made by vectors, host cells, and methods described in WO 2007/084342, incorporated by reference in its entirety.

TABLE 11
Amino acid and nucleotide sequences of exemplary
IL-15/IL-15Ra complexes
NIZ985

| | | |
|---|---|---|
| SEQ ID NO: | Human IL-15 | NWVNVISDLKKIEDLIQSMHIDATLYTESDVHPSCKVTA |
| 1001 | | MKCFLLELQVISLESGDASIHDTVENLIILANNSLSSNGNV |
| | | TESGCKECEELEEKNIKEFLQSFVHIVQMFINTS |
| SEQ ID NO: | Human | ITCPPPMSVEHADIWVKSYSLYSRERYICNSGFKRKAGTS |
| 1002 | Soluble IL- | SLTECVLNKATNVAHWTTPSLKCIRDPALVHQRPAPPST |
| | 15Ra | VTTAGVTPQPESLSPSGKEPAASSPSSNNTAATTAAIVPG |
| | | SQLMPSKSPSTGTTEISSHESSHGTPSQTTAKNWELTASA |
| | | SHQPPGVYPQG |

Without wishing to be bound by theory, it is believed that in microsatellite stable CRCs with low T cell infiltration, IL-15 may promote, e.g., increase, T cell priming (e.g., as described in Lou, K. J. SciBX 7(16); 10.1038/SCIBX.2014.449). In some embodiments, the combination comprises a PD-1 inhibitor (e.g., a PD-1 inhibitor described herein), an IL-15/IL15RA complex (e.g., an IL-15/IL15RA complex described herein) and one or more of a MEK inhibitor (e.g., a MEK inhibitor described herein), an IL-1b inhibitor (e.g., a IL-1b inhibitor described herein) or an A2aR antagonist (e.g., an A2aR antagonist described herein). In some embodiments, the combination promotes, e.g., increases T cell priming Without wishing to be bound by theory, it is further believed that IL-15 may induce NK cell infiltration. In some embodiments, response to a PD-1 inhibitor, an IL-15/IL-15RA complex and one or more of a MEK inhibitor, an IL-1b inhibitor, or an A2Ar antagonist can result in NK cell infiltration.

Other Exemplary IL-15/IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated by reference in its entirety. In one embodiment, the IL-15/IL-15Ra Fc fusion protein comprises the sequences as disclosed in Table 12.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated by reference in their entirety. In one embodiment, the IL-15/IL-15Ra sushi domain fusion comprises the sequences as disclosed in Table 12.

TABLE 12
Amino acid sequences of other exemplary IL-15/IL-15Ra complexes

ALT-803 (Altor)

| | | |
|---|---|---|
| SEQ ID NO: | IL-15N72D | NWVNVISDLKKIEDLIQSMHIDATLYTESDVHPSCKVTAM |
| 1003 | | KCFLLELQVISLESGDASIHDTVENLIILANDSLSSNGNVTE |
| | | SGCKECEELEEKNIKEFLQSFVHIVQMFINTS |
| SEQ ID NO: | IL-15RaSu/Fc | ITCPPPMSVEHADIWVKSYSLYSRERYICNSGFKRKAGTSS |
| 1004 | | LTECVLNKATNVAHWTTPSLKCIREPKSCDKTHTCPPCPA |
| | | PELLGGPSVFLFPPKPKDTLMISRTPEVTCVVVDVSHEDPE |
| | | VKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |

IL-15/IL-15Ra sushi domain fusion (Cytune)

| | | |
|---|---|---|
| SEQ ID | Human IL-15 | NWVNVISDLKKIEDLIQSMHIDATLYTESDVHPSCKVTAM |
| NO: 1005 | | KCFLLELQVISLESGDASIHDTVENLIILANNSLSSNGNVTE |
| | | SGCKECEELEXKNIKEFLQSFVHIVQMFINTS |
| | | Where X is E or K |
| SEQ ID | Human IL- | ITCPPPMSVEHADIWVKSYSLYSRERYICNSGFKRKAGTSS |
| NO: 1006 | 15Ra sushi | LTECVLNKATNVAHWTTPSLKCIRDPALVHQRPAPP |
| | and hinge | |
| | domains | |

### MDM2 Inhibitors

In certain embodiments, a combination described herein comprises a mouse double minute 2 homolog (MDM2) inhibitor. The human homolog of MDM2 is also known as HDM2. In some embodiments, an MDM2 inhibitor described herein is also known as a HDM2 inhibitor. In some embodiments, the MDM2 inhibitor is chosen from HDM201 or CGM097.

In an embodiment the MDM2 inhibitor comprises (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one (also known as CGM097) or a compound disclosed in PCT Publication No. WO 2011/076786 to treat a disorder, e.g., a disorder described herein). In one embodiment, a therapeutic agent disclosed herein is used in combination with CGM097.

In one embodiment, the MDM2 inhibitor (e.g., CGM097) is administered at a dose of about 400 to 700 mg, e.g., administered three times weekly, 2 weeks on and one week off. In some embodiments, the dose is about 400, 500, 600, or 700 mg; about 400-500, 500-600, or 600-700 mg, e.g., administered three times weekly.

In an embodiment, an MDM2 inhibitor comprises an inhibitor of p53 and/or a p53/Mdm2 interaction. In an embodiment, the MDM2 inhibitor comprises (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (also known as HDM201), or a compound disclosed in PCT Publication No. WO2013/111105 to treat a disorder, e.g., a disorder described herein. In one embodiment, a therapeutic agent disclosed herein is used in combination with HDM201. In some embodiments, HDM201 is administered orally. In one embodiment, oral administration comprises administration by solid form, e.g., as a capsule or tablet. In some embodiments, oral administration of HDM201 comprises a high-dose intermittent regimen, e.g., as described herein, or a low-dose extended regimen, e.g., as described herein. In some embodiments, the high-dose intermittent regimen is chosen from: (i) Regimen A (e.g., 50 mg-400 mg HDM201 administered on day 1 of a 3-week cycle); Regimen B (e.g., 50 mg-150 mg HDM201 administered on days 1 and 8 of a 4 week cycle); Regimen C (e.g., 50 mg-500 mg HDM201 administered on day 1 of a 4 week cycle). In some embodiments, the low-dose extended regimen is chosen from: Regimen D (e.g., 10 mg-30 mg HDM201 once daily for weeks 1 and 2 of a 4-week cycle); or Regimen E (e.g., 15 mg-50 mg HDM201 once daily for the first week of a 4-week cycle).

In some embodiments, the combination comprises a PD-1 inhibitor, e.g., PDR001, and an MDM2 inhibitor, e.g., HDM201 or CGM097. In some embodiments, the combination comprises PDR001 and HDM201. In some embodiments, the combination comprises PDR001 and CGM097. In some embodiments, the combination is administered to a subject in a therapeutically effective amount to treat, e.g., a breast cancer, e.g., a triple negative breast cancer.

In some embodiments, the combination comprises a PD-1 inhibitor, e.g., PDR001, and an MDM2 inhibitor, e.g., HDM201 or CGM097. In some embodiments, the combination comprises PDR001 and HDM201. In some embodiments, the combination comprises PDR001 and CGM097. In some embodiments, the combination is administered to a subject in a therapeutically effective amount to treat, e.g., a breast cancer, e.g., a triple negative breast cancer.

In some embodiments, the combination comprises a PD-L1 inhibitor, e.g., FAZ053, and an MDM2 inhibitor, e.g., HDM201 or CGM097. In some embodiments, the combination comprises FAZ053 and HDM201. In some embodiments, the combination comprises FAZ053 and CGM097.

In some embodiments, the combination is administered to a subject in a therapeutically effective amount to treat, e.g., a breast cancer, e.g., a triple negative breast cancer.

### Methods of Treating Cancer

In one aspect, the disclosure relates to treatment of a subject in vivo using a combination comprising three or more (e.g., four, five, six, or more) therapeutic agents disclosed herein, or a composition or formulation comprising a combination disclosed herein, such that growth of cancerous tumors is inhibited or reduced.

In certain embodiments, the combination comprises a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a MET inhibitor, a TGF-β inhibitor, an A2aR antagonist, an IDO inhibitor, or any combination thereof. In some embodiments, the PD-1 inhibitor, LAG-3 inhibitor, TIM-3 inhibitor, GITR agonist, SERD, CDK4/6 inhibitor, CXCR2 inhibitor, CSF-1/1R binding agent, MET inhibitor, TGF-β inhibitor, A2aR antagonist, or IDO inhibitor, a STING agonist, a Galectin inhibitor, a MEK inhibitor, an IL-1b inhibitor, an IL-15/IL15RA complex, an IL-1β inhibitor, or an MDM2 inhibitor, is administered or used in accordance with a dosage regimen disclosed herein.

In one embodiment, the combination disclosed herein is suitable for the treatment of cancer in vivo. For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a radiation therapy, surgery, or any other therapeutic agent or modality, to treat a disorder herein. For example, to achieve antigen-specific enhancement of immunity, the combination can be administered together with an antigen of interest. A combination disclosed herein can be administered in either order or simultaneously.

In another aspect, a method of treating a subject, e.g., reducing or ameliorating, a hyperproliferative condition or disorder (e.g., a cancer), e.g., solid tumor, a hematological cancer, soft tissue tumor, or a metastatic lesion, in a subject is provided. The method includes administering to the subject a combination comprising three or more (e.g., four or more) therapeutic agents disclosed herein, or a composition or formulation comprising a combination disclosed herein, e.g., in accordance with a dosage regimen disclosed herein.

As used herein, the term “cancer” is meant to include all types of cancerous growths or oncogenic processes, metastatic tissues or malignantly transformed cells, tissues, or organs, irrespective of histopathological type or stage of invasiveness. Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, gastrointestinal (e.g., colon), genitourinary tract (e.g., renal, urothelial, bladder cells), prostate, CNS (e.g., brain, neural or glial cells), skin, pancreas, and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. Metastatic lesions of the aforementioned cancers can also be treated or prevented using the methods and compositions of the invention.

In some embodiments, the cancer is chosen from a breast cancer, a pancreatic cancer, a colorectal cancer, a skin cancer, a gastric cancer, or an ER+ cancer. In some embodiments, the skin cancer is a melanoma (e.g., a refractory melanoma). In some embodiments, the ER+ cancer is an ER+ breast cancer. In some embodiments, the cancer is an Epstein Barr Virus (EBV) positive cancer.

Exemplary cancers whose growth can be inhibited using the combinations disclosed herein, include cancers typically responsive to immunotherapy. Non-limiting examples of typical cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). Additionally, refractory or recurrent malignancies can be treated using the antibody molecules described herein.

Examples of other cancers that can be treated include, but are not limited to, basal cell carcinoma, biliary tract cancer; bladder cancer; bone cancer; brain and CNS cancer; primary CNS lymphoma; neoplasm of the central nervous system (CNS); breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia (including acute myeloid leukemia, chronic myeloid leukemia, acute lymphoblastic leukemia, chronic lymphocytic leukemia, chronic or acute leukemia); liver cancer; lung cancer (e.g., small cell and non-small cell); lymphoma including Hodgkin's and non-Hodgkin's lymphoma; lymphocytic lymphoma; melanoma, e.g., cutaneous or intraocular malignant melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system, hepatocarcinoma, cancer of the anal region, carcinoma of the fallopian tubes, carcinoma of the vagina, carcinoma of the vulva, cancer of the small intestine, cancer of the endocrine system, cancer of the parathyroid gland, cancer of the adrenal gland, sarcoma of soft tissue, cancer of the urethra, cancer of the penis, solid tumors of childhood, spinal axis tumor, brain stem glioma, pituitary adenoma, Kaposi's sarcoma, epidermoid cancer, squamous cell cancer, T-cell lymphoma, environmentally induced cancers including those induced by asbestos, as well as other carcinomas and sarcomas, and combinations of said cancers.

In some embodiments, the disorder is a cancer, e.g., a cancer described herein. In certain embodiments, the cancer is a solid tumor. In some embodiments, the cancer is brain tumor, e.g., a glioblastoma, a gliosarcoma, or a recurrent brain tumor. In some embodiments, the cancer is a pancreatic cancer, e.g., an advanced pancreatic cancer. In some embodiments, the cancer is a skin cancer, e.g., a melanoma (e.g., a stage II-IV melanoma, an HLA-A2 positive melanoma, an unresectable melanoma, or a metastatic melanoma), or a Merkel cell carcinoma. In some embodiments, the cancer is a renal cancer, e.g., a renal cell carcinoma (RCC) (e.g., a metastatic renal cell carcinoma) or a treatment-naïve metastatic kidney cancer. In some embodiments, the cancer is a breast cancer, e.g., a metastatic breast carcinoma or a stage IV breast carcinoma, e.g., a triple negative breast cancer (TNBC). In some embodiments, the cancer is a virus-associated cancer. In some embodiments, the cancer is an anal canal cancer (e.g., a squamous cell carcinoma of the anal canal). In some embodiments, the cancer is a cervical cancer (e.g., a squamous cell carcinoma of the cervix). In some embodiments, the cancer is a gastric cancer (e.g., an Epstein Barr Virus (EBV) positive gastric cancer, or a gastric or gastro-esophageal junction carcinoma). In some embodiments, the cancer is a head and neck cancer (e.g., an HPV positive and negative squamous cell cancer of the head and neck (SCCHN)). In some embodiments, the cancer is a nasopharyngeal cancer (NPC). In some embodiments, the cancer is a penile cancer (e.g., a squamous cell carcinoma of the penile). In some embodiments, the cancer is a vaginal or vulvar cancer (e.g., a squamous cell carcinoma of the vagina or vulva). In some embodiments, the cancer is a colorectal cancer, e.g., a relapsed colorectal cancer, a metastatic colorectal cancer, e.g., a microsatellite unstable colorectal cancer, a microsatellite stable colorectal cancer, a mismatch repair proficient colorectal cancer, or a mismatch repair deficient colorectal cancer. In some embodiments, the cancer is a lung cancer, e.g., a non-small cell lung cancer (NSCLC). In certain embodiments, the cancer is a hematological cancer. In some embodiments, the cancer is a leukemia. In some embodiments, the cancer is a lymphoma, e.g., a Hodgkin lymphoma (HL) or a diffuse large B cell lymphoma (DLBCL) (e.g., a relapsed or refractory HL or DLBCL). In some embodiments, the cancer is a myeloma. In some embodiments, the cancer is an MSI-high (MSI-H) cancer. In some embodiments, the cancer is a metastatic cancer. In other embodiments, the cancer is an advanced cancer. In other embodiments, the cancer is a relapsed or refractory cancer.

In one embodiment, the cancer is a Merkel cell carcinoma. In other embodiments, the cancer is a melanoma. In other embodiments, the cancer is a breast cancer, e.g., a triple negative breast cancer (TNBC) or a HER2-negative breast cancer. In other embodiments, the cancer is a renal cell carcinoma (e.g., a clear cell renal cell carcinoma (CCRCC) or a non-clear cell renal cell carcinoma (nccRCC)). In other embodiments, the cancer is a thyroid cancer, e.g., an anaplastic thyroid carcinoma (ATC). In other embodiments, the cancer is a neuroendocrine tumor (NET), e.g., an atypical pulmonary carcinoid tumor or an NET in pancreas, gastrointestinal (GI) tract, or lung. In certain embodiments, the cancer is a non-small cell lung cancer (NSCLC) (e.g., a squamous NSCLC or a non-squamous NSCLC). In certain embodiments, the cancer is a fallopian tube cancer. In certain embodiments, the cancer is a microsatellite instability-high colorectal cancer (MSI-high CRC) or a microsatellite stable colorectal cancer (MSS CRC).

In other embodiments, the cancer is a hematological malignancy or cancer including but is not limited to a leukemia or a lymphoma. For example, the combination can be used to treat cancers and malignancies including, but not limited to, e.g., an acute leukemia, e.g., B-cell acute lymphoid leukemia (“BALL”), T-cell acute lymphoid leukemia (“TALL”), acute lymphoid leukemia (ALL); a chronic leukemia, e.g., chronic myelogenous leukemia (CML), chronic lymphocytic leukemia (CLL); an additional hematologic cancer or hematologic condition, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenström macroglobulinemia, and “preleukemia” which are a diverse collection of hematological conditions united by ineffective production (or dysplasia) of myeloid blood cells, and the like.

In some embodiments, the cancer is a cancer disclosed in any of Tables 13-18. In some embodiments, the combination therapy (e.g., the therapeutic agent, the type of cancer, or both) is chosen according to the results (e.g., RNA expression of the compound target(s)) shown in Example 5.

As used herein, the term “subject” is intended to include human and non-human animals. In some embodiments, the subject is a human subject, e.g., a human patient having a disorder or condition characterized by abnormal PD-1 functioning. For example, the subject has at least some PD-1 protein, including the PD-1 epitope that is bound by an anti-PD-1 antibody molecule disclosed herein, e.g., a high enough level of the protein and epitope to support antibody binding to PD-1. The term “non-human animals” includes mammals and non-mammals, such as non-human primates. In some embodiments, the subject is a human In some embodiments, the subject is a human patient in need of enhancement of an immune response. The methods and compositions described herein are suitable for treating human patients having a disorder that can be treated by modulating (e.g., augmenting or inhibiting) an immune response.

Methods and compositions disclosed herein are useful for treating metastatic lesions associated with the aforementioned cancers.

In some embodiments, the method further comprises determining whether a tumor sample is positive for one or more of PD-L1, CD8, and IFN-γ, and if the tumor sample is positive for one or more, e.g., two, or all three, of the markers, then administering to the patient a therapeutically effective amount of a combination of therapeutic agents, as described herein.

In some embodiments, the combination is used to treat a cancer that expresses one or more of the biomarkers disclosed herein. In certain embodiments, the subject or cancer is treated responsive to the determination of the presence of one or more biomarkers disclosed herein.

In other embodiments, the combination is used to treat a cancer that is characterized by microsatellite instability-high (MSI-H) or mismatch repair deficient (dMMR). The identification of MSI-H or dMMR tumor status for patients can be determined using, e.g., polymerase chain reaction (PCR) tests for MSI-H status or immunohistochemistry (IHC) tests for dMMR. Methods for identification of MSI-H or dMMR tumor status are described, e.g., in Ryan et al. Crit Rev Oncol Hematol. 2017; 116:38-57; Dietmaier and Hofstadter. Lab Invest 2001, 81:1453-1456; Kawakami et al. Curr Treat Options Oncol. 2015; 16(7): 30).

The combination therapies described herein can include a composition of the present invention co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the combination is further administered or used in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

When administered in combination, the therapeutic agent can be administered in an amount or dose that is higher or lower than, or the same as, the amount or dosage of each agent used individually, e.g., as a monotherapy. In certain embodiments, the administered amount or dosage of the therapeutic agent is lower (e.g., at least 20%, at least 30%, at least 40%, or at least 50%) than the amount or dosage of each agent used individually, e.g., as a monotherapy. In other embodiments, the amount or dosage of the therapeutic agent that results in a desired effect (e.g., treatment of cancer) is lower (e.g., at least 20%, at least 30%, at least 40%, or at least 50% lower).

### Pharmaceutical Compositions

In another aspect, the present invention provides compositions, e.g., pharmaceutically acceptable compositions, which includes one or more of, e.g., two, three, four, five, six, seven, eight, or more of, a therapeutic agent described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions of this invention may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In certain embodiments, the mode of administration is parenteral (e.g., intravenous, subcutaneous, intraperitoneal, or intramuscular). In an embodiment, the composition is administered by intravenous infusion or injection. In another embodiment, the composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In some embodiments, a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a STING agonist, a Galectin inhibitor, a MEK inhibitor, an IL-15/IL-15RA complex, an IL-1β inhibitor, or an MDM2 inhibitor, or any combination thereof, can be formulated into a formulation (e.g., a dose formulation or dosage form) suitable for administration (e.g., intravenous administration) to a subject as described herein.

In some embodiments, a PD-1 inhibitor (e.g., anti-PD-1 antibody molecule) or a composition described herein can be formulated into a formulation (e.g., a dose formulation or dosage form) suitable for administration (e.g., intravenous administration) to a subject as described herein.

In certain embodiments, the formulation is a drug substance formulation. In other embodiments, the formulation is a lyophilized formulation, e.g., lyophilized or dried from a drug substance formulation. In other embodiments, the formulation is a reconstituted formulation, e.g., reconstituted from a lyophilized formulation. In other embodiments, the formulation is a liquid formulation. In some embodiments, the formulation (e.g., drug substance formulation) comprises a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a MEK inhibitor, an IL-15/IL-15RA complex, an IL-1β inhibitor, or any combination thereof.

In some embodiments, the formulation is a drug substance formulation. In some embodiments, the formulation (e.g., drug substance formulation) comprises the PD-1 inhibitor (e.g., the anti-PD-1 antibody molecule) and a buffering agent.

In some embodiments, the formulation (e.g., drug substance formulation) comprises a PD-1 inhibitor (e.g., an anti-PD-1 antibody molecule) present at a concentration of 10 to 50 mg/mL, e.g., 15 to 50 mg/mL, 20 to 45 mg/mL, 25 to 40 mg/mL, 30 to 35 mg/mL, 25 to 35 mg/mL, or 30 to 40 mg/mL, e.g., 15 mg/mL, 20 mg/mL, 25 mg/mL, 30 mg/mL, 33.3 mg/mL, 35 mg/mL, 40 mg/mL, 45 mg/mL, or 50 mg/mL. In certain embodiments, the PD-1 inhibitor (e.g., the anti-PD-1 antibody molecule) is present at a concentration of 30 to 35 mg/mL, e.g., 33.3 mg/mL.

In some embodiments, the formulation (e.g., drug substance formulation) comprises a buffering agent comprising histidine (e.g., a histidine buffer). In certain embodiments, the buffering agent (e.g., histidine buffer) is present at a concentration of 1 mM to 20 mM, e.g., 2 mM to 15 mM, 3 mM to 10 mM, 4 mM to 9 mM, 5 mM to 8 mM, or 6 mM to 7 mM, e.g., 1 mM, 2 mM, 3 mM, 4 mM, 5 mM, 6 mM, 6.7 mM, 7 mM, 8 mM, 9 mM, 10 mM, 11 mM, 12 mM, 13 mM, 14 mM, 15 mM, 16 mM, 17 mM, 18 mM, 19 mM, or 20 mM. In some embodiments, the buffering agent (e.g., histidine buffer) is present at a concentration of 6 mM to 7 mM, e.g., 6.7 mM. In other embodiments, the buffering agent (e.g., a histidine buffer) has a pH of 4 to 7, e.g., 5 to 6, e.g., 5, 5.5, or 6. In some embodiments, the buffering agent (e.g., histidine buffer) has a pH of 5 to 6, e.g., 5.5. In certain embodiments, the buffering agent comprises histidine at a concentration of 6 mM to 7 mM (e.g., 6.7 mM) and has a pH of 5 to 6 (e.g., 5.5).

In some embodiments, the formulation (e.g., drug substance formulation) comprises a PD-1 inhibitor (e.g., an anti-PD-1 antibody molecule) present at a concentration of 30 to 35 mg/mL, e.g., 33.3 mg/mL; and a buffering agent that comprises histidine at a concentration of 6 mM to 7 mM (e.g., 6.7 mM) and has a pH of 5 to 6 (e.g., 5.5).

In some embodiments, the formulation (e.g., drug substance formulation) further comprises a carbohydrate. In certain embodiments, the carbohydrate is sucrose. In some embodiments, the carbohydrate (e.g., sucrose) is present at a concentration of 50 mM to 150 mM, e.g., 25 mM to 150 mM, 50 mM to 100 mM, 60 mM to 90 mM, 70 mM to 80 mM, or 70 mM to 75 mM, e.g., 25 mM, 50 mM, 60 mM, 70 mM, 73.3 mM, 80 mM, 90 mM, 100 mM, or 150 mM. In some embodiments, the formulation comprises a carbohydrate or sucrose present at a concentration of 70 mM to 75 mM, e.g., 73.3 mM.

In some embodiments, the formulation (e.g., drug substance formulation) comprises a PD-1 inhibitor (e.g., an anti-PD-1 antibody molecule) present at a concentration of 30 to 35 mg/mL, e.g., 33.3 mg/mL; a buffering agent that comprises histidine at a concentration of 6 mM to 7 mM (e.g., 6.7 mM) and has a pH of 5 to 6 (e.g., 5.5); and a carbohydrate or sucrose present at a concentration of 70 mM to 75 mM, e.g., 73.3 mM.

In some embodiments, the formulation is a drug substance formulation. In some embodiments, the formulation (e.g., drug substance formulation) comprises a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a STING agonist, a Galectin inhibitor, a MEK inhibitor, an IL-15/IL-15RA complex, an IL-1β inhibitor, or an IL-1β inhibitor, or any combination thereof and a buffering agent.

In some embodiments, the formulation (e.g., drug substance formulation) further comprises a surfactant. In certain embodiments, the surfactant is polysorbate 20. In some embodiments, the surfactant or polysorbate 20) is present at a concentration of 0.005% to 0.025% (w/w), e.g., 0.0075% to 0.02% or 0.01% to 0.015% (w/w), e.g., 0.005%, 0.0075%, 0.01%, 0.013%, 0.015%, or 0.02% (w/w). In some embodiments, the formulation comprises a surfactant or polysorbate 20 present at a concentration of 0.01% to 0.015%, e.g., 0.013% (w/w).

In some embodiments, the formulation (e.g., drug substance formulation) comprises a PD-1 inhibitor (e.g., an anti-PD-1 antibody molecule) present at a concentration of 30 to 35 mg/mL, e.g., 33.3 mg/mL; a buffering agent that comprises histidine at a concentration of 6 mM to 7 mM (e.g., 6.7 mM) and has a pH of 5 to 6 (e.g., 5.5); and a surfactant or polysorbate 20 present at a concentration of 0.01% to 0.015%, e.g., 0.013% (w/w).

In some embodiments, the formulation (e.g., drug substance formulation) comprises a PD-1 inhibitor (e.g., an anti-PD-1 antibody molecule) present at a concentration of 30 to 35 mg/mL, e.g., 33.3 mg/mL; a buffering agent that comprises histidine at a concentration of 6 mM to 7 mM (e.g., 6.7 mM) and has a pH of 5 to 6 (e.g., 5.5); a carbohydrate or sucrose present at a concentration of 70 mM to 75 mM, e.g., 73.3 mM; and a surfactant or polysorbate 20 present at a concentration of 0.01% to 0.015%, e.g., 0.013% (w/w).

In some embodiments, the formulation (e.g., drug substance formulation) comprises a PD-1 inhibitor (e.g., an anti-PD-1 antibody molecule) present at a concentration of 33.3 mg/mL; a buffering agent that comprises histidine at a concentration of 6.7 mM and has a pH of 5.5; sucrose present at a concentration of 73.3 mM; and polysorbate 20 present at a concentration of 0.013% (w/w).

In some embodiments, the formulation is a lyophilized formulation. In certain embodiments, the lyophilized formulation is lyophilized from a drug substance formulation described herein. For example, 2 to 5 mL, e.g., 3 to 4 mL, e.g., 3.6 mL, of the drug substance formulation described herein can be filled per container (e.g., vial) and lyophilized.

In certain embodiments, the formulation is a reconstituted formulation. For example, a reconstituted formulation can be prepared by dissolving a lyophilized formulation in a diluent such that the protein is dispersed in the reconstituted formulation. In some embodiments, the lyophilized formulation is reconstituted with 0.5 mL to 2 mL, e.g., 1 mL, of water or buffer for injection. In certain embodiments, the lyophilized formulation is reconstituted with 1 mL of water for injection, e.g., at a clinical site.

In some embodiments, the formulation (e.g., reconstituted formulation) comprises a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a MEK inhibitor, an IL-15/IL-15RA complex, an IL-1β inhibitor, or any combination thereof, and a buffering agent.

In some embodiments, the formulation (e.g., reconstituted formulation) comprises a PD-1 inhibitor (e.g., an anti-PD-1 antibody molecule) present at a concentration of 20 mg/mL to 200 mg/mL, e.g., 50 mg/mL to 150 mg/mL, 80 mg/mL to 120 mg/mL, or 90 mg/mL to 110 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL. In certain embodiments, the PD-1 inhibitor (e.g., the anti-PD-1 antibody molecule) is present at a concentration of 80 to 120 mg/mL, e.g., 100 mg/mL.

In some embodiments, the formulation (e.g., reconstituted formulation) comprises a buffering agent comprising histidine (e.g., a histidine buffer). In certain embodiments, the buffering agent (e.g., histidine buffer) is present at a concentration of 5 mM to 100 mM, e.g., 10 mM to 50 mM, 15 mM to 25 mM, e.g., 5 mM, 10 mM, 20 mM, 30 mM, 40 mM, 50 mM, 60 mM, 70 mM, 80 mM, 90 mM, or 100 mM. In some embodiments, the buffering agent (e.g., histidine buffer) is present at a concentration of 15 mM to 25 mM, e.g., 20 mM. In other embodiments, the buffering agent (e.g., a histidine buffer) has a pH of 4 to 7, e.g., 5 to 6, e.g., 5, 5.5 or 6. In some embodiments, the buffering agent (e.g., histidine buffer) has a pH of 5 to 6, e.g., 5.5. In certain embodiments, the buffering agent comprises histidine at a concentration of 15 mM to 25 mM (e.g., 20 mM) and has a pH of 5 to 6 (e.g., 5.5).

In some embodiments, the formulation (e.g., reconstituted formulation) comprises a PD-1 inhibitor (e.g., an anti-PD-1 antibody molecule) present at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5).

In some embodiments, the formulation (e.g., reconstituted formulation) further comprises a carbohydrate. In certain embodiments, the carbohydrate is sucrose. In some embodiments, the carbohydrate (e.g., sucrose) is present at a concentration of 100 mM to 500 mM, e.g., 150 mM to 400 mM, 175 mM to 300 mM, or 200 mM to 250 mM, e.g., 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, or 300 mM. In some embodiments, the formulation comprises a carbohydrate or sucrose present at a concentration of 200 mM to 250 mM, e.g., 220 mM.

In some embodiments, the formulation (e.g., reconstituted formulation) comprises a PD-1 inhibitor (e.g., an anti-PD-1 antibody molecule) present at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5); and a carbohydrate or sucrose present at a concentration of 200 mM to 250 mM, e.g., 220 mM.

In some embodiments, the formulation (e.g., reconstituted formulation) further comprises a surfactant. In certain embodiments, the surfactant is polysorbate 20. In some embodiments, the surfactant or polysorbate 20 is present at a concentration of 0.01% to 0.1% (w/w), e.g., 0.02% to 0.08%, 0.025% to 0.06% or 0.03% to 0.05% (w/w), e.g., 0.01%, 0.025%, 0.03%, 0.04%, 0.05%, 0.06%, 0.07%, 0.08%, 0.09%, or 0.1% (w/w). In some embodiments, the formulation comprises a surfactant or polysorbate 20 present at a concentration of 0.03% to 0.05%, e.g., 0.04% (w/w).

In some embodiments, the formulation (e.g., reconstituted formulation) comprises a PD-1 inhibitor (e.g., an anti-PD-1 antibody molecule) present at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5); and a surfactant or polysorbate 20 present at a concentration of 0.03% to 0.05%, e.g., 0.04% (w/w).

In some embodiments, the formulation (e.g., reconstituted formulation) comprises a PD-1 inhibitor (e.g., an anti-PD-1 antibody molecule) present at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5); a carbohydrate or sucrose present at a concentration of 200 mM to 250 mM, e.g., 220 mM; and a surfactant or polysorbate 20 present at a concentration of 0.03% to 0.05%, e.g., 0.04% (w/w).

In some embodiments, the formulation (e.g., reconstituted formulation) comprises a PD-1 inhibitor (e.g., an anti-PD-1 antibody molecule) present at a concentration of 100 mg/mL; and a buffering agent that comprises histidine at a concentration of 6.7 mM and has a pH of 5.5; sucrose present at a concentration of 220 mM; and polysorbate 20 present at a concentration of 0.04% (w/w).

In some embodiments, the formulation is reconstituted such that an extractable volume of at least 1 mL (e.g., at least 1.5 mL, 2 mL, 2.5 mL, or 3 mL) of the reconstituted formulation can be withdrawn from the container (e.g., vial) containing the reconstituted formulation. In certain embodiments, the formulation is reconstituted and/or extracted from the container (e.g., vial) at a clinical site. In certain embodiments, the formulation (e.g., reconstituted formulation) is injected to an infusion bag, e.g., within 1 hour (e.g., within 45 minutes, 30 minutes, or 15 minutes) before the infusion starts to the patient.

In certain embodiments, the formulation is a liquid formulation. In some embodiments, the liquid formulation is prepared by diluting a drug substance formulation described herein. For example, a drug substance formulation can be diluted, e.g., with 10 to 30 mg/mL (e.g., 25 mg/mL) of a solution comprising one or more excipients (e.g., concentrated excipients). In some embodiments, the solution comprises one, two, or all of histidine, sucrose, or polysorbate 20. In certain embodiments, the solution comprises the same excipient(s) as the drug substance formulation. Exemplary excipients include, but are not limited to, an amino acid (e.g., histidine), a carbohydrate (e.g., sucrose), or a surfactant (e.g., polysorbate 20). In certain embodiments, the liquid formulation is not a reconstituted lyophilized formulation. In other embodiments, the liquid formulation is a reconstituted lyophilized formulation. In some embodiments, the formulation is stored as a liquid. In other embodiments, the formulation is prepared as a liquid and then is dried, e.g., by lyophilization or spray-drying, prior to storage.

In some embodiments, the formulation (e.g., liquid formulation) comprises a PD-1 inhibitor (e.g., an anti-PD-1 antibody molecule) present at a concentration of 5 mg/mL to 50 mg/mL, e.g., 10 mg/mL to 40 mg/mL, 15 mg/mL to 35 mg/mL, or 20 mg/mL to 30 mg/mL, e.g., 5 mg/mL, 10 mg/mL, 15 mg/mL, 20 mg/mL, 25 mg/mL, 30 mg/mL, 35 mg/mL, 40 mg/mL, 45 mg/mL, or 50 mg/mL. In certain embodiments, the PD-1 inhibitor (e.g., the anti-PD-1 antibody molecule) is present at a concentration of 20 to 30 mg/mL, e.g., 25 mg/mL.

In some embodiments, the formulation (e.g., liquid formulation) comprises a buffering agent comprising histidine (e.g., a histidine buffer). In certain embodiments, the buffering agent (e.g., histidine buffer) is present at a concentration of 5 mM to 100 mM, e.g., 10 mM to 50 mM, 15 mM to 25 mM, e.g., 5 mM, 10 mM, 20 mM, 30 mM, 40 mM, 50 mM, 60 mM, 70 mM, 80 mM, 90 mM, or 100 mM. In some embodiments, the buffering agent (e.g., histidine buffer) is present at a concentration of 15 mM to 25 mM, e.g., 20 mM. In other embodiments, the buffering agent (e.g., a histidine buffer) has a pH of 4 to 7, e.g., 5 to 6, e.g., 5, 5.5 or 6. In some embodiments, the buffering agent (e.g., histidine buffer) has a pH of 5 to 6, e.g., 5.5. In certain embodiments, the buffering agent comprises histidine at a concentration of 15 mM to 25 mM (e.g., 20 mM) and has a pH of 5 to 6 (e.g., 5.5).

In some embodiments, the formulation (e.g., liquid formulation) comprises a PD-1 inhibitor (e.g., an anti-PD-1 antibody molecule) present at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5).

In some embodiments, the formulation (e.g., liquid formulation) further comprises a carbohydrate. In certain embodiments, the carbohydrate is sucrose. In some embodiments, the carbohydrate (e.g., sucrose) is present at a concentration of 100 mM to 500 mM, e.g., 150 mM to 400 mM, 175 mM to 300 mM, or 200 mM to 250 mM, e.g., 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, or 300 mM. In some embodiments, the formulation comprises a carbohydrate or sucrose present at a concentration of 200 mM to 250 mM, e.g., 220 mM.

In some embodiments, the formulation (e.g., liquid formulation) comprises a PD-1 inhibitor (e.g., an anti-PD-1 antibody molecule) present at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5); and a carbohydrate or sucrose present at a concentration of 200 mM to 250 mM, e.g., 220 mM.

In some embodiments, the formulation (e.g., liquid formulation) further comprises a surfactant. In certain embodiments, the surfactant is polysorbate 20. In some embodiments, the surfactant or polysorbate 20 is present at a concentration of 0.01% to 0.1% (w/w), e.g., 0.02% to 0.08%, 0.025% to 0.06% or 0.03% to 0.05% (w/w), e.g., 0.01%, 0.025%, 0.03%, 0.04%, 0.05%, 0.06%, 0.07%, 0.08%, 0.09%, or 0.1% (w/w). In some embodiments, the formulation comprises a surfactant or polysorbate 20 present at a concentration of 0.03% to 0.05%, e.g., 0.04% (w/w).

In some embodiments, the formulation (e.g., liquid formulation) comprises a PD-1 inhibitor (e.g., an anti-PD-1 antibody molecule) present at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5); and a surfactant or polysorbate 20 present at a concentration of 0.03% to 0.05%, e.g., 0.04% (w/w).

In some embodiments, the formulation (e.g., liquid d formulation) comprises a PD-1 inhibitor (e.g., an anti-PD-1 antibody molecule) present at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5); a carbohydrate or sucrose present at a concentration of 200 mM to 250 mM, e.g., 220 mM; and a surfactant or polysorbate 20 present at a concentration of 0.03% to 0.05%, e.g., 0.04% (w/w).

In some embodiments, the formulation (e.g., liquid formulation) comprises a PD-1 inhibitor (e.g., an anti-PD-1 antibody molecule) present at a concentration of 25 mg/mL; and a buffering agent that comprises histidine at a concentration of 6.7 mM and has a pH of 5.5; sucrose present at a concentration of 220 mM; and polysorbate 20 present at a concentration of 0.04% (w/w).

In certain embodiments, 1 mL to 10 mL (e.g., 2 mL to 8 mL, 3 mL to 7 mL, or 4 mL to 5 mL, e.g., 3 mL, 4 mL, 4.3 mL, 4.5 mL, 5 mL, or 6 mL) of the liquid formulation is filled per container (e.g., vial). In other embodiments, the liquid formulation is filled into a container (e.g., vial) such that an extractable volume of at least 2 mL (e.g., at least 3 mL, at least 4 mL, or at least 5 mL) of the liquid formulation can be withdrawn per container (e.g., vial). In certain embodiments, the liquid formulation is diluted from the drug substance formulation and/or extracted from the container (e.g., vial) at a clinical site. In certain embodiments, the formulation (e.g., liquid formulation) is injected to an infusion bag, e.g., within 1 hour (e.g., within 45 minutes, 30 minutes, or 15 minutes) before the infusion starts to the patient.

A formulation described herein can be stored in a container. The container used for any of the formulations described herein can include, e.g., a vial, and optionally, a stopper, a cap, or both. In certain embodiments, the vial is a glass vial, e.g., a 6R white glass vial. In other embodiments, the stopper is a rubber stopper, e.g., a grey rubber stopper. In other embodiments, the cap is a flip-off cap, e.g., an aluminum flip-off cap. In some embodiments, the container comprises a 6R white glass vial, a grey rubber stopper, and an aluminum flip-off cap. In some embodiments, the container (e.g., vial) is for a single-use container. In certain embodiments, 50 mg to 150 mg, e.g., 80 mg to 120 mg, 90 mg to 110 mg, 100 mg to 120 mg, 100 mg to 110 mg, 110 mg to 120 mg, or 110 mg to 130 mg, of the PD-1 inhibitor (e.g., the anti-PD-1 antibody molecule), is present in the container (e.g., vial).

Other exemplary buffering agents that can be used in the formulation described herein include, but are not limited to, an arginine buffer, a citrate buffer, or a phosphate buffer. Other exemplary carbohydrates that can be used in the formulation described herein include, but are not limited to, trehalose, mannitol, sorbitol, or a combination thereof. The formulation described herein may also contain a tonicity agent, e.g., sodium chloride, and/or a stabilizing agent, e.g., an amino acid (e.g., glycine, arginine, methionine, or a combination thereof).

The therapeutic agents, e.g., inhibitors, antagonist or binding agents, can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic agent or compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the disclosure by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic agent is 0.1-30 mg/kg, more preferably 1-25 mg/kg. Dosages and therapeutic regimens of the anti-PD-1 antibody molecule can be determined by a skilled artisan. In certain embodiments, the anti-PD-1 antibody molecule is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the anti-PD-1 antibody molecule is administered at a dose from about 10 to 20 mg/kg every other week.

As another example, non-limiting range for a therapeutically or prophylactically effective amount of an antibody molecule is 200-500 mg, more preferably 300-400 mg/kg. Dosages and therapeutic regimens of the anti-PD-1 antibody molecule can be determined by a skilled artisan. In certain embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. The dosing schedule (e.g., flat dosing schedule) can vary from e.g., once a week to once every 2, 3, 4, 5, or 6 weeks. In one embodiment the anti-PD-1 antibody molecule is administered at a dose from about 300 mg to 400 mg once every three or once every four weeks. In one embodiment, the anti-PD-1 antibody molecule is administered at a dose from about 300 mg once every three weeks. In one embodiment, the anti-PD-1 antibody molecule is administered at a dose from about 400 mg once every four weeks. In one embodiment, the anti-PD-1 antibody molecule is administered at a dose from about 300 mg once every four weeks. In one embodiment, the anti-PD-1 antibody molecule is administered at a dose from about 400 mg once every three weeks. While not wishing to be bound by theory, in some embodiments, flat or fixed dosing can be beneficial to patients, for example, to save drug supply and to reduce pharmacy errors.

In some embodiments, the clearance (CL) of the anti-PD-1 antibody molecule is from about 6 to 16 mL/h, e.g., about 7 to 15 mL/h, about 8 to 14 mL/h, about 9 to 12 mL/h, or about 10 to 11 mL/h, e.g., about 8.9 mL/h, 10.9 mL/h, or 13.2 mL/h.

In some embodiments, the exponent of weight on CL of the anti-PD-1 antibody molecule is from about 0.4 to 0.7, about 0.5 to 0.6, or 0.7 or less, e.g., 0.6 or less, or about 0.54.

In some embodiments, the volume of distribution at steady state (Vss) of the anti-PD-1 antibody molecule is from about 5 to 10 V, e.g., about 6 to 9 V, about 7 to 8 V, or about 6.5 to 7.5 V, e.g., about 7.2 V.

In some embodiments, the half-life of the anti-PD-1 antibody molecule is from about 10 to 30 days, e.g., about 15 to 25 days, about 17 to 22 days, about 19 to 24 days, or about 18 to 22 days, e.g., about 20 days.

In some embodiments, the Cmin (e.g., for a 80 kg patient) of the anti-PD-1 antibody molecule is at least about 0.4 μg/mL, e.g., at least about 3.6 μg/mL, e.g., from about 20 to 50 μg/mL, e.g., about 22 to 42 μg/mL, about 26 to 47 μg/mL, about 22 to 26 μg/mL, about 42 to 47 μg/mL, about 25 to 35 μg/mL, about 32 to 38 μg/mL, e.g., about 31 μg/mL or about 35 μg/mL. In one embodiment, the Cmin is determined in a patient receiving the anti-PD-1 antibody molecule at a dose of about 400 mg once every four weeks. In another embodiment, the Cmin is determined in a patient receiving the anti-PD-1 antibody molecule at a dose of about 300 mg once every three weeks. In some embodiments, In certain embodiments, the Cmin is at least about 50-fold higher, e.g., at least about 60-fold, 65-fold, 70-fold, 75-fold, 80-fold, 85-fold, 90-fold, 95-fold, or 100-fold, e.g., at least about 77-fold, higher than the EC50 of the anti-PD-1 antibody molecule, e.g., as determined based on IL-2 change in an SEB ex-vivo assay. In other embodiments, the Cmin is at least 5-fold higher, e.g., at least 6-fold, 7-fold, 8-fold, 9-fold, or 10-fold, e.g., at least about 8.6-fold, higher than the EC90 of the anti-PD-1 antibody molecule, e.g., as determined based on IL-2 change in an SEB ex-vivo assay.

The antibody molecule can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the modified antibody or antibody fragment is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

### Kits

A combination of therapeutic agents disclosed herein can be provided in a kit. The therapeutic agents are generally provided in a vial or a container. As appropriate, the therapeutic agents can be in liquid or dried (e.g., lyophilized) form. The kits can comprise two or more (e.g., three, four, five, or all) of the therapeutic agents of a combination disclosed herein. In some embodiments, the kit further contains a pharmaceutically acceptable diluent. The therapeutic agents can be provided in the kit in the same or separate formulations (e.g., as mixtures or in separate containers). The kits can contain aliquots of the therapeutic agents that provide for one or more doses. If aliquots for multiple administrations are provided, the doses can be uniform or varied. Varied dosing regimens can be escalating or decreasing, as appropriate. The dosages of the therapeutic agents in the combination can be independently uniform or varying. The kit can include one or more other elements including: instructions for use; other reagents, e.g., a label, or an agent useful for chelating, or otherwise coupling, a therapeutic agent to a label or therapeutic agent, or a radioprotective composition; devices or other materials for preparing the antibody for administration; pharmaceutically acceptable carriers; and devices or other materials for administration to a subject.

### EXAMPLES

### Example 1: Loss of Galectin-1 and Galectin-3 Inhibit Tumor Growth and Prevent Immune Infiltration

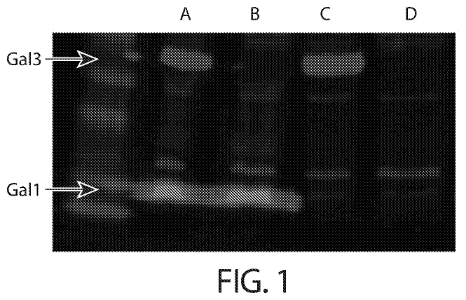

Immunocompetent mouse models were developed using the following cell lines: MC38 (A), MC38 with Galectin-3 deletion (B), MC38 with Galectin-1 deletion (C), or MC38 with Galectin-1 and Galectin-3 deletion (D). As shown in FIG. 1, MC38 cell lines depleted of Galectin-3 (B), Galectin-1 (C) or both Galectin-3 and Galectin-1 (D) do not express the corresponding protein.

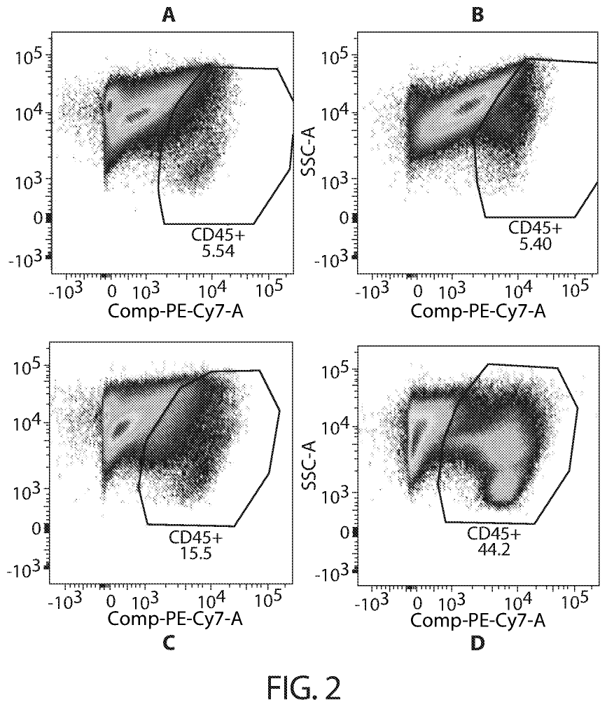

The MC38 derived tumor cell lines (A-D) were implanted subcutaneously in immune-competent mice, and the animals were monitored for tumor growth. At the endpoint of the study, tumors were removed, digested to single cells, and stained with CD45 antibodies to assess immune infiltrate. FIG. 2 shows higher CD45 immune cell infiltration in tumors generated from cell lines without Galectin-3 (B) or Galectin-1 (C) compared to wild type MC38 cells (A). Tumors from cells depleted of both Galectin-1 and Galectin-3 (D) showed the highest immune infiltration.

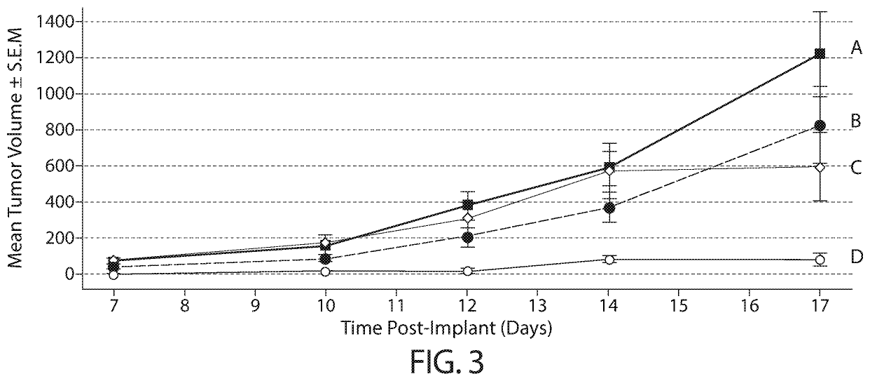

MC38 cells deleted for Galectin-3 (B) also grew more slowly after implantation into mice and demonstrated more CD45+ cell-infiltration (FIG. 2 and FIG. 3). Galectin-1 deleted MC38 cells showed a marginal decrease in tumor growth, and a corresponding increase in CD45+ cell infiltration (FIG. 2 and FIG. 3). When both Galectin-1 and Galectin-3 were deleted from MC38 tumor cells, a synergy was observed, resulting in significantly delayed tumor growth, and abundant immune infiltrate compared to wild type MC38 tumors (FIG. 2 and FIG. 3). These data provide a rationale for targeting both Galectin-1 and Galectin-3 in tumors to decrease tumor growth, and enhance immune infiltrate.

### Example 2: SEB Activation Studies for the Triple Combination of a PD-1 Inhibitor, LAG-3 Inhibitor and GITR Agonist

It has previously been established that the PD-1 inhibitor PDR001 exhibits an EC₅₀ of 0.5 μg/ml/3.68 nM in the superantigen Staphylococcal enterotoxin B (SEB) activation assay. For the triple combination studies, fixed EC50 of PDR001, and either LAG525 (LAG-3 inhibitor) or GWN323 (GITR agonist) also fixed at 0.5 ug/ml, and titrated concentrations of GWN323 or LAG525 respectively were used to assess the production of IL-2 on whole blood activated by SEB. Six parameters were tested:

### Group #1—Titrated GWN323:

Titrated hIgG1 (isotype control for GWN323) and fixed PDR001 at 0.5 ug/ml and hIgG4 (Isotype control for LAG525) at 0.5 ug/ml
Titrated hIgG1 (isotype control for GWN323) and fixed PDR001 at 0.5 ug/ml and fixed LAG525 at 0.5 ug/ml
Titrated hIgG1 (isotype control for GWN323) and fixed hIgG4 (Isotype control for PDR001) at 0.5 ug/ml and LAG525 at 0.5 ug/ml
Titrated GWN323 and fixed PDR001 at 0.5 ug/ml and hIgG4 (Isotype control for LAG525) at 0.5 ug/ml
Titrated GWN323 and fixed PDR001 at 0.5 ug/ml and LAG525 at 0.5 ug/ml
Titrated GWN323 and fixed hIgG4 (Isotype control for PDR001) at 0.5 ug/ml and LAG525 at 0.5 ug/ml
SEB at 1 ng/ml alone
No SEB

### Group #2—Titrated LAG525:

Titrated hIgG4 (Isotype control for LAG525) and fixed PDR001 at 0.5 ug/ml and hIgG1 (Isotype control for GWN323) at 0.5 ug/ml
Titrated hIgG4 (Isotype control for LAG525) and fixed PDR001 at 0.5 ug/ml and GWN323 at 0.5 ug/ml
Titrated hIgG4 (Isotype control for LAG525) and fixed hIgG4 (Isotype control for PDR001) at 0.5 ug/ml and GWN323 at 0.5 ug/ml
Titrated LAG525 and fixed PDR001 at 0.5 ug/ml and hIgG1 (Isotype control for GWN323) at 0.5 ug/ml
Titrated LAG525 and fixed PDR001 at 0.5 ug/ml and GWN323 at 0.5 ug/ml
Titrated LAG525 and fixed hIgG4 (Isotype control for PDR001) at 0.5 ug/ml and GWN323 at 0.5 ug/ml
SEB at 1 ng/ml alone
No SEB

Fresh T-cell culture media was prepared based on the IMDM media from Gibco (12440-053) with the following additional supplements: 10% Fetal Bovine Serum (Life Technologies Cat. No. 26140-079), 1% Sodium Pyruvate (Gibco, Cat. No. 11360-070), 1% L-Glutamine (Gibco, Cat. No. 25030-081), 1% HEPES (Gibco, Cat. No. 15630-080), 1% Pen-Strep (Gibco, Cat. No. 15140-122) and 1% MEM NAA (Gibco, Cat. No. 11140-050).

For the assay, PBMCs were isolated from whole blood of 3 human donors (E-411, E490, and 1876) using Leucocep (Greiner Bio-one, Cat #227-290). After a final wash, the cells were re-suspended in 10 ml of T-cell culture media. A single cell suspension was generated by straining the cells and a 1:20 dilution prepared in 1 ml T cell culture media. Cell counts were made using Vi-Cell XR (Cell Viability Analyzer). Cells were diluted to 4×10⁶ cells/ml in T-cell culture media and 50 μl cells were added to each of the inner wells of a 96-well flat bottom plate (Costar, Cat #3596). 4×30 μg/ml GWN323 (10 mg/ml Clinical Grade, MAT #887078, Batch #1010008367) or hIgG1 (1 mg/ml, Sigma, Lot # SLBR0500V) for Group #1 or LAG525 (1 mg/ml, Batch 205265.LMA) or hIgG4 (3.63 mg/ml anti-chi-lysozyme-MOR03207-hIgG4-S228P-Lys; IPROT Batch ID 104543) for Group #2 was prepared in T-cell culture media and a 1:3 dose titration was performed with 9-point dose-responses across the plate. 50 μl of titrated above antibody was added to the appropriate wells. 4×0.5 μg/ml of fixed combo of either PDR001 with LAG525 (or GWN323) or their appropriate isotype controls was prepared in T-cell media. 50 μl of media alone or the prepared combo stock was added to the appropriate groups/wells. The plates were incubated for 1 hr in a tissue culture incubator and followed by the addition of 1 ng/ml SEB. Specifically, 4×1 ng/ml of SEB was prepared in fresh T-cell culture media by first diluting a SEB stock of 1 mg/ml to 10 μg/ml (1:100), which was then used to prepare 4 ng/ml stock. 50 μl of 4×SEB was added to the appropriate wells to a final concentration of 1 ng/ml. Control groups were prepared including: no SEB (3 wells), media alone plus SEB (3 wells). All samples in the tested groups were done in duplicate. The plates were incubated at 37° C. in 5% CO₂ for 4 days. On day 4, the plates were spun at 2000 rpm for 2 min. Approximately 120 μl cell supernatants were collected into 96-well polypropylene V-bottomed plates (Greiner Bio-one, Cat #651261, Lot E150935P).

IL-2 measurement was performed using V-PLEX (MSD, Cat # K151QQD-4) according to the manufacturer's protocol. Samples were diluted to 1:5 in Diluent 2 from the kit and ran in duplicate (fixed LAG525+hIgG4 in group #1 or fixed GWN323+hIgG4 in group #2) or triplicate (all other groups). Data was analyzed using the MSD analysis software. Copied and pasted data (IL-2 in pg/ml) into Excel. Rearranged data and then transferred to GraphPad Prism6 for curves.

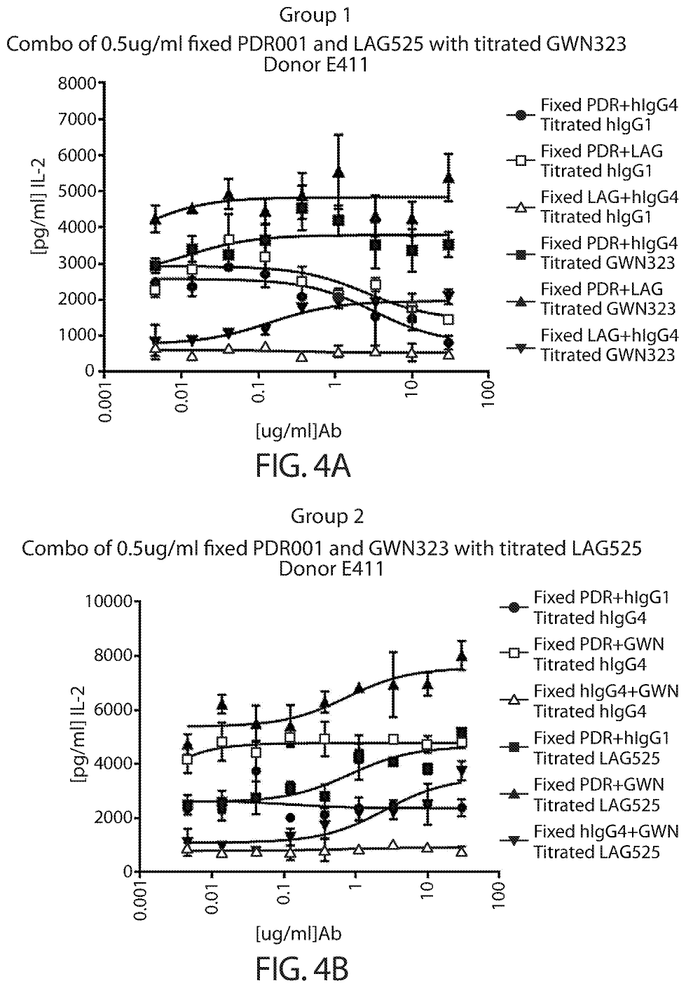

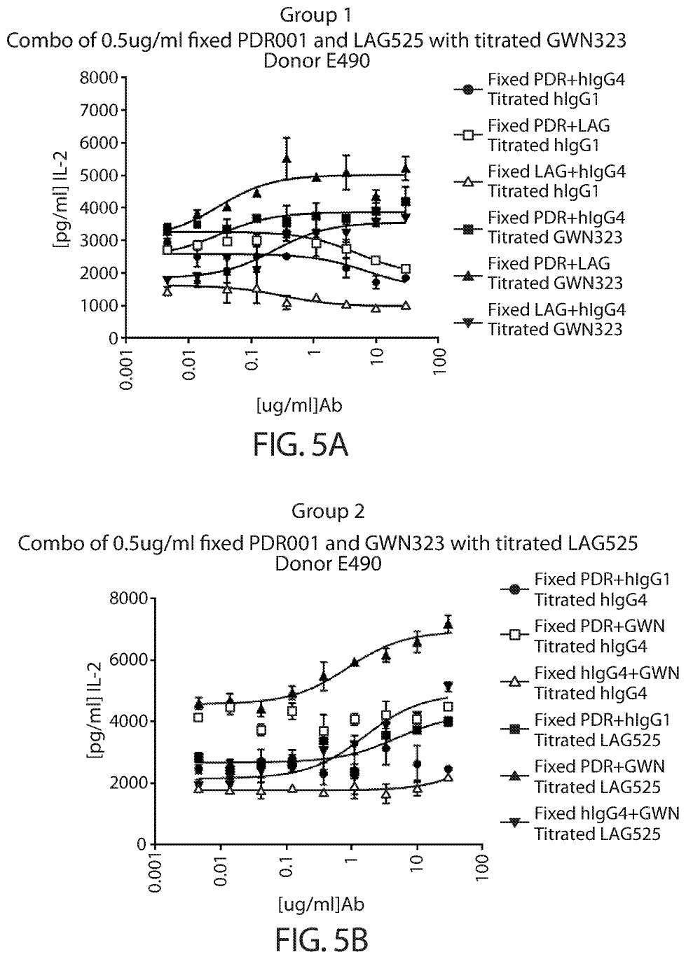

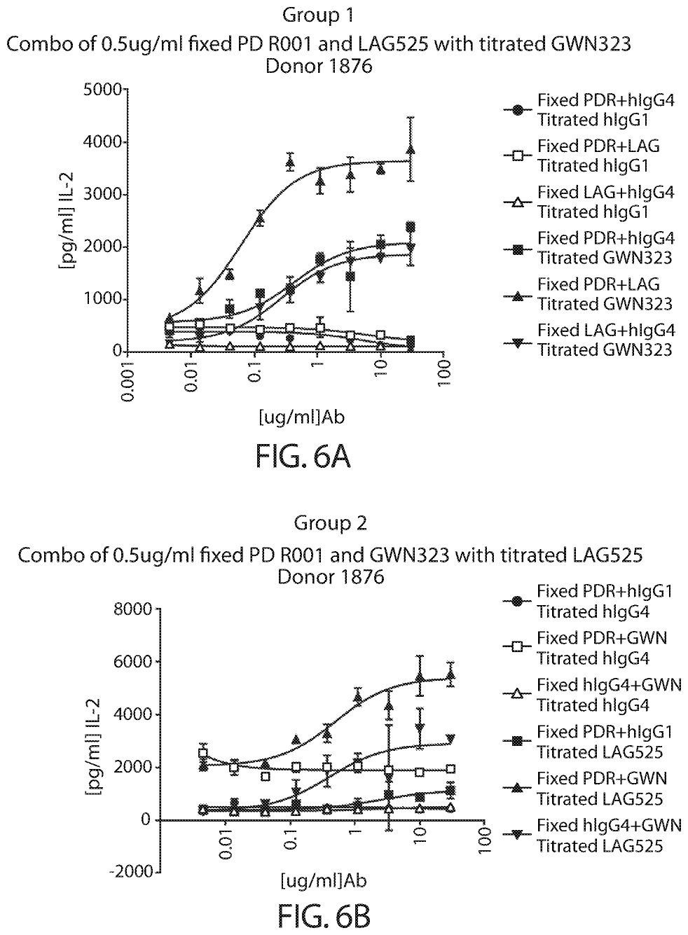

As shown in FIGS. 4A-4B, 5A-5B and 6A-6B, the triple combinations showed the largest increase in IL-2 secretion in the SEB assay. FIGS. 4B, 5B and 6B further demonstrated that titrating increasing doses of LAG525 with fixed doses of 0.5 ug/ml each of PDR001 and GWN323, results in a dose-responsive production of IL-2 in the SEB assay. This data provides a rationale for using a triple combination therapy of a PD-1 inhibitor, LAG-3 inhibitor and GITR agonist.

### Example 3: Effect of TIM-3 Inhibitor, and CSF-1R Binding Agent Combination Therapy on PD-L1 Levels in a Colon Carcinoma Mouse Model

C57BL/6 mice (Charles River Laboratories) were implanted with 1×10⁶ MC38 cells subcutaneously. When tumors were 70-100 mm3 in size, mice were randomized into groups of 8 animals and treated with an anti-TIM-3 antibody (5D12), a CSF-1R binding agent (BLZ945), both 5D12 antibody and BLZ945, or with vehicle control. Mice were dosed orally (p.o.) or intraperitoneally (i.p.). The groups of mice were dosed as follows:

Group 1) Vehicle p.o+antibody Isotype (mIgG1) i.p;
Group 2) BLZ945 200 mg/kg p.o+antibody isotype (mIgG1) i.p;
Group 3) Vehicle p.o+5D12 (mouse anti-TIM-3) 10 mg/kg i.p;
Group 4) BLZ945 200 mg/kg p.o+5D12 (mouse anti-TIM-3) 10 mg/kg i.p. BLZ945 was dosed weekly and 5D12 was dosed biweekly.

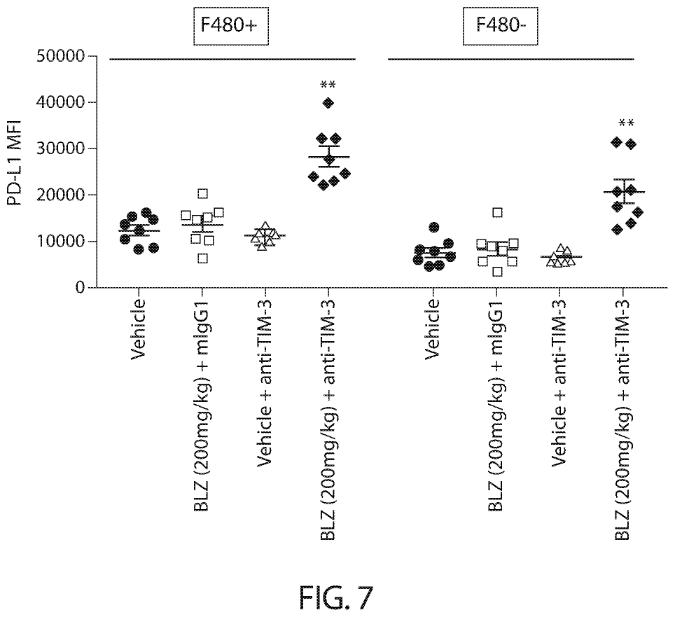

At Day 9 post-treatment initiation, mice were sacrificed. Tumors were harvested and processed into single cell suspensions for analysis by flow cytometry. The single cell suspension was generated using a combination of dispase, collagenase and DNase 1. The tumor infiltrating immune cells were analyzed by flow cytometry. Samples were acquired on a BD Fortessa and data was analyzed using Flowjo. Populations were defined as follows: dendritic cells (CD45+CD11b+Ly6C-MHC-II+Ly6G-F480−) and macrophages (CD45+CD11b+Ly6C-MHC-II+Ly6G-F480+). Levels of PD-L1 expression were analyzed as the mean fluorescence intensity of signal in the PD-L1 channel. MFI values for dendritic cells and macrophages and dendritic cells were exported from Flowjo and visualized in Graphpad prism V6. A shown in FIG. 7, an enhancement in PD-L1 expression was observed in response to combination treatment with anti-TIM-3 antibody 5D12 and BLZ945. This data provides a rationale for using a PD-1 inhibitor in combination with a TIM-3 inhibitor and a CSF-1R binding agent in colorectal cancer (CRC).

### Example 4: TIM-3 Expression on CD103+ DCs and Increased CD103+ DC Infiltration in TIM-3 Deficient Colon Carcinoma

Wild type (WT; HAVCR2+/+ BALB/c) and TIM-3 knockout (KO; HAVCR2−/− BALB/c) mice (Taconic Biosciences) were implanted with 1×10⁶ Colon26 cells subcutaneously. TIM-3 protein is encoded by the HAVCR2 gene. Day 21 post-implantations, 8 mice from each strain were euthanized. Tumors were harvested and processed into single cell suspensions for analysis by flow cytometry. The single cell suspension was generated using a combination of dispase, collagenase and DNase 1. The tumor infiltrating immune cells were analyzed by flow cytometry. Samples were acquired on a BD Fortessa and data was analyzed using Flowjo. The number of cells infiltrating the tumor was calculated by the number of events acquired on the flow cytometer normalized to tumor volume. Levels of TIM-3 expression were analyzed in myeloid subpopulations.

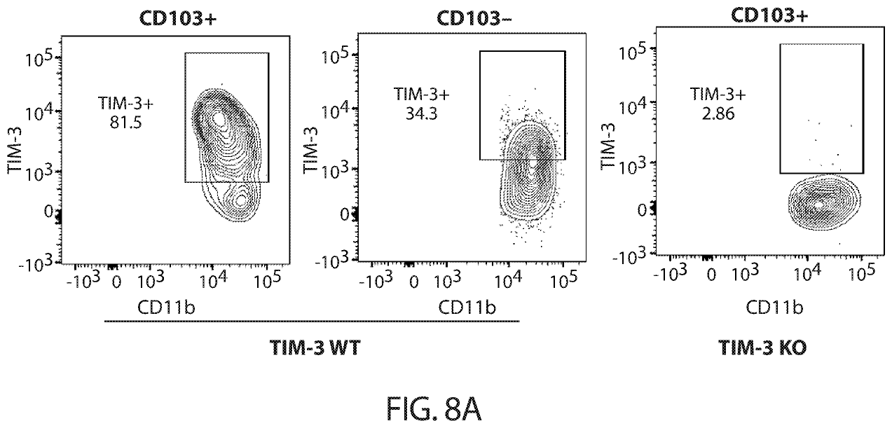

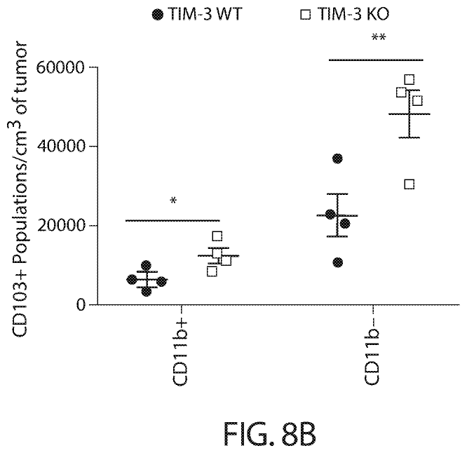

As shown in FIG. 8A, the highest frequency of TIM-3+ cells was observed on CD103+ antigen cross-presenting dendritic cells (DC) as compared to the CD103− population in TIM-3 WT mice. Cells were defined as CD45+CD11b+Ly6C-MHC-II+Ly6G-F480-CD11c+. Follow up analysis demonstrated that the prevalence of CD103+ DC was found within the CD11b-population. An increase in CD11b-CD103+ DC infiltrate was observed in tumors from TIM-3 KO mice as compared to tumors from TIM-3 WT mice (FIG. 8B). This data provides a rationale for using a combination of a STING agonist, which could increase immune infiltrate in “cold” tumors, with a PD-1 inhibitor and a TIM-3 inhibitor.

### Example 5: TCGA Analysis for the Development of TIM-3 (MBG453) Combination Therapies

### Objective

The primary objective of this analysis was to identify potential therapeutic combinations with MBG453. The aim was to use RNA expression of the compound target(s) as a basis for selection with the assumption that higher the RNA expression of the target or gene signature is correlated with sensitivity to the therapeutic targeting that protein.

### Materials and Methods

### Data and Data Processing

Transcriptomic (RNA sequence) data from patients who participated in the TCGA consortium as disclosed in The Cancer Genome Atlas Pan-Cancer analysis project Nature Genetics 45, 1113-1120 (2013), were downloaded from Omicsoft (Qiagen, CA USA). The 75th percentile value of each target of interest (TIM-3/HAVCR2, PDCD1 (also known as PD-1), LAG-3, and CD73) was calculated across all tumor samples, excluding DLBC and THYM, yielding a global pan-cancer 75th percentile expression level for each target. For PDCD1, target gene expression and gene set score (i.e. the average expression across a set of genes) were used. Genes in the gene set were IDO1, CXCL10, CXCL9, HAL-DRA, STAT1, and IFNG. Each sample was then classified as either a “high” or “low” expressor of each target. To determine which indications would potentially benefit from a MBG453 combination, the number of samples that were “high” for TIM3 and “high” for the combination drug target were calculated per indication and tabulated as a percentage of the total number of samples in that indication.

### Results

To determine potential combinations partners for targeted TIM-3 therapy, RNA expression from patients who participated in the TCGA consortium was used. Table 13 specifies the cancer type corresponding to the acronyms used to describe the data.

TABLE 13
List of indications

Abbreviation

| | |
|---|---|
| ACC | Adrenocortical carcinoma |
| BLCA | Bladder Urothelial Carcinoma |
| BRCA | Breast invasive carcinoma |
| CD73 | 5′-Nucleotidase Ecto |
| CESC | Cervical squamous cell carcinoma |
| | and endocervical adenocarcinoma |
| CHOL | Cholangiocarcinoma |
| CNTL | Controls |
| COAD | Colon adenocarcinoma |
| DLBC | Lymphoid Neoplasm Diffuse Large B-cell Lymphoma |
| ESCA | Esophageal carcinoma |
| GBM | Glioblastoma multiforme |
| HAVCR2 | Hepatitis A Virus Cellular Receptor 2 (TIM3) |
| HNSC | Head and Neck squamous cell carcinoma |
| RICH | Kidney Chromophobe |
| KIRC | Kidney renal clear cell carcinoma |
| KIRP | Kidney renal papillary cell carcinoma |
| LAG3 | Lymphocyte activating 3 |
| LAML | Acute Myeloid Leukemia |
| LCML | Chronic Myelogenous Leukemia |
| LGG | Brain Lower Grade Glioma |
| LIHC | Liver hepatocellular carcinoma |
| LUAD | Lung adenocarcinoma |
| LUSC | Lung squamous cell carcinoma |
| MESO | Mesothelioma |
| MET | MET Proto-Oncogene, Receptor Tyrosine Kinase |
| OV | Ovarian serous cystadenocarcinoma |
| PAAD | Pancreatic adenocarcinoma |
| PCPG | Pheochromocytoma and Paraganglioma |
| PDCD1 | Programmed Cell Death 1 |
| PRAD | Prostate adenocarcinoma |
| READ | Rectum adenocarcinoma |
| SARC | Sarcoma |
| SKCM | Skin Cutaneous Melanoma |
| STAD | Stomach adenocarcinoma |
| TGCT | Testicular Germ Cell Tumors |
| THCA | Thyroid carcinoma |
| THYM | Thymoma |
| UCEC | Uterine Corpus Endometrial Carcinoma |
| UCS | Uterine Carcinosarcoma |
| UVM | Uveal Melanoma |

Tables 14-16 summarize the results of three potential combination partners: Table 14: TIM3 and PDCD1; Table 15: TIM3 and LAG3; and Table 16: TIM3 and CD73. Notably, the combination of TIM-3 with PDCD1 or LAG-3 have the highest percentage of patients in across many indications where both TIM-3 and PDCD1 or LAG-3 are “high” expressers. Table 14 and 15 indicates that the indications of KIRC and MESO would benefit the most from the combination of TIM-3 with either PDCD1 or LAG-3 where more than 30% of the patient cohort had high expression of these targets. Table 14 and 15 further highlight LUAD, LUSC, SARC, TCGT, CESC, HNSC, STAD, SKCM, BLCA, and BRCA as indications that would benefit from the combination of TIM3 with PDCD1 or LAG3 in more than 10% of the patient population. In addition, 15% of ovarian cancer patients (OV) express high levels of TIM-3 and LAG-3, while approximately 11% of CHOL and KIRP patients express high levels of TIM-3 and PDCD1. Table 16 summarizes the indications that could benefit the most from combining TIM-3 with CD73 therapy, with GBM, SARC and LUAD ranking in the top three.

TABLE 14
Percent of samples with high TIM-3 and PDCD1 expression
(above global 75^{th} percentile) for double combination
therapy, across TCGA indications.

| Indication | % samples |
|---|---|
| KIRC | 40.71 |
| MESO | 32.18 |
| LUAD | 29.25 |
| LUSC | 22.36 |
| SARC | 17.56 |
| TGCT | 17.31 |
| PAAD | 15.17 |
| CESC | 15.13 |
| HNSC | 13.27 |
| BLCA | 12.41 |
| STAD | 12.26 |
| SKCM | 11.54 |
| BRCA | 11.29 |
| CHOL | 11.11 |
| KIRP | 11.00 |
| OV | 8.00 |
| ESCA | 5.98 |
| THCA | 5.74 |
| LIHC | 5.61 |
| ACC | 5.06 |
| UCEC | 4.36 |
| COAD | 4.03 |
| UCS | 3.51 |
| UVM | 2.5 |
| RICH | 1.52 |
| READ | 1.2 |
| LGG | 0.94 |
| PCPG | 0.55 |
| GBM | 0.54 |
| PRAD | 0.40 |

TABLE 15
Percent of samples with high TIM-3 and LAG-3 expression
(above global 75^{th} percentile) for double combination
therapy, across TCGA indications.

| Indication | % samples |
|---|---|
| KIRC | 33.09 |
| MESO | 32.18 |
| LUAD | 25.09 |
| LUSC | 21.56 |
| SARC | 19.08 |
| TGCT | 17.31 |
| CESC | 15.46 |
| OV | 15.12 |
| HNSC | 13.85 |
| STAD | 13.46 |
| BLCA | 12.90 |
| BRCA | 12.10 |
| SKCM | 11.54 |
| CHOL | 8.33 |
| PAAD | 5.62 |
| ESCA | 5.43 |
| THCA | 5.15 |
| LIHC | 5.08 |
| UCEC | 4.72 |
| KIRP | 4.47 |
| ACC | 3.8 |
| COAD | 3.6 |
| UCS | 3.51 |
| UVM | 2.5 |
| GBM | 1.61 |
| LGG | 0.56 |
| PCPG | 0.55 |
| PRAD | 0.4 |

TABLE 16
Percent of samples with high TIM-3 and CD73 expression
(above global 75^{th} percentile) for double combination
therapy, across TCGA indications.

| Indication | % samples |
|---|---|
| GBM | 27.96 |
| SCRC | 22.52 |
| LUAD | 21.89 |
| MESO | 14.94 |
| KIRC | 10.78 |
| PAAD | 10.11 |
| KIRP | 9.28 |
| LGG | 7.49 |
| HNSC | 6.54 |
| STAD | 6.25 |
| BLCA | 6.08 |
| THCA | 4.75 |
| COAD | 4.66 |
| LUSC | 4.39 |
| LIHC | 3.21 |
| SKCM | 2.88 |
| CHOL | 2.78 |
| ACC | 2.53 |
| OV | 2.33 |
| CESC | 1.64 |
| READ | 1.2 |
| ESCA | 1.09 |
| BRCA | 0.99 |
| UCEC | 0.75 |

Tables 17 and 18 highlights indications that would benefit from TIM-3 with PDCD1 and LAG-3 (Table 17) or MET (Table 18). In either scenario, the triple combination would most benefit KIRC and Lung carcinomas (LUAD and LUSC) and MESO. The results of the analysis for triple combination with TIM3 with PDCD1 and LAG3 resembles the double combination analysis with LAG3 or PDCD1 in that the triple would benefit similar indications but with 20-30% lower percentage representation.

Similar to what we observed for double combinations with TIM-3 and PDCD1 or LAG-3, a combination targeting all three would most benefit KIRC and Lung carcinomas (LUAD and LUSC and MESO (Tables 17 and 18), but for a lower percent of the patient population than with the double combinations.

TABLE 17
Percent of samples with high TIM-3, PDCD1 and LAG-3
expression (above global 75^{th} percentile) nominated
for triple combination therapy across TCGA.

| Indications | % samples |
|---|---|
| KIRC | 29.18 |
| LUAD | 23.21 |
| LUSC | 20.96 |
| MESO | 19.54 |
| TGCT | 17.31 |
| SARC | 15.27 |
| CESC | 15.13 |
| HNSC | 13.65 |
| STAD | 12.74 |
| OV | 11.63 |
| BLCA | 11.44 |
| BRCA | 11.29 |
| SKCM | 10.58 |
| CHOL | 8.33 |
| ESCA | 5.43 |
| THCA | 4.95 |
| LIHC | 4.81 |
| UCEC | 4.72 |
| COAD | 3.39 |
| PAAD | 3.37 |
| ACC | 2.53 |
| UVM | 2.50 |
| KIRP | 2.41 |
| UCS | 1.75 |
| GBM | 0.54 |
| PRAD | 0.4 |

TABLE 18
Percent of samples with high TIM-3, PDCD1 and MET
expression (above global 75^{th} percentile) nominated
for triple combination therapy across TCGA.

| Indication | % sample |
|---|---|
| KIRC | 38.48 |
| LUAD | 18.11 |
| MESO | 10.34 |
| THCA | 4.16 |
| STAD | 3.85 |
| LUSC | 3.39 |
| PAAD | 3.37 |
| CHOL | 2.78 |
| KIRP | 2.75 |
| COAD | 2.54 |
| UVM | 2.50 |
| BLCA | 1.95 |
| UCEC | 1.45 |
| CESC | 1.32 |
| READ | 1.2 |
| HNSC | 1.15 |
| ESCA | 1.09 |
| SKCM | 0.96 |
| SARC | 0.76 |
| LIHC | 0.53 |
| BRCA | 0.18 |

### Summary

Using RNA expression as a basis of selection, we determined that targeting TIM-3 with LAG-3 or PDCD1 or all three could benefit more than 10% of at least 13 indications represented in TCGA and more than 30% of patients affected by KIRC, LUAD, LUSC or MESO. The high representation of these indications could be a consequence of high expression of TIM3 in normal kidney and lung cells. Similarly, the combination of TIM-3 wish CD73 may benefit approximately 28% of GBM patients, however TIM-3 is also found to be expressed in brain tissue at moderate levels compared to other organs.

### Example 6: Clinical Study of PDR001 in Combination with a CXCR2 Inhibitor

### Objective

The primary objective of this study is to combine the PDR001 checkpoint inhibitor with the CXCR2 inhibitor, 6-chloro-3-((3,4-dioxo-2-(pentan-3-ylamino)cyclobut-1-en-1-yl)amino)-2-hydroxy-N-methoxy-N-methylbenzenesulfonamide choline salt, to identify the doses and schedule for combination therapy and to preliminarily assess the safety, tolerability, pharmacological and clinical activity of these combinations.

### Materials and Methods

Patients are dosed on a flat scale and not by body weight or body surface area. Dosing of the CXCR2 inhibitor occurs immediately after completion of the PDR001 infusion during clinic visits. Patients are treated with 400 mg PDR001 every four weeks (i.e., Q4W). The CXCR2 inhibitor is administered orally twice daily, approximately 12 hours apart, with approximately 240 mL of water on an empty stomach at least 1 hour before or 2 hours after a meal, at about the same time every day. Patients are instructed not to chew the medication but to swallow it whole. The CXCR2 inhibitor is administered at a starting dose of 75 mg BID, two weeks on/two weeks off in each 28-day cycle or one week on/two weeks off in each 21-day cycle. If tolerated, the dose may be escalated to 150 mg BID two weeks on/two weeks off or one week on/two weeks off. It is possible for additional and/or intermediate dose levels to be added during the course of the study. Cohorts may be added at any dose level below the MTD in order to better understand safety, PK or PD. Multiple dose levels below the MTD may be evaluated simultaneously in order to obtain PK and PD data across a range of doses and to establish the MTD/RDE. Less frequent schedules may also be explored for the CXCR2 inhibitor if deemed necessary.

### Results

In the first cohort of this arm of the study, seven patients have been administered PDR001 400 mg Q4W and the CXCR2 inhibitor, 6-chloro-3-((3,4-dioxo-2-(pentan-3-ylamino)cyclobut-1-en-1-yl)amino)-2-hydroxy-N-methoxy-N-methylbenzenesulfonamide choline salt, at 75 mg BID, 2 weeks on/2 week off. Two patients have completed the DLT evaluation period (56 days), and no patient has developed a DLT.

[symbol]

### EMBODIMENTS OF THE APPLICATION

The following are embodiments disclosed in the present application. The embodiments include, but are not limited to:

1. A combination comprising a PD-1 inhibitor, a SERD, and a CDK4/6 inhibitor for use in treating an Estrogen Receptor positive (ER+) cancer in a subject.

2. A method of treating an Estrogen Receptor positive (ER+) cancer in a subject, comprising administering to the subject a combination of a PD-1 inhibitor, a SERD, and a CDK4/6 inhibitor.

3. The combination for use of embodiment 1, or the method of embodiment 2, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

4. The combination for use of embodiment 1 or 3, or the method of embodiment 2 or 3, wherein the SERD is chosen from LSZ102, fulvestrant, brilanestrant, or elacestrant.

5. The combination for use of embodiment 1, 3, or 4, or the method of any of embodiments 2-4, wherein the CDK4/6 inhibitor is chosen from ribociclib, abemaciclib, or palbociclib.

6. A combination comprising a PD-1 inhibitor, a CXCR2 inhibitor, and a CSF-1/1R binding agent for use in treating a pancreatic cancer or a colorectal cancer in a subject.

7. A method of treating a pancreatic cancer or a colorectal cancer in a subject, comprising administering to the subject a combination of PD-1 inhibitor, a CXCR2 inhibitor, and a CSF-1/1R binding agent.

8. The combination for use of embodiment 6, or the method of embodiment 7, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

9. The combination for use of embodiment 6 or 8, or the method of embodiment 7 or 8, wherein the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof, danirixin, reparixin, or navarixin.

10. The combination for use of embodiment 6, 8, or 9, or the method of any of embodiments 7-9, wherein the CSF-1/1R binding agent is chosen from MCS110, BLZ945, pexidartinib, emactuzumab, or FPA008.

11. A combination comprising a PD-1 inhibitor, a CXCR2 inhibitor, a CSF-1/1R binding agent, and an additional therapeutic agent, for use in treating a cancer in a subject.

12. A method of treating a cancer in a subject comprising administering to the subject a combination of a PD-1 inhibitor, a CXCR2 inhibitor, a CSF-1/1R binding agent, and a fourth therapeutic agent.

13. The combination for use of embodiment 11, or the method of embodiment 12, wherein the cancer is a pancreatic cancer or a colorectal cancer.

14. The combination for use of embodiment 11 or 13, or the method of embodiment 12 or 13, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

15. The combination for use of any of embodiments 11, 13, or 14, or the method of any of embodiments 12-14, wherein the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof, danirixin, reparixin, or navarixin.

16. The combination for use of any of embodiments 11 or 13-15, or the method of any of embodiments 12-15, wherein the CSF-1/1R binding agent is chosen from MCS110, BLZ945, pexidartinib, emactuzumab, or FPA008.

17. The combination for use of any of embodiments 11 or 13-16, or the method of any of embodiments 12-16, wherein the additional therapeutic agent is chosen from one, two, or all of a TIM-3 inhibitor, a c-MET inhibitor, or an A2aR antagonist.

18. The combination for use of any of embodiments 11 or 13-17, or the method of any of embodiments 12-17, wherein the additional therapeutic agent comprises a TIM-3 inhibitor.

19. The combination for use of embodiment 18, or the method of embodiment 18, wherein the TIM-3 inhibitor is chosen from MBG453 or TSR-022.

20. The combination for use of any of embodiments 11 or 13-19, or the method of any of embodiments 12-19, wherein the additional therapeutic agent comprises a c-MET inhibitor.

21. The combination for use of embodiment 20, or the method of embodiment 20, wherein the c-MET inhibitor is chosen from JNJ-3887605, AMG 337, LY2801653, MSC2156119J, crizotinib, capmatinib, tivantinib or golvatinib.

22. The combination for use of any of embodiments 11 or 13-21, or the method of any of embodiments 12-21, wherein the additional therapeutic agent comprises an A2aR antagonist.

23. The combination for use of embodiment 22, or the method of embodiment 22, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

24. A combination comprising a PD-1 inhibitor, a CXCR2 inhibitor, and an additional therapeutic agent chosen from one, two, or all, of a TIM-3 inhibitor, a c-MET inhibitor, or an A2aR antagonist for use in treating a pancreatic cancer or a colorectal cancer in a subject.

25. A method of treating a pancreatic cancer or a colorectal cancer in a subject comprising administering to the subject a combination of a PD-1 inhibitor, a CXCR2 inhibitor, and an additional therapeutic agent chosen from one, two, or all, of a TIM-3 inhibitor, a c-MET inhibitor, or an A2aR antagonist.

26. The combination for use of embodiment 24, or the method of embodiment 25, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

27. The combination for use of embodiment 24 or 26, or the method of embodiment 25 or 26, wherein the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof, danirixin, reparixin, or navarixin.

28. The combination for use of any of embodiments 24, 26, or 27, or the method of any of embodiments 25-27, wherein the additional therapeutic agent comprises a TIM-3 inhibitor.

29. The combination for use of embodiment 28, or the method of embodiment 28, wherein the TIM-3 inhibitor is MBG453 or TSR-02.

30. The combination for use of any of embodiments 24 or 26-29, or the method of any of embodiments 25-29, wherein the additional therapeutic agent comprises a c-MET inhibitor.

31. The combination for use of embodiment 30, or the method of embodiment 30, wherein the c-MET inhibitor is chosen from capmatinib (INC280), JNJ-3887605, AMG 337, LY2801653, MSC2156119J, crizotinib, tivantinib, or golvatinib.

32. The combination for use of any of embodiments 24 or 26-31, or the method of any of embodiments 25-31, wherein the additional therapeutic agent comprises an A2aR antagonist.

33. The combination for use of embodiment 32, or the method of embodiment 32, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

34. A combination comprising a PD-1 inhibitor, a GITR agonist, and an additional therapeutic agent chosen from one, two, three, four, or all, of a TGF-β inhibitor, an A2aR antagonist, a c-MET inhibitor, a TIM-3 inhibitor, or a LAG-3 inhibitor for use in treating a pancreatic cancer, a colorectal cancer, or a melanoma in a subject.

35. A method of treating a pancreatic cancer, a colorectal cancer, or a melanoma in a subject comprising administering to the subject a combination of a PD-1 inhibitor, a GITR agonist, and an additional therapeutic agent chosen from one, two, three, four, or all, of a TGF-β inhibitor, an A2aR antagonist, a c-MET inhibitor, a TIM-3 inhibitor, or a LAG-3 inhibitor.

36. The combination for use of embodiment 34, or the method of embodiment 35, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

37. The combination for use of embodiment 34 or 36, or the method of embodiment 35 or 36, wherein the GITR agonist is chosen from GWN323, BMS-986156, MK-4166, MK-1248, TRX518, INCAGN1876, AMG 228, or INBRX-110.

38. The combination for use of any of embodiments 34, 36, or 37, or the method of any of embodiments 35-37, wherein the additional therapeutic agent comprises a TGF-β inhibitor.

39. The combination for use of embodiment 38, or the method of embodiment 38, wherein the TGF-β inhibitor is XOMA 089 or fresolimumab.

40. The combination for use of any of embodiments 34 or 36-39, or the method of any of embodiments 35-39, wherein the additional therapeutic agent comprises an A2Ar antagonist.

41. The combination for use of embodiment 40, or the method of embodiment 40, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

42. The combination for use of any of embodiments 34 or 36-41, or the method of any of embodiments 35-41, wherein the additional therapeutic agent comprises a c-MET inhibitor.

43. The combination for use of embodiment 42, or the method of embodiment 42, wherein the c-MET inhibitor is chosen from capmatinib (INC280), JNJ-3887605, AMG 337, LY2801653, MSC2156119J, crizotinib, tivantinib, or golvatinib.

44. The combination for use of any of embodiments 34 or 36-43, or the method of any of embodiments 35-43, wherein the additional therapeutic agent comprises a TIM-3 inhibitor.

45. The combination for use of embodiment 44, or the method of embodiment 44, wherein the TIM-3 inhibitor is chosen from MBG453 or TSR-022.

46. The combination for use of any of embodiments 34 or 36-45, or the method of any of embodiments 35-45, wherein the additional therapeutic agent comprises a LAG-3 inhibitor.

47. The combination for use of embodiment 46, or the method of embodiment 46, wherein the LAG-3 inhibitor is chosen from LAG525, BMS-986016, or TSR-033.

48. A combination comprising a PD-1 inhibitor, a LAG-3 inhibitor, a GITR agonist, and an additional therapeutic agent chosen from one, two, or all, of a TGF-β inhibitor, an A2aR antagonist, or a c-MET inhibitor for treating a cancer.

49. A method of treating a cancer in a subject comprising administering to the subject a combination of a PD-1 inhibitor, a LAG-3 inhibitor, a GITR agonist, and an additional therapeutic agent chosen from one, two, or all, of a TGF-β inhibitor, an A2aR antagonist, or a c-MET inhibitor.

50. The combination for use of embodiment 48, or the method of embodiment 49, wherein the cancer is chosen from a pancreatic cancer, a colorectal cancer, or a melanoma.

51. The combination for use of embodiment 48 or 49, or the method of embodiment 49 or 50, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

52. The combination for use of any of embodiments 48, 49, or 51, or the method of any of embodiments 49-51, wherein the LAG-3 inhibitor is chosen from LAG52, BMS-986016, or TSR-033.

53. The combination for use of any of embodiments 48 or 50-52, or the method of any of embodiments 49-52, wherein the GITR agonist is chosen from GWN323, BMS-986156, MK-4166, MK-1248, TRX518, INCAGN1876, AMG 228, or INBRX-110.

54. The combination for use of any of embodiments 49 or 50-53, or the method of any of embodiments 49-53, wherein the additional therapeutic agent comprises a TGF-β inhibitor.

55. The combination for use of embodiment 54, or the method of embodiment 54, wherein the TGF-β inhibitor is XOMA 089 or fresolimumab.

56. The combination for use of any of embodiments 48 or 49-54, or the method of any of embodiments 49-54, wherein the additional therapeutic agent comprises an A2aR antagonist.

57. The combination for use of embodiment 56, or the method of embodiment 56, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

58. The combination for use of any of embodiments 48 or 50-55, or the method of any of embodiments 49-55, wherein the additional therapeutic agent comprises a c-MET inhibitor.

59. The combination for use of embodiment 58, or the method of embodiment 58, wherein the c-MET inhibitor is chosen from capmatinib (INC280), JNJ-3887605, AMG 337, LY2801653, MSC2156119J, crizotinib, tivantinib, or golvatinib.

60. A combination comprising a PD-1 inhibitor, an A2aR antagonist, and an additional therapeutic agent chosen from one or more of a TGF-β inhibitor or a CSF-1/1R binding agent for use in treating a pancreatic cancer, a colorectal cancer, or a melanoma in a subject.

61. A method of treating a pancreatic cancer, a colorectal cancer, or a melanoma in a subject comprising administering to the subject a combination of a PD-1 inhibitor, an A2aR antagonist, and an additional therapeutic agent chosen from one or more of a TGF-β inhibitor or a CSF-1/1R binding agent.

62. The combination for use of embodiment 60, or the method of embodiment 61, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

63. The combination for use of embodiment 60 or 62, or the method of embodiment 61 or 62, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

64. The combination for use of any of embodiments 60, 62, or 63, or the method of any of embodiments 61-63, wherein the additional therapeutic agent comprises a TGF-β inhibitor.

65. The combination for use of embodiment 64, or the method of embodiment 64, wherein the TGF-β inhibitor is XOMA 089 or fresolimumab.

66. The combination for use of any of embodiments 60 or 62-65, or the method of any of embodiments 61-65, wherein the additional therapeutic agent comprises a CSF-1/1R binding agent.

67. The combination for use of embodiment 66, or the method of embodiment 66, wherein the CSF-1/1R binding agent is chosen from MCS110, BLZ945, pexidartinib, emactuzumab, or FPA008.

68. A combination comprising a PD-1 inhibitor, a c-MET inhibitor, and an additional therapeutic agent chosen from one, two, or all of a TGF-β inhibitor, an A2aR antagonist, or a CSF-1/1R binding agent for use in treating a pancreatic cancer, a colorectal cancer, a gastric cancer, or a melanoma in a subject.

69. A method of treating a pancreatic cancer, a colorectal cancer, a gastric cancer, or a melanoma in a subject comprising administering to the subject a combination of a PD-1 inhibitor, a c-MET inhibitor, and an additional therapeutic agent chosen from one, two, or all of a TGF-β inhibitor, an A2aR antagonist, or a CSF-1/1R binding agent.

70. The combination for use of embodiment 68, or the method of embodiment 69, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

71. The combination for use of embodiment 68 or 70, or the method of embodiment 69 or 70, wherein the MET inhibitor is chosen from capmatinib (INC280), JNJ-3887605, AMG 337, LY2801653, MSC2156119J, crizotinib, tivantinib, or golvatinib.

72. The combination for use of any of embodiments 68, 70, or 71, or the method of any of embodiments 69-71, wherein the additional therapeutic agent comprises a TGF-β inhibitor.

73. The combination for use of embodiment 72, or the method of embodiment 72, wherein the TGF-β inhibitor is XOMA 089 or fresolimumab.

74. The combination for use of any of embodiments 68 or 70-73, or the method of any of embodiments 69-73, wherein the additional therapeutic agent comprises an A2aR antagonist.

75. The combination for use of embodiment 74, or the method of embodiment 74, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

76. The combination for use of any of embodiments 68 or 70-75, or the method of any of embodiments 69-75, wherein the additional therapeutic agent comprises a CSF-1/1R binding agent.

77. The combination for use of embodiment 76, or the method of embodiment 76, wherein the CSF-1/1R binding agent is chosen from MCS110, BLZ945, pexidartinib, emactuzumab, or FPA008.

78. A combination comprising a PD-1 inhibitor, an IDO inhibitor, and an additional therapeutic agent chosen from one, two, three, four, or all of a TGF-β inhibitor, an A2aR antagonist, a CSF-1/1R binding agent, a c-MET inhibitor, or a GITR agonist for use in treating a pancreatic cancer, a colorectal cancer, a gastric cancer, or a melanoma in a subject.

79. A method of treating a pancreatic cancer, a colorectal cancer, a gastric cancer, or a melanoma in a subject comprising administering to the subject a combination of a PD-1 inhibitor, an IDO inhibitor, and an additional therapeutic agent chosen from one, two, three, four, or all of a TGF-β inhibitor, an A2aR antagonist, a CSF-1/1R binding agent, a c-MET inhibitor, or a GITR agonist.

80. The combination for use of embodiment 78, or the method of embodiment 79, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

81. The combination for use of embodiment 78 or 80, or the method of embodiment 79 or 80, wherein the IDO inhibitor is chosen from epacadostat (INCB24360), indoximod, α-NLG919, or F001287.

82. The combination for use of any of embodiment 78, 80, or 81, or the method of any of embodiments 79-81, wherein the additional therapeutic agent comprises a TGF-β inhibitor.

83. The combination for use of embodiment 82, or the method of embodiment 82, wherein the TGF-β inhibitor is XOMA 089 or fresolimumab.

84. The combination for use of any of embodiments 78 or 80-83, or the method of any of embodiments 79-83, wherein the additional therapeutic agent comprises an A2aR antagonist.

85. The combination for use of embodiment 84, or the method of embodiment 84, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

86. The combination for use of any of embodiments 78 or 80-85, or the method of any of embodiments 79-85, wherein the additional therapeutic agent comprises a CSF-1/1R binding agent.

87. The combination for use of embodiment 86, or the method of embodiment 86, wherein the CSF-1/1R binding agent is chosen from MCS110, BLZ945, pexidartinib, emactuzumab, or FPA008.

88. The combination for use of any of embodiments 78 or 80-86, or the method of any of embodiments 79-86, wherein the additional therapeutic agent comprises a c-MET inhibitor.

89. The combination for use of embodiment 88, or the method of embodiment 88, wherein the c-MET inhibitor is chosen from capmatinib (INC280), JNJ-3887605, AMG 337, LY2801653, MSC2156119J, crizotinib, tivantinib, or golvatinib.

90. The combination for use of any of embodiments 78 or 80-89, or the method of any of embodiments 75-85, wherein the additional therapeutic agent comprises a GITR agonist.

91. The combination for use of embodiment 90, or the method of embodiment 90, wherein the GITR agonist is chosen from GWN323, BMS-986156, MK-4166, MK-1248, TRX518, INCAGN1876, AMG 228, or INBRX-110.

92. A combination comprising a PD-1 inhibitor, a TIM-3 inhibitor, an A2aR antagonist, and an additional therapeutic agent chosen from one, two or all of a TGF-β inhibitor or a CSF-1/1R binding agent for treating a cancer.

93. A method of treating a cancer in a subject comprising administering to the subject a combination of a PD-1 inhibitor, a TIM-3 inhibitor, an A2aR antagonist, and an additional therapeutic agent chosen from one, two or all of a TGF-β inhibitor or a CSF-1/1R binding agent.

94. The combination for use of embodiment 92, or the method of embodiment 93, wherein the cancer is chosen from a pancreatic cancer or a colon cancer.

95. The combination for use of embodiment 92 or 94, or the method of embodiment 93 or 94, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

96. The combination for use of any of embodiments 92 or 94-95, or the method of any of embodiments 93-95, wherein the TIM-3 inhibitor is chosen from MBG453 or TSR-022.

97. The combination for use of any of embodiments 92 or 94-96, or the method of any of embodiments 93-96, wherein the A2aR antagonist chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

98. The combination for use of any of embodiments 92 or 94-97, or the method of any of embodiments 93-97, wherein the additional therapeutic agent comprises a TGF-β inhibitor.

99. The combination for use of embodiment 98, or the method of embodiment 98, wherein the TGF-β inhibitor is XOMA 089 or fresolimumab.

100. The combination for use of any of embodiments 92 or 94-99, or the method of any of embodiments 93-99, wherein the additional therapeutic agent comprises a CSF-1/1R binding agent.

101. The combination for use of embodiment 100, or the method of embodiment 100, wherein the CSF-1/1R binding agent is chosen from MCS110, BLZ945, pexidartinib, emactuzumab, or FPA008.

102. A combination comprising a PD-1 inhibitor, a TIM-3 inhibitor, and an additional therapeutic agent chosen from one, two or all, of a STING agonist, or a CSF-1/1R binding agent for use in treating a colon cancer in a subject.

103. A method of treating a colon cancer in a subject comprising administering to the subject a combination of a PD-1 inhibitor, a TIM-3 inhibitor, and an additional therapeutic agent chosen from one, two or all, of a STING agonist, or a CSF-1/1R binding agent.

104. The combination for use of embodiment 102, or the method of embodiment 103, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

105. The combination for use of embodiment 102 or 104, or the method of embodiments 103 or 104, wherein the TIM-3 inhibitor is chosen from MBG453 or TSR-022.

106. The combination for use of any of embodiments 102 or 104-105, or the method of use of any of embodiments 103-105, wherein the additional therapeutic agent comprises a STING agonist.

107. The combination for use of embodiment 106, or the method of embodiment 106, wherein the STING agonist is MK-1454.

108. The combination for use of any of embodiments 102 or 104-107, or the method of use of any of embodiments 103-107, wherein the additional therapeutic agent comprises a CSF-1/1R binding agent.

109. The combination for use of embodiment 108, or the method of embodiment 108, wherein the CSF-1/1R binding agent is chosen from MCS110, BLZ945, pexidartinib, emactuzumab, or FPA008.

110. A combination comprising one or more of a Galectin inhibitor described herein, and one or more of an additional therapeutic agent, e.g., a therapeutic agent described herein, for use in treating a cancer in a subject.

111. A method of treating a cancer in a subject comprising administering to the subject in need thereof a combination of one ore more of a Galectin inhibitor described herein, and one or more of an additional therapeutic agent, e.g., a therapeutic agent described herein.

112. The composition for use of embodiment 110, or the method of embodiment 111, wherein the Galectin inhibitor is chosen from an anti-Galectin (e.g., anti-Galectin-1 or anti-Galectin-3) antibody molecule, GR-MD-02, Galectin-3C, Anginex, or OTX-008.

113. The composition for use of any of embodiments 110 or 112, or the method of embodiment 111 or 112, wherein the anti-Galectin antibody molecule is chosen from: a monospecific anti-Galectin-1 antibody, a monospecific anti-Galectin-3 antibody, or a bispecific anti-Galectin-1 and anti-Galectin-3 antibody.

114. The composition for use of any of embodiments 110 or 112-113, or the method of any of embodiments 111-113, wherein the Galectin inhibitor comprises a monospecific anti-Galectin-1 antibody and a monospecific anti-Galectin-3 antibody molecule.

115. The composition for use of any of embodiments 110 or 112-114, or the method of any of embodiments 111-114, wherein the Galectin inhibitor is a bispecific anti-Galectin-1 and anti-Galectin-3 antibody.

116. The composition for use of any of embodiments 110 or 112-115, or the method of any of embodiments 111-115, wherein the additional therapeutic agent comprises a PD-1 inhibitor.

117. The composition for use of any of embodiments 110 or 112-116, or the method of any of embodiments 111-116, wherein the PD-1 inhibitor is PDR001.

118. A combination comprising a PD-1 inhibitor, a LAG-3 inhibitor, and an additional therapeutic agent chosen from one, two, three, four, five, six, seven or all, of a TGF-β inhibitor, a TIM-3 inhibitor, a c-MET inhibitor, an IL-1b inhibitor, a MEK inhibitor, a GITR agonist, an A2aR antagonist, or a CSF-1/1R binding agent for use in treating a breast cancer in a subject.

119. A method of treating a breast cancer in a subject comprising administering to the subject a combination of a PD-1 inhibitor, a LAG-3 inhibitor, and an additional therapeutic agent chosen from one, two, three, four, five, six, seven, or all, of a TGF-β inhibitor, a TIM-3 inhibitor, a c-MET inhibitor, an IL-1b inhibitor, a MEK inhibitor, a GITR agonist, an A2aR antagonist, or a CSF-1/1R binding agent.

120. The combination for use of embodiment 118, or the method of embodiment 119, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

121. The combination for use of embodiment 118 or 120, or the method of embodiment 119 or 120, wherein the LAG-3 inhibitor is chosen from LAG525, BMS-986016, or TSR-033.

122. The combination for use of any of embodiments 118 or 120-121, or the method of any of embodiments 119-121, wherein the additional therapeutic agent comprises a TGF-β inhibitor.

123. The combination for use of embodiment 122, or the method of embodiment 122, wherein the TGF-β inhibitor is chosen from XOMA 089 or fresolimumab.

124. The combination for use of any of embodiments 118 or 120-123, or the method of any of embodiments 119-123, wherein the additional therapeutic agent comprises a TIM-3 inhibitor.

125. The combination for use of embodiment 124, or the method of embodiment 124, wherein the TIM-3 inhibitor is chosen from MBG453 or TSR-022.

126. The combination for use of any of embodiments 118 or 120-125, or the method of any of embodiments 119-125, wherein the additional therapeutic agent comprises a c-MET inhibitor.

127. The combination for use of embodiment 126, or the method of embodiment 126, wherein the c-MET inhibitor is chosen from capmatinib (INC280), JNJ-3887605, AMG 337, LY2801653, MSC2156119J, crizotinib, tivantinib, or golvatinib.

128. The combination for use of any of embodiments 118 or 120-127, or the method of any of embodiments 119-127, wherein the additional therapeutic agent comprises an IL-1b inhibitor.

129. The combination for use of embodiment 128, or the method of embodiment 128, wherein the IL-1b inhibitor is chosen from canakinumab, gevokizumab, Anakinra, or Rilonacept.

130. The combination for use of any of embodiments 118 or 120-129, or the method of any of embodiments 119-129, wherein the additional therapeutic agent comprises a MEK inhibitor.

131. The combination for use of embodiment 130, or the method of embodiment 130, wherein the MEK inhibitor is chosen from Trametinib, selumetinib, AS703026, BIX 02189, BIX 02188, CI-1040, PD0325901, PD98059, U0126, XL-518, G-38963, or G02443714.

132. The combination for use of any of embodiments 118 or 120-129, or the method of any of embodiments 119-129, wherein the additional therapeutic agent comprises a GITR agonist, optionally wherein the GITR agonist is chosen from GWN323, BMS-986156, MK-4166, MK-1248, TRX518, INCAGN1876, AMG 228, or INBRX-110.

133. The combination for use of any of embodiments 118 or 120-129, or the method of any of embodiments 119-129, wherein the additional therapeutic agent comprises an A2aR antagonist, optionally wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

134. The combination for use of c1 any of embodiments 118 or 120-133, or the method of any of embodiments 119-133, wherein the breast cancer is a triple negative breast cancer (TNBC), e.g., advanced or metastatic TNBC.

135. The combination for use of any of embodiments 118 or 120-129, or the method of any of embodiments 119-129, wherein the additional therapeutic agent comprises a CSF-1/1R binding agent.

136. The combination for use of embodiment 135, or the method of embodiment 135, wherein the CSF-1/1R binding agent is chosen from MCS110, BLZ945, pexidartinib, emactuzumab, or FPA008.

137. A combination comprising a PD-1 inhibitor, a CSF-1/1R binding agent, and an additional therapeutic agent chosen from one, two, three, or all of a TGF-β inhibitor, a TIM-3 inhibitor, a c-MET inhibitor, or an IL-1b inhibitor for use in treating a breast cancer in a subject.

138. A method of treating a breast cancer in a subject comprising administering to the subject a combination of a a PD-1 inhibitor, a CSF-1/1R binding agent, and an additional therapeutic agent chosen from one, two, three, or all, of a TGF-β inhibitor, a TIM-3 inhibitor, a c-MET inhibitor, or an IL-1b inhibitor.

139. The combination for use of embodiment 137, or the method of embodiment 138, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

140. The combination for use of embodiment 137 or 139, or the method of embodiment 138 or 139 wherein the CSF-1/1R binding agent is chosen from MCS110, BLZ945, pexidartinib, emactuzumab, or FPA008.

141. The combination for use of any of embodiments 137 or 139-140, or the method of any of embodiments 138-140, wherein the additional therapeutic agent comprises a TGF-β inhibitor.

142. The combination for use of embodiment 141, or the method of embodiment 141, wherein the TGF-β inhibitor is chosen from XOMA 089 or fresolimumab.

143. The combination for use of any of embodiments 137 or 139-142, or the method of any of embodiments 138-142, wherein the additional therapeutic agent comprises a TIM-3 inhibitor.

144. The combination for use of embodiment 143, or the method of embodiment 143, wherein the TIM-3 inhibitor is chosen from MBG453 or TSR-022.

145. The combination for use of any of embodiments 137 or 139-144, or the method of any of embodiments 138-144, wherein the additional therapeutic agent comprises a c-MET inhibitor.

146. The combination for use of embodiment 145, or the method of embodiment 145, wherein the c-MET inhibitor is chosen from capmatinib (INC280), JNJ-3887605, AMG 337, LY2801653, MSC2156119J, crizotinib, tivantinib, or golvatinib.

147. The combination for use of any of embodiments 137 or 139-146, or the method of any of embodiments 138-146, wherein the additional therapeutic agent comprises an IL-1b inhibitor.

148. The combination for use of embodiment 147, or the method of embodiment 147, wherein the IL-1b inhibitor is chosen from canakinumab, gevokizumab, Anakinra, or Rilonacept.

149. The combination for use of any of embodiments 137 or 139-148, or the method of any of embodiments 138-148, wherein the breast cancer is a triple negative breast cancer.

150. A combination comprising a PD-1 inhibitor, an A2aR antagonist, and an additional therapeutic agent chosen from one, two, three, four, five, or all, of a TGF-β inhibitor, a TIM-3 inhibitor, a c-MET inhibitor, an IL-1b inhibitor, IL-15/IL15RA complex, or a CSF-1/1R binding agent for use in treating a breast cancer, a colorectal cancer, a pancreatic cancer or a gastroesophageal cancer in a subject.

151. A method of treating a breast cancer, a colorectal cancer, a pancreatic cancer or a gastroesophageal cancer in a subject comprising administering to the subject a combination of a PD-1 inhibitor, an A2aR antagonist, and an additional therapeutic agent chosen from one, two, three, four, five, or all, of a TGF-β inhibitor, a TIM-3 inhibitor, a c-MET inhibitor, an IL-1b inhibitor, IL-15/IL15RA complex, or a CSF-1/1R binding agent.

152. The combination for use of embodiment 150, or the method of embodiment 151, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

153. The combination for use of embodiment 150 or 151, or the method of embodiment 151 or 152, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

154. The combination for use of any of embodiments 150 or 152-153, or the method of any of embodiments 151-153, wherein the additional therapeutic agent comprises a TGF-β inhibitor.

155. The combination for use of embodiment 154, or the method of embodiment 154, wherein the TGF-β inhibitor is chosen from XOMA 089 or fresolimumab.

156. The combination for use of any of embodiments 150 or 152-155, or the method of any of embodiments 151-155, wherein the additional therapeutic agent comprises a TIM-3 inhibitor.

157. The combination for use of embodiment 156, or the method of embodiment 156, wherein the TIM-3 inhibitor is chosen from MBG453 or TSR-022.

158. The combination for use of any of embodiments 150 or 152-157, or the method of any of embodiments 151-157, wherein the additional therapeutic agent comprises a c-MET inhibitor.

159. The combination for use of embodiment 158, or the method of embodiment 158, wherein the c-MET inhibitor is chosen from capmatinib (INC280), JNJ-3887605, AMG 337, LY2801653, MSC2156119J, crizotinib, tivantinib, or golvatinib.

160. The combination for use of any of embodiments 150 or 152-159, or the method of any of embodiments 151-159, wherein the additional therapeutic agent comprises an IL-1b inhibitor.

161. The combination for use of embodiment 160, or the method of embodiment 160, wherein the IL-1b inhibitor is chosen from canakinumab, gevokizumab, Anakinra, or Rilonacept.

162. The combination for use of any of embodiments 150 or 152-161, or the method of any of embodiments 151-161, wherein the additional therapeutic agent comprises an IL-15/IL-15RA complex.

163. The combination for use of embodiment 162, or the method of embodiment 162, wherein the IL-15/IL-15RA complex is chosen from NIZ985, ATL-803 or CYP0150.

164. The combination for use of any of embodiments 150 or 152-163, or the method of any of embodiments 151-163, wherein the additional therapeutic agent comprises a CSF-1/1R binding agent.

165. The combination for use of embodiment 164, or the method of embodiment 164, wherein the CSF-1/1R binding agent is chosen from MCS110, BLZ945, pexidartinib, emactuzumab, or FPA008.

166. The combination for use of any of embodiments 150 or 152-165, or the method of any of embodiments 151-165, wherein the breast cancer is a triple negative breast cancer.

167. The combination for use of any of embodiments 150 or 151-165, or the method of any of embodiments 151-165, wherein the colorectal cancer is an MSS colorectal cancer.

168. A combination comprising a PD-1 inhibitor, an IL-1b inhibitor, and an additional therapeutic agent chosen from one, two, three, four, or all of a TGF-β inhibitor, a TIM-3 inhibitor, a c-MET inhibitor, a IL-15/IL15RA complex, or a CSF-1/1R binding agent for use in treating a colorectal cancer, a pancreatic cancer or a gastroesophageal cancer in a subject.

169. A method of treating a colorectal cancer, a pancreatic cancer or a gastroesophageal cancer in a subject comprising administering to the subject a combination of a PD-1 inhibitor, an IL-1b inhibitor, and an additional therapeutic agent chosen from one, two, three, four, or all of a TGF-β inhibitor, a TIM-3 inhibitor, a c-MET inhibitor, a IL-15/IL15RA complex, or a CSF-1/1R binding agent.

170. The combination for use of embodiment 168, or the method of embodiment 169, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

171. The combination for use of embodiment 168 or 170, or the method of embodiment 169 or 170, wherein the IL-1b inhibitor is chosen from canakinumab, gevokizumab, Anakinra, or Rilonacept.

172. The combination for use of any of embodiments 168 or 170-171, or the method of any of embodiments 169-171, wherein the additional therapeutic agent comprises a TGF-β inhibitor.

173. The combination for use of embodiment 172, or the method of embodiment 172, wherein the TGF-β inhibitor is chosen from XOMA 089 or fresolimumab.

174. The combination for use of any of embodiments 168 or 170-173, or the method of any of embodiments 169-173, wherein the additional therapeutic agent comprises an IL-15/IL-15RA complex.

175. The combination for use of embodiment 174, or the method of embodiment 174, wherein the IL-15/IL-15RA complex is chosen from NIZ985, ATL-803 or CYP0150.

176. The combination for use of any of embodiments 168 or 170-175, or the method of any of embodiments 169-175, wherein the additional therapeutic agent comprises a c-MET inhibitor.

177. The combination for use of embodiment 176, or the method of embodiment 176, wherein the c-MET inhibitor is chosen from capmatinib (INC280), JNJ-3887605, AMG 337, LY2801653, MSC2156119J, crizotinib, tivantinib, or golvatinib.

178. The combination for use of any of embodiments 168 or 170-177, or the method of any of embodiments 169-177, wherein the additional therapeutic agent comprises a CSF-1/1R binding agent.

179. The combination for use of embodiment 178, or the method of embodiment 178, wherein the CSF-1/1R binding agent is chosen from MCS110, BLZ945, pexidartinib, emactuzumab, or FPA008.

180. The combination for use of any of embodiments 168 or 170-177, or the method of any of embodiments 169-177, wherein the additional therapeutic agent comprises a TIM-3 inhibitor.

181. The combination for use of embodiment 178, or the method of embodiment 178, wherein the TIM-3 inhibitor is chosen from MBG453 or TSR-022.

182. The combination for use of any of embodiments 168 or 170-181, or the method of any of embodiments 169-181, wherein the colorectal cancer is an MSS colorectal cancer.

183. A combination comprising a PD-1 inhibitor, a MEK inhibitor, and an additional therapeutic agent chosen from one, two, three, four, or all of a TGF-β inhibitor, a TIM-3 inhibitor, a c-MET inhibitor, an IL-15/IL15RA complex, or a CSF-1/1R binding agent for use in treating a colorectal cancer, a pancreatic cancer or a gastroesophageal cancer in a subject.

184. A method of treating a colorectal cancer, a pancreatic cancer or a gastroesophageal cancer in a subject comprising administering to the subject a combination of a PD-1 inhibitor, a MEK inhibitor, and an additional therapeutic agent chosen from one, two, three, four, or all of a TGF-β inhibitor, a TIM-3 inhibitor, a c-MET inhibitor, an IL-15/IL15RA complex, or a CSF-1/1R binding agent.

185. The combination for use of embodiment 183, or the method of embodiment 184, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

186. The combination for use of embodiment 183 or 185, or the method of embodiment 184 or 185, wherein the MEK inhibitor is chosen from Trametinib, selumetinib, AS703026, BIX 02189, BIX 02188, CI-1040, PD0325901, PD98059, U0126, XL-518, G-38963, or G02443714.

187. The combination for use of embodiment 183 or 185-186, or the method of embodiments 184-186, wherein the additional therapeutic agent comprises a TGF-β inhibitor.

188. The combination for use or the method of embodiment 187, wherein the TGF-β inhibitor is chosen from XOMA 089 or fresolimumab.

189. The combination for use of any of embodiments 183 or 185-188, or the method of any of embodiments 184-188, wherein the additional therapeutic agent comprises an IL-15/IL-15RA complex.

190. The combination for use of embodiment 189, or the method of embodiment 189, wherein the IL-15/IL-15RA complex is chosen from NIZ985, ATL-803 or CYP0150.

191. The combination for use of any of embodiments 183 or 185-190, or the method of any of embodiments 184-190, wherein the additional therapeutic agent comprises a c-MET inhibitor.

192. The combination for use of embodiment 191, or the method of embodiment 191, wherein the c-MET inhibitor is chosen from capmatinib (INC280), JNJ-3887605, AMG 337, LY2801653, MSC2156119J, crizotinib, tivantinib, or golvatinib.

193. The combination for use of any of embodiments 183 or 185-192, or the method of any of embodiments 184-192, wherein the additional therapeutic agent comprises a CSF-1/1R binding agent.

194. The combination for use of embodiment 193, or the method of embodiment 193, wherein the CSF-1/1R binding agent is chosen from MCS110, BLZ945, pexidartinib, emactuzumab, or FPA008.

195. The combination for use of any of embodiments 183 or 185-194, or the method of any of embodiments 184-194, wherein the additional therapeutic agent comprises a TIM-3 inhibitor.

196. The combination for use of embodiment 195, or the method of embodiment 195, wherein the TIM-3 inhibitor is chosen from MBG453 or TSR-022.

197. The combination for use of any of embodiments 183 or 185-196, or the method of any of embodiments 184-196, wherein the colorectal cancer is an MSS colorectal cancer.

198. The combination for use or the method of any of the preceding embodiments, wherein the inhibitor, binding agent, agonist, antagonist, or additional therapeutic agent are administered together in a single composition or administered separately in two or more different compositions or dosage forms.

199. The combination for use or the method of any of the preceding embodiments, wherein the PD-1 inhibitor is used at a dose of about 200 mg to about 400 mg once every three weeks.

200. The combination for use of embodiment 199, or the method of embodiment 199, wherein the PD-1 inhibitor is used at a dose of about 300 mg once every three weeks.

201. The combination for use or the method of any of the preceding embodiments, wherein the PD-1 inhibitor is used at a dose of about 300 mg to about 500 mg once every four weeks.

202. The combination for use or the method of any of the preceding embodiments, wherein the PD-1 inhibitor is used at a dose of about 400 mg once every four weeks.

203. A combination comprising an IL-1b inhibitor, an A2AR antagonist, and an additional therapeutic agent, e.g., an IL-15/IL15Ra complex, for use in treating a colorectal cancer, a pancreatic cancer or a gastroesophageal cancer in a subject.

204. A method of treating a colorectal cancer, a pancreatic cancer or a gastroesophageal cancer in a subject comprising administering to the subject a combination of an IL-1b inhibitor, an A2AR antagonist, and an additional therapeutic agent, e.g., an IL-15/IL15Ra complex.

205. The combination for use of embodiment 203, or the method of embodiment 204, wherein the IL-1b inhibitor is chosen from canakinumab, gevokizumab, Anakinra, or Rilonacept.

206. The combination for use of embodiment 203, or the method of embodiment 204, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

207. The combination for use of any of embodiments 203 or 205-206, or the method of any of embodiments 202-204, wherein the additional agent comprises an IL-15/IL15Ra complex.

208. The combination for use of embodiment 207, or the method of embodiment 207, wherein the IL-15/IL-15Ra complex is chosen from NIZ985, ATL-803 or CYP0150.

209. A combination comprising an IL-1b inhibitor, an A2AR antagonist, and an additional therapeutic agent chosen from, one or both of an IL-15/IL-15Ra complex or a TGF-β inhibitor for use in treating a cancer.

210. A method of treating a cancer in a subject comprising administering to the subject a combination of an IL-1b inhibitor, an A2AR antagonist, and an additional therapeutic agent chosen from, one or both of an IL-15/IL-15Ra complex or a TGF-β inhibitor.

211. The combination for use of embodiment 209, or the method of embodiment 210, wherein the IL-1b inhibitor is chosen from canakinumab, gevokizumab, Anakinra, or Rilonacept.

212. The combination for use of embodiment 209, or the method of embodiment 210, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

213. The combination for use of any of embodiments 209 or 210-212, or the method of any of any of embodiments 208-210, wherein the additional therapeutic agent comprises an IL-15/IL-15Ra complex.

214. The combination for use of embodiment 213, or the method of embodiment 213, wherein the IL-15/IL-15Ra complex is chosen from NIZ985, ATL-803 or CYP0150.

215. The combination for use of any of embodiments 209, or 211-212, or the method of any of any of embodiments 208-210, wherein the additional therapeutic agent comprises a TGF-β inhibitor.

216. The combination for use of embodiment 215, or the method of embodiment 215, wherein the TGF-β inhibitor is chosen from XOMA 089 or fresolimumab.

217. A combination comprising an IL-15/IL-15Ra complex, a TGF-β inhibitor, and an additional therapeutic agent chosen from one, or both of an IL-1b inhibitor or a CSF-1/1R binding agent, for use in treating a cancer in a subject.

218. A method of treating a cancer in a subject comprising administering to the subject a combination of an IL-15/IL-15Ra complex, a TGF-β inhibitor, and an additional therapeutic agent chosen from one, two, three or all of an IL-1b inhibitor, a CSF-1/1R binding agent, a c-MET inhibitor, or an A2aR antagonist.

219. The combination for use of embodiment 217, or the method of embodiment 218, wherein the IL-15/IL-15Ra complex is chosen from XOMA 089 or fresolimumab.

220. The combination for use of embodiment 217, or the method of embodiment 218, wherein the TGF-β inhibitor is chosen from NIZ985, ATL-803 or CYP0150.

221. The combination for use of any of embodiments 217 or 219-220, or the method of any of any of embodiments 218-220, wherein the additional therapeutic agent comprises an IL-1b inhibitor.

222. The combination for use of embodiment 221, or the method of embodiment 221, wherein the IL-1b inhibitor is chosen from canakinumab, gevokizumab, Anakinra, or Rilonacept.

223. The combination for use of any of embodiments 217 or 219-220, or the method of any of embodiments 216-218, wherein the additional therapeutic agent comprises a CSF-1/1R binding agent.

224. The combination for use of embodiment 223, or the method of embodiment 223, wherein the CSF-1/1R binding agent is chosen from MCS110, BLZ945, pexidartinib, emactuzumab, or FPA008.

225. The combination for use any of embodiments 217 or 219-220, or the method of any of embodiments 216-218, wherein the additional therapeutic agent comprises a c-MET inhibitor.

226. The combination for use of embodiment 225, or the method of embodiment 225, wherein the c-MET inhibitor is chosen from capmatinib (INC280), JNJ-3887605, AMG 337, LY2801653, MSC2156119J, crizotinib, tivantinib, or golvatinib.

227. The combination for use of any of embodiments 217 or 219-220, or the method of any of embodiments 216-218, wherein the additional therapeutic agent comprises an A2aR antagonist.

228. The combination for use of embodiment 227, or the method of embodiment 227, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

229. The combination for use of any of embodiments 209, 211-217, or 219-228, or the method of any of embodiments 208-214, or 216-226, wherein the cancer is chosen from a colorectal cancer, a pancreatic cancer or a gastroesophageal cancer.

230. The combination for use of embodiment 229, or the method of embodiment 229, wherein the colorectal cancer is an MSS colorectal cancer.

231. A combination comprising a PD-1 inhibitor and a CXCR2 inhibitor for use in treating a colorectal cancer, a lung cancer, a pancreatic cancer, or a breast cancer in a subject.

232. A method of treating a colorectal cancer, a lung cancer, a pancreatic cancer, or a breast cancer in a subject comprising administering to the subject a combination of a PD-1 inhibitor, and a CXCR2 inhibitor.

233. The combination for use of embodiment 231, or the method of embodiment 232, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

234. The combination for use of embodiment 231 or 233, or the method of embodiment 232 or 233, wherein the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof, danirixin, reparixin, or navarixin.

235. The combination for use of any of embodiments 231, 233 or 234, or the method of any of embodiments 232-234, wherein the CXCR2 inhibitor is 6-chloro-3-((3,4-dioxo-2-(pentan-3-ylamino)cyclobut-1-en-1-yl)amino)-2-hydroxy-N-methoxy-N-methylbenzenesulfonamide choline salt.

236. The combination for use of any of embodiments 231 or 233-235, or the method of any of embodiments 232-235, wherein the CXCR2 inhibitor is administered twice daily for 2 weeks in a 4 week cycle, wherein each dose is 75 mg.

237. The combination for use of any of embodiments 231 or 233-235, or the method of any of embodiments 232-235, wherein the CXCR2 inhibitor is administered twice daily for 2 weeks in a 4 week cycle, wherein each dose is 150 mg.

238. The combination for use of any of embodiments 231 or 233-237, or the method of any of embodiments 232-237, wherein the CXCR2 inhibitor is administered orally.

239. The combination for use of any of embodiments 231 or 233-238, or the method of any of embodiments 232-238, wherein the colorectal cancer is an MSS colorectal cancer.

240. The combination for use of any of embodiments 231 or 233-238, or the method of any of embodiments 232-238, wherein the lung cancer is a non-small cell lung cancer (NSCLC).

241. The combination for use of any of embodiments 231 or 233-238, or the method of any of embodiments 232-238, wherein the breast cancer is a triple negative breast cancer (TNBC).

242. The combination for use of any of embodiments 231 or 233-241, or the method of any of embodiments 232-241, wherein the combination further comprises a CSF-1/1R binding agent.

243. The combination for use or the method of embodiment 242, wherein the CSF-1/1R binding agent is MCS110, BLZ945, pexidartinib, emactuzumab, or FPA008.

244. The combination for use or the method of embodiment 242, wherein the CSF-1/1R binding agent is MCS110.

245. The combination for use or the method of embodiment 242, wherein the CSF-1/1R binding agent is BLZ945.

246. The combination for use or the method of any of the preceding embodiments, wherein the inhibitor, binding agent, agonist, antagonist, or additional therapeutic agent comprises an antibody molecule.

247. A pharmaceutical composition or dose formulation comprising a combination of any of the preceding embodiments.

248. The pharmaceutical composition or dose formulation of embodiment 247, for use in the treatment of a cancer chosen from a breast cancer, a pancreatic cancer, a colorectal cancer, a melanoma, a gastric cancer, a lung cancer, or an ER+ cancer.

249. The pharmaceutical composition or dose formulation of embodiment 248, wherein the breast cancer is a triple negative breast cancer (TNBC), e.g., advanced or metastatic TNBC.

250. The pharmaceutical composition or dose formulation of embodiment 248, wherein the colorectal cancer is a MSS colorectal cancer.

251. The pharmaceutical composition or dose formulation of embodiment 248, wherein the lung cancer is NSCLC.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A combination comprising a PD-1 inhibitor, a CXCR2 inhibitor, and a CSF-1/1R binding agent for use in treating a pancreatic cancer or a colorectal cancer in a subject.

2. A method of treating a pancreatic cancer or a colorectal cancer in a subject, comprising administering to the subject a combination of PD-1 inhibitor, a CXCR2 inhibitor, and a CSF-1/1R binding agent.

3. The combination for use of claim 1, or the method of claim 2, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

4. The combination for use of claim 1 or 3, or the method of claim 2 or 3, wherein the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof, danirixin, reparixin, or navarixin.

5. The combination for use of claim 1, 3, or 4, or the method of any of claims 2-4, wherein the CSF-1/1R binding agent is chosen from MCS110, BLZ945, pexidartinib, emactuzumab, or FPA008.

6. A combination comprising a PD-1 inhibitor, a CXCR2 inhibitor, a CSF-1/1R binding agent, and an additional therapeutic agent, for use in treating a cancer in a subject.

7. A method of treating a cancer in a subject comprising administering to the subject a combination of a PD-1 inhibitor, a CXCR2 inhibitor, a CSF-1/1R binding agent, and a fourth therapeutic agent.

8. The combination for use of claim 6, or the method of claim 7, wherein the cancer is a pancreatic cancer or a colorectal cancer.

9. The combination for use of claim 6 or 8, or the method of claim 7 or 8, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

10. The combination for use of any of claim 6, 8, or 9, or the method of any of claims 7-9, wherein the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof, danirixin, reparixin, or navarixin.

11. The combination for use of any of claim 6 or 8-10, or the method of any of claims 7-10, wherein the CSF-1/1R binding agent is chosen from MCS110, BLZ945, pexidartinib, emactuzumab, or FPA008.

12. The combination for use of any of claim 6 or 8-11, or the method of any of claims 7-11, wherein the additional therapeutic agent is chosen from one, two, or all of a TIM-3 inhibitor, a c-MET inhibitor, or an A2aR antagonist.

13. The combination for use of any of claim 6 or 8-12, or the method of any of claims 7-12, wherein the additional therapeutic agent comprises a TIM-3 inhibitor.

14. The combination for use of claim 13, or the method of claim 13, wherein the TIM-3 inhibitor is chosen from MBG453 or TSR-022.

15. The combination for use of any of claim 6 or 8-14, or the method of any of claims 7-14, wherein the additional therapeutic agent comprises a c-MET inhibitor.

16. The combination for use of claim 15, or the method of claim 15, wherein the c-MET inhibitor is chosen from JNJ-3887605, AMG 337, LY2801653, MSC2156119J, crizotinib, capmatinib, tivantinib or golvatinib.

17. The combination for use of any of claim 6 or 8-16, or the method of any of claims 7-16, wherein the additional therapeutic agent comprises an A2aR antagonist.

18. The combination for use of claim 17, or the method of claim 17, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

19. A combination comprising a PD-1 inhibitor, a CXCR2 inhibitor, and an additional therapeutic agent chosen from one, two, or all, of a TIM-3 inhibitor, a c-MET inhibitor, or an A2aR antagonist for use in treating a pancreatic cancer or a colorectal cancer in a subject.

20. A method of treating a pancreatic cancer or a colorectal cancer in a subject comprising administering to the subject a combination of a PD-1 inhibitor, a CXCR2 inhibitor, and an additional therapeutic agent chosen from one, two, or all, of a TIM-3 inhibitor, a c-MET inhibitor, or an A2aR antagonist.

21. The combination for use of claim 19, or the method of claim 20, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

22. The combination for use of claim 19 or 21, or the method of claim 20 or 21, wherein the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof, danirixin, reparixin, or navarixin.

23. The combination for use of any of claim 19, 21, or 22, or the method of any of claims 20-22, wherein the additional therapeutic agent comprises a TIM-3 inhibitor.

24. The combination for use of claim 23, or the method of claim 23, wherein the TIM-3 inhibitor is MBG453 or TSR-02.

25. The combination for use of any of claim 19 or 21-24, or the method of any of claims 20-24, wherein the additional therapeutic agent comprises a c-MET inhibitor.

26. The combination for use of claim 25, or the method of claim 25, wherein the c-MET inhibitor is chosen from capmatinib (INC280), JNJ-3887605, AMG 337, LY2801653, MSC2156119J, crizotinib, tivantinib, or golvatinib.

27. The combination for use of any of claim 19 or 21-26, or the method of any of claims 20-26, wherein the additional therapeutic agent comprises an A2aR antagonist.

28. The combination for use of claim 27, or the method of claim 27, wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

29. A combination comprising a PD-1 inhibitor, a LAG-3 inhibitor, and an additional therapeutic agent chosen from one, two, three, four, five, six, seven or all, of a TGF-β inhibitor, a TIM-3 inhibitor, a c-MET inhibitor, an IL-1b inhibitor, a MEK inhibitor, a GITR agonist, an A2aR antagonist, or a CSF-1/1R binding agent for use in treating a breast cancer in a subject.

30. A method of treating a breast cancer in a subject comprising administering to the subject a combination of a PD-1 inhibitor, a LAG-3 inhibitor, and an additional therapeutic agent chosen from one, two, three, four, five, six, seven, or all, of a TGF-β inhibitor, a TIM-3 inhibitor, a c-MET inhibitor, an IL-1b inhibitor, a MEK inhibitor, a GITR agonist, an A2aR antagonist, or a CSF-1/1R binding agent.

31. The combination for use of claim 29, or the method of claim 30, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

32. The combination for use of claim 29 or 31, or the method of claim 30 or 31, wherein the LAG-3 inhibitor is chosen from LAG525, BMS-986016, or TSR-033.

33. The combination for use of any of claim 29 or 31-32, or the method of any of claims 30-32, wherein the additional therapeutic agent comprises a TGF-β inhibitor.

34. The combination for use of claim 33, or the method of claim 33, wherein the TGF-β inhibitor is chosen from XOMA 089 or fresolimumab.

35. The combination for use of any of claim 29 or 31-34, or the method of any of claims 30-34, wherein the additional therapeutic agent comprises a TIM-3 inhibitor.

36. The combination for use of claim 35, or the method of claim 35, wherein the TIM-3 inhibitor is chosen from MBG453 or TSR-022.

37. The combination for use of any of claim 29 or 31-36, or the method of any of claims 30-36, wherein the additional therapeutic agent comprises a c-MET inhibitor.

38. The combination for use of claim 37, or the method of claim 37, wherein the c-MET inhibitor is chosen from capmatinib (INC280), JNJ-3887605, AMG 337, LY2801653, MSC2156119J, crizotinib, tivantinib, or golvatinib.

39. The combination for use of any of claim 29 or 31-38, or the method of any of claims 30-38, wherein the additional therapeutic agent comprises an IL-1b inhibitor.

40. The combination for use of claim 39, or the method of claim 39, wherein the IL-1b inhibitor is chosen from canakinumab, gevokizumab, Anakinra, or Rilonacept.

41. The combination for use of any of claim 29 or 31-40, or the method of any of claims 30-40, wherein the additional therapeutic agent comprises a MEK inhibitor.

42. The combination for use of claim 41, or the method of claim 41, wherein the MEK inhibitor is chosen from Trametinib, selumetinib, AS703026, BIX 02189, BIX 02188, CI-1040, PD0325901, PD98059, U0126, XL-518, G-38963, or G02443714.

43. The combination for use of any of claim 29 or 31-40, or the method of any of claims 30-40, wherein the additional therapeutic agent comprises a GITR agonist, optionally wherein the GITR agonist is chosen from GWN323, BMS-986156, MK-4166, MK-1248, TRX518, INCAGN1876, AMG 228, or INBRX-110.

44. The combination for use of any of claim 29 or 31-40, or the method of any of claims 30-40, wherein the additional therapeutic agent comprises an A2aR antagonist, optionally wherein the A2aR antagonist is chosen from PBF509 (NIR178), CPI444/V81444, AZD4635/HTL-1071, Vipadenant, GBV-2034, AB928, Theophylline, Istradefylline, Tozadenant/SYN-115, KW-6356, ST-4206, or Preladenant/SCH 420814.

45. The combination for use of c1 any of claim 29 or 31-44, or the method of any of claims 30-44, wherein the breast cancer is a triple negative breast cancer (TNBC), e.g., advanced or metastatic TNBC.

46. The combination for use of any of claim 29 or 31-40, or the method of any of claims 30-40, wherein the additional therapeutic agent comprises a CSF-1/1R binding agent.

47. The combination for use of claim 46, or the method of claim 46, wherein the CSF-1/1R binding agent is chosen from MCS110, BLZ945, pexidartinib, emactuzumab, or FPA008.

48. A combination comprising a PD-1 inhibitor and a CXCR2 inhibitor for use in treating a colorectal cancer, a lung cancer, a pancreatic cancer, or a breast cancer in a subject.

49. A method of treating a colorectal cancer, a lung cancer, a pancreatic cancer, or a breast cancer in a subject comprising administering to the subject a combination of a PD-1 inhibitor, and a CXCR2 inhibitor.

50. The combination for use of claim 48, or the method of claim 49, wherein the PD-1 inhibitor is chosen from PDR001, Nivolumab, Pembrolizumab, Pidilizumab, MEDI0680, REGN2810, TSR-042, PF-06801591, BGB-A317, BGB-108, INCSHR1210, or AMP-224.

51. The combination for use of claim 48 or 50, or the method of claim 49 or 50, wherein the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof, danirixin, reparixin, or navarixin.

52. The combination for use of any of claim 48, 50 or 51, or the method of any of claims 49-51, wherein the CXCR2 inhibitor is 6-chloro-3-((3,4-dioxo-2-(pentan-3-ylamino)cyclobut-1-en-1-yl)amino)-2-hydroxy-N-methoxy-N-methylbenzenesulfonamide choline salt.

53. The combination for use of any of claim 48 or 50-52, or the method of any of claims 49-52, wherein the CXCR2 inhibitor is administered twice daily for 2 weeks in a 4 week cycle, wherein each dose is 75 mg.

54. The combination for use of any of claim 48 or 50-52, or the method of any of claims 49-52, wherein the CXCR2 inhibitor is administered twice daily for 2 weeks in a 4 week cycle, wherein each dose is 150 mg.

55. The combination for use of any of claim 48 or 50-54, or the method of any of claims 49-54, wherein the CXCR2 inhibitor is administered orally.

56. The combination for use of any of claim 48 or 50-55, or the method of any of claims 49-55, wherein the colorectal cancer is an MSS colorectal cancer.

57. The combination for use of any of claim 48 or 50-55, or the method of any of claims 49-55, wherein the lung cancer is a non-small cell lung cancer (NSCLC).

58. The combination for use of any of claim 48 or 50-55, or the method of any of claims 49-55, wherein the breast cancer is a triple negative breast cancer (TNBC).

59. The combination for use of any of claim 48 or 50-58, or the method of any of claims 49-58, wherein the combination further comprises a CSF-1/1R binding agent.

60. The combination for use or the method of claim 59, wherein the CSF-1/1R binding agent is MCS110, BLZ945, pexidartinib, emactuzumab, or FPA008.

61. The combination for use or the method of claim 59, wherein the CSF-1/1R binding agent is MCS110.

62. The combination for use or the method of claim 59, wherein the CSF-1/1R binding agent is BLZ945.

63. The combination for use or the method of any of the preceding claims, wherein the inhibitor, binding agent, agonist, antagonist, or additional therapeutic agent comprises an antibody molecule.

64. A pharmaceutical composition or dose formulation comprising a combination of any of the preceding claims.

65. The pharmaceutical composition or dose formulation of claim 64, for use in the treatment of a cancer chosen from a breast cancer, a pancreatic cancer, a colorectal cancer, a melanoma, a gastric cancer, a lung cancer, or an ER+ cancer.

66. The pharmaceutical composition or dose formulation of claim 65, wherein the breast cancer is a triple negative breast cancer (TNBC), e.g., advanced or metastatic TNBC.

67. The pharmaceutical composition or dose formulation of claim 65, wherein the colorectal cancer is a MSS colorectal cancer.

68. The pharmaceutical composition or dose formulation of claim 65, wherein the lung cancer is NSCLC.


SEQUENCE LISTING (2002 sequences)

SEQ ID NO: 1-237 - skipped (237 entries)

SEQ ID NO: 238 - protein, 447 aa, Artificial Sequence
  source: /note=“Description of Artificial Sequence: Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 239 - protein, 215 aa, Artificial Sequence
  source: /note=“Description of Artificial Sequence: Synthetic polypeptide”
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 121 aa, Artificial Sequence
  source: /note=“Description of Artificial Sequence: Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSS

SEQ ID NO: 241 - protein, 109 aa, Artificial Sequence
  source: /note=“Description of Artificial Sequence: Synthetic polypeptide”
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLG

SEQ ID NO: 242-500 - skipped (259 entries)

SEQ ID NO: 501 - protein, 5 aa, Artificial Sequence
  source: /note=“Description of Artificial Sequence: Synthetic peptide”
TYWMH

SEQ ID NO: 502 - protein, 17 aa, Artificial Sequence
  source: /note=“Description of Artificial Sequence: Synthetic peptide”
NIYPGTGGSNFDEKFKN

SEQ ID NO: 503 - protein, 8 aa, Artificial Sequence
  source: /note=“Description of Artificial Sequence: Synthetic peptide”
WTTGTGAY

SEQ ID NO: 504 - protein, 7 aa, Artificial Sequence
  source: /note=“Description of Artificial Sequence: Synthetic peptide”
GYTFTTY

SEQ ID NO: 505 - protein, 6 aa, Artificial Sequence
  source: /note=“Description of Artificial Sequence: Synthetic peptide”
YPGTGG

SEQ ID NO: 506 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 508 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 509 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 510 - protein, 17 aa, Artificial Sequence
  source: /note=“Description of Artificial Sequence: Synthetic peptide”
KSSQSLLDSGNQKNFLT

SEQ ID NO: 511 - protein, 7 aa, Artificial Sequence
  source: /note=“Description of Artificial Sequence: Synthetic peptide”
WASTRES

SEQ ID NO: 512 - protein, 9 aa, Artificial Sequence
  source: /note=“Description of Artificial Sequence: Synthetic peptide”
QNDYSYPYT

SEQ ID NO: 513 - protein, 13 aa, Artificial Sequence
  source: /note=“Description of Artificial Sequence: Synthetic peptide”
SQSLLDSGNQKNF

SEQ ID NO: 514 - protein, 3 aa, Artificial Sequence
  source: /note=“Description of Artificial Sequence: Synthetic peptide”
WAS

SEQ ID NO: 515 - protein, 6 aa, Artificial Sequence
  source: /note=“Description of Artificial Sequence: Synthetic peptide”
DYSYPY

SEQ ID NO: 516 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 518 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 520 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 522 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 524 - DNA, 15 bp, Artificial Sequence
  source: /note=“Description of Artificial Sequence: Synthetic oligonucleotide”
acctactggatgcac

SEQ ID NO: 525 - DNA, 51 bp, Artificial Sequence
  source: /note=“Description of Artificial Sequence: Synthetic oligonucleotide”
aatatctaccccggcaccggcggctctaacttcgacgagaagtttaagaat

SEQ ID NO: 526 - DNA, 24 bp, Artificial Sequence
  source: /note=“Description of Artificial Sequence: Synthetic oligonucleotide”
tggactaccggcacaggcgcctac

SEQ ID NO: 527 - DNA, 21 bp, Artificial Sequence
  source: /note=“Description of Artificial Sequence: Synthetic oligonucleotide”
ggctacaccttcactacctac

SEQ ID NO: 528 - DNA, 18 bp, Artificial Sequence
  source: /note=“Description of Artificial Sequence: Synthetic oligonucleotide”
taccccggcaccggcggc

SEQ ID NO: 529 - DNA, 51 bp, Artificial Sequence
  source: /note=“Description of Artificial Sequence: Synthetic oligonucleotide”
aaatctagtcagtcactgctggatagcggtaatcagaagaacttcctgacc

SEQ ID NO: 530 - DNA, 21 bp, Artificial Sequence
  source: /note=“Description of Artificial Sequence: Synthetic oligonucleotide”
tgggcctctactagagaatca

SEQ ID NO: 531 - DNA, 27 bp, Artificial Sequence
  source: /note=“Description of Artificial Sequence: Synthetic oligonucleotide”
cagaacgactatagctacccctacacc

SEQ ID NO: 532 - DNA, 39 bp, Artificial Sequence
  source: /note=“Description of Artificial Sequence: Synthetic oligonucleotide”
agtcagtcactgctggatagcggtaatcagaagaacttc

SEQ ID NO: 533 - DNA, 9 bp, Artificial Sequence
  source: /note=“Description of Artificial Sequence: Synthetic oligonucleotide”
tgggcctct

SEQ ID NO: 534 - DNA, 18 bp, Artificial Sequence
  source: /note=“Description of Artificial Sequence: Synthetic oligonucleotide”
gactatagctacccctac

SEQ ID NO: 535 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 536 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 447 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 538 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 539 - protein, 447 aa, Artificial Sequence
  source: /note=“Description of Artificial Sequence: Synthetic polypeptide”
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 213 aa, Artificial Sequence
  source: /note=“Description of Artificial Sequence: Synthetic polypeptide”
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 541 - protein, 10 aa, Artificial Sequence
  source: /note=“Description of Artificial Sequence: Synthetic peptide”
GYTFTTYWMH

SEQ ID NO: 542-600 - skipped (59 entries)

SEQ ID NO: 601 - protein, 5 aa, Artificial Sequence
  source: /note=“Description of Artificial Sequence: Synthetic peptide”
SYWMY

SEQ ID NO: 602 - protein, 17 aa, Artificial Sequence
  source: /note=“Description of Artificial Sequence: Synthetic peptide”
RIDPNSGSTKYNEKFKN

SEQ ID NO: 603 - protein, 11 aa, Artificial Sequence
  source: /note=“Description of Artificial Sequence: Synthetic peptide”
DYRKGLYAMDY

SEQ ID NO: 604 - protein, 7 aa, Artificial Sequence
  source: /note=“Description of Artificial Sequence: Synthetic peptide”
GYTFTSY

SEQ ID NO: 605 - protein, 6 aa, Artificial Sequence
  source: /note=“Description of Artificial Sequence: Synthetic peptide”
DPNSGS

SEQ ID NO: 606 - protein, 120 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 607 - DNA, 360 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 608 - protein, 446 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 609 - protein, 11 aa, Artificial Sequence
  source: /note=“Description of Artificial Sequence: Synthetic peptide”
KASQDVGTAVA

SEQ ID NO: 610 - protein, 7 aa, Artificial Sequence
  source: /note=“Description of Artificial Sequence: Synthetic peptide”
WASTRHT

SEQ ID NO: 611 - protein, 9 aa, Artificial Sequence
  source: /note=“Description of Artificial Sequence: Synthetic peptide”
QQYNSYPLT

SEQ ID NO: 612 - protein, 7 aa, Artificial Sequence
  source: /note=“Description of Artificial Sequence: Synthetic peptide”
SQDVGTA

SEQ ID NO: 613 - protein, 3 aa, Artificial Sequence
  source: /note=“Description of Artificial Sequence: Synthetic peptide”
WAS

SEQ ID NO: 614 - protein, 6 aa, Artificial Sequence
  source: /note=“Description of Artificial Sequence: Synthetic peptide”
YNSYPL

SEQ ID NO: 615 - DNA, 1338 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 616 - protein, 107 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 617 - DNA, 321 bp, Artificial Sequence
  source: /note=“Description of Artificial Sequence: Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 618 - protein, 214 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 619 - DNA, 642 bp, Artificial Sequence
  source: /note=“Description of Artificial Sequence: Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 620 - protein, 120 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 621 - DNA, 360 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 622 - protein, 446 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 623 - DNA, 1338 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 624 - protein, 107 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 625 - DNA, 321 bp, Artificial Sequence
  source: /note=“Description of Artificial Sequence: Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 626 - protein, 214 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 627 - DNA, 642 bp, Artificial Sequence
  source: /note=“Description of Artificial Sequence: Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 628 - DNA, 15 bp, Artificial Sequence
  source: /note=“Description of Artificial Sequence: Synthetic oligonucleotide”
agctactggatgtac

SEQ ID NO: 629 - DNA, 51 bp, Artificial Sequence
  source: /note=“Description of Artificial Sequence: Synthetic oligonucleotide”
agaatcgaccctaatagcggctctactaagtataacgagaagtttaagaat

SEQ ID NO: 630 - DNA, 33 bp, Artificial Sequence
  source: /note=“Description of Artificial Sequence: Synthetic oligonucleotide”
gactatagaaagggcctgtacgctatggactac

SEQ ID NO: 631 - DNA, 21 bp, Artificial Sequence
  source: /note=“Description of Artificial Sequence: Synthetic oligonucleotide”
ggctacaccttcactagctac

SEQ ID NO: 632 - DNA, 18 bp, Artificial Sequence
  source: /note=“Description of Artificial Sequence: Synthetic oligonucleotide”
gaccctaatagcggctct

SEQ ID NO: 633 - DNA, 33 bp, Artificial Sequence
  source: /note=“Description of Artificial Sequence: Synthetic oligonucleotide”
aaagcctctcaggacgtgggcaccgccgtggcc

SEQ ID NO: 634 - DNA, 21 bp, Artificial Sequence
  source: /note=“Description of Artificial Sequence: Synthetic oligonucleotide”
tgggcctctactagacacacc

SEQ ID NO: 635 - DNA, 27 bp, Artificial Sequence
  source: /note=“Description of Artificial Sequence: Synthetic oligonucleotide”
cagcagtataatagctaccccctgacc

SEQ ID NO: 636 - DNA, 21 bp, Artificial Sequence
  source: /note=“Description of Artificial Sequence: Synthetic oligonucleotide”
tctcaggacgtgggcaccgcc

SEQ ID NO: 637 - DNA, 9 bp, Artificial Sequence
  source: /note=“Description of Artificial Sequence: Synthetic oligonucleotide”
tgggcctct

SEQ ID NO: 638 - DNA, 18 bp, Artificial Sequence
  source: /note=“Description of Artificial Sequence: Synthetic oligonucleotide”
tataatagctaccccctg

SEQ ID NO: 639 - protein, 448 aa, Artificial Sequence
  source: /note=“Description of Artificial Sequence: Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 640 - protein, 214 aa, Artificial Sequence
  source: /note=“Description of Artificial Sequence: Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 641 - protein, 450 aa, Artificial Sequence
  source: /note=“Description of Artificial Sequence: Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 642 - protein, 216 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 643 - protein, 451 aa, Artificial Sequence
  source: /note=“Description of Artificial Sequence: Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 644 - protein, 215 aa, Artificial Sequence
  source: /note=“Description of Artificial Sequence: Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 645 - protein, 123 aa, Artificial Sequence
  source: /note=“Description of Artificial Sequence: Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 646 - protein, 106 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 647 - protein, 10 aa, Artificial Sequence
  source: /note=“Description of Artificial Sequence: Synthetic peptide”
GYTFTSYWMY

SEQ ID NO: 648-700 - skipped (53 entries)

SEQ ID NO: 701 - protein, 5 aa, Artificial Sequence
  source: /note=“Description of Artificial Sequence: Synthetic peptide”
NYGMN

SEQ ID NO: 702 - protein, 17 aa, Artificial Sequence
  source: /note=“Description of Artificial Sequence: Synthetic peptide”
WINTDTGEPTYADDFKG

SEQ ID NO: 703 - protein, 16 aa, Artificial Sequence
  source: /note=“Description of Artificial Sequence: Synthetic peptide”
NPPYYYGTNNAEAMDY

SEQ ID NO: 704 - protein, 7 aa, Artificial Sequence
  source: /note=“Description of Artificial Sequence: Synthetic peptide”
GFTLTNY

SEQ ID NO: 705 - protein, 6 aa, Artificial Sequence
  source: /note=“Description of Artificial Sequence: Synthetic peptide”
NTDTGE

SEQ ID NO: 706 - protein, 125 aa, Artificial Sequence
  source: /note=“Description of Artificial Sequence: Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 707 - DNA, 375 bp, Artificial Sequence
  source: /note=“Description of Artificial Sequence: Synthetic polynucleotide”
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 708 - DNA, 375 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 709 - protein, 451 aa, Artificial Sequence
  source: /note=“Description of Artificial Sequence: Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 710 - protein, 11 aa, Artificial Sequence
  source: /note=“Description of Artificial Sequence: Synthetic peptide”
SSSQDISNYLN

SEQ ID NO: 711 - protein, 7 aa, Artificial Sequence
  source: /note=“Description of Artificial Sequence: Synthetic peptide”
YTSTLHL

SEQ ID NO: 712 - protein, 9 aa, Artificial Sequence
  source: /note=“Description of Artificial Sequence: Synthetic peptide”
QQYYNLPWT

SEQ ID NO: 713 - protein, 7 aa, Artificial Sequence
  source: /note=“Description of Artificial Sequence: Synthetic peptide”
SQDISNY

SEQ ID NO: 714 - protein, 3 aa, Artificial Sequence
  source: /note=“Description of Artificial Sequence: Synthetic peptide”
YTS

SEQ ID NO: 715 - protein, 6 aa, Artificial Sequence
  source: /note=“Description of Artificial Sequence: Synthetic peptide”
YYNLPW

SEQ ID NO: 716 - DNA, 1353 bp, Artificial Sequence
  source: /note=“Description of Artificial Sequence: Synthetic polynucleotide”
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 717 - DNA, 1353 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 718 - protein, 107 aa, Artificial Sequence
  source: /note=“Description of Artificial Sequence: Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 719 - DNA, 321 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 720 - DNA, 321 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 721 - protein, 214 aa, Artificial Sequence
  source: /note=“Description of Artificial Sequence: Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 722 - DNA, 642 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 723 - DNA, 642 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 724 - protein, 125 aa, Artificial Sequence
  source: /note=“Description of Artificial Sequence: Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 725 - DNA, 375 bp, Artificial Sequence
  source: /note=“Description of Artificial Sequence: Synthetic polynucleotide”
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 726 - DNA, 375 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 727 - protein, 451 aa, Artificial Sequence
  source: /note=“Description of Artificial Sequence: Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 728 - DNA, 1353 bp, Artificial Sequence
  source: /note=“Description of Artificial Sequence: Synthetic polynucleotide”
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 729 - DNA, 1353 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 730 - protein, 107 aa, Artificial Sequence
  source: /note=“Description of Artificial Sequence: Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 731 - DNA, 321 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 732 - DNA, 321 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 733 - protein, 214 aa, Artificial Sequence
  source: /note=“Description of Artificial Sequence: Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 734 - DNA, 642 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 735 - DNA, 642 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 736 - DNA, 15 bp, Artificial Sequence
  source: /note=“Description of Artificial Sequence: Synthetic oligonucleotide”
aattacgggatgaac

SEQ ID NO: 737 - DNA, 15 bp, Artificial Sequence
  source: /note=“Description of Artificial Sequence: Synthetic oligonucleotide”
aactacggcatgaac

SEQ ID NO: 738 - DNA, 51 bp, Artificial Sequence
  source: /note=“Description of Artificial Sequence: Synthetic oligonucleotide”
tggattaacaccgacaccggggagcctacctacgcggacgatttcaaggga

SEQ ID NO: 739 - DNA, 51 bp, Artificial Sequence
  source: /note=“Description of Artificial Sequence: Synthetic oligonucleotide”
tggatcaacaccgacaccggcgagcctacctacgccgacgacttcaagggc

SEQ ID NO: 740 - DNA, 48 bp, Artificial Sequence
  source: /note=“Description of Artificial Sequence: Synthetic oligonucleotide”
aacccgccctactactacggaaccaacaacgccgaagccatggactac

SEQ ID NO: 741 - DNA, 48 bp, Artificial Sequence
  source: /note=“Description of Artificial Sequence: Synthetic oligonucleotide”
aaccccccttactactacggcaccaacaacgccgaggccatggactat

SEQ ID NO: 742 - DNA, 21 bp, Artificial Sequence
  source: /note=“Description of Artificial Sequence: Synthetic oligonucleotide”
ggattcaccctcaccaattac

SEQ ID NO: 743 - DNA, 21 bp, Artificial Sequence
  source: /note=“Description of Artificial Sequence: Synthetic oligonucleotide”
ggcttcaccctgaccaactac

SEQ ID NO: 744 - DNA, 18 bp, Artificial Sequence
  source: /note=“Description of Artificial Sequence: Synthetic oligonucleotide”
aacaccgacaccggggag

SEQ ID NO: 745 - DNA, 18 bp, Artificial Sequence
  source: /note=“Description of Artificial Sequence: Synthetic oligonucleotide”
aacaccgacaccggcgag

SEQ ID NO: 746 - DNA, 33 bp, Artificial Sequence
  source: /note=“Description of Artificial Sequence: Synthetic oligonucleotide”
agctctagtcaggatatctctaactacctgaac

SEQ ID NO: 747 - DNA, 33 bp, Artificial Sequence
  source: /note=“Description of Artificial Sequence: Synthetic oligonucleotide”
tcctccagccaggacatctccaactacctgaac

SEQ ID NO: 748 - DNA, 21 bp, Artificial Sequence
  source: /note=“Description of Artificial Sequence: Synthetic oligonucleotide”
tacactagcaccctgcacctg

SEQ ID NO: 749 - DNA, 21 bp, Artificial Sequence
  source: /note=“Description of Artificial Sequence: Synthetic oligonucleotide”
tacacctccaccctgcacctg

SEQ ID NO: 750 - DNA, 27 bp, Artificial Sequence
  source: /note=“Description of Artificial Sequence: Synthetic oligonucleotide”
cagcagtactataacctgccctggacc

SEQ ID NO: 751 - DNA, 27 bp, Artificial Sequence
  source: /note=“Description of Artificial Sequence: Synthetic oligonucleotide”
cagcagtactacaacctgccctggacc

SEQ ID NO: 752 - DNA, 21 bp, Artificial Sequence
  source: /note=“Description of Artificial Sequence: Synthetic oligonucleotide”
agtcaggatatctctaactac

SEQ ID NO: 753 - DNA, 21 bp, Artificial Sequence
  source: /note=“Description of Artificial Sequence: Synthetic oligonucleotide”
agccaggacatctccaactac

SEQ ID NO: 754 - DNA, 9 bp, Artificial Sequence
  source: /note=“Description of Artificial Sequence: Synthetic oligonucleotide”
tacactagc

SEQ ID NO: 755 - DNA, 9 bp, Artificial Sequence
  source: /note=“Description of Artificial Sequence: Synthetic oligonucleotide”
tacacctcc

SEQ ID NO: 756 - DNA, 18 bp, Artificial Sequence
  source: /note=“Description of Artificial Sequence: Synthetic oligonucleotide”
tactataacctgccctgg

SEQ ID NO: 757 - DNA, 18 bp, Artificial Sequence
  source: /note=“Description of Artificial Sequence: Synthetic oligonucleotide”
tactacaacctgccctgg

SEQ ID NO: 758 - DNA, 15 bp, Artificial Sequence
  source: /note=“Description of Artificial Sequence: Synthetic oligonucleotide”
aactacgggatgaac

SEQ ID NO: 759 - DNA, 51 bp, Artificial Sequence
  source: /note=“Description of Artificial Sequence: Synthetic oligonucleotide”
tggattaacaccgacaccggcgagcctacctacgccgacgactttaagggc

SEQ ID NO: 760 - DNA, 48 bp, Artificial Sequence
  source: /note=“Description of Artificial Sequence: Synthetic oligonucleotide”
aaccccccctactactacggcactaacaacgccgaggctatggactac

SEQ ID NO: 761 - DNA, 21 bp, Artificial Sequence
  source: /note=“Description of Artificial Sequence: Synthetic oligonucleotide”
ggcttcaccctgactaactac

SEQ ID NO: 762 - protein, 447 aa, Artificial Sequence
  source: /note=“Description of Artificial Sequence: Synthetic polypeptide”
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 763 - protein, 214 aa, Artificial Sequence
  source: /note=“Description of Artificial Sequence: Synthetic polypeptide”
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 446 aa, Artificial Sequence
  source: /note=“Description of Artificial Sequence: Synthetic polypeptide”
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 220 aa, Artificial Sequence
  source: /note=“Description of Artificial Sequence: Synthetic polypeptide”
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766 - protein, 10 aa, Artificial Sequence
  source: /note=“Description of Artificial Sequence: Synthetic peptide”
GFTLTNYGMN

SEQ ID NO: 767-800 - skipped (34 entries)

SEQ ID NO: 801 - protein, 5 aa, Artificial Sequence
  source: /note=“Description of Artificial Sequence: Synthetic peptide”
SYNMH

SEQ ID NO: 802 - protein, 17 aa, Artificial Sequence
  source: /note=“Description of Artificial Sequence: Synthetic peptide”
DIYPGNGDTSYNQKFKG

SEQ ID NO: 803 - protein, 9 aa, Artificial Sequence
  source: /note=“Description of Artificial Sequence: Synthetic peptide”
VGGAFPMDY

SEQ ID NO: 804 - protein, 7 aa, Artificial Sequence
  source: /note=“Description of Artificial Sequence: Synthetic peptide”
GYTFTSY

SEQ ID NO: 805 - protein, 6 aa, Artificial Sequence
  source: /note=“Description of Artificial Sequence: Synthetic peptide”
YPGNGD

SEQ ID NO: 806 - protein, 118 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807 - DNA, 354 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 808 - protein, 444 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 809 - DNA, 1332 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 810 - protein, 15 aa, Artificial Sequence
  source: /note=“Description of Artificial Sequence: Synthetic peptide”
RASESVEYYGTSLMQ

SEQ ID NO: 811 - protein, 7 aa, Artificial Sequence
  source: /note=“Description of Artificial Sequence: Synthetic peptide”
AASNVES

SEQ ID NO: 812 - protein, 9 aa, Artificial Sequence
  source: /note=“Description of Artificial Sequence: Synthetic peptide”
QQSRKDPST

SEQ ID NO: 813 - protein, 11 aa, Artificial Sequence
  source: /note=“Description of Artificial Sequence: Synthetic peptide”
SESVEYYGTSL

SEQ ID NO: 814 - protein, 3 aa, Artificial Sequence
  source: /note=“Description of Artificial Sequence: Synthetic peptide”
AAS

SEQ ID NO: 815 - protein, 6 aa, Artificial Sequence
  source: /note=“Description of Artificial Sequence: Synthetic peptide”
SRKDPS

SEQ ID NO: 816 - protein, 111 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817 - DNA, 333 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 818 - protein, 218 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 819 - DNA, 654 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 820 - protein, 17 aa, Artificial Sequence
  source: /note=“Description of Artificial Sequence: Synthetic peptide”
DIYPGQGDTSYNQKFKG

SEQ ID NO: 821 - protein, 6 aa, Artificial Sequence
  source: /note=“Description of Artificial Sequence: Synthetic peptide”
YPGQGD

SEQ ID NO: 822 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823 - DNA, 354 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 824 - protein, 444 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 825 - DNA, 1332 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 826 - protein, 111 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827 - DNA, 333 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 828 - protein, 218 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 829 - DNA, 654 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 830 - protein, 114 aa, Artificial Sequence
  source: /note=“Description of Artificial Sequence: Synthetic polypeptide”
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, Artificial Sequence
  source: /note=“Description of Artificial Sequence: Synthetic polypeptide”
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, Artificial Sequence
  source: /note=“Description of Artificial Sequence: Synthetic polypeptide”
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, Artificial Sequence
  source: /note=“Description of Artificial Sequence: Synthetic polypeptide”
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834-900 - skipped (67 entries)

SEQ ID NO: 901 - protein, 121 aa, Artificial Sequence
  source: /note=“Description of Artificial Sequence: Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 902 - protein, 107 aa, Artificial Sequence
  source: /note=“Description of Artificial Sequence: Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 903 - protein, 451 aa, Artificial Sequence
  source: /note=“Description of Artificial Sequence: Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 904 - protein, 214 aa, Artificial Sequence
  source: /note=“Description of Artificial Sequence: Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 905 - DNA, 363 bp, Artificial Sequence
  source: /note=“Description of Artificial Sequence: Synthetic polynucleotide”
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 906 - DNA, 321 bp, Artificial Sequence
  source: /note=“Description of Artificial Sequence: Synthetic polynucleotide”
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 907 - DNA, 1353 bp, Artificial Sequence
  source: /note=“Description of Artificial Sequence: Synthetic polynucleotide”
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 908 - DNA, 642 bp, Artificial Sequence
  source: /note=“Description of Artificial Sequence: Synthetic polynucleotide”
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 909 - protein, 5 aa, Artificial Sequence
  source: /note=“Description of Artificial Sequence: Synthetic peptide”
SYGVD

SEQ ID NO: 910 - protein, 7 aa, Artificial Sequence
  source: /note=“Description of Artificial Sequence: Synthetic peptide”
GFSLSSY

SEQ ID NO: 911 - protein, 16 aa, Artificial Sequence
  source: /note=“Description of Artificial Sequence: Synthetic peptide”
VIWGGGGTYYASSLMG

SEQ ID NO: 912 - protein, 5 aa, Artificial Sequence
  source: /note=“Description of Artificial Sequence: Synthetic peptide”
WGGGG

SEQ ID NO: 913 - protein, 13 aa, Artificial Sequence
  source: /note=“Description of Artificial Sequence: Synthetic peptide”
HAYGHDGGFAMDY

SEQ ID NO: 914 - protein, 11 aa, Artificial Sequence
  source: /note=“Description of Artificial Sequence: Synthetic peptide”
RASESVSSNVA

SEQ ID NO: 915 - protein, 7 aa, Artificial Sequence
  source: /note=“Description of Artificial Sequence: Synthetic peptide”
SESVSSN

SEQ ID NO: 916 - protein, 7 aa, Artificial Sequence
  source: /note=“Description of Artificial Sequence: Synthetic peptide”
GASNRAT

SEQ ID NO: 917 - protein, 3 aa, Artificial Sequence
  source: /note=“Description of Artificial Sequence: Synthetic peptide”
GAS

SEQ ID NO: 918 - protein, 9 aa, Artificial Sequence
  source: /note=“Description of Artificial Sequence: Synthetic peptide”
GQSYSYPFT

SEQ ID NO: 919 - protein, 6 aa, Artificial Sequence
  source: /note=“Description of Artificial Sequence: Synthetic peptide”
SYSYPF

SEQ ID NO: 920 - protein, 124 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 921 - protein, 107 aa, Artificial Sequence
  source: /note=“Description of Artificial Sequence: Synthetic polypeptide”
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 922-999 - skipped (78 entries)

SEQ ID NO: 1000 - protein, 143 aa, Homo sapiens
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDITSASYTMI

SEQ ID NO: 1001 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1002 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 1003 - protein, 114 aa, Artificial Sequence
  source: /note=“Description of Artificial Sequence: Synthetic polypeptide”
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 1004 - protein, 297 aa, Artificial Sequence
  source: /note=“Description of Artificial Sequence: Synthetic polypeptide”
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1005 - protein, 114 aa, Homo sapiens
  VARIANT (93)..(93): /replace=“Glu”
  SITE (1)..(114): /note=“Variant residues given in the sequence have no preference with respect to those in the annotations for variant positions”
NWVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTS

SEQ ID NO: 1006 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 1007 - protein, 448 aa, Artificial Sequence
  source: /note=“Description of Artificial Sequence: Synthetic polypeptide”
QVQLQESGPGLVKPSQTLSLTCTVSDYSITSDYAWNWIRQFPGKGLEWMGYISYSGSTSYNPSLKSRITISRDTSKNQFSLQLNSVTAADTAVYYCASFDYAHAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1008 - protein, 214 aa, Artificial Sequence
  source: /note=“Description of Artificial Sequence: Synthetic polypeptide”
DIVLTQSPAFLSVTPGEKVTFTCQASQSIGTSIHWYQQKTDQAPKLLIKYASESISGIPSRFSGSGSGTDFTLTISSVEAEDAADYYCQQINSWPTTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1009 - protein, 6 aa, Artificial Sequence
  source: /note=“Description of Artificial Sequence: Synthetic peptide”
SDYAWN

SEQ ID NO: 1010 - protein, 16 aa, Artificial Sequence
  source: /note=“Description of Artificial Sequence: Synthetic peptide”
YISYSGSTSYNPSLKS

SEQ ID NO: 1011 - protein, 9 aa, Artificial Sequence
  source: /note=“Description of Artificial Sequence: Synthetic peptide”
FDYAHAMDY

SEQ ID NO: 1012 - protein, 11 aa, Artificial Sequence
  source: /note=“Description of Artificial Sequence: Synthetic peptide”
QASQSIGTSIH

SEQ ID NO: 1013 - protein, 7 aa, Artificial Sequence
  source: /note=“Description of Artificial Sequence: Synthetic peptide”
YASESIS

SEQ ID NO: 1014 - protein, 9 aa, Artificial Sequence
  source: /note=“Description of Artificial Sequence: Synthetic peptide”
QQINSWPTT

SEQ ID NO: 1015-2000 - skipped (986 entries)

SEQ ID NO: 2001 - protein, 137 aa, Artificial Sequence
  source: /note=“Description of Artificial Sequence: Synthetic polypeptide”
MEFGLSWVFLVALLRGVQCQVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSS

SEQ ID NO: 2002 - protein, 126 aa, Artificial Sequence
  source: /note=“Description of Artificial Sequence: Synthetic polypeptide”
MLPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIK